(19) United States
(12) Patent Application Publication
(10) Pub. No.: US 2022/0110919 A1
(43) Pub. Date: Apr. 14, 2022

(54) METHODS OF MODULATING LEUKOCYTES ACTIVATION AND THROMBOCYTE CLEARANCE WITH INHIBITORS OF SPECIFIC NEURAMINIDASE ISOENZYMES

(71) Applicants: The Governors of the University of Alberta, Edmonton (CA); Valorisation HSJ, Limited Partnership, Montreal (CA)

(72) Inventors: Alexey Pchejetski, Mont-Royal (CA); Christopher Cairo, Edmonton (CA); Tianlin Guo, Montreal (CA); Md. Amran Howlader, Edmonton (CA); Ekterina Demina, Montreal (CA)

(21) Appl. No.: 17/309,450

(22) PCT Filed: Nov. 29, 2019
(86) PCT No.: PCT/CA2019/051715

§ 371 Date: May 27, 2021

Related U.S. Application Data
(60) Provisional application No. 62/772,704, filed on Nov. 29, 2018.

(51) Int. Cl. A61K 31/4192; A61K 31/351; A61P 31/04

(52) U.S. Cl. CPC A61K 31/4192; A61P 31/04; A61K 31/351

(57) ABSTRACT

The present invention provides a method of modulating leukocytes activation (e.g., adhesion and/or transmigration and/or cytokine response) and a method of modulating thrombocyte clearance comprising administering to a subject in need thereof a specific inhibitor of neuraminidase 1 (neu1); neuraminidase 3 (neu3) or neuraminidase 4 (neu4); or a bispecific inhibitor of neu1, neu3 or neu4 of formula (I): (I) Also provided are pharmaceutical compositions comprising the inhibitor compound of formula (1), and uses thereof for modulating leukocytes activation or modulating thrombocyte clearance.

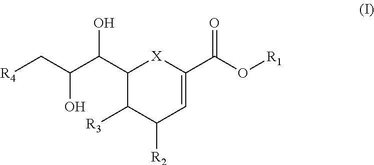

### CROSS REFERENCE TO RELATED APPLICATIONS

This application claims the benefit of priority from U.S. Provisional patent application No. 62/772,704, filed on Nov. 29, 2018, which is incorporated herein by reference.

### FIELD OF THE DISCLOSURE

The present disclosure relates to methods of modulating leukocytes activation or thrombocyte clearance with inhibitors of specific neuraminidase isoenzymes. More specifically, the present disclosure is concerned with the use of specific inhibitors of neu1, neu3 and neu4 for modulating leukocytes activation or thrombocyte clearance.

### REFERENCE TO SEQUENCE LISTING

N.A.

### BACKGROUND OF THE DISCLOSURE

### Infection

The response of white blood cells (leukocytes) to infection is critical to human health. Protecting the body from the constant presence of external and internal (gut) microbes requires active surveillance by white blood cells, or leukocytes, which travel from the blood stream to the periphery. The process of leukocyte transmigration to a site of infection is carefully choreographed by chemoattractants (chemokines), cellular receptors, and leukocytes themselves.

The process of leukocyte rolling, extravasation, and homing to sites of inflammation is critical to cellular immunity, and is known as the leukocyte adhesion cascade (Ley et al., 2007). Along each step of this process, different cell adhesion molecules and their ligands mediate recognition between the leukocyte and endothelial cells. The initial attachment of the leukocyte to the endothelial wall, usually referred to as rolling, is mediated by selectins and their carbohydrate ligands (e.g., sialyl Lewis-X; CD15s) (Varki, 1994). Later steps of the process must arrest the cell (firm adhesion) to allow for transmigration. These later steps of the process are largely mediated by integrin receptors and their ligands. The first of these in the cascade is LFA-1 (the αLβ2 integrin; CD11a, CD18), which binds to ICAM-1 (Inter-cellular adhesion molecule-1; CD54) and conveys an outside-in intracellular signal to the leukocyte (Hogg et al., 2004). These and subsequent integrin-mediated processes, including interactions of the very-late antigens (VLA-4, the α4β1 integrin or VLA-5, the α5β1 integrin), allow cells to migrate to the site of inflammation. (Simmons, 2005; Cox et al., 2010; Hogg et al., 2003).

### Thrombocytopenia

Immune thrombocytopenia purpura (ITP) is an autoimmune condition characterized by a low platelet count in the absence of bone marrow-related abnormalities. It is a frequent cause of thrombocytopenia in children, resulting in significant reduction in quality of life and increased risk of bleeding. ITP is mediated by platelet antibodies that accelerate platelet destruction and inhibit their production. The dominant clinical manifestation is bleeding, which correlates generally with severity of the thrombocytopenia. ITP is a common acquired cause for low platelet counts in childhood affecting between 4-8 per 100,000 children each year with a mean age at presentation of 5.7 yrs (Blanchette, 2010; Yong et al., 2010; Kuhne, 2003). Variability in natural history and response to therapy suggest that ITP comprises heterogeneous disorders arising through diverse mechanisms of the production of platelet autoantibodies. Management of childhood ITP remains controversial. Each patient requires an individualized treatment plan that takes into consideration the platelet count, bleeding symptoms, health-related quality of life, and medication side effects. Traditional first-line agents including corticosteroids, intravenous immunoglobulins (IVIg), and anti-D immunoglobulin (anti-D) are usually effective but may be associated with multiple side effects reviewed in Neunert, 2013. Splenectomy, historically used as second-line therapy for both adults and children with ITP unresponsive to first-line agents, is considered the only “curative” therapy (Neunert, 2013). However, up to 25% of patients may not respond to such therapeutic approaches, and are defined as having refractory ITP (Neunert, 2013). Furthermore, splenectomy leads to a loss of immune protection against encapsulated bacteria and is associated with a lifelong risk of overwhelming sepsis and infection, as well as an increased risk of thromboembolism (Crary et al., 2009).

A host of human diseases involve mis-regulation of the inflammatory response. There is a need for new strategies and mechanisms for anti-adhesive and anti-inflammatory therapeutic.

The present description refers to a number of documents, the content of which is herein incorporated by reference in their entirety.

### SUMMARY OF THE DISCLOSURE

The present disclosure provides a method for modulating leukocytes activation in response to infection and inflammatory signals. It also provides compounds for use in the modulation of inflammatory responses in e.g., leukocytes transmigration and adhesion and cytokine response.

The present disclosure also provides a method for modulating thrombocyte clearance, comprising inhibiting of the expression or activity of neuraminidase 1 (neu1), neuraminidase 3 (neu3) or neuraminidase 4 (neu4) in a subject in need thereof.

More specifically, in accordance with the present disclosure, there are provided the following items:

Item 1. A method of modulating leukocyte activation, comprising specifically inhibiting expression or activity of neuraminidase 1 (neu1), neuraminidase 3 (neu3) or neuraminidase 4 (neu4) in a subject in need thereof.
Item 2. The method of item 1, wherein the inhibiting comprises administering a therapeutically effective amount of a specific or a bispecific neu1, neu3, and/or neu4 inhibitor to the subject.
Item 3. The method of item 1 or 2, wherein the leukocyte activation comprises transmigration of monocytes, neutrophils, macrophages, NK cells or T-cells.
Item 4. The method of item 1 or 2, wherein the inhibiting comprises the administration of a therapeutically effective amount of a specific or a bispecific neu1 and/or neu4 inhibitor
Item 5. The method of item 4, wherein the leukocyte activation comprises leukocyte adhesion, leukocyte transmigration and/or cytokine response.
Item 6. The method of item 5, wherein the cytokine is G-CSF/CSF-3, IL-21, IL-6, IFN-γ and/or RANTES.
Item 7. A method of modulating thrombocyte clearance, comprising specifically inhibiting of the expression or activity of neuraminidase 1 (neu1), neuraminidase 3 (neu3) or neuraminidase 4 (neu4) in a subject in need thereof.
Item 8. The method of item 7, wherein the inhibiting comprises the administration of a therapeutically effective amount of a specific or a bispecific neu1, neu3, and/or neu4 inhibitor.
Item 9. The method of items 1 to 8, wherein the inhibitor is (i) compound 5c;

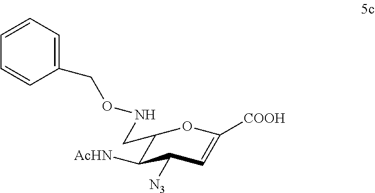

or (ii) a compound of formula I

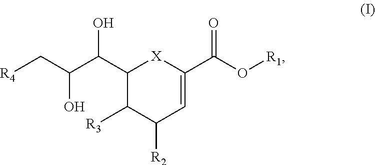

wherein R₁ is H; a C1-C10 alkyl; C1-C10 heteroalkyl; C3-C7 cycloalkyl; C3-C7 heterocycloalkyl; C3-C8 aryl; or C3-C8 heteroaryl; wherein the alkyl, heteroalkyl, cycloalkyl, heterocycloalkyl, aryl and heteroaryl are optionally substituted by at least one substituent, each substituent being independently a C1-C10 alkyl, a C3-C8 cycloalkyl, a C3-C7 aryl, a halogen, an amide or a hydroxyl;
R₂ is H; —OH, —NHC(═NH)NH₂; or azide;
R₃ is —NHC(O)(CH₂)nR₅,
wherein R₅ is H; —OH; C1-C10 alkyl; C1-C10 heteroalkyl; C3-C7 cycloalkyl; C3-C7 heterocycloalkyl; C3-C8 aryl or azide; wherein the alkyl, heteroalkyl, cycloalkyl, heterocycloalkyl, and aryl are optionally substituted by at least one substituent, each substituent being independently a C1-C10 alkyl, a C3-C8 cycloalkyl, a C3-C7 aryl, an halogen, an amide or an hydroxyl; and
n is 0 or 1;
R₄ is H; —OH; —O-alkyl; —C(O)-alkyl-NHC(O)-aryl; —NHC(O)R₆; or

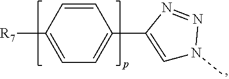

wherein the alkyl and aryl are optionally substituted by at least one substituent, each substituent being independently a C1-C10 alkyl, a C3-C8 cycloalkyl, a C3-C7 aryl, an halogen, an amine, an amide or an hydroxyl,
wherein: R₆ is H, C1-C10 alkyl; or C3-C7 aryl, wherein the C1-C10 alkyl and C3-C7 aryl are optionally substituted by at least one substituent, each substituent being independently a C1-C10 alkyl, a C3-C8 cycloalkyl, a C3-C7 aryl, an halogen, an amide, an amine or an hydroxyl;
R₇ is H; halogen; —O-alkyl; —C(O)OH; amine; acetamide; —C1-C10 alkyl; —O—C3-C7 aryl; or —(CH₂)qNH(CO)aryl, wherein the C1-C10 alkyl and C3-C7 aryl are optionally substituted by at least one substituent, each substituent being independently a C1-C10 alkyl, a C3-C8 cycloalkyl, a C3-C7 aryl, a halogen, an amide, an amine or an hydroxyl, wherein q is 0 or 1; and
p is 0, 1, 2 or 3; and

### X is O, CH₂ or S,

with the proviso that when R₂ and R₄ are OH, R₃ is not —NHC(O)CH₃,
or is an ester, solvate, hydrate or pharmaceutical salt of the compound of formula I.
Item 10. The method of item 9, wherein R₃ is —NHC(O)(CH₂)nR₅ and n is 1.
Item 11. The method of item 10, wherein R₅ is H or azide.
Item 12. The method of any one of items 9 to 11, wherein R₂ is —NHC(═NH)NH₂.
Item 13. The method of any one of items 9 to 12, wherein R₄ is

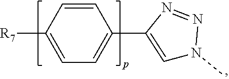

where R₇ and p are as defined in item 9.
Item 14. The method of item 13, wherein p is 2 and R₇ is H.
Item 15. The method of item 10, wherein R₅ is C1-C5 alkyl.
Item 16. The method of any one of items 9, 10, and 15, wherein R₂ is OH.
Item 17. The method of any one of items 9, 10, 15 and 16, wherein R₄ is —OH.
Item 18. The method of any one of items 9 to 11 and 16, wherein R₄ is —NHC(O)R₆.
Item 19. The method of item 18, wherein R₆ is C3-C6 alkyl.
Item 20. The method of item 13, wherein p is 0.
Item 21. The method of item 15, wherein R₇ is -hydroxy C1-C10 alkyl.
Item 22. The method of any one of items 9, 10 or 11, wherein R₄ is

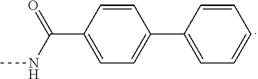

Item 23. The method of any one of items 9 to 22, wherein X is O.
Item 24. The method of any one of items 1 to 6, wherein the inhibiting comprises the administration of a specific or bispecific neu4 inhibitor.
Item 25. The method of item 24, wherein the specific or bispecific inhibitor is compound 28.
Item 26. The method of item 22 or 23, wherein the inhibiting increases monocytes and neutrophils transmigration and decreases T cell transmigration in response to bacterial infection.
Item 27. The method of any one of items 1 to 6, wherein the inhibiting comprises the administration of a specific or bispecific neu3 inhibitor.
Item 28. The method of item 27, wherein the specific or bispecific neu3 inhibitor is compound 8b or 5c.
Item 29. The method of item 27 or 28, wherein the inhibiting increases monocytes and neutrophils transmigration and decreases T cell transmigration in response to bacterial infection.
Item 30. The method of any one of items 1 to 6, wherein the inhibiting comprises the administration of a specific or bispecific neu1 inhibitor.
Item 31. The method of item 30, wherein the specific or bispecific neu1 inhibitor is compound 32 or 50.
Item 32. The method of item 30 or 31, wherein the inhibiting decreases monocytes transmigration and increases neutrophils transmigration in response to bacterial infection.
Item 33. The method of any one of items 1 to 6, wherein the inhibiting comprises the administration of a specific or bispecific neu1 or neu4 inhibitor.
Item 34. The method of item 33, wherein the inhibiting modulates cytokine response.
Item 35. The method of any one of items 1 to 6 and 9 to 34, wherein the inhibiting modulates an inflammatory response to bacterial infection in a subject in need thereof.
Item 36. The method of item 7 or 8, wherein the inhibiting comprises the administration of a therapeutically effective amount of a specific or a bispecific neu1 inhibitor.
Item 37. The method of item 36, wherein the inhibitor is compound 50.

More specifically, in accordance with the present disclosure, there are also provided the following items:

Item 1. A method of preventing or treating an inflammatory response to a bacterial infection, comprising specifically inhibiting the expression or activity of neuraminidase 1 (neu1), neuraminidase 3 (neu3) or neuraminidase 4 (neu4) in a subject in need thereof.
Item 2. The method of item 1, wherein the inhibitor is a compound of formula I

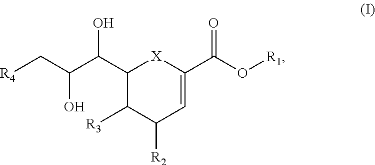

wherein R₁ is H; a C1-C10 alkyl; C1-C10 heteroalkyl; C3-C7 cycloalkyl; C3-C7 heterocycloalkyl; C3-C8 aryl; or C3-C8 heteroaryl; wherein the alkyl, heteroalkyl, cycloalkyl, heterocycloalkyl, aryl and heteroaryl are optionally substituted by at least one substituent, each substituent being independently a C1-C10 alkyl, a C3-C8 cycloalkyl, a C3-C7 aryl, a halogen, an amide or a hydroxyl;
R₂ is H; —OH, —NHC(═NH)NH₂; or azide;
R₃ is —NHC(O)(CH₂)nR₅,
wherein R₅ is H; —OH; C1-C10 alkyl; C1-C10 heteroalkyl; C3-C7 cycloalkyl; C3-C7 heterocycloalkyl; C3-C8 aryl; or azide; wherein the alkyl, heteroalkyl, cycloalkyl, heterocycloalkyl, and aryl are optionally substituted by at least one substituent, each substituent being independently a C1-C10 alkyl, a C3-C8 cycloalkyl, a C3-C7 aryl, an halogen, an amide or an hydroxyl; and
n is 0 or 1;
R₄ is H; —OH; —O-alkyl; —C(O)-alkyl-NHC(O)-aryl; —NHC(O)R₆; or

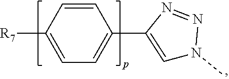

wherein the alkyl and aryl are optionally substituted by at least one substituent, each substituent being independently a C1-C10 alkyl, a C3-C8 cycloalkyl, a C3-C7 aryl, an halogen, an amine, an amide or an hydroxyl,
wherein: R₆ is H, C1-C10 alkyl; or C3-C7 aryl, wherein the C1-C10 alkyl and C3-C7 aryl are optionally substituted by at least one substituent, each substituent being independently a C1-C10 alkyl, a C3-C8 cycloalkyl, a C3-C7 aryl, an halogen, an amide, an amine or an hydroxyl;
R₇ is H; halogen; —O-alkyl; —C(O)OH; amine; acetamide; —C1-C10 alkyl; —O—C3-C7 aryl; or —(CH₂)qNH(CO)aryl, wherein the C1-C10 alkyl and C3-C7 aryl are optionally substituted by at least one substituent, each substituent being independently a C1-C10 alkyl, a C3-C8 cycloalkyl, a C3-C7 aryl, a halogen, an amide, an amine or a hydroxyl, and wherein q is 0 or 1; and
p is 0, 1, 2 or 3; and

### X is O, CH₂ or S,

with the proviso that when R₂ and R₄ are OH, R₃ is not —NHC(O)CH₃,
or is an ester, solvate, hydrate or pharmaceutical salt of the compound of formula I.
Item 3. The method of item 2, wherein R₃ is —NHC(O)(CH₂)nR₅.
Item 4. The method of item 2, wherein n is 0.
Item 5. The method of item 4, wherein R₅ is cycloalkyl.
Item 6. The method of item 4, wherein R₅ is aryl.
Item 7. The method of item 4, wherein R₅ is C1-C10 alkyl.
Item 8. The method of item 4, wherein R₅ is C1-C10 alkyl substituted with a C1-C10 alkyl.
Item 9. The method of item 2, wherein n is 1.
Item 10. The method of item 9, wherein R₅ is H or azide.
Item 11. The method of item 9, wherein R₅ is C1-C5 alkyl.
Item 12. The method of item 11, wherein the C1-C5 alkyl is branched.
Item 13. The method of any one of items 2-12, wherein R₂ is OH.
Item 14. The method of any one of items 2-11, wherein R₂ is —NHC(═NH)NH₂.
Item 15. The method of any one of items 2-11, wherein R₂ is azido.
Item 16. The method of any one of items 2-15, wherein R₄ is —OH.
Item 17. The method of any one of items 2-15, wherein R₄ is —NHC(O)R₆.
Item 18. The method of item 17, wherein R₆ is C1-C10 alkyl.
Item 19. The method of item 18, wherein the C1-C10 alkyl is branched.
Item 20. The method of item 17, wherein R₆ is C3-C7 aryl.
Item 21. The method of item 20, wherein the C3-C7 aryl is substituted with an amine or an amide.
Item 22. The method of any one of items 2-15, wherein R₄ is

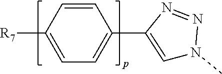

wherein R₇ and p are as defined in item 2.
Item 23. The method of item 22, wherein p is 0.
Item 24. The method of item 23, wherein R₇ is —(CH₂)qNH(CO)aryl.
Item 25. The method of item 23, wherein R₇ is -hydroxy C1-C10 alkyl.
Item 26. The method of item 20, wherein R₇ is C1-C10 alkyl.
Item 27. The method of item 22, wherein p is 1.
Item 28. The method of item 27, wherein R₇ is halogen.
Item 29. The method of item 27, wherein R₇ is O-alkyl.
Item 30. The method of item 27, wherein R₇ is —C(O)OH.
Item 31. The method of item 27, wherein R₇ is amine.
Item 32. The method of item 27, wherein R₇ is acetamide.
Item 33. The method of item 27, wherein R₇ is —C1-C10 alkyl.
Item 34. The method of item 27, wherein R₇ is —CH₂NH(CO)aryl.
Item 35. The method of item 27, wherein R₇ is —O—C3-C7 aryl.
Item 36. The method of item 22, wherein p is 2.
Item 37. The method of item 36, wherein R₇ is H.
Item 38. The method of any one of items 2-15, wherein R₄ is —C(O)-alkyl-NHC(O)-aryl.
Item 39. The method of item 38, wherein the alkyl is C1-C10 alkyl.
Item 40. The method of item 38 or 39, wherein the aryl is C3-C7 aryl.
Item 41. The method of item 38, wherein the C3-C7 aryl is substituted with an amide.
Item 42. The method of item 2, wherein:
(i) R₃ is —NHC(O)(CH₂)nR₅, wherein n is 0 to 7 and wherein R₅ is C1-C10 alkyl, C3-C7 cycloalkyl, or C3-C8 aryl, wherein the alkyl, cycloalkyl, and aryl are optionally substituted by at least one substituent, each substituent being independently a C1-C10 alkyl, a C3-C8 cycloalkyl, a C3-C7 aryl, an halogen, an amide or an hydroxyl;
(ii) R₂ is —OH, —NHC(═NH)NH₂ or azide; and
(iii) R₄ is —OH; —NHC(O)R₆, wherein R₆ is C1-C10 alkyl or C1-C5 aryl; —(CH₂)qNH(CO)aryl, wherein q is 0 or 1; or

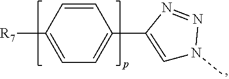

wherein p is 0, 1, 2 or 3, and R₇ is H, —C(═O)OH, phenyl, or phenyloxy,
with the proviso that when R₂ and R₄ are OH, R₃ is not —NHC(O)CH₃.
   Item 43. The method of item 2, wherein:
(i) R₃ is —NHC(O)(CH₂)nCH₃, wherein n is 0 to 7;
(ii) R₂ is —OH or —NHC(═NH)NH₂; and
(iii) R₄ is —OH; —NHC(O)R₆, wherein R₆ is C3-C7 aryl or C1-C10 alkyl; or

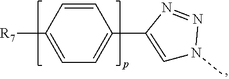

wherein p is 1, 2 or 3, and R₇ is H, —C(═O)OH, phenyl, or phenyloxy,
with the proviso that when R₂ and R₄ are OH, R₃ is not —NHC(O)CH₃.
   Item 44. The method of any one of items 2 to 43, wherein X is O.
   Item 45. The method of any one of items 2 to 44, wherein R₁ is H or alkyl.
   Item 46. The method of item 2, wherein the compound is of formula I, wherein X is O, R₁ is H, and R₃, R₂ and R₄ are as set forth below:

R₃ (at position C5) is CH₃C(O)NH— —

| | R₂ (at position | |
|---|---|---|
| compound | C4) | R₄ (at position C9) |
| 6 | 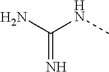 | 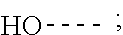 |
| 7a | 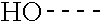 | 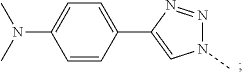 |
| 7b | 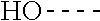 | 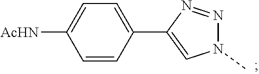 |
| 7c | 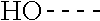 | 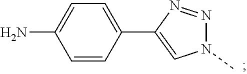 |
| 7d | 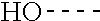 | 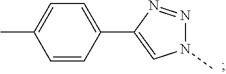 |
| 7e | 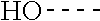 | 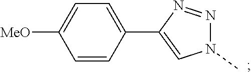 |
| 7f | 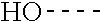 | 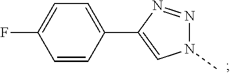 |
| 7g | 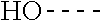 | 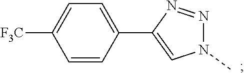 |
| 7h | 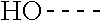 | 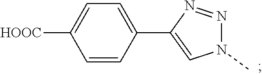 |
| 7i | 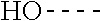 | 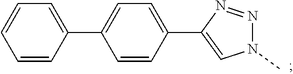 |
| 7j | 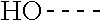 | 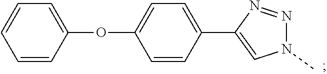 |
| 8a | 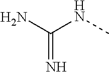 | 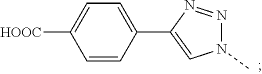 |
| 8b | 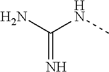 | 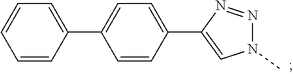 |
| 13 | 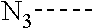 | 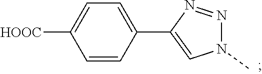 |
| 26 | 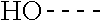 | 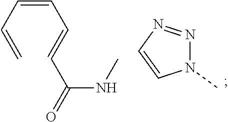 |
| 27 | 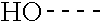 | 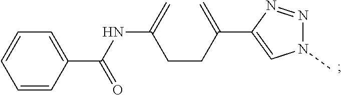 |
| C9-4HMT- DANA (28) | 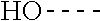 | 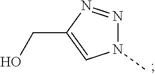 |
| 58 | 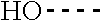 | 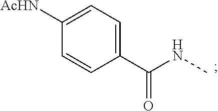 |
| 59 | 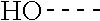 | 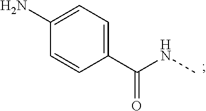 |
| 60 | 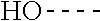 | 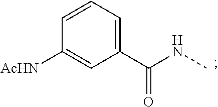 |
| 61 | 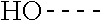 | 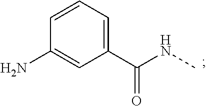 |
| 62 | 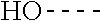 | 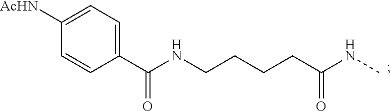 |
| 63 | 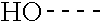 | 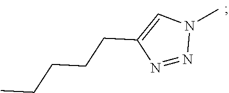 |

R₂ (at position C₄) is

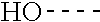

| | R₃ (at position | |
|---|---|---|
| | C5) | R₄ (at position C9) |
| 29 | 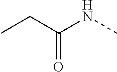 | |
| 30 | 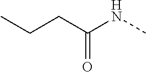 | 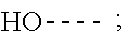 |
| 31 | 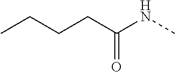 | 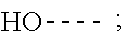 |
| 32 | 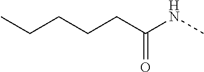 | 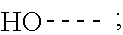 |
| 33 | 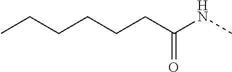 | 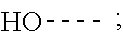 |
| 34 | 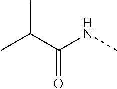 | 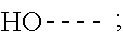 |
| 35 | 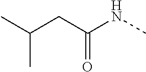 | 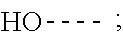 |
| 36 | 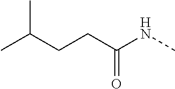 | 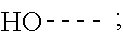 |
| 37 | 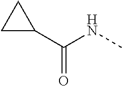 | 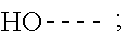 |
| 38 | 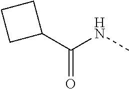 | 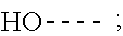 |
| 39 | 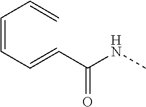 | 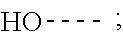 |
| 40 | 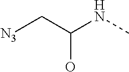 | 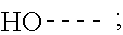 |
| 49 | 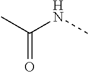 | 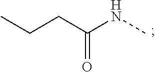 |
| 50 | 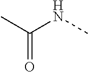 | 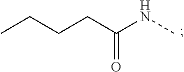 |
| 51 | 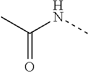 | 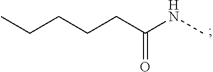 |
| 52 | 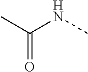 | 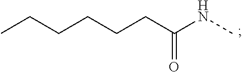 |
| 53 | 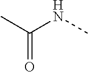 | 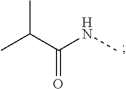 |
| 54 | 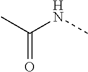 | 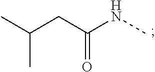 |
| 55 | 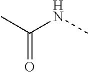 | 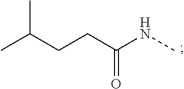 |
| 56 | 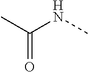 | 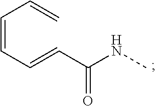 |
| 57 | 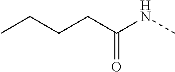 | 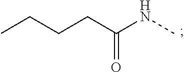 |
| 64 | 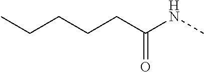 | 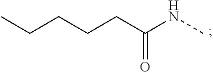 |
| 65 | 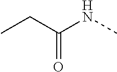 | 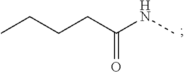 |
| 66 | 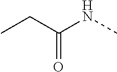 | 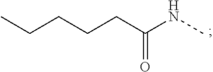 |
| 67 | 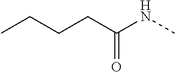 | 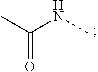 |
| 68 | 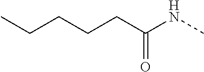 | 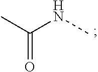 |
| 69 | 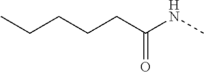 | 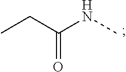 |
| 70 | 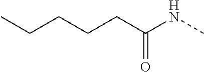 | 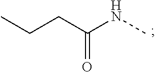 |
| 72 | 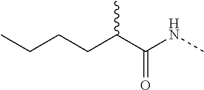 | 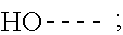 |
| 73 | 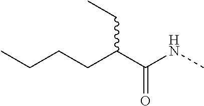 | 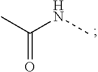 |
| 74 | 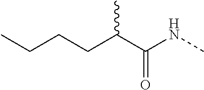 | 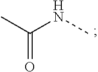 |
| 75 | 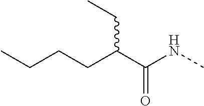 | 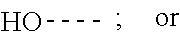 |
| 79 | 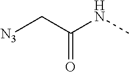 | 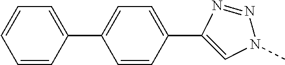 |
| 80 | 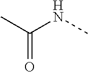 | 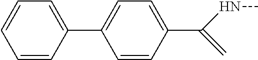 |

R₂ (at position C4) is NH₂C(═NH)NH— —

| | R₃ (at position C5) | R₄ (at position C9) |
|---|---|---|
| 71 | 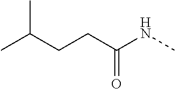 | 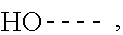 |

or an ester, solvate, hydrate or pharmaceutical salt of the compound of formula I.
Item 47. The method of item 2, wherein the compound is of formula I, wherein X is O, R₁ is H, and R₃, R₂ and R₄ are set forth below:

R₃ (at position C5) is CH₃C(O)NH— —

| | R₂ (at position | |
|---|---|---|
| compound | C4) | R₄ (at position C9) |
| 7h | 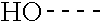 | 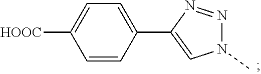 |
| 7i | 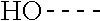 | 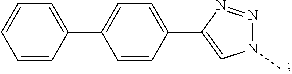 |
| 7j | 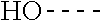 | 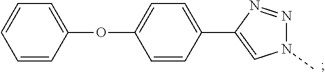 |
| 8a | 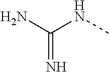 | 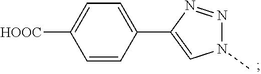 |
| 8b | 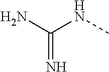 | 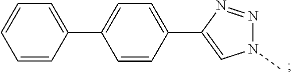 |

R₂ (at position C4) is HO— —

| | R₃ (at position | |
|---|---|---|
| | C5) | R₄ (at position C9) |
| 31 | 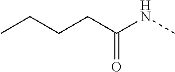 | 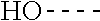 |
| 32 | 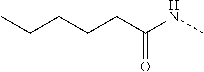 | 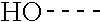 |
| 33 | 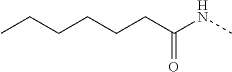 | 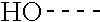 |
| 36 | 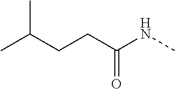 | 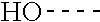 |
| 51 | 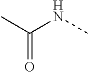 | 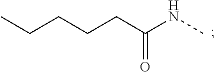 |
| 54 | 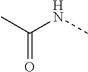 | 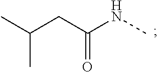 |
| 56 | 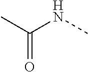 | 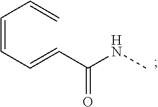 |
| 57 | 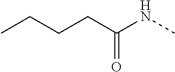 | 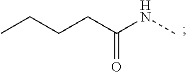 |
| 58 | 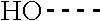 | 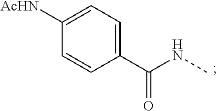 |
| 65 | 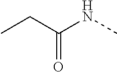 | 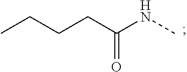 |
| 66 | 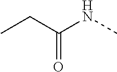 | 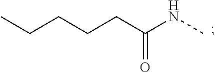 |
| 67 | 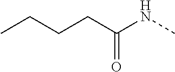 | 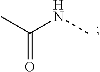 |
| 68 | 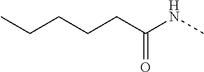 | 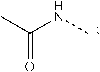 |
| 69 | 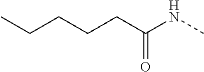 | 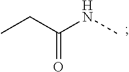 |
| 70 | 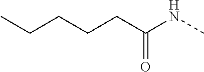 | 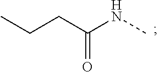 |
| 72 | 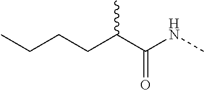 | 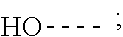 |
| 73 | 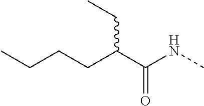 | 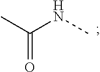 |
| 74 | 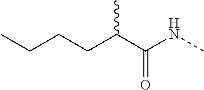 | 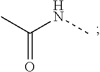 |
| 75 | 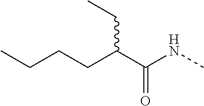 | 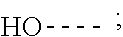 |
| 79 | 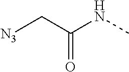 | 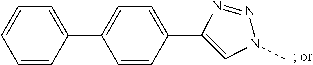 |
| 80 | 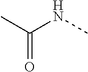 | 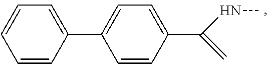 |

or an ester, solvate, hydrate or pharmaceutical salt of the compound of formula I.
Item 48. The method of any one of items 2 to 47, wherein the compound is of formula I is of formula Ia:

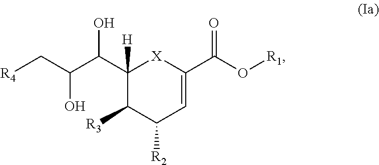

wherein R₁, R₂, R₃, R₄ and X are as defined in any one of items 2 to 47.
Item 49. The method of any one of items 2 to 47, wherein the compound of formula I is of formula Ib:

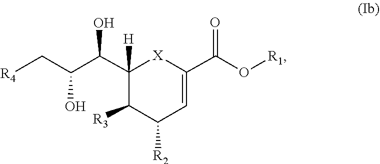

wherein R₁, R₂, R₃, R₄ and X are as defined in any one of items 2 to 47.
Item 50. The method of any one of items 1 to 49, wherein the inhibitor is a specific or bispecific inhibitor of neu1.
Item 51. The method of any one of items 1 to 49, wherein the inhibitor is a specific or bispecific inhibitor of neu3.
Item 52. The method of any one of items 1 to 49, wherein the inhibitor is a specific or bispecific inhibitor of neu4.

In another specific embodiment, the specific inhibitor is a compound of formula III

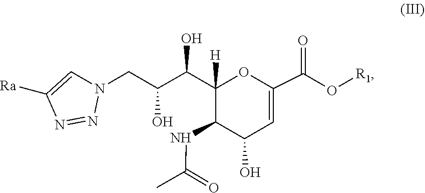

wherein R1 is as defined above or H, a linear alkyl group C1-C12 (i.e. Me, Et, Pr, But, Pent, Hex, etc.), a branched alkyl group C1-C12, or an aryl group; and Rₐ is the group shown at that position in any one of 7a-7j, and 26 to 28,
or an ester, solvate, hydrate or pharmaceutical salt thereof.

In another specific embodiment, the specific inhibitor is a compound of formula IV

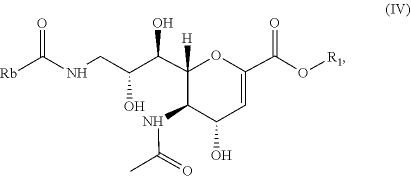

where R1 is as defined above or H, a linear alkyl group C1-C12 (i.e. Me, Et, Pr, But, Pent, Hex, etc.), a branched alkyl group C1-C12, or an aryl group; and R_{b} is the group shown at that position in any one of compounds 49 to 56,
or an ester, solvate, hydrate or pharmaceutical salt thereof.

In another specific embodiment, the specific inhibitor is a compound of formula V

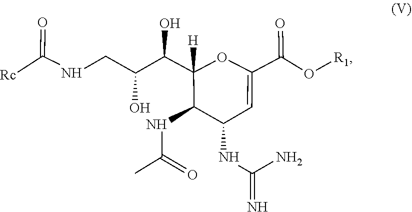

where R1 is as defined above or H, a linear alkyl group C1-C12 (i.e. Me, Et, Pr, But, Pent, Hex, etc.), a branched alkyl group C1-C12, or an aryl group; and R_{c} is the group shown at that position in any one of compounds 58-61,
or an ester, solvate, hydrate or pharmaceutical salt thereof.

In another specific embodiment, the specific inhibitor is a compound of formula VI

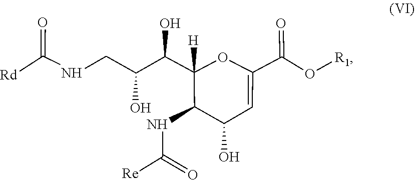

R1 where R1 is as defined above or H, a linear alkyl group C1-C12 (i.e. Me, Et, Pr, But, Pent, Hex, etc.), a branched alkyl group C1-C12, or an aryl group; and R_{d} and Rₑ are the groups shown at these positions in any one of compounds 49-52, 54-57, 64-70, 74-74,
or an ester, solvate, hydrate or pharmaceutical salt thereof.

In another specific embodiment, the specific inhibitor is a compound of formula VII

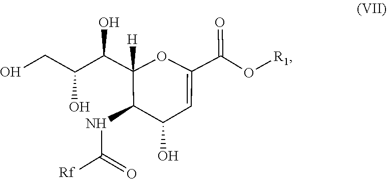

where R1 is as defined above or H, a linear alkyl group C1-C12 (i.e. Me, Et, Pr, But, Pent, Hex, etc.), a branched alkyl group C1-C12, or an aryl group; and R_{f} is the group shown at that position in any one of compounds 29 to 48,
or an ester, solvate, hydrate or pharmaceutical salt thereof.

In another specific embodiment, the specific inhibitor is a compound of formula VIII

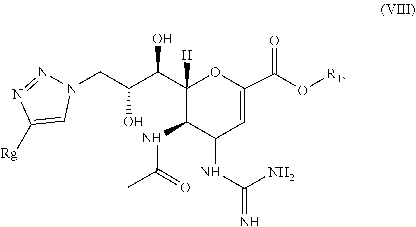

where R1 is as defined above or H, a linear alkyl group C1-C12 (i.e. Me, Et, Pr, But, Pent, Hex, etc.), a branched alkyl group C1-C12, or an aryl group; and R_{g} is an C3-C7 aryl group substituted or not with a C3-C10 aryl group (substituted or not with an halogen, an amine, a C1-C10 alkyl, a C1-C10 alkyloxy, a trifluoromethyl, a —COOH, a C3-C7 aryl); a C1-C10 alkyl group; or a —COOH group. In a more specific embodiment, it is a group as shown at that position in any one of compounds 8a and 8b,
or an ester, solvate, hydrate or pharmaceutical salt thereof.

In another specific embodiment, there is provided a pharmaceutical composition comprising a neu1/neu3 specific inhibitor that is a compound of any one of formulas I, Ia, Ib, and II-VIII, or any specific inhibitor disclosed in Table I, or an ester, solvate, hydrate or pharmaceutical salt thereof, and a pharmaceutically acceptable carrier. In a specific embodiment, the specific inhibitor has an IC50 against a neu1/neu3 that is lower than 1 μM (e.g., 5c, 7i, 7i, 7j, 8a, 8b, 28 31-32, 50, 67-69, 72, 74 and 75, preferably compound 5c, 8b, 28, 32 or 50).

In another specific embodiment, there is provided a pharmaceutical composition comprising a neu1 specific inhibitor that is a compound of any one of formulas I, Ia, Ib, and II-VIII, or any specific inhibitor disclosed in Table I, or an ester, solvate, hydrate or pharmaceutical salt thereof, and a pharmaceutically acceptable carrier. In a specific embodiment, the specific inhibitor has an IC50 against a neu1 that is lower than 1 μM (e.g., compounds 31-32, 50, 67-69, 72, 74 and 75, preferably compound 32 or 50).

In another specific embodiment, there is provided a pharmaceutical composition comprising a neu3 specific inhibitor that is a compound of any one of formulas I, Ia, Ib, and II-VIII, or any specific inhibitor disclosed in Table I, or an ester, solvate, hydrate or pharmaceutical salt thereof, and a pharmaceutically acceptable carrier. In a specific embodiment, the specific inhibitor has an IC50 against a neu3 that is lower than 1 μM (e.g., compounds 5c, 7i, 8a, 8b, preferably compound 5c or 8b).

In another specific embodiment, there is provided a pharmaceutical composition comprising a neu4 specific inhibitor that is a compound of any one of formulas I, Ia, Ib, and II-VIII, or any specific inhibitor disclosed in Table I, or an ester, solvate, hydrate or pharmaceutical salt thereof, and a pharmaceutically acceptable carrier. In a specific embodiment, the specific inhibitor has an IC50 against a neu4 that is lower than 1 μM (e.g., compounds 7i, 7j, 28, preferably compound 28).

In another specific embodiment, there is provided a method of modulating leukocytes adhesion and/or transmigration (e.g., in response to bacterial infection) comprising administering to a subject in need thereof a therapeutically effective amount of (i) a specific neu1/neu3/neu4 inhibitor of the present disclosure that is a compound of any one of formulas I, Ia, Ib, and II-VIII, or any specific neu1/neu3 inhibitor disclosed in Table I, or an ester, solvate, hydrate or pharmaceutical salt thereof; or (ii) a pharmaceutical composition comprising (i) and a pharmaceutically acceptable carrier. (e.g., 5c, 7i, 7i, 7j, 8a, 8b, 28 31-32, 50, 67-69, 72, 74 and 75, preferably compound 5c, 8b, 28, 32 or 50).

In another specific embodiment, there is provided a method of modulating leukocytes adhesion and/or transmigration (e.g., in response to bacterial infection) comprising administering to a subject in need thereof a therapeutically effective amount of (i) a specific neu1 inhibitor of the present disclosure that is a compound of any one of formulas I, Ia, Ib, and II-VIII, or any specific neu1 inhibitor disclosed in Table I, or an ester, solvate, hydrate or pharmaceutical salt thereof; or (ii) a pharmaceutical composition comprising (i) and a pharmaceutically acceptable carrier. (e.g., compounds 31-32, 50, 67-69, 72, 74 and 75, preferably compound 32 or 50).

In another specific embodiment, there is provided a method of modulating leukocytes adhesion and/or transmigration (e.g., in response to bacterial infection) comprising administering to a subject in need thereof a therapeutically effective amount of (i) a specific neu3 inhibitor of the present disclosure that is a compound of any one of formulas I, Ia, Ib, and II-VIII, or any specific neu3 inhibitor disclosed in Table I, or an ester, solvate, hydrate or pharmaceutical salt thereof; or (ii) a pharmaceutical composition comprising (i) and a pharmaceutically acceptable carrier. (e.g., compounds 5c, 7i, 8a, 8b, preferably compound 5c or 8b).

In another specific embodiment, there is provided a method of modulating leukocytes adhesion and/or transmigration (e.g., in response to bacterial infection) comprising administering to a subject in need thereof a therapeutically effective amount of (i) a specific neu3 inhibitor of the present disclosure that is a compound of any one of formulas I, Ia, Ib, and II-VIII, or any specific neu4 inhibitor disclosed in Table I, or an ester, solvate, hydrate or pharmaceutical salt thereof; or (ii) a pharmaceutical composition comprising (i) and a pharmaceutically acceptable carrier. (e.g., compounds 7i, 7j, 28, preferably compound 28).

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1A: Neuraminidase inhibitors and their synthetic routes. FIG. 1A presents the general form of compounds 7a-j, 8a and 8b. FIG. 1B presents the general synthetic route for generation of compounds 7a-j. FIG. 1C: presents the synthetic route for compounds 8a, 13, 15, 18. FIG. 1D: Presents the synthetic route for compound 8b. FIG. 1E: Presents the synthetic routes for compounds 25a-d. FIG. 1F presents the synthetic routes for compounds 49, 50, 51, 52, 53, 54, 55 and 56. FIG. 1G presents the synthetic routes for compounds 29, 30, 31, 32, 33, 34, 35, 36, 37, 38, 39, 40. FIG. 1H presents the synthetic routes for compounds 41, 42, 43, 44, 45, 46, 47, 48; and FIG. 1I presents the synthetic routes for compound 57.

FIGS. 2A-E. Static adhesion assays show T cells response to Neu treatment. (FIG. 2A) Adhesion of Jurkat cells to ICAM-1 was determined using flow cytometry and fluorescent beads (1 μm) under the indicated conditions. Control samples were treated with DMSO (0.05%), PMA, or DANA for 30 min. (FIG. 2B) Adhesion of Jurkat cells to ICAM-1 was determined using flow cytometry and fluorescent beads; samples were treated with buffer or enzyme for 3 hrs, followed by incubation with PMA or DANA (100 μM) for 30 min. All cytometry data were normalized to the appropriate control after background subtraction (BSA coated beads.) Values shown in FIGS. 2A-B are the average of N=6-12 replicates, error is the standard error of the mean. (FIG. 2C) Homotypic aggregation of Jurkat cells was determined using microscopy. Cells were incubated under the indicated conditions for 3 hrs. Aggregation was determined by imaging and analysis with CellProfiler™ (version 2.1.1) to determine the total number of cells and the number of cells found within aggregates. Aggregation is expressed as the percentage of cells in all samples found within an aggregate (N=24, from two separate experiments), and error is shown as the standard error of the mean. (FIG. 2D) Changes in the endocytosis of β2-integrin (N=4) and (FIG. 2E) β1-integrin (N=2) in Jurkat cells after treatment with NanI or NEU3 (30 min at 37° C.). Error bars for aggregation data is shown as standard error of the mean, all data were compared to the indicated control using a t-test; *, p≤0.05; **, p≤0.01; ***, p≤0.005; ****, p≤0.0001.

FIG. 3: Static adhesion assays of PBMC confirm involvement of NEU3 in leukocyte adhesion. Data shown are from at least three healthy donors for each condition. Values shown are the average of N=6 replicates.

FIGS. 4A-D. Alteration of LFA-1 epitopes by neuraminidase treatment. Jurkat T cells (FIGS. 4A and C) or PBMCs (FIGS. 4B and D) were treated with the indicated conditions. Treated cells were labelled with primary antibodies (TS1/22 or MEM148), followed by an AF647-conjugated secondary antibody. Cells were fixed with 1% PFA, analyzed by flow cytometry, and normalized to control (buffer) treatment. Data shown are the mean of three replicates for each sample and error is shown as the standard error of mean. Data were compared to the appropriate control using a t-test to determine p values; *, p≤0.05; **, p≤0.01; ***, p≤0.005; ****, p≤0.0001.

FIGS. 5A-C: Inhibitors of NEU3 block T cell (jurkat) transmigration in vitro. (FIG. 5A) Treatment with two NEU3 inhibitors shows inhibition of transmigration at higher concentrations. (FIG. 5B) Treatment with two NEU1 inhibitors shows inhibition of transmigration at higher concentrations. (FIG. 5C) Treatment with a NEU4 inhibitor shows inhibition of transmigration at higher concentrations. Error bars for aggregation data is shown as standard error of the mean, all data were compared to the indicated control using a t-test; *, p≤0.01; ***, p≤0.005; ****, p≤0.0001.

FIGS. 6A-D: In vivo leukocyte recruitment is affected by NEU enzymes. FIGS. 6A-B: using an air pouch model (Sin et al., 1986), murine C57BL/6 wt, and NEU KO or DKO (NEU1, NEU3, NEU4, NEU3/NEU4) mice, were analyzed for changes in leukocyte transmigration. The air pouch was loaded with saline or LPS to induce leukocyte transmigration. Cells were washed out of the pouch and counted by FACs or hemocytometer. FACs (FIG. 6A) or hematocytometer (FIG. 6B) counts of leukocytes from saline- or LPS-treated-mice are shown for at least three replicate animals. Significance was calculated using one-way Anova with Dunnett's multiple comparison test: Neu4 KO mice have higher counts and Neu1 KO lower counts of immune cells in the poach in response to LPS. FIGS. 6C-D: using an air pouch model (Sin et al., 1986), murine C57BL/6 wt were analyzed for changes in leukocyte transmigration. The air pouch was loaded with saline or LPS to induce leukocyte transmigration. Cells were washed out of the pouch and counted by hemocytometer. The mice were pre-treated with inhibitors of human neuraminidase (i.e. compounds 32, 8b and 28) 48 h, 24 h prior to and in conjunction to injection of LPS. (FIG. 6C) Counts of cells from saline-treated mice and control are shown for at least three replicate animals and in neu1 and neu3 inhibitors. (FIG. 6D) Counts of cells from LPS-treated mice and control are shown for at least three replicate animals and in neu1 and neu3 inhibitors. Error bars for aggregation data is shown as standard error of the mean, all data were compared to the indicated control using a t-test; *, p≤0.01; ***, p≤0.005.

FIGS. 7A-M: In vivo leukocyte recruitment in NEU KO animals has differential effects by cell type measured by FACs. FIGS. 7A-H: Using an air pouch model (Sin et al., 1986), murine C57BL/6 wt, and NEU KO or DKO (NEU3/4, NEU4, NEU3, NEU1) animals were analyzed for changes in leukocyte transmigration. The air pouch was loaded with saline or LPS to induce leukocyte transmigration. Cells were washed out of the pouch and counted by hemocytometer. Comparisons of cell populations of saline (FIGS. 7A, 7C, 7E and 7G,) vs. LPS-treated (FIGS. 7B, 7D, 7F and 7H) animals are shown for monocytes (FIGS. 7A-B), neutrophils (FIGS. 7C-D), macrophages (FIGS. 7E-F), and NK cells (FIGS. 7G-H). FIGS. 7I-M: Using an air pouch model (Sin et al., 1986), murine C57BL/6 wt, were analyzed for changes in leukocyte transmigration. The air pouch was loaded with saline or LPS to induce leukocyte transmigration. Cells were washed out of the pouch and counted by hemocytometer. The mice were pre-treated with inhibitors of human neuraminidase (i.e. compounds 32, 8b and 28) 48 h, 24 h prior to and in conjunction to injection of saline or LPS. Cell populations of saline or LPS-treated animals are shown for monocytes (FIG. 7I, saline-treated), neutrophils (FIG. 7J, saline-treated), macrophages (FIG. 7K, LPS-treated), NK cells (FIG. 7L, LPS-treated) and T cells (FIG. 7M, LPS-treated).

FIGS. 8A-I: Presents cytokine plasma level (pg/ml) of wt C57BL/6 mice and neu1 and neu4 knock-out (KO) mice treated with LPS (mimicking bacterial infection) or saline (negative control) in the air pouch model. FIG. 8A: G-CSF/CSF-3 level in wt and neu1 KO and neu4 KO when treated with saline (negative control) or LPS; FIG. 8B: IL-1 alpha level in wt and neu1 KO and neu4 KO when treated with saline (negative control) or LPS; FIG. 8C: IL-1 beta level in wt and neu1 KO and neu4 KO when treated with saline (negative control) or LPS; FIG. 8D: IL-15/IL-15R level in wt and neu1 KO and neu4 KO when treated with saline (negative control) or LPS; FIG. 8E: IL-21 level in wt and neu1 KO and neu4 KO when treated with saline (negative control) or LPS; FIG. 8F: IL-6 level in wt and neu1 KO and neu4 KO when treated with saline (negative control) or LPS; FIG. 8G: IFN-γ alpha level in wt and neu1 KO and neu4 KO when treated with saline (negative control) or LPS; FIG. 8H: IL-10 level in wt and neu1 KO and neu4 KO when treated with saline (negative control) or LPS; and FIG. 8I: RANTS (or Chemokine (C-C motif) ligand 5) level in wt and neu1 KO and neu4 KO when treated with saline (negative control) or LPS. Error bars for aggregation data is shown as standard error of the mean, all data were compared to the indicated control(s) (wt saline, wt LPS or corresponding neu KO saline bars) using a t-test; *, p≤0.01; ***, p≤0.005; ****, p≤0.0001.

FIGS. 9A-D: Presents impact of neu1 deficiency on platelet depletion rate in the passive mouse model of immunothrombocytopenia. FIGS. 9A-B present the linear regression analysis of platelet counts (FIG. 9A) and platelet decrease rate (FIG. 9B) in wt mice (C57B16) and neu1 KO mice (i.e. CathA^{s190Neo} and neu1^{-/-} MΘ); and FIGS. 9C-D present the linear regression analysis of platelet counts (FIG. 9C) and platelet decrease rate (FIG. 9D) in wt mice (C57B16) and neu1 KO mice (i.e. CathA^{s190Neo} and neu1^{ΔE3Geo}). In FIGS. 9B and D, error bars for aggregation data is shown as standard error of the mean, all data were compared to the indicated control using a t-test; ***, p≤0.005.

FIGS. 10A-B: Presents impact of neu1 inhibitor on platelet (PLT) depletion rate in the passive mouse model of immunothrombocytopenia. FIG. 10A presents the linear regression analysis of platelet counts in neu1 inhibitor (compound 50)-treated wt mice and in saline-treated wt mice (C57B16, control); and FIG. 10B presents the platelet decrease rate in neu1 inhibitor (compound 50)-Treated wt mice and in saline-treated wt mice (C57B16, control). In FIG. 10B, error bars for aggregation data is shown as standard error of the mean, all data were compared to the indicated control using a t-test; **, p≤0.01.

FIG. 11: Amino acid of human neuraminidase 1 (SEQ ID NO: *); human neuraminidase 2 (SEQ ID NO: *); human neuraminidase 3, isoform 1 (SEQ ID NO: *); human neuraminidase 3, isoform 2 (SEQ ID NO: *); human neuraminidase 4, isoform 1 (SEQ ID NO: *); human neuraminidase 4, isoform 2 (SEQ ID NO: *); and human neuraminidase 4, isoform 3 (SEQ ID NO: *).

FIGS. 12A-B: Alignment of neuraminidase proteins of FIG. 11, and consensus sequence derived therefrom (SEQ ID NO: *). In this alignment, “*” denotes that the residues in that column are identical in all sequences of the alignment, “:” denotes that conserved substitutions have been observed, and “.” denotes that semi-conserved substitutions have been observed. Consensus sequences derived from these alignments are also presented wherein X is any amino acid.

### DETAILED DESCRIPTION OF THE DISCLOSURE

The present disclosure relates to the use of specific inhibitors of neuraminidase enzymes for modulating leukocyte activation (e.g., transmigration, adhesion or cytokine response), and in turn, inflammation (e.g., inflammation response to bacterial infection). More specifically, it relates to the use of a therapeutically effective amount of a specific neuraminidase 1 (neu1) inhibitor, a specific neuraminidase 3 (neu3) inhibitor, or a specific neuraminidase 4 (neu4) inhibitor, or a bispecific neu1 inhibitor (e.g., neu1/neu2 or neu1/neu4) or a bispecific neu3 inhibitor (e.g., neu3/neu2 or neu3/neu4); or a bispecific neu4 inhibitor (e.g., neu4/neu1 or neu4/neu3) for modulating leukocyte activation (e.g., transmigration, adhesion or cytokine response), and in turn, inflammation in a subject in need thereof.

The present disclosure also relates to the use of specific inhibitors of neuraminidase enzymes (e.g. neu1, neu3 or neu4 enzyme, more specifically neu1 enzyme) for modulating thrombocyte clearance. Without being so limited, in a more specific embodiment, it relates to the use of specific inhibitors of neuraminidase enzymes (e.g. neu1, neu3 or neu4 enzyme, more specifically neu1 enzyme) for preventing or treating immune thrombocytopenia or a symptom thereof.

As used herein, the term “specific inhibitor” encompasses bispecific inhibitors and refers to any inhibitor that specifically inhibits the activity or expression of one or two of neu1, neu3 and neu4. In another specific embodiment, it relates to any inhibitor that specifically inhibits neu1 or neu4. In another specific embodiment, it relates to any inhibitor that specifically inhibits neu1. In another specific embodiment, it relates to any inhibitor that specifically inhibits neu3. In another specific embodiment, it relates to any inhibitor that specifically inhibits neu1.

As used herein, the term “specific neu1/neu3/neu4 inhibitor” is used herein to refer to “a specific inhibitor of neuraminidase 1 (neu1); neuraminidase 3 (neu3); neuraminidase 4 (neu4); or a bispecific inhibitor of neu1, neu3 or neu4”. It refers to at least one of a “specific neuraminidase 1 inhibitor”, “specific neuraminidase 3 inhibitor”, “specific neuraminidase 4 inhibitor”, “bispecific neuraminidase 1 inhibitor”, “bispecific neuraminidase 3 inhibitor” and “bispecific neuraminidase 4 inhibitor”. For convenience, it refers to an agent able to reduce, the case being, neu1, neu3 and/or neu4 expression and/or activity. Without being so limited, such inhibitors include small molecules including but not limited those of any one of formulas I, Ia, Ib, and II-VIII and those identified as such in in Table I, dsRNA (e.g., RNAi, siRNA, miRNA), peptides, antibodies or antibody fragments (e.g., antibodies that specifically binds to neu1, neu3 or neu4 or are bispecific against neu1, neu3 or neu4 and against another neuraminidase enzyme, and antibody fragments that specifically binds to neu1, neu3 or neu4 or are bispecific against neu1, neu3 or neu4 and another neuraminidase enzyme). In more specific embodiments, such inhibitors are those identified as such in Table I.

Typically, specific inhibitors advantageously avoid certain deleterious side effects that could be present when using inhibitors with less selectivity.

As used herein the terms “specific neuraminidase 1 inhibitor” refer to an inhibitor that is more active against neuraminidase 1 than against neuraminidase 2, 3, or 4. In a specific embodiment, the inhibitor has an IC50 against neu1 that is at least 2× lower than the IC50 against at least one of neu2, neu3, and neu4 (in a specific embodiment, against at least two of neu2, neu3, and neu4 and in another specific embodiment against all three of neu2, neu3, and neu4). In another specific embodiment, its IC50 against neu1 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu2, neu3, and neu4 (in a specific embodiment, against at least two of neu2, neu3, and neu4 and in another specific embodiment against all three of neu2, neu3, and neu4).

As used herein the terms “specific neuraminidase 3 inhibitor” refer to an inhibitor that is more active against neuraminidase 3 than against neuraminidase 1, 2 or 4. In a specific embodiment, it has an IC50 against neu3 that is at least 2× lower than the IC50 against at least one of neu1, neu2 and neu4 (in a specific embodiment, against at least two of neu1, neu2, and neu4 and in another specific embodiment against all three of neu1, neu2, and neu4). In another specific embodiment, its IC50 against neu3 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu1, neu2 and neu4 (in a specific embodiment, against at least two of neu1, neu2, and neu4 and in another specific embodiment against all three of neu1, neu2, and neu4).

As used herein the terms “specific neuraminidase 4 inhibitor” refer to an inhibitor that is more active against neuraminidase 3 than against neuraminidase 1, 2 or 3. In a specific embodiment, it has an IC50 against neu4 that is at least 2× lower than the IC50 against at least one of neu1, neu2 and neu3 (in a specific embodiment, against at least two of neu1, neu2, and neu3 and in another specific embodiment against all three of neu1, neu2, and neu3). In another specific embodiment, its IC50 against neu4 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu1, neu2 and neu3 (in a specific embodiment, against at least two of neu1, neu2, and neu3 and in another specific embodiment against all three of neu1, neu2, and neu3).

As used herein the terms “bispecific neuraminidase 1 inhibitor” refer to “bispecific neuraminidase 1/neuraminidase 2 inhibitor” (or “bispecific neu1/2 inhibitor”), “bispecific neuraminidase 1/neuraminidase 3 inhibitor” (or “bispecific neu1/3 inhibitor”) or “bispecific neuraminidase 1/neuraminidase 4 inhibitor” (or “bispecific neu1/4 inhibitor”).

As used herein the terms “bispecific neuraminidase 1/neuraminidase 2 inhibitor” or “bispecific neu1/2 inhibitor” refer to an inhibitor that has activity against neuraminidase 1 and neuraminidase 2 and is less active against neuraminidase 3 and/or 4. In a specific embodiment, such an inhibitor has an IC50 against neu1 that is of from 3:1 to 1:3 against neu2. In a specific embodiment, such an inhibitor has an IC50 against neu1 and neu2 that is at least 2× lower than the IC50 against at least one of neu3 and neu4 (in a specific embodiment, against both of neu3 and neu4). In another specific embodiment, its IC50 against neu1 and neu2 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu3 and neu4 (in a specific embodiment, against both of neu3 and neu4).

As used herein the terms “bispecific neuraminidase 1/neuraminidase 3 inhibitor” or “bispecific neu1/3 inhibitor” refer to an inhibitor that has activity against neuraminidase 1 and neuraminidase 3 and is less active against neuraminidase 2 and/or 4. In a specific embodiment, such an inhibitor has an IC50 against neu1 that is of from 3:1 to 1:3 against neu3. In a specific embodiment, such an inhibitor has an IC50 against neu1 and neu3 that is at least 2× lower than the IC50 against at least one of neu2 and neu4 (in a specific embodiment, against both of neu2 and neu4). In another specific embodiment, its IC50 against neu1 and neu3 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu2 and neu4 (in a specific embodiment, against both of neu2 and neu4).

As used herein the terms “bispecific neuraminidase 1/neuraminidase 4 inhibitor” or “bispecific neu1/4 inhibitor” refer to an inhibitor that has activity against neuraminidase 1 and neuraminidase 4 and is less active against neuraminidase 2 and/or 3. In a specific embodiment, such an inhibitor has an IC50 against neu1 that is of from 3:1 to 1:3 against neu4. In a specific embodiment, such an inhibitor has an IC50 against neu1 and neu4 that is at least 2× lower than the IC50 against at least one of neu2 and neu3 (in a specific embodiment, against both of neu2 and neu3). In another specific embodiment, its IC50 against neu1 and neu4 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu2 and neu3 (in a specific embodiment, against both of neu2 and neu3).

As used herein the terms “bispecific neuraminidase 3 inhibitor” refer to “bispecific neuraminidase 3/neuraminidase 1 inhibitor” (or “bispecific neu3/1 inhibitor”) or “bispecific neuraminidase 3/neuraminidase 2 inhibitor” (or “bispecific neu3/2 inhibitor”) or “bispecific neuraminidase 3/neuraminidase 4 inhibitor” (or “bispecific neu3/4 inhibitor”).

As used herein the terms “bispecific neuraminidase 3/neuraminidase 1 inhibitor” or “bispecific neu3/1 inhibitor” refer to an inhibitor that has activity against neuraminidase 3 and neuraminidase 1 and is less active against neuraminidase 2 and/or 4. In a specific embodiment, such an inhibitor has an IC50 against neu3 that is of from 3:1 to 1:3 against neu1. In a specific embodiment, such an inhibitor has an IC50 against neu3 and neu1 that is at least 2× lower than the IC50 against at least one of neu2 and neu4 (in a specific embodiment, against both of neu2 and neu4). In another specific embodiment, its IC50 against neu3 and neu1 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu2 and neu4 (in a specific embodiment, against both of neu2 and neu4).

As used herein the terms “bispecific neu3/2 inhibitor” refer to an inhibitor that has activity against neuraminidase 3 and neuraminidase 2 and is less active against neuraminidase 1 and/or 4. In a specific embodiment, such an inhibitor has an IC50 against neu3 that is of from 3:1 to 1:3 against neu2. In a specific embodiment, such an inhibitor has an IC50 against neu3 and neu2 that is at least 2× lower than the IC50 against at least one of neu1 and neu4 (in a specific embodiment, against both of neu1 and neu4). In another specific embodiment, its IC50 against neu3 and neu2 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu1 and neu4 (in a specific embodiment, against both of neu1 and neu4).

As used herein the terms “bispecific neu3/4 inhibitor” refer to an inhibitor that has activity against neuraminidase 3 and neuraminidase 4 and is less active against neuraminidase 1 and/or 2. In a specific embodiment, such an inhibitor has an IC50 against neu3 that is of from 3:1 to 1:3 against neu4. In a specific embodiment, such an inhibitor has an IC50 against neu3 and neu4 that is at least 2× lower than the IC50 against at least one of neu1 and neu2 (in a specific embodiment, against both of neu1 and neu2). In another specific embodiment, its IC50 against neu3 and neu4 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu1 and neu2 (in a specific embodiment, against both of neu1 and neu2).

As used herein the terms “bispecific neuraminidase 4 inhibitor” refer to “bispecific neuraminidase 4/neuraminidase 1 inhibitor” (or “bispecific neu4/1 inhibitor”) or “bispecific neuraminidase 4/neuraminidase 2 inhibitor” (or “bispecific neu4/2 inhibitor”) or “bispecific neuraminidase 4/neuraminidase 3 inhibitor” (or “bispecific neu4/3 inhibitor”).

As used herein the terms “bispecific neuraminidase 4/neuraminidase 1 inhibitor” or “bispecific neu4/1 inhibitor” refer to an inhibitor that has activity against neuraminidase 4 and neuraminidase 1 and is less active against neuraminidase 2 and/or 3. In a specific embodiment, such an inhibitor has an IC50 against neu4 that is of from 3:1 to 1:3 against neu1.

In a specific embodiment, such an inhibitor has an IC50 against neu4 and neu1 that is at least 2× lower than the IC50 against at least one of neu2 and neu3 (in a specific embodiment, against both of neu2 and neu3). In another specific embodiment, its IC50 against neu4 and neu1 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu2 and neu3 (in a specific embodiment, against both of neu2 and neu3).

As used herein the terms “bispecific neu4/2 inhibitor” refer to an inhibitor that has activity against neuraminidase 4 and neuraminidase 2 and is less active against neuraminidase 1 and/or 3. In a specific embodiment, such an inhibitor has an IC50 against neu4 that is of from 3:1 to 1:3 against neu2. In a specific embodiment, such an inhibitor has an IC50 against neu4 and neu2 that is at least 2× lower than the IC50 against at least one of neu1 and neu3 (in a specific embodiment, against both of neu1 and neu3). In another specific embodiment, its IC50 against neu4 and neu2 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu1 and neu3 (in a specific embodiment, against both of neu1 and neu3).

As used herein the terms “bispecific neu4/3 inhibitor” refer to an inhibitor that has activity against neuraminidase 4 and neuraminidase 3 and is less active against neuraminidase 1 and/or 2. In a specific embodiment, such an inhibitor has an IC50 against neu4 that is of from 3:1 to 1:3 against neu3. In a specific embodiment, such an inhibitor has an IC50 against neu4 and neu3 that is at least 2× lower than the IC50 against at least one of neu1 and neu2 (in a specific embodiment, against both of neu1 and neu2). In another specific embodiment, its IC50 against neu4 and neu3 is at least 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, or 46× lower than its IC50 against at least one of neu1 and neu2 (in a specific embodiment, against both of neu1 and neu2).

As used herein, the term “leukocyte activation” includes leukocyte transmigration, adhesion and/or cytokine response. As used herein the term “leukocyte transmigration or adhesion” refers to events occurring in inflammation. More specifically, it refers to the specific steps of the inflammatory cascade leading to inflammation. These steps include rolling, firm adhesion, and transmigration. As used herein the term cytokine response, refers to, without being so limited, secretion by e.g., one or more leukocytes of at least one cytokine in the circulation (e.g., blood or plasma). Without being so limited, the response of the following cytokines has been modulated by at least one neuraminidase depletion (e.g., neu1 and neu4) in the present disclosure: CSF/CSF-3, IL-21, IL-6, IFN-γ and RANTES.

The present disclosure also relates to the use of specific neu1, neu3 and neu4 inhibitors, and bispecific neu1 neu3 or neu4 inhibitors (e.g., neu3/neu4 inhibitors) to reduce inflammation in a subject in need thereof.

The present disclosure also relates to the use of specific inhibitors of neuraminidase enzymes (e.g. neu1, neu3 or neu4 enzyme, more specifically neu1 enzyme) for modulating thrombocyte clearance. Without being so limited, in a more specific embodiment, it relates to the use of specific inhibitors of neuraminidase enzymes (e.g. neu1, neu3 or neu4 enzyme, more specifically neu1 enzyme) for preventing or treating immune thrombocytopenia or a symptom thereof.

As used herein, the term “thrombocyte clearance” relates to reduction of thrombocyte in the bloodstream (e.g., through thrombocyte phagocytosis by leukocytes).

As used herein the term “a symptom thereof” in the term “immune thrombocytopenia or a symptom thereof” refers to, without being so limited, a reduced thrombocyte (platelet) level in the blood.

As used herein, the term “prevent/preventing/prevention” or “treat/treating/treatment”, refers to eliciting the desired biological response, i.e., a prophylactic and therapeutic effect, respectively in a subject. In accordance with the present disclosure, the therapeutic effect comprises one or more of a decrease/reduction in the severity, intensity and/or duration of the immune thrombocytopenia or symptom thereof or of the inflammation (following bacterial infection) following administration of the inhibitor of the present disclosure when compared to its severity, intensity and/or duration in the subject prior to treatment or as compared to that/those in a non-treated control subject having the infection or any symptom thereof. In accordance with the disclosure, a prophylactic effect may comprise a delay in the onset of the immune thrombocytopenia or symptom thereof or of the inflammation (following bacterial infection) in an asymptomatic subject at risk of experiencing the immune thrombocytopenia or symptom thereof or of the inflammation (following bacterial infection) at a future time; or a decrease/reduction in the severity, intensity and/or duration of immune thrombocytopenia or symptom thereof or of the inflammation (following bacterial infection) occurring following administration of the inhibitor of the present disclosure, when compared to the timing of their onset or their severity, intensity and/or duration in a non-treated control subject (i.e. asymptomatic subject at risk of experiencing the immune thrombocytopenia or symptom thereof or of the inflammation (following bacterial infection); and/or a decrease/reduction in the progression of any pre-existing immune thrombocytopenia or symptom thereof or of the inflammation (following bacterial infection) in a subject following administration of the agent of the present disclosure when compared to the progression of an immune thrombocytopenia or symptom thereof or of the inflammation (following bacterial infection) in a non-treated control subject having such pre-existing immune thrombocytopenia or symptom thereof or of the inflammation (following bacterial infection). As used herein, in a therapeutic treatment, the inhibitor of the present disclosure is administered after the onset of the immune thrombocytopenia or symptom thereof or of the inflammation (following bacterial infection). As used herein, in a prophylactic treatment, the agent of the present disclosure is administered before the immune thrombocytopenia or symptom thereof or of the inflammation (following bacterial infection) or after the onset thereof but before the progression thereof.

A “therapeutically effective amount” or “effective amount” or “therapeutically effective dosage” of a specific inhibitor of the disclosure or composition thereof can result in a modulation of inflammation (e.g., leukocyte transmigration or adhesion or cytokine response) (e.g., decrease of inflammation) in a subject.

### Small Molecule Inhibitors

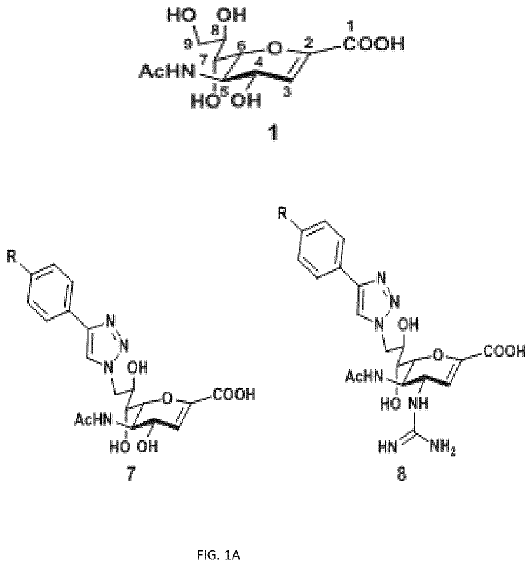

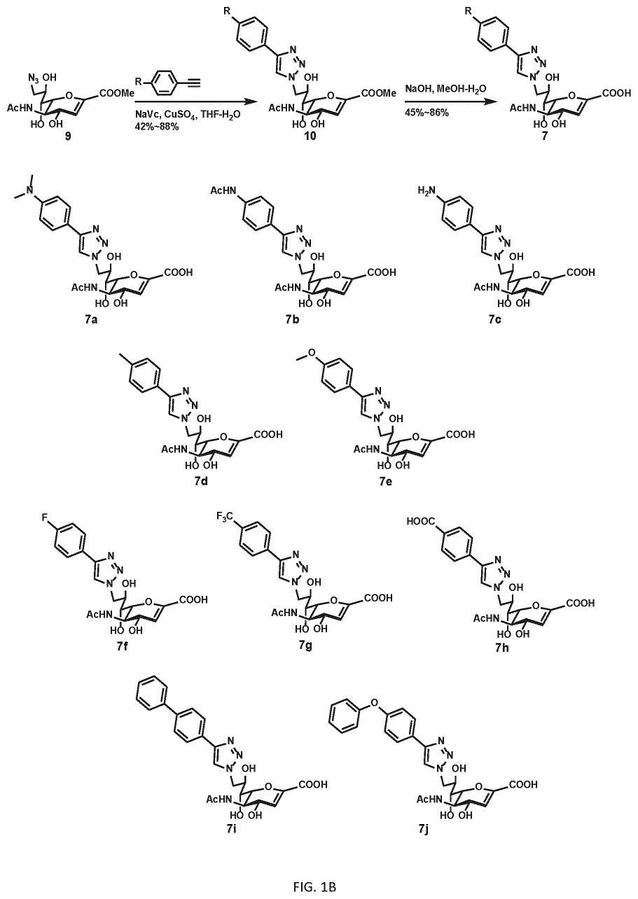

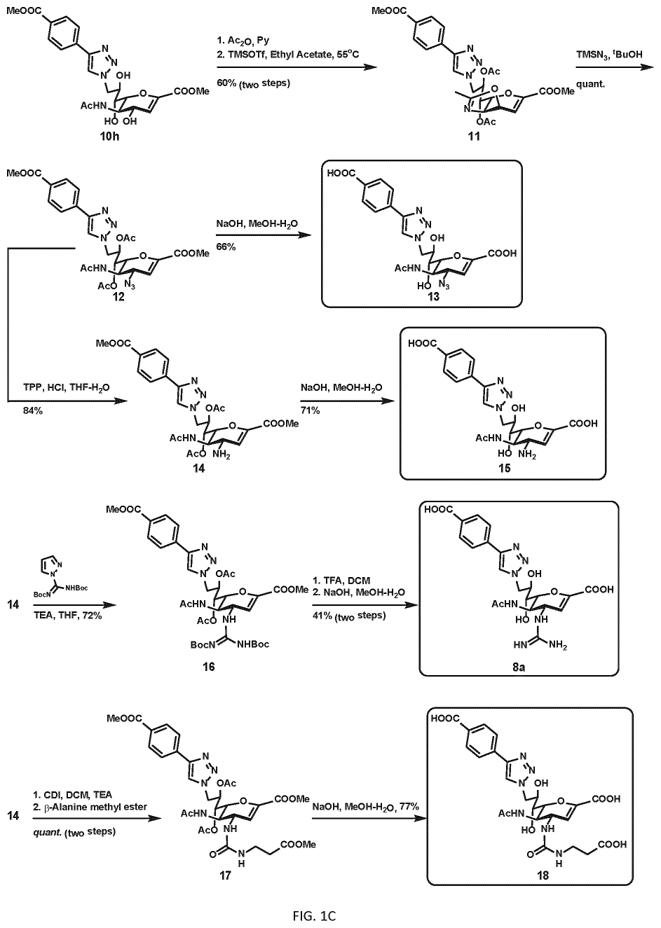

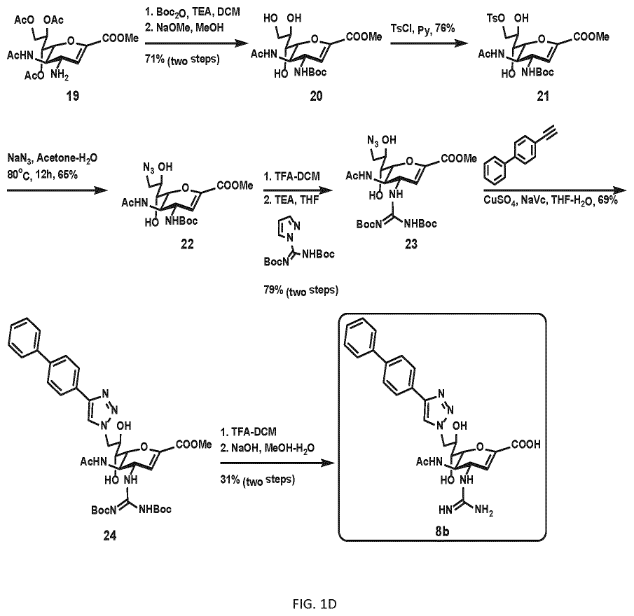

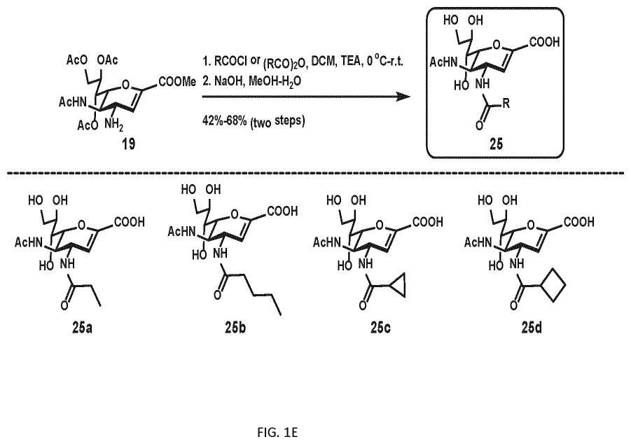

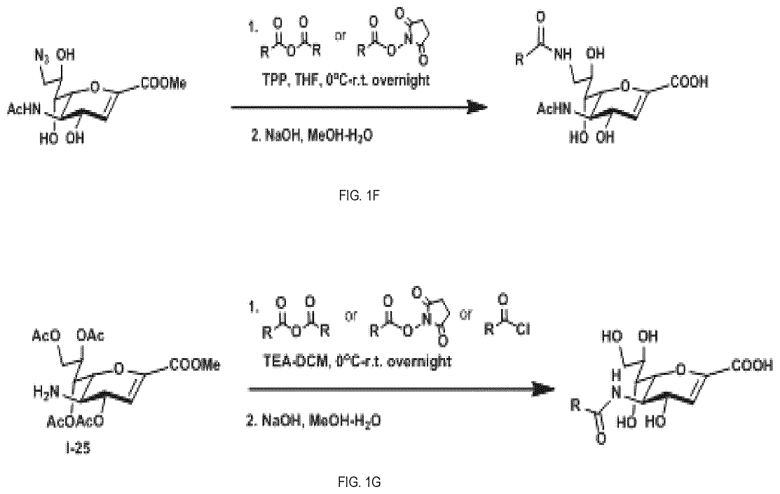

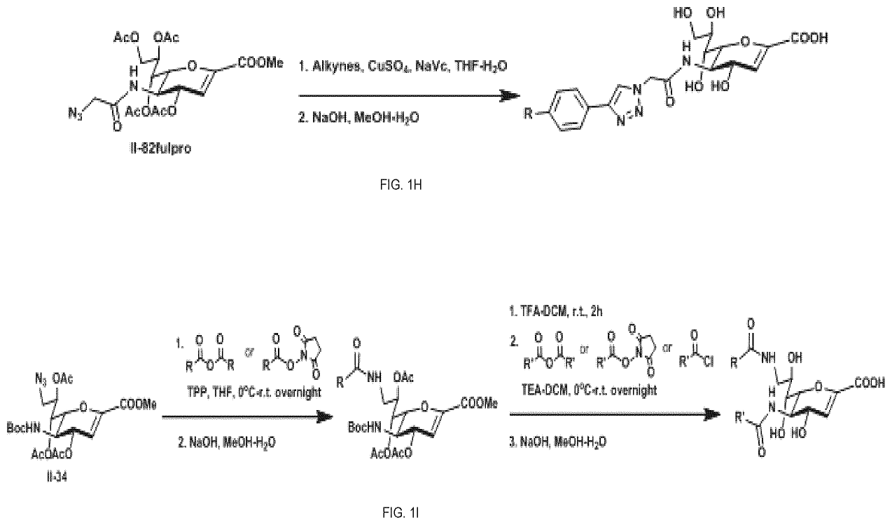

The structure of specific small molecules of the present disclosure are shown in FIGS. 1A-1I and/or Examples 1 to 93. Their names are also indicated in the Examples. In case of discrepancies between the name and structure presented, the structure shall prevail.

In specific embodiments of the present disclosure, small molecule inhibitors of the present disclosure (e.g., formulas I, Ia, Ib, and II-VIII) have an IC50 against neu1, neu3 or neu4 that is of 100 μM or lower, 20 μM or lower, 10 μM or lower, 3 μM or lower, 1 μM or lower or lower than 1 μM. In specific embodiments, small molecule inhibitors of the present disclosure are the compounds of Table I that have an IC50 against neu1, neu3 or neu4 that is lower than 1 M (e.g., compounds with neu1 specificity or bispecificity: compounds 31, 32, 67-69, 72, 74 and 75; compounds with neu3 specificity or bispecificity: 5c, 7i, 8a and 8b; and compounds with neu4 specificity or bispecificity: 7i, 7j and 28); 1 M or lower (e.g., the foregoing compounds and compound 7j (neu3 specificity or bispecificity); 3 μM or lower (e.g., the foregoing compounds and compounds 54, 56, 33, 57, 36, 51, 58, 65, 66, 70 and 73 (neu1 specificity or bispecificity), compounds 7h and 27 (neu3 specificity or bispecificity), and compounds 7e, 7f, 7h, 26 and 63); and 10 μM or lower (e.g., the foregoing compounds and compounds 55, 50, 30, 34, 37, 49, 52, 60-61, 64 (neu1 specificity or bispecificity)), compounds 7e, 7c, 7a, 7b, 7d, 7g, zanamivir (6), 40, 58, 60 and 63 (neu3 specificity or bispecificity), and compounds 7d, 7g, 8b, 27, 30 and 40 (neu4 specificity or bispecificity); 20 μM or lower (e.g., the foregoing compounds and compounds 29, 38, 39 and 71 (neu1 specificity or bispecificity)), compounds 7f, 26, and 13 (neu3 specificity or bispecificity), and compounds 7b and 7c (neu4 specificity or bispecificity); or 100 μM or lower (e.g., the foregoing compounds and compounds 35 and 59 (neu1 specificity or bispecificity), compounds 53, 28, 59 and 62 (neu3 specificity or bispecificity), and compounds 7b and 7c (neu4 specificity or bispecificity)).

### Chemical Groups

As used herein, the term “alkyl” refers to a monovalent straight or branched chain, saturated or unsaturated aliphatic hydrocarbon radical having a number of carbon atoms in the specified range. Thus, for example, “C1-10 alkyl” (or “C1-C10 alkyl”) refers to any of the hexyl alkyl and pentyl alkyl isomers as well as n-, iso-, sec- and t-butyl, n- and isopropyl, ethyl, and methyl. As another example, “C1-4 alkyl” refers to n-, iso-, sec- and t-butyl, n- and isopropyl, ethyl, and methyl. As another example, “C1-3 alkyl” refers to n-propyl, isopropyl, ethyl, and methyl. Alkyl include unsaturated aliphatic hydrocarbon including alkyne (R—C≡C—R); and/or alkene (R—C═C—R).

The term “halogen” (or “halo”) refers to fluorine, chlorine, bromine and iodine (alternatively referred to as fluoro, chloro, bromo, and iodo). The term “haloalkyl” refers to an alkyl group as defined above in which one or more of the hydrogen atoms have been replaced with a halogen (i.e., F, Cl, Br and/or I). Thus, for example, “C1-10 haloalkyl” (or “C1-C6 haloalkyl”) refers to a C1 to C10 linear or branched alkyl group as defined above with one or more halogen substituents.

The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “heteroalkyl” is given its ordinary meaning in the art and refers to alkyl groups as described herein in which one or more carbon atoms is replaced with a heteroatom (e.g., oxygen, nitrogen, sulfur, or derivatives thereof, and the like). Examples of heteroalkyl groups include, but are not limited to, alkoxy, alkyl-substituted amino, thiol such as methionine side group. Up to two heteroatoms may be consecutive. When a prefix such as C2-6 is used to refer to a heteroalkyl group, the number of carbons (2-6, in this example) is meant to include the heteroatoms as well.

The term “aminoalkyl” refers to an alkyl group as defined above in which one or more of the hydrogen or carbon atoms has been replaced with a nitrogen or an amino derivative. Thus, for example, “C1-6 aminoalkyl” (or “C1-C6 aminoalkyl”) refers to a C1 to C6 linear or branched alkyl group as defined above with one or more amino derivatives (e.g., NH, amide, diazirin, azide, etc.).

The term “thioalkyl” refers to an alkyl group as defined above in which one or more of the hydrogen or carbon atoms has been replaced with a sulfur atom or thiol derivative. Thus, for example, “C1-6 aminoalkyl” (or “C1-C6 aminoalkyl”) refers to a C1 to C6 linear or branched alkyl group as defined above with one or more sulfur atoms or thiol derivatives (e.g., S, SH, etc.).

Aminoalkyl and thioalkyls are specific embodiments of and encompassed by the term “heteroalkyl” or substituted alkyl depending on the heteroatom replaces a carbon atom or an hydrogen atom.

The term “cycloalkyl” refers to saturated alicyclic hydrocarbon consisting of saturated 3-8 membered rings optionally fused with additional (1-3) aliphatic (cycloalkyl) or aromatic ring systems, each additional ring consisting of a 3-8 membered ring. It includes without being so limited cyclopropane, cyclobutane, cyclopentane, and cyclohexane.

The term “heterocyclyl” refers to (i) a 4- to 7-membered saturated heterocyclic ring containing from 1 to 3 heteroatoms independently selected from N, O and S, or (ii) is a heterobicyclic ring (e.g., benzocyclopentyl). Examples of 4- to 7-membered, saturated heterocyclic rings within the scope of this disclosure include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Examples of 4- to 7-membered, unsaturated heterocyclic rings within the scope of this disclosure include mono-unsaturated heterocyclic rings corresponding to the saturated heterocyclic rings listed in the preceding sentence in which a single bond is replaced with a double bond (e.g., a carbon-carbon single bond is replaced with a carbon-carbon double bond).

The terms “C(O)” and —CO refer to carbonyl. The terms “S(O)₂” and “SO₂” each refer to sulfonyl. The term “S(O)” refers to sulfinyl.

The term “aryl” refers to aromatic (unsaturated) compounds consisting of 3-8 membered rings, optionally fused with additional (1-3) aliphatic (cycloalkyl) or aromatic ring systems, each additional ring consisting of 3-8 membered ring. In a specific embodiment, it refers to phenyl, benzocyclopentyl, or naphthyl. The aryl of particular interest is phenyl. The term “heteroaryl” refers to (i) a 3-, 4-, 5- or 6-membered heteroaromatic ring containing from 1 to 4 heteroatoms independently selected from N, O and S, or (ii) is a heterobicyclic ring selected from quinolinyl, isoquinolinyl, and quinoxalinyl. Suitable 3-, 4-, 5- and 6-membered heteroaromatic rings include, for example, diazirin, pyridyl (also referred to as pyridinyl), pyrrolyl, diazine (e.g., pyrazinyl, pyrimidinyl, pyridazinyl), triazinyl, thienyl, furanyl, imidazolyl, pyrazolyl, triazolyl (e.g., 1, 2, 3 triazolyl), tetrazolyl (e.g., 1, 2, 3, 4 tetrazolyl), oxazolyl, iso-oxazolyl, oxadiazolyl, oxatriazolyl, thiazolyl, isothiazolyl, and thiadiazolyl. Heteroaryls of particular interest are pyrrolyl, imidazolyl, pyridyl, pyrazinyl, quinolinyl (or quinolyl), isoquinolinyl (or isoquinolyl), and quinoxalinyl. Suitable heterobicyclic rings include indolyl.

As used herein, and unless otherwise specified, the terms “alkyl”, “haloalkyl”, “aminoalkyl”, “cycloalkyl”, “heterocyclyl”, “aryl”, “heteroalkyl” and “heteroaryl” and the terms designating their specific embodiments (e.g., butyl, fluoropropyl, aminobutyl, cyclopropane, morpholine, phenyl, pyrazole, etc.) encompass the substituted (i.e. in the case of haloalkyl and aminoalkyl, in addition to their halogen and nitrogen substituents, respectively) and unsubstituted embodiments of these groups. Hence for example, the term “phenyl” encompasses unsubstituted phenyl as well as fluorophenyl, hydroxyphenyl, methylsulfonyl phenyl (or biphenyl), trifluoromethyl-diazirin-phenyl, isopropyl-phenyl, trifluorohydroxy-phenyl. Similarly, the term pyrazole, encompass unsubstituted pyrazole as well as methylpyrazole. The one or more substituents may be an amine, halogen, hydroxyl, C1-6 aminoalkyl, C1-6 heteroalkyl, C1-6 alkyl, C3-8 cycloalkyl, C1-6 haloalkyl, aryl, heteroaryl and heterocyclyl groups (etc.).

It is understood that the specific rings listed above are not a limitation on the rings which can be used in the present disclosure. These rings are merely representative.

Unless expressly stated to the contrary in a particular context, any of the various cyclic rings and ring systems described herein may be attached to the rest of the compound at any ring atom (i.e., any carbon atom or any heteroatom) provided that a stable compound results.

### Isomers, Tautomers and Polymorphs

As used herein, the term “isomers” refers to optical isomers (enantiomers), diastereoisomers as well as the other known types of isomers.

The compounds of the disclosure have at least five asymmetric carbon atoms and can therefore exist in the form of optically pure enantiomers (optical isomers), as racemates and as mixtures thereof. Some of the compounds have at least two asymmetric carbon atoms and can therefore exist in the form of pure diastereoisomers and as mixtures thereof. It is to be understood, that, unless otherwise specified, the present disclosure embraces the racemates, the enantiomers and/or the diastereoisomers of the small molecule inhibitors of the disclosure as well as mixtures thereof.

In addition, the present disclosure embraces all geometric isomers. For example, when a compound of the disclosure incorporates a double bond or a fused ring, both the cis- and trans-forms, as well as mixtures, are embraced within the scope of the disclosure.

Within the present disclosure, it is to be understood that a compound of the disclosure may exhibit the phenomenon of tautomerism and that the formula drawings within this specification can represent only one of the possible tautomeric forms. It is to be understood that the disclosure encompasses any tautomeric form and is not to be limited merely to any one tautomeric form utilized within the formula drawings.

It is also to be understood that certain small molecule inhibitors of the disclosure may exhibit polymorphism, and that the present disclosure encompasses all such forms.

### Salts

The present disclosure relates to the small molecule inhibitors of the disclosure as hereinbefore defined as well as to salts thereof. The term “salt(s)”, as employed herein, denotes basic salts formed with inorganic and/or organic bases. Salts for use in pharmaceutical compositions will be pharmaceutically acceptable salts, but other salts may be useful in the production of the compounds of the disclosure. The term “pharmaceutically acceptable salts” refers to salts of compounds of the present disclosure that are pharmacologically acceptable and substantially non-toxic to the subject to which they are administered. More specifically, these salts retain the biological effectiveness and properties of the anti-atherosclerosis compounds of the disclosure and are formed from suitable non-toxic organic or inorganic acids or bases.

For example, where the small molecule inhibitors of the disclosure are sufficiently acidic, the salts of the disclosure include base salts formed with an inorganic or organic base. Such salts include alkali metal salts such as sodium, lithium, and potassium salts; alkaline earth metal salts such as calcium and magnesium salts; metal salts such as aluminum salts, iron salts, zinc salts, copper salts, nickel salts and a cobalt salts; inorganic amine salts such as ammonium or substituted ammonium salts, such as e.g., trimethylammonium salts; and salts with organic bases (for example, organic amines) such as chloroprocaine salts, dibenzylamine salts, dicyclohexylamine salts, dicyclohexylamines, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amines salts, tetramethylammonium salts, t-octylamine salts, tris-(2-hydroxyethyl)amine salts, and tris(hydroxymethyl)aminomethane salts. Preferred salts include those formed with sodium, lithium, potassium, calcium and magnesium.

Such salts can be formed routinely by those skilled in the art using standard techniques. Indeed, the chemical modification of a pharmaceutical compound (i.e. drug) into a salt is a technique well known to pharmaceutical chemists, (See, e.g., H. Ansel et. al., Pharmaceutical Dosage Forms and Drug Delivery Systems (6th Ed. 1995) at pp. 196 and 1456-1457, incorporated herein by reference). Salts of the compounds of the disclosure may be formed, for example, by reacting a compound of the disclosure with an amount of acid or base, such as an equivalent amount, in a medium such as one in which the salt precipitates or in an aqueous medium followed by lyophilization.

### Esters

The present disclosure relates to the small molecule inhibitors of the disclosure as hereinbefore defined as well as to the esters thereof. The term “ester(s)”, as employed herein, refers to compounds of the disclosure or salts thereof in which hydroxy groups have been converted to the corresponding esters using, for example, inorganic or organic anhydrides, acids, or acid chlorides. Esters for use in pharmaceutical compositions will be pharmaceutically acceptable esters, but other esters may be useful in the production of the compounds of the disclosure.

The term “pharmaceutically acceptable esters” refers to esters of the compounds of the present disclosure that are pharmacologically acceptable and substantially non-toxic to the subject to which they are administered. More specifically, these esters retain the biological effectiveness and properties of the anti-atherosclerosis small molecule inhibitors of the disclosure and act as prodrugs which, when absorbed into the bloodstream of a warm-blooded animal, cleave in such a manner as to produce the parent alcohol small molecule inhibitor.

Esters of the small molecule inhibitors of the present disclosure include among others the following groups (1) carboxylic acid esters obtained by esterification of the hydroxy groups, in which the non-carbonyl moiety of the carboxylic acid portion of the ester grouping is selected from straight or branched chain alkyl (for example, ethyl, n-propyl, t-butyl, n-butyl, methyl, propyl, isopropyl, butyl, isobutyl, or pentyl), alkoxyalkyl (for example, methoxymethyl, acetoxymethyl, and 2,2-dimethylpropionyloxymethyl), aralkyl (for example, benzyl), aryloxyalkyl (for example, phenoxymethyl), aryl (for example, phenyl optionally substituted with, for example, halogen, C1-4 alkyl, or C1-4 alkoxy, or amino); (2) sulfonate esters, such as alkyl- or aralkylsulfonyl (for example, methanesulfonyl); (3) amino acid esters (for example, L-valyl or L-isoleucyl); (4) phosphonate esters; (5) mono-, di- or triphosphate esters (including phosphoramidic cyclic esters). The phosphate esters may be further esterified by, for example, a C1-20 alcohol or reactive derivative thereof, or by a 2,3-di(C₆₋₂₄)acyl glycerol. (6) Carbamic acid ester (for example N-methylcarbamic ester); and (7) Carbonic acid ester (for example methylcabonate).

Further information concerning examples of and the use of esters for the delivery of pharmaceutical compounds is available in Design of Prodrugs. Bundgaard H ed. (Elsevier, 1985) incorporated herein by reference. See also, H. Ansel et. al., Pharmaceutical Dosage Forms and Drug Delivery Systems (6th Ed. 1995) at pp. 108-109; Krogsgaard-Larsen, et. al., Textbook of Drug Design and Development (2d Ed. 1996) at pp. 152-191; Jarkko Rautio et al., Nat. Rev. Drug Discov., 7, pp. 255-270 (2008); and Pen-Wei Hsieh et al., Curr. Pharm. Des., 15 (19), pp. 2236-2250 (2009), all incorporated herein by reference.

The small molecule inhibitors of this disclosure may be esterified by a variety of conventional procedures including reacting the appropriate anhydride, carboxylic acid or acid chloride with an alcohol group of a compound of this disclosure. For example, an appropriate anhydride may be reacted with an alcohol in the presence of a base, such as 1,8-bis[dimethylamino]naphthalene or N,N-dimethylaminopyridine, to facilitate acylation. Also, an appropriate carboxylic acid can be reacted with an alcohol in the presence of a dehydrating agent such as dicyclohexylcarbodiimide, 1-[3-dimethylaminopropyl]-3-ethylcarbodiimide or other water-soluble dehydrating agents which are used to drive the reaction by the removal of water, and, optionally, an acylation catalyst. Esterification can also be effected using the appropriate carboxylic acid. Reaction of an acid chloride with an alcohol can also be carried out. When a compound of the disclosure contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities. One skilled in the art would readily know how to successfully carry out these as well as other known methods of esterification of alcohols.

Esters of the small molecule inhibitors of the disclosure may form salts. Where this is the case, this is achieved by conventional techniques as described above.

In a specific embodiment, esters of the present disclosure are compounds of formulas I, Ia, Ib, and II-VIII of the present disclosure with a methyl, ethyl, propyl, or butyl at position R1.

### Solvates

The small molecule inhibitors of the disclosure may exist in unsolvated as well as solvated forms with solvents such as water, ethanol, and the like, and it is intended that the disclosure embrace both solvated and unsolvated forms.

“Solvate” means a physical association of a small molecule inhibitor of this disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Solvates for use in pharmaceutical compositions will be pharmaceutically acceptable solvates, but other solvates may be useful in the production of the compounds of the disclosure.

As used herein, the term “pharmaceutically acceptable solvates” means solvates of small molecule inhibitors of the present disclosure that are pharmacologically acceptable and substantially non-toxic to the subject to which they are administered. More specifically, these solvates retain the biological effectiveness and properties of the anti-atherosclerosis small molecule inhibitors of the disclosure and are formed from suitable non-toxic solvents.

Non-limiting examples of suitable solvates include ethanolates, methanolates, and the like, as well as hydrates, which are solvates wherein the solvent molecules are H₂O.

Preparation of solvates is generally known. Thus, for example, Ciara, 2004, incorporated herein by reference, describe the preparation of the solvates of the antifungal fluconazole in ethyl acetate as well as from water. Similar preparations of solvates, hemisolvate, hydrates and the like are described by van Tonder, 2004; Bingham, 2001, both incorporated herein by reference.

A typical, non-limiting, process for preparing a solvate involves dissolving the inventive compound in desired amounts of the desired solvent (organic or water or mixtures thereof) at a higher than ambient temperature and cooling the solution at a rate sufficient to form crystals which are then isolated by standard methods. Analytical techniques such as, for example IR spectroscopy, can be used to show the presence of the solvent (or water) in the crystals as a solvate (or hydrate).

### Antibodies

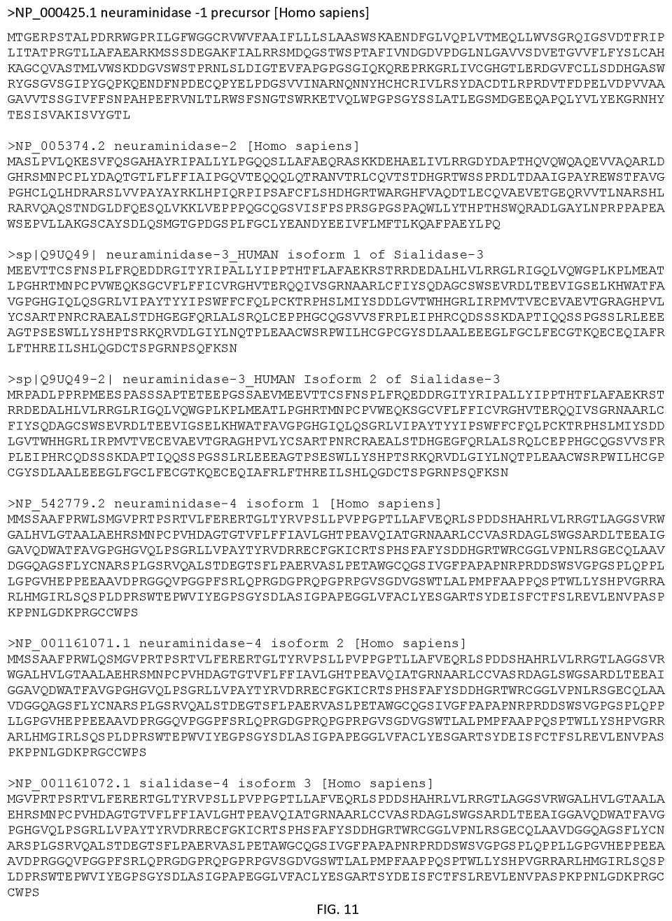

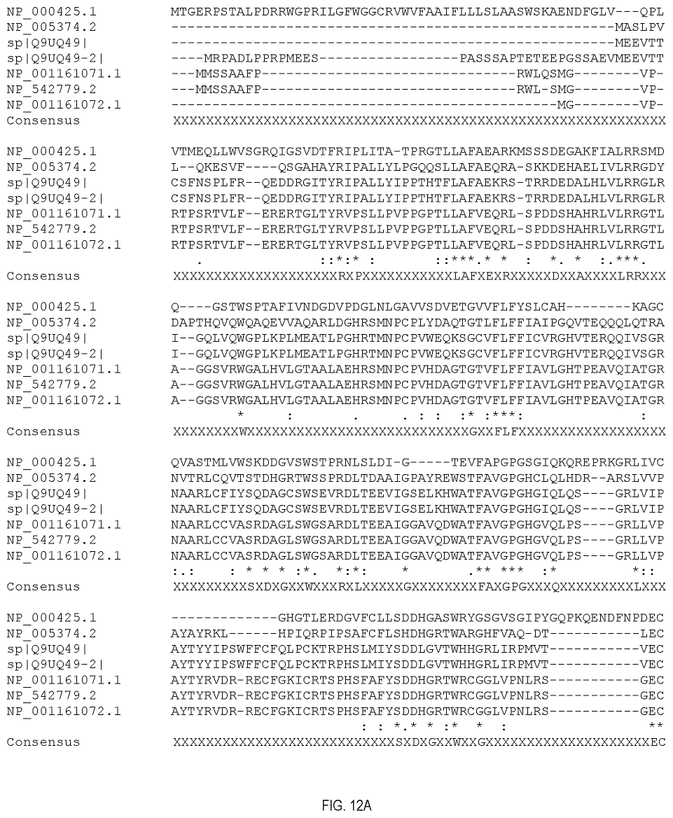

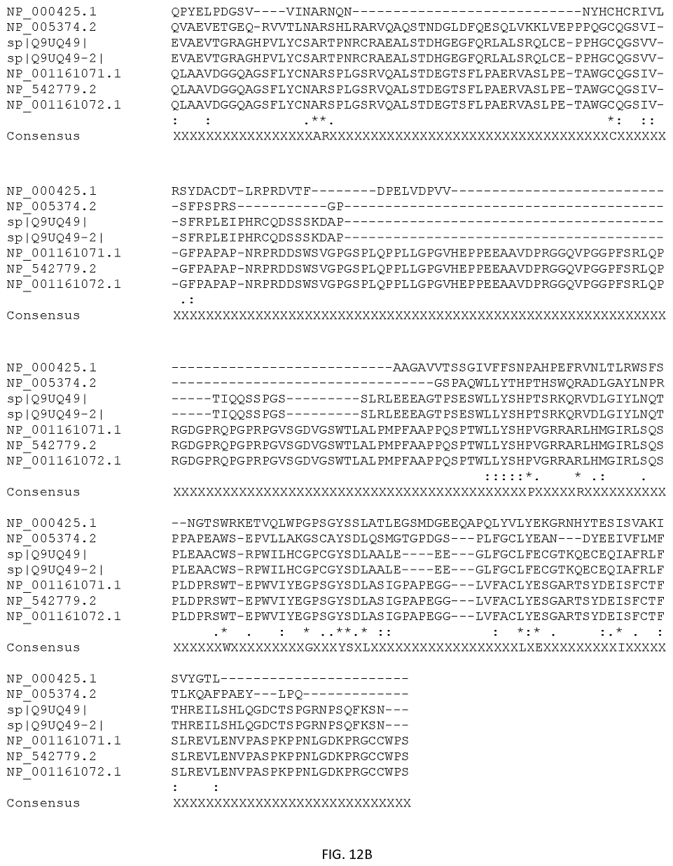

The present disclosure also encompasses the use of antibodies that specifically bind to either of neuraminidase 1 (NP_000425.1); to neuraminidase 3 (isoform 1 (Q9UQ49-1); or 2 (Q9UQ49-2) or to neuraminidase 4 (isoform 1 (NP_542779.2); 2 (NP_001161071.1); or 3 (NP_001161072.1). (see FIG. 11). Antibodies that specifically bind to either of neu1, neu3 or neu4 can be prepared by using epitopes present specifically in either of these proteins. See alignments of neuraminidase 1 to 4 in FIGS. 12A-B.

As indicated above, illustrative human neuraminidase amino acid sequences are presented in FIGS. 11 and 12A-B.

Antibodies that specifically bind to neuraminidase 1, 3 or 4 may be devised by targeting epitope regions of these neuraminidases that are specifically found in each of these enzymes. An epitope of a protein/polypeptide is defined as a fragment of said protein/polypeptide of at least about 4 or 5 amino acids in length, capable of eliciting a specific antibody and/or an immune cell (e.g., a T cell or B cell) bearing a receptor capable of specifically binding said epitope. Two different kinds of epitopes exist: linear epitopes and conformational epitopes. A linear epitope comprises a stretch of consecutive amino acids. A conformational epitope is typically formed by several stretches of consecutive amino acids that are folded in position and together form an epitope in a properly folded protein. An immunogenic fragment as used herein refers to either one, or both, of said types of epitopes. Without being so limited, epitopes in a sequence may be predicted with softwares such as BCPred™, AAP™, FBCPred™ and ABCPred™.

Methods for making antibodies are well known in the art. Polyclonal antibodies can be prepared by immunizing a suitable subject (e.g., rabbit, goat, mouse, or other mammal) with the polypeptide/protein of interest or a fragment thereof as an immunogen. A polypeptide/protein “fragment” “portion” or “segment” is a stretch of amino acid residues of at least about 5, 7, 10, 14, 15, 20, 21 or more amino acids of the polypeptide noted above. The antibody titer in the immunized subject can be monitored over time by standard techniques, such as with an enzyme linked immunosorbent assay (ELISA) using immobilized exosomal marker polypeptide or a fragment thereof. At an appropriate time after immunization, e.g., when the antibody titers are highest, antibody-producing cells can be obtained from the animal, usually a mouse, and can be used to prepare monoclonal antibodies by standard techniques, such as the hybridoma technique originally described by Kohler and Milstein (1975) Nature 256: 495-497, the human B cell hybridoma technique (Kozbor et al. (1983) Immunol. Today 4: 72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology, John Wiley & Sons, Inc., New York, N.Y.).

Alternatively, to preparing monoclonal antibody-secreting hybridomas, a monoclonal antibody can be identified and isolated by screening a recombinant combinatorial immunoglobulin library (e.g., an antibody phage display library) with a polypeptide or a fragment thereof to thereby isolate immunoglobulin library members that bind the polypeptide. Kits for generating and screening phage display libraries are commercially available (e.g., the Pharmacia Recombinant Phage Antibody System™, Catalog No. 27-9400-01; and the Stratagene SurfZAP™ Phage Display Kit, Catalog No. 240612).

Without being so limited, anti-neuraminidase 1 (lysosomal sialidase) antibodies include: Anti-NEU1/NEU Antibody (aa172-221) IHC-plus™ from LifeSpan BioScience; Human NEU-1/Sialidase-1 Antibody (MAB6860) from R & D Systems; Human NEU-1/Sialidase-1 Antibody (MAB6860-SP) from R & D systems; NEU-1/Sialidase-1 Antibody (3D4) (NBP2-46152) from Novus Biologicals; NEU-1/Sialidase-1 Antibody (H00004758-B02P-50 ug) from Novus Biologicals; anti-Neuraminidase, NEU (NEU) (Internal Region) antibody (ABIN964880); Monoclonal Antibody to Neuraminidase (NEU) (MAB611Hu21) from Cloud-Clone; Anti-NEU1 (HPA015634) from Atlas antibody.

Without being so limited, anti-neuraminidase 3 (membrane sialidase) antibodies include Anti-NEU3 Antibody (clone 11B) (LS-C179421-100) from Lifespans BioScience; anti-Neu3 antibody (ABIN1449196) from Antibodies online; NEU3 Antibody (NBP2-48694) from Novus Biologicals; Anti-NEU3 Antibody (HPA038730) from Atlas Antibodies; Anti-NEU3 (Human) mAb (D164-3) from MBL International; Sialidase 3 antibody (orb186135) from Biorbyt, etc.

Without being so limited, anti-neuraminidase 4 antibodies include anti-neu4 antibody (ab107258) from Abcam; anti-neu4 antibody (NBP2-32682) from Novus biotechnologicals; anti-neu4 antibody (OAAB00394) from Aviva Systems Biology; anti-neu4 antibody (AP52856PU-N) from Origen; anti-neu4 antibodies ((HPA037394) from Atlas Antibodies, etc.

### Nucleic Acid Inhibitors

In a specific embodiment, the specific inhibitor of the present disclosure is a double-stranded RNA (dsRNA) molecule (or a molecule comprising region of double-strandedness). The dsRNA comprises a subsequence of a neu1 and/or neu3 polynucleotide (e.g., a subsequence of the sequence encoding neu1, neu3 or neu4 disclosed in FIGS. 11 and 12A-B). In some embodiments, the dsRNA is about 15, 16, 17, 18, 19, 20, 21, 22, 23, 24, 25 or more duplex nucleotides in length, in some embodiments, the dsRNA is a small interfering RNA (siRNA), a short-hairpin RNA (shRNA), or a micro RNA (miRNA). Also provided herein are double-stranded RNA (dsRNA) molecules, comprising a portion of the mature polypeptide coding sequence of any one of the coding sequences of the polypeptides disclosed herein of inhibiting expression of that polypeptide in a cell. While the present disclosure is not limited by any particular mechanism of action, in some embodiments, the dsRNA enters a cell and causes the degradation of a single-stranded RNA (ssRNA) of similar or identical sequences, including endogenous mRNAs. When a cell is exposed to dsRNA, mRNA from the homologous gene is selectively degraded by a process called RNA interference (RNAi). In some embodiments, dsRNAs provided herein are used in gene-silencing methods. In one aspect, methods are provided to selectively degrade RNA using the dsRNAi's disclosed herein. In some embodiments, the specific inhibitor of the present disclosure is a shRNA expressed by a DNA vector transfected or transduced into a target cell. In some embodiments, the specific inhibitor of the present disclosure is a virus encoding a shRNA. In some embodiments, the specific inhibitor of the present disclosure is a vector encoding a shRNA. The process is alternatively practiced in vitro, ex vivo or in vivo. In one aspect, the dsRNA molecules are used to generate a loss-of-function mutation in a cell, an organ or an organism. Methods for making and using dsRNA molecules to selectively degrade RNA are described in the art, see, for example, U.S. Pat. Nos. 6,506,559; 6,511,824; 6,515,109; and 6,489,127.

Generation of Anti-Neu1, Anti-Neu3 or Anti-Neu4 dsRNA Molecules

In some embodiments, an anti-neu1, anti-neu3 or anti-neu4 double stranded RNA molecule with sequences complementary to a target is generated. The synthesis of an anti-neu1, anti-neu3 or anti-neu4 dsRNA molecule comprises: (a) synthesis of two complementary strands of the dsRNA molecule; and (b) annealing the two complementary strands together under conditions suitable to obtain a double-stranded RNA molecule. In another embodiment, synthesis of the two complementary strands of the RNA molecule is by solid phase oligonucleotide synthesis. In yet another embodiment, synthesis of the two complementary strands of the RNA molecule is by solid phase tandem oligonucleotide synthesis. In some embodiments, a nucleic acid molecule described herein is synthesized separately and joined together post-synthetically, for example, by ligation or by hybridization following synthesis and/or deprotection. Oligonucleotides (e.g., certain modified oligonucleotides or portions of oligonucleotides lacking ribonucleotides) are synthesized using any suitable method. dsRNA constructs can be purified by gel electrophoresis or can be purified by high pressure liquid chromatography.

### Design of RNAi Molecules

In some embodiments, an anti-neu1, anti-neu3 or anti-neu4 dsRNA molecule (e.g., RNAi molecules, siRNA molecules, miRNA molecules, and analogues thereof) is about 20-25 bp. In some embodiments, the 20-25 bp dsRNA molecule (e.g., RNAi molecules, siRNA molecules, miRNA molecules, and analogues thereof) has 2-5 bp overhangs on the 3′ end of each strand, and a 5′ phosphate terminus and a 3′ hydroxyl terminus. In some embodiments, the 20-25 bp dsRNA molecule (e.g., RNAi molecules, siRNA molecules, miRNA molecules, and analogues thereof) has blunt ends.

In some embodiments, an anti-neu1, anti-neu3 or anti-neu4 dsRNA molecule (e.g., RNAi molecules, siRNA molecules, miRNA molecules, and analogues thereof) is assembled from two separate oligonucleotides, where one strand is the sense strand and the other is the anti-sense strand, wherein the anti-sense and sense strands are self-complementary (i.e. each strand comprises nucleotide sequence that is complementary to nucleotide sequence in the other strand; such as where the anti-sense strand and sense strand form a duplex or double stranded structure, for example wherein the double stranded region is about 19 base pairs). In some embodiments, the anti-sense strand of an anti-neu1 or anti-neu3 dsRNA molecule (e.g., siRNA molecules, miRNA molecules, and analogues thereof) comprises a nucleotide sequence that is complementary to a nucleotide sequence in a target nucleic acid molecule or a portion thereof, and the sense strand comprises a nucleotide sequence corresponding to the target nucleic acid sequence or a portion thereof. In some embodiments, an anti-neu1 or anti-neu3 dsRNA molecule (e.g., RNAi molecules, siRNA molecules, miRNA molecules, and analogues thereof) is assembled from a single oligonucleotide, where the self-complementary sense and anti-sense regions of the dsRNA molecule (e.g., RNAi molecules, siRNA molecules, miRNA molecules, and analogues thereof) are linked by means of a nucleic acid-based or non-nucleic acid-based linker(s).

In some embodiments, an anti-neu1, anti-neu3 or anti-neu4 dsRNA molecule (e.g., RNAi molecules, siRNA molecules, miRNA molecules, and analogues thereof) comprises a single stranded polynucleotide having nucleotide sequence complementary to nucleotide sequence in a target nucleic acid molecule or a portion thereof (for example, where such dsRNA molecule (e.g., RNAi molecules, siRNA molecules, miRNA molecules, and analogues thereof) does not require the presence within the dsRNA molecule (e.g., RNAi molecules, siRNA molecules, miRNA molecules, and analogues thereof) of nucleotide sequence corresponding to the target nucleic acid sequence or a portion thereof), wherein the single stranded polynucleotide further comprises a terminal phosphate group, such as a 5′-phosphate, or 5′,3′-diphosphate.

In other embodiments, an anti-neu1, anti-neu3 or anti-neu4 dsRNA molecule (e.g., RNAi molecules, siRNA molecules, miRNA molecules, and analogues thereof) comprises separate sense and anti-sense sequences or regions, wherein the sense and anti-sense regions are covalently linked by nucleotide or non-nucleotide linker molecules, or are alternately non-covalently linked by ionic interactions, hydrogen bonding, van der Waals interactions, hydrophobic interactions, and/or stacking interactions.

The terminal structure of dsRNA molecules described herein is either blunt or cohesive (overhanging). In some embodiments, the cohesive (overhanging) end structure is a 3′ overhang or a 5′ overhang. In some embodiments, the number of overhanging nucleotides is any length as long as the overhang does not impair gene silencing activity. In some embodiments, an overhang sequence is not complementary (anti-sense) or identical (sense) to the neu1, neu3 or neu4 sequence. In some embodiments, the overhang sequence contains low molecular weight structures (for example a natural RNA molecule such as tRNA, rRNA or tumor or CTC RNA, or an artificial RNA molecule).

The total length of dsRNA molecules having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp.

In some embodiments, the terminal structure of an anti-neu1, anti-neu3 or anti-neu4 dsRNA molecule (e.g., RNAi molecules, siRNA molecules, miRNA molecules, and analogues thereof) has a stem-loop structure in which ends of one side of the double-stranded nucleic acid are connected by a linker nucleic acid, e.g., a linker RNA. In some embodiments, the length of the double-stranded region (stem-loop portion) is 15 to 49 bp, often 15 to 35 bp, and more commonly about 21 to 30 bp long.

In some embodiments, an anti-neu1, anti-neu3 or anti-neu4 dsRNA molecule is a polynucleotide with a duplex, asymmetric duplex, hairpin or asymmetric hairpin secondary structure, having self-complementary sense and anti-sense regions, wherein the anti-sense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

In some embodiments, an anti-neu1, anti-neu3 or anti-neu4 dsRNA molecule (e.g., RNAi molecules, siRNA molecules, miRNA molecules, and analogues thereof) comprises a circular nucleic acid molecule, wherein the dsRNA molecule (e.g., RNAi molecules, siRNA molecules, miRNA molecules, and analogues thereof) is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

In some embodiments, a circular dsRNA molecule (e.g., RNAi molecules, siRNA molecules, miRNA molecules, and analogues thereof) contains two loop motifs, wherein one or both loop portions of the dsRNA molecule (e.g., RNAi molecules, siRNA molecules, miRNA molecules, and analogues thereof) is biodegradable. In some embodiments, degradation of the loop portions of a circular dsRNA molecule (e.g., RNAi molecules, siRNA molecules, miRNA molecules, and analogues thereof) generates a double-stranded dsRNA molecule (e.g., RNAi molecules, siRNA molecules, miRNA molecules, and analogues thereof) with 3′-terminal overhangs, such as 3-terminal nucleotide overhangs comprising about 2 nucleotides.

The sense strand of a double stranded dsRNA molecule (e.g., RNAi molecules, siRNA molecules, miRNA molecules, and analogues thereof) may have a terminal cap moiety such as an inverted deoxybasic moiety, at the 3-end, 5′-end, or both 3′ and 5′-ends of the sense strand.

In some embodiments, the 3′-terminal nucleotide overhangs of an anti-neu1, anti-neu3 or anti-neu4 dsRNA molecule (e.g., RNAi molecules, siRNA molecules, miRNA molecules, and analogues thereof) comprise ribonucleotides or deoxyribonucleotides that are chemically-modified at a nucleic acid sugar, base, or backbone. In some embodiments, the 3′-terminal nucleotide overhang comprises one or more universal base ribonucleotides. In some embodiments, the 3′-terminal nucleotide overhang comprises one or more acyclic nucleotides.

### Selection of RNAi Molecules

In some embodiments, an anti- anti-neu1, anti-neu3 or anti-neu4 dsRNA molecule (e.g., RNAi molecules, siRNA molecules, miRNA molecules, and analogues thereof) disclosed herein is capable of specifically binding to desired neu1 or neu3 variants while being incapable of specifically binding to non-desired neu1 or neu3 variants.

In some embodiments, an anti-neu1, anti-neu3 or anti-neu4 dsRNA molecule (e.g., RNAi molecules, siRNA molecules, miRNA molecules, and analogues thereof) is selected for use in a method disclosed herein based on predictions of the stability of molecule. In some embodiments, a prediction of stability is achieved by employing a theoretical melting curve wherein a higher theoretical melting curve indicates an increase in the molecule's stability and a concomitant decrease in cytotoxic effects. In some embodiments, stability of an anti-neu1 or anti-neu3 dsRNA molecule (e.g., RNAi molecules, siRNA molecules, miRNA molecules, and analogues thereof) is determined empirically by measuring the hybridization of a single modified RNA strand containing one or more universal-binding nucleotide(s) to a complementary neu1 or neu3 sequence within, for example, a polynucleotide array. In some embodiments, the melting temperature (i.e., the Tm value) for each modified RNA and complementary RNA immobilized on the array is determined and, from this Tm value, the relative stability of the modified RNA pairing with a complementary RNA molecule determined.

In some embodiments, an anti-neu1, anti-neu3 or anti-neu4 dsRNA molecule (e.g., RNAi molecules, siRNA molecules, miRNA molecules, and analogues thereof) is selected for use in a method disclosed herein based on “off-target” profiling whereby one or more dsRNA molecules is administered to a cell(s), either in vivo or in vitro, and total mRNA is collected, and used to probe a microarray comprising oligonucleotides having one or more nucleotide sequence from a panel of known genes, including non-target genes. The “off-target” profile of the modified dsRNA molecule (e.g., RNAi molecules, siRNA molecules, miRNA molecules, and analogues thereof) is quantified by determining the number of non-target genes having reduced expression levels in the presence of the RNAi molecule. The existence of “off target” binding indicates an anti-neu1 or anti-neu3 dsRNA molecule (e.g., RNAi molecules, siRNA molecules, miRNA molecules, and analogues thereof) that is capable of specifically binding to one or more non-target gene. Ideally, an anti-neu1 or anti-neu3 dsRNA molecule (e.g., RNAi molecules, siRNA molecules, miRNA molecules, and analogues thereof) applicable to therapeutic use will exhibit a high Tm value while exhibiting little or no “off-target” binding.

In some embodiments, an anti-neu1, anti-neu3 or anti-neu4 dsRNA molecule (e.g., RNAi molecules, siRNA molecules, miRNA molecules, and analogues thereof) is selected for use in a method disclosed herein by use of a report gene assay. In some embodiments, a reporter gene construct comprises a constitutive promoter, for example the cytomegalovirus (CMV) or phosphoglycerate kinase (PGK) promoter, operably fused to, and capable of modulating the expression of, one or more reporter gene such as, for example, a luciferase gene, a chloramphenicol (CAT) gene, and/or a β-galactosidase gene, which, in turn, is operably fused in-frame with an oligonucleotide (typically between about 15 base-pairs and about 40 base-pairs, more typically between about 19 base-pairs and about 30 base-pairs, most typically 20, 21, 22, 23, 24, 25, 26, 27, 28, or 29 base-pairs) that contains a target sequence for the one or more RNAi molecules. In some embodiments, individual reporter gene expression constructs are co-transfected with one or more RNAi molecules. In some embodiments, the capacity of a given dsRNA molecule (e.g., RNAi molecules, siRNA molecules, miRNA molecules, and analogues thereof) to reduce the expression level of each of the contemplated gene variants is determined by comparing the measured reporter gene activity from cells transfected with and without the modified RNAi molecule.

In some embodiments, an anti-neu1, anti-neu3 or anti-neu4 dsRNA molecule (e.g., RNAi molecules, siRNA molecules, miRNA molecules, and analogues thereof) is selected for use in a method disclosed herein by assaying its ability to specifically bind to an mRNA, such as an mRNA expressed by a target cell.

The present disclosure also relates to the use of the above-mentioned inhibitors of the disclosure and in the case of small molecule inhibitors, their pharmaceutically acceptable salts, esters, and solvates thereof in the preparation of a medicament, a combination or a kit.

### Compositions, Combination and Kits

### Compositions

The present disclosure also relates to pharmaceutical compositions comprising the above-mentioned inhibitors of the disclosure or, in the case of small molecule inhibitors, their pharmaceutically acceptable salts, esters and solvates thereof.

Without being so limited, the medicaments/pharmaceutical compositions of the disclosure may be administered orally, for example in the form of tablets, coated tablets, dragees, hard or soft gelatin capsules, solutions, emulsions or suspensions. Administration can also be carried out rectally, for example using suppositories; locally, topically, or percutaneously, for example using ointments, creams, gels or solutions; or parenterally, e.g., intravenously, intramuscularly, subcutaneously, intrathecally or transdermally, using for example injectable solutions. Furthermore, administration can be carried out sublingually, nasally, or as ophthalmological preparations or an aerosol, for example in the form of a spray, such as a nasal spray.

For the preparation of tablets, coated tablets, dragees or hard gelatin capsules, the compounds of the present disclosure may be admixed with any known pharmaceutically inert, inorganic or organic excipient and/or carrier. Examples of suitable excipients/carriers include lactose, maize starch or derivatives thereof, talc or stearic acid or salts thereof.

Suitable excipients for use with soft gelatin capsules include for example vegetable oils, waxes, fats, semi-solid or liquid polyols etc. According to the nature of the active ingredients it may however be the case that no excipient is needed at all for soft gelatin capsules.

For the preparation of solutions and syrups, excipients which may be used include for example water, polyols, saccharose, invert sugar and glucose.

For injectable solutions, excipients which may be used include for example water, saline, alcohols, polyols, glycerin, vegetable oils and other appropriate excipients.

For suppositories, and local or percutaneous application, excipients which may be used include for example natural or hardened oils, waxes, fats and semi-solid or liquid polyols.

The medicaments/pharmaceutical compositions may also contain preserving agents, solubilizing agents, stabilizing agents, wetting agents, emulsifiers, sweeteners, colorants, odorants, salts for the variation of osmotic pressure, buffers, coating agents or antioxidants. They may also contain other therapeutically active agents.

Intravenous, or oral administrations are preferred forms of use. The dosages in which the inhibitors of the disclosure are administered in effective amounts depend on the nature of the specific active ingredient, the age and the requirements of the patient and the mode of application.

As mentioned above, the pharmaceutical compositions of the disclosure can contain a pharmaceutically acceptable carrier including, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions.

Pharmaceutically acceptable carriers also can include physiologically acceptable aqueous vehicles (e.g., physiological saline) or other known carriers appropriate to specific routes of administration.

The inhibitors of the disclosure may be incorporated into dosage forms in conjunction with any of the vehicles which are commonly employed in pharmaceutical preparations, e.g., talc, gum arabic, lactose, starch, magnesium stearate, cocoa butter, aqueous or non-aqueous solvents, oils, paraffin derivatives or glycols. Emulsions such as those described in U.S. Pat. No. 5,434,183, incorporated herein by reference, may also be used in which vegetable oil (e.g., soybean oil or safflower oil), emulsifying agent (e.g., egg yolk phospholipid) and water are combined with glycerol. Methods for preparing appropriate formulations are well known in the art (see e.g., Remington's Pharmaceutical Sciences, 16th Ed., 1980, A. Oslo Ed., Easton, Pa. incorporated herein by reference).

In cases where parenteral administration is elected as the route of administration, preparations containing the inhibitors of the disclosure may be provided to patients in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

It is a prerequisite that all adjuvants used in the manufacture of the preparations, such as carriers, are non-toxic and more generally pharmaceutically acceptable.

As used herein, “pharmaceutically acceptable” such as pharmaceutically acceptable carrier, excipient, etc., means pharmacologically acceptable and substantially non-toxic to the subject to which the particular inhibitor is administered.

Any amount of a pharmaceutical composition can be administered to a subject. The dosages will depend on many factors including the mode of administration. Typically, the amount of the inhibitor of the disclosure contained within a single dose will be an amount that effectively prevent, delay or treat the disease or condition to be treated, delayed or prevented without inducing significant toxicity.

The effective amount of the inhibitors of the disclosure may also be measured directly. The effective amount may be given daily or weekly or fractions thereof. Typically, a pharmaceutical composition of the disclosure can be administered in an amount from about 0.001 mg up to about 500 mg per kg of body weight per day (e.g., 10 mg, 50 mg, 100 mg, or 250 mg). Dosages may be provided in either a single or multiple dosage regimen. For example, in some embodiments the effective amount may range from about 1 mg to about 25 grams of the composition per day, about 50 mg to about 10 grams of the composition per day, from about 100 mg to about 5 grams of the composition per day, about 1 gram of the composition per day, about 1 mg to about 25 grams of the composition per week, about 50 mg to about 10 grams of the composition per week, about 100 mg to about 5 grams of the composition every other day, and about 1 gram of the composition once a week.

These are simply guidelines since the actual dose must be carefully selected and titrated by the attending physician based upon clinical factors unique to each patient. The optimal daily dose will be determined by methods known in the art and will be influenced by factors such as the age of the patient and other clinically relevant factors. In addition, patients may be taking medications for other diseases or conditions. The other medications may be continued during the time that the pharmaceutical composition of the disclosure is given to the patient, but it is particularly advisable in such cases to begin with low doses to determine if adverse side effects are experienced.

### Combinations

In accordance with another aspect, there is provided a combination of at least one of the inhibitors described herein (e.g., a specific neu1 inhibitor, a specific neu3 inhibitor, a specific neu4 inhibitor, or a bispecific neu1, neu3 or neu4 inhibitor (e.g., neu3/neu4 inhibitor)) with another of the inhibitors described herein and/or with another agent that modulates inflammation (e.g., anti-inflammatory agent) and/or with a non-pharmaceutical treatment/regimen. Without being so limited, such anti-inflammatory agents include NSAIDs, etc.

In accordance with another aspect, when the targeted disease is ITP, there is provided a combination of at least one of the inhibitors described herein (e.g., a specific neu1 inhibitor, a specific neu3 inhibitor, a specific neu4 inhibitor, or a bispecific neu1, neu3 or neu4 inhibitor (e.g., neu3/neu4 inhibitor)) with another of the inhibitors described herein and/or with another agent that treats or prevents ITP or a symptom thereof including a IVIg, a corticosteroid, eltrombopag, romiplostim, and/or fostamatinib.

In accordance with an aspect, there is provided a composition comprising at least one of the inhibitors as defined herein, and (i) another of the inhibitors described herein; (ii) another agent that modulates inflammation; (iii) a pharmaceutically acceptable carrier; or (iv) a combination of at least two of (i) to (iii). In accordance with another aspect, there is provided a method of modulating leukocytes activation (e.g., adhesion and/or transmigration and/or cytokine response (e.g., in response to bacterial infection)) comprising administering an effective amount of at least one of the inhibitors described herein (e.g., a specific neu1 inhibitor, a specific neu3 inhibitor, a specific neu3 inhibitor, or a bispecific neu1, neu3 or neu4 inhibitor (e.g., neu3/neu4 inhibitor)); and (i) another of the inhibitors described herein (e.g., a specific neu1, neu3 or neu4 (or bispecific neu1, neu3 or neu4 inhibitor (e.g., neu3/neu4 inhibitor)); (ii) another that modulates inflammation; and/or (iii) a non-pharmaceutical means.

In a specific embodiment, said composition is a pharmaceutical composition. In another specific embodiment, the composition comprises (i) an inhibitor as defined herein; and (ii) another agent that modulates inflammation.

In accordance with an aspect, there is provided a composition comprising at least one of the inhibitors as defined herein, and (i) another of the inhibitors described herein; (ii) another agent that modulates thrombocyte clearance (or that prevents or treats ITP or a symptom thereof); (iii) a pharmaceutically acceptable carrier; or (iv) a combination of at least two of (i) to (iii). In accordance with another aspect, there is provided a method of modulating inflammation (or preventing or treating ITP or a symptom thereof) comprising administering an effective amount of at least one of the inhibitors described herein (e.g., a specific neu1 inhibitor, a specific neu3 inhibitor, a specific neu3 inhibitor, or a bispecific neu1, neu3 or neu4 inhibitor (e.g., neu3/neu4 inhibitor)); and (i) another of the inhibitors described herein (e.g., a specific neu1, neu3 or neu4 (or bispecific neu1, neu3 or neu4 inhibitor (e.g., neu3/neu4 inhibitor)); (ii) another that modulates thrombocyte clearance (or that prevents or treats ITP or a symptom thereof); and/or (iii) a non-pharmaceutical means.

In a specific embodiment, said composition is a pharmaceutical composition. In another specific embodiment, the composition comprises (i) an inhibitor as defined herein; and (ii) another agent that modulates inflammation. In another specific embodiment, the composition comprises (i) an inhibitor as defined herein; and (ii) another agent that modulates thrombocyte clearance (or that prevents or treats ITP or a symptom thereof).

### Kits

In accordance with another aspect of the present disclosure, there is provided a kit comprising the inhibitor defined herein or the above-mentioned composition, and instructions to use same in the modulation of leukocytes activation (e.g., leukocytes adhesion and/or transmigration and/or cytokine response (e.g., in response to bacterial infection)) of the instant disclosure.

In a specific embodiment of the kit, the kit comprises: (i) another of the inhibitors described herein; (ii) another agent that modulates inflammation; (iii) instructions to use same in the modulation of leukocytes activation (e.g., leukocytes adhesion and/or transmigration and/or cytokine response (e.g., in response to bacterial infection)); or (iv) a combination of at least two of (i) to (iii).

In accordance with another aspect of the present disclosure, there is provided a kit comprising the inhibitor defined herein or the above-mentioned composition, and instructions to use same in the modulation of thrombocyte clearance (or that prevents or treats ITP or a symptom thereof) of the instant disclosure.

In a specific embodiment of the kit, the kit comprises: (i) another of the inhibitors described herein; (ii) another agent that modulates thrombocyte clearance (or that prevents or treats ITP or a symptom thereof); (iii) instructions to use same in the modulation of thrombocyte clearance (or that prevents or treats ITP or a symptom thereof); or (iv) a combination of at least two of (i) to (iii).

### Screening Methods

In accordance with another aspect of the present disclosure, there is provided a method of identifying an agent that modulates leukocytes activation (e.g., leukocytes adhesion and/or transmigration and/or cytokine response (e.g., in response to bacterial infection)) (and optionally modulates inflammation), said method comprising contacting a neuraminidase 1, a neuraminidase 3 or a neuraminidase 4 (or a cell expressing same) with a candidate compound (and eventually a neuraminidase 1, neuraminidase 3 or neuraminidase 4 substrate)) and determining the effect of said candidate compound on the neuraminidase 1, 3 or 4 expression and/or activity (e.g., ability of compound to prevent neuraminidase 1, neuraminidase 3 or a neuraminidase 4 modulate leukocyte activation), wherein a decrease in the expression and/or activity of the neuraminidase 1, 3 or 4 in the presence as compared to in the absence of said candidate compound is an indication that said candidate compound may modulate leukocytes activation (e.g., leukocytes adhesion and/or transmigration and/or cytokine response (e.g., in response to bacterial infection)).

As used herein the terms “neuraminidase 1, neuraminidase 3 or neuraminidase 4 activity” refers to ApoB desialylation (e.g., in plasma) by one or more of these enzymes and to events downstream of this desialylation such as LDL uptake by macrophages, formation of foam cells, LDL incorporation in arterial walls, increase of fatty streak regions number on arterial walls, increase of fatty streak regions size on arterial walls, infiltration of T cell in atherosclerotic lesions, infiltration of macrophages, vascular smooth muscle cells or leukocytes in atherosclerotic lesions, production of extracellular matrix molecules, collagen and elastin, formation of a fibrous cap that covers the plaque, cellular necrosis, plaque rupture and thrombosis; and leukocyte transmigration and adhesion. Neuraminidase 1, neuraminidase 4 or neuraminidase 3 activity can further be measured in vitro and in situ using substrates such as sialylated ApoB, 4-Mu-5NeuAc, sialyllactose or other knows substrates of neuraminidases/sialidases. The terms “neuraminidase 1, neuraminidase 3 or neuraminidase 4 activity” may also refer to modification of glycoproteins found on leukocytes and/or thrombocytes leading to hyposialylation of any of these cells, and events downstream of this event including activation of leukocytes and/or or thrombocytes receptors, transmigration (e.g., of leukocytes), adhesion (e.g., of leukocytes), opsonization, cytokines response or clearance of thrombocytes from the bloodstream.

As used herein the terms “subject in need thereof” refer to a subject who would benefit from receiving an effective amount of the inhibitor of the present disclosure (e.g., subject having a bacterial infection or subject having IPT). It refers to an animal and to a human. The inhibitors of the present disclosure may be used for veterinary applications and be used in pets or other animals (e.g., pets such as cats, dogs, horses, etc.; and cattle, fishes, swine, poultry, etc.).

The use of the terms “a” and “an” and “the” and similar referents in the context of describing the disclosure (especially in the context of the following items) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. In embodiments, it may mean plus or minus 10% of the numerical value qualified.

Unless otherwise defined, all technical and scientific terms used herein have the same meaning as commonly understood by one of ordinary skill in the art to which this disclosure belongs.

Other objects, advantages and features of the present disclosure will become more apparent upon reading of the following non-restrictive description of specific embodiments thereof, given by way of example only with reference to the accompanying drawings.

### DESCRIPTION OF ILLUSTRATIVE EMBODIMENTS

The present disclosure is illustrated in further details by the following non-limiting examples.

### Example 1: Inhibitors Design and Synthesis

A series of compounds were designed, synthesized and their inhibitory effects were tested against the four isoenzymes of human neuraminidases. The inventors first varied the aromatic ring of a C9-triazole DANA derivative, including electron-withdrawing and electron-donating groups, negatively and positively charged groups, as well as larger phenyl and phenoxyl groups (7, FIG. 1A).

The inventors also synthesized compounds with different phenyltriazole groups at C9; nitrogen-containing groups at C4, including guanidine (6), azido, amino groups; and combinations with modifications at both C9 and C4 (8, FIGS. 1A; and 1D, 1F, etc.).

Compounds with phenyltriazole groups at C9 were synthesized using C9-azido-DANA methyl ester (9), which could be obtained from Neu5Ac in 6 steps (Zou, 2010). CuCAAC (copper-catalyzed azide-alkyne cycloaddition) was applied with para-substituted phenylalkynes to introduce the various C9 modifications (7a-7j, FIG. 1B).

For compounds with combined C4 and C9 modifications, two strategies were adopted (synthetic routes shown in FIGS. 1C-1D). Compound 8a started from the C9-modified derivative (10h, FIG. 1C). C4 modifications were then realized through a previously reported strategy (Shidmoossavee, 2013): an azide was introduced to the C4 position via nucleophilic ring opening of an oxazoline intermediate (11). The Staudinger reduction (Staudinger, 1919) then gave the C4-amino derivative (14), which could be converted to a guanidino group using N,N′-di-Boc-1H-pyrazole-1-carboxamidine, providing the final C4, C9 modified compound 8a after deprotection. The C4-azido and C4-amino derivatives were also deprotected to give corresponding compounds (13 and 15) for testing. Compound 14 was treated with 1,1′-Carbonyldiimidazole and β-alanine methyl ester to form a urea moiety at C4. The methyl esters were then hydrolyzed to give compound 18. To prepare compound 8b, the inventors started from a fully protected C4-amino-DANA derivative (19, FIG. 1C), which can be synthesized from Neu5Ac in several steps as reported (Shidmoossavee, 2013). C9-azido group was introduced via a two-step strategy of tosylation (20, 21), followed by displacement with NaN₃ to give the C9-azido, C4-amino derivative, 22. At this point, the guanidino group was introduced at C4, then a subsequent CuAAC was applied to introduce the biphenyltriazole group at C9, 24. The final product (8b) was obtained after deprotection.

To generate compounds with only C4 amide moieties, compound 19 was treated with different anhydrides or acyl chlorides to form the desired amides (synthetic route shown in FIG. 1E; 25). Final products (25a-d) were obtained by hydrolyzing the C1-methyl ester with sodium hydroxide.

General Synthetic Procedures. All reagents and solvents were purchased from Sigma-Aldrich unless otherwise noted and used without further purification. Reactions were monitored with TLC (Merck TLC Silica gel 60 F₂₅₄) and spots were visualized under UV light (254 nm) or by charring with 0.5% H₂SO₄/EtOH. Compounds were purified by flash column chromatography with silica gel (SiliaFlash™ F60, 40-63 μm particle size) or recrystallization with the solvent mixtures specified in the corresponding experiments. Proton (¹H) and carbon (¹³C) NMR spectra were recorded on Varian™ 400 (400 MHz for ¹H; 100 MHz for ¹³C) or Varian™ 500 (500 MHz for ¹H; 125 MHz for ¹³C). High-resolution mass spectrometry (HR-MS) analysis was performed on Agilent Technologies™ 6220 TOF spectrometer. Purity of all final products used for inhibitor assays was determined to be ≥95% by HPLC.

### Example 2: 5-Acetamido-2,6-anhydro-3,5-dideoxy-D-glycero-D-galacto-non-2-enonic acid (DANA, 1)

Was synthesized as previously reported. (Zou, 2010) ¹H NMR (500 MHz, cd₃od) δ 5.67 (d, J=2.3 Hz, 1H, H-3), 4.36 (dd, J=8.6, 2.3 Hz, 1H, H-4), 4.10 (dd, J=10.9, 1.1 Hz, 1H, H-6), 3.99 (dd, J=10.9, 8.6 Hz, 1H, H-5), 3.87 (ddd, J=9.1, 5.4, 3.1 Hz, 1H, H-8), 3.80 (dd, J=11.4, 3.1 Hz, 1H, H-9), 3.65 (dd, J=11.4, 5.4 Hz, 1H, H-9′), 3.52 (dd, J=9.1, 1.1 Hz, 1H, H-7), 2.02 (s, 3H, COCH₃). ¹³C NMR (125 MHz, cd₃od) δ 174.68, 170.02 (C═O), 149.95 (C-2), 108.34 (C-3), 77.24 (C-6), 71.29 (C-8), 70.22 (C-7), 68.70 (C-4), 64.94 (C-9), 51.96 (C-5), 22.82 (COCH₃). HR-MS (ESI) calcd. for C₁₁H₁₆NO₈ [M−H]⁻, 290.0876; found 290.0879.

### Example 3: 5-Acetamido-2,6-anhydro-4-guanidino-3,4,5-trideoxy-D-glycero-D-galacto-non-2-enonic acid (Zanamivir (6))

Was synthesized as previously reported (von Itzstein, 1994; von Itzstein, 1993). ¹H NMR (500 MHz, d₂o) δ 5.70 (d, J=1.9 Hz, 1H, H-3), 4.54 (dd, J=9.3, 1.9 Hz, 1H, H-4), 4.46 (m, 1H, H-6), 4.29 (dd, J=10.5, 9.3 Hz, 1H, H-5), 4.02 (ddd, J=9.1, 6.2, 2.5 Hz, 1H, H-8), 3.96 (dd, J=11.9, 2.5 Hz, 1H, H-9), 3.77-3.69 (m, 2H, H-7, H-9′), 2.11 (s, 3H, COCH₃). ¹³C NMR (125 MHz, d₂o) δ 175.38, 170.10 (C═O), 157.99 (C═N), 150.19 (C-2), 104.79 (C-3), 76.33 (C-6), 70.74 (C-8), 69.11 (C-7), 64.03 (C-9), 52.11 (C-4), 48.71 (C-5), 22.93 (COCH₃). HR-MS (ESI) calcd. for C₁₂H₂₁N₄O₇ [M+H]⁺, 333.1405; found 333.1400.

### Example 4: General Procedure of CuAAC Reaction and Hydrolyzation of Methyl Ester

To a solution of Methyl 5-acetamido-9-azido-2,6-anhydro-3,5-dideoxy-D-glycero-D-galacto-non-2-enonate (9) (Zou, 2010) (1 eq) and the corresponding alkyne (1.5 eq) in THF-H₂O (2:1), sodium L-ascorbate (0.5 eq) and copper (II) sulfate (0.5 eq) were added sequentially. The reaction was kept stirring at room temperature and monitored by TLC until no azide was remained. Silica gel was then added to the reaction mixture and the solvent was removed under reduced pressure. The residue was separated by flash chromatography to provide the desired products with yields of 42%-88%. To hydrolyze the C1-methyl ester, the product was dissolved in MeOH, and 0.5 M NaOH was added. The mixture was kept stirring at room temperature. After completion, the mixture was neutralized with Amberlite™ IR-120 (H⁺ form), filtered and purified by flash chromatography to provide the desired products with yields of 45%-88%.

### Example 5: 5-Acetamido-9-(4-(dimethylamino)phenyl)-2,6-anhydro-3,5-dideoxy-D-glycero-D-galacto-non-2-enonic acid (7a)

Compound 7a was prepared as above in 75% yield (86 mg). ¹H NMR (500 MHz, CD₃OD) δ 8.11 (s, 1H, Triazole-H5), 7.64 (d, J=8.9 Hz, 2H, Ar—H), 6.81 (d, J=8.9 Hz, 2H, Ar—H), 5.90 (d, J=2.3 Hz, 1H, H-3), 4.49 (dd, J=14.0, 7.5 Hz, 1H, H-9′), 4.40 (dd, J=8.7, 2.3 Hz, 1H, H-4), 4.31-4.25 (m, 1H, H-8), 4.14 (dd, J=10.9, 1.0 Hz, 1H, H-6), 3.99 (dd, J=10.9, 8.7 Hz, 1H, H-5), 3.41 (dd, J=9.2, 1.0 Hz, 1H, H-7), 2.96 (s, 6H, N(CH₃)₂), 1.99 (s, 3H, COCH₃). ¹³C NMR (125 MHz, CD₃OD) δ 175.16 (C═O), 152.12, 127.59, 120.10, 113.93 (Ar—C), 149.18 (Triazole-C4), 121.83 (Triazole-C5), 77.67 (C-6), 71.23 (C-7), 69.79 (C-4), 68.01 (C-8), 55.13 (C-9), 51.96 (C-5), 40.78 (N—CH₃), 22.65 (COCH₃). HR-MS (ESI) calcd. for C₂₁H₂₆N₅O₇ [M−H]⁻, 460.1832; found 460.1834.

### Example 6: 5-Acetamido-9-(4-acetamidophenyl)-2,6-anhydro-3,5-dideoxy-D-glycero-D-galacto-non-2-enonic acid (7b)

Compound 7b was prepared as above in 79% yield (90 mg). ¹H NMR (500 MHz, CD₃OD) δ 8.28 (s, 1H, Triazole-H5), 7.76 (d, J=8.2 Hz, 2H, Ar—H), 7.62 (d, J=8.2 Hz, 2H, Ar—H), 5.70 (s, 1H, H-3), 4.50 (dd, J=13.5, 7.7 Hz, 1H, H-9′), 4.35 (d, J=8.6 Hz, 1H, H-4), 4.30-4.27 (m, 1H, H-8), 4.10 (d, J=10.7 Hz, 1H, H-6), 4.04-3.96 (m, 1H, H-5), 3.39 (d, J=7.7 Hz, 1H, H-7), 2.13, 1.98 (2×s, 3H, 2×COCH₃). ¹³C NMR (125 MHz, d₆-DMSO) δ 172.10, 168.34, 168.25 (C═O), 165.18 (C═O), 147.63 (C-2), 145.74 (Triazole-C4), 121.67 (Triazole-C5), 138.80, 138.69, 125.72, 125.44, 119.20, 119.11 (Ar—C), 108.60 (C-3), 75.66 (C-6), 69.95 (C-7), 68.10 (C-4), 65.90 (C-8), 53.70 (C-9), 50.81 (C-5), 22.97, 22.49 (COCH₃). HR-MS (ESI) calcd. for C₂₁H₂₄N₅O₈ [M−H]⁻, 474.1625; found 474.1636.

### Example 7: 5-Acetamido-9-(4-amidophenyl)-2,6-anhydro-3,5-dideoxy-D-glycero-D-galacto-non-2-enonic acid (7c)

Compound 7c was prepared as above in 77% yield (80 mg). ¹H NMR (500 MHz, CD₃OD) δ 8.09 (s, 1H, Triazole-H5), 7.55 (d, J=8.6 Hz, 2H), 6.79 (d, J=8.6 Hz, 2H), 5.88 (d, J=2.4 Hz, 1H, H-3), 4.82 (dd, J=14.1, 2.6 Hz, 1H, H-9), 4.48 (dd, J=14.1, 7.6 Hz, 1H, H-9′), 4.41 (dd, J=8.7, 2.4 Hz, 1H, H-4), 4.28 (ddd, J=9.5, 7.6, 2.6 Hz, 1H, H-8), 4.14 (dd, J=10.9, 1.0 Hz, 1H, H-6), 4.00 (dd, J=10.9, 8.7 Hz, 1H, H-5), 3.41 (dd, J=9.5, 1.0 Hz, 1H, H-7), 1.98 (s, 3H, COCH₃). ¹³C NMR (125 MHz, CD₃OD) δ 175.13, 166.94 (C═O), 149.13, 148.42 (Ar—C, Triazole-C4), 146.69 (C-2), 127.77, 116.96 (Ar—C), 122.01, 121.94 (Ar—C, Triazole-C5), 112.19 (C-3), 77.57 (C-6), 71.24 (C-7), 69.81 (C-4), 68.08 (C-8), 55.14 (C-9), 51.93 (C-5), 22.72 (COCH₃). HR-MS (ESI) calcd. for C₁₉H₂₂N₅O₇ [M−H]⁻, 432.1529; found 432.1513.

### Example 8: 5-Acetamido-9-(4-methylphenyl)-2,6-anhydro-3,5-dideoxy-D-glycero-D-galacto-non-2-enonic acid (7d)

Compound 7d was prepared as above in 86% yield (100 mg). ¹H NMR (500 MHz, CD₃OD) δ 8.24 (s, 1H, Trizole-H), 7.68 (d, J=8.0 Hz, 2H, Ar—H), 7.22 (d, J=8.0 Hz, 2H, Ar—H), 5.79 (s, 1H, H-3), 4.50 (dd, J=13.9, 7.4 Hz, 1H, H-9′), 4.39 (d, J=8.2 Hz, 1H, H-4), 4.29 (brs, 1H, H-8), 4.13 (d, J=10.9 Hz, 1H, H-6), 4.06-3.97 (m, 1H, H-5), 3.42 (d, J=9.0 Hz, 1H, H-7), 2.34 (s, 3H, PhCH₃), 1.98 (s, 3H, COCH₃). ¹³C NMR (125 MHz, CD₃OD) δ 175.00 (C═O), 148.64 (Triazole-C4), 123.08 (Triazole-C5), 139.31, 130.58, 129.01, 126.62 (Ar—C), 110.40 (C-3), 77.27 (C-6), 71.24 (C-7), 69.84 (C-4), 68.35 (C-8), 55.16 (C-9), 51.95 (C-5), 22.76 (COCH₃), 21.31 (PhCH₃). HR-MS (ESI) calcd. for C₂₀H₂₃N₄O₇ [M−H]⁻, 431.1567; found 431.1568.

### Example 9: 5-Acetamido-9-(4-methoxyphenyl)-2,6-anhydro-3,5-dideoxy-D-glycero-D-galacto-non-2-enonic acid (7e)

Compound 7e was prepared as above in 74% yield (80 mg). ¹H NMR (500 MHz, CD₃OD) δ 8.20 (s, 1H, Triazole-H5), 7.72 (d, J=8.9 Hz, 2H), 6.97 (d, J=8.9 Hz, 2H), 5.77 (d, J=2.0 Hz, 1H, H-3), 4.49 (dd, J=14.0, 7.6 Hz, 1H, H-9′), 4.39 (dd, J=8.7, 2.0 Hz, 1H, H-4), 4.32-4.25 (m, 1H, H-8), 4.12 (d, J=10.8 Hz, 1H, H-6), 4.00 (dd, J=10.8, 8.7 Hz, 1H, H-5), 3.81 (s, 3H, COCH₃), 3.41 (d, J=9.2 Hz, 1H, H-7), 1.98 (s, 3H, COCH₃). ¹³C NMR (125 MHz, CD₃OD) δ 174.96 (COCH₃), 161.27, 128.00, 124.40, 115.37 (Ar—C), 148.49 (Triazole-C4), 122.59 (Triazole-C5), 109.95 (C-3), 77.20 (C-6), 71.26 (C-7), 69.86 (C-4), 68.38 (C-8), 55.80 (PhOCH₃), 55.15 (C-9), 51.97 (C-5), 22.74 (COCH₃). HR-MS (ESI) calcd. for C₂₀H₂₃N₄O₈ [M−H]⁻, 447.1516; found 447.1527.

### Example 10: 5-Acetamido-9-(4-fluorophenyl)-2,6-anhydro-3,5-dideoxy-D-glycero-D-galacto-non-2-enonic acid (7f)

Compound 7f was prepared as above in 83% yield (80 mg). ¹H NMR (500 MHz, CD₃OD) δ 8.28 (s, 1H, Triazole-H5), 7.87-7.80 (m, 2H, Ar—H), 7.20-7.13 (m, 2H, Ar—H), 5.92 (d, J=2.4 Hz, 1H, H-3), 4.86 (dd, J=14.0, 2.6 Hz, 1H, H-9), 4.51 (dd, J=14.0, 7.7 Hz, 1H, H-9′), 4.41 (dd, J=8.7, 2.4 Hz, 1H, H-4), 4.29 (ddd, J=9.6, 7.7, 2.6 Hz, 1H, H-8), 4.14 (dd, J=10.8, 1.1 Hz, 1H, H-6), 3.99 (dd, J=10.8, 8.7 Hz, 1H, H-5), 3.43 (dd, J=9.6, 1.1 Hz, 1H, H-7), 1.99 (s, 3H, COCH₃). ¹³C NMR (125 MHz, CD₃OD) δ 175.15 (C═O), 164.09 (d, J=245.9 Hz, Ar—C), 147.63 (Triazole-C4), 123.31 (Triazole-C5), 128.61 (d, J=8.2 Hz, Ar—C), 128.34 (d, J=3.2 Hz, Ar—C), 116.77 (d, J=22.0 Hz, Ar—C), 112.86 (C-3), 77.70 (C-6), 71.30 (C-7), 69.80 (C-4), 67.95 (C-8), 55.25 (C-9), 51.96 (C-5), 22.64 (COCH₃). HR-MS (ESI) calcd. for C₁₉H₂₀FN₄O₇ [M−H]⁻, 435.1316; found 435.1324.

### Example 11: 5-Acetamido-9-(4-(trifluoromethyl)phenyl)-2,6-anhydro-3,5-dideoxy-D-glycero-D-galacto-non-2-enonic acid (7g)

Compound 7g was prepared as above in 45% yield (40 mg). ¹H NMR (500 MHz, CD₃OD) δ 8.44 (s, 1H, Triazole-C), 8.02 (d, J=8.2 Hz, 2H, Ar—H), 7.72 (d, J=8.2 Hz, 2H, Ar—H), 5.95 (d, J=2.4 Hz, 1H, H-3), 4.90 (dd, J=14.0, 2.6 Hz, 1H, H-9), 4.53 (dd, J=14.0, 7.7 Hz, 1H, H-9′), 4.42 (dd, J=8.7, 2.4 Hz, 1H, H-4), 4.37-4.21 (m, 1H, H-8), 4.16 (dd, J=10.8, 1.0 Hz, 1H, H-6), 4.00 (dd, J=10.8, 8.7 Hz, 1H, H-5), 3.45 (dd, J=9.1, 1.0 Hz, 1H, H-7), 1.99 (s, 3H, COCH₃). ¹³C NMR (125 MHz, CD₃OD) δ 175.20 (C═O), 147.04 (Triazole-C4), 124.50 (Triazole-C5), 135.81 (Ar—C), 130.90 (q, J=32.3 Hz, Ar—C), 127.01 (Ar—C), 126.89 (q, J=3.8 Hz), 113.43 (C-3), 77.81 (C-6), 71.35 (C-7), 69.80 (C-4), 67.88 (C-8), 55.34 (C-9), 51.95 (C-9), 22.65 (COCH₃). HR-MS (ESI) calcd. for C₂₀H₂₀F₃N₄O₇[M−H]⁻, 485.1284; found 485.1282.

### Example 12: 5-Acetamido-9-(4-carboxyphenyl)-2,6-anhydro-3,5-dideoxy-D-glycero-D-galacto-non-2-enonic acid (7h)

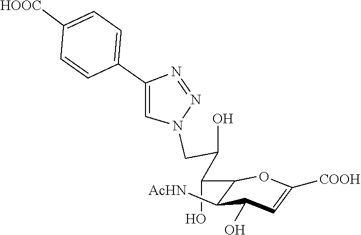

Compound 7h was prepared as above in 83% yield (100 mg). ¹H NMR (500 MHz, CD₃OD) δ 8.42 (s, 1H, C═CH—N), 8.06 (d, J=8.5 Hz, 2H, Ar—H), 7.90 (d, J=8.5 Hz, 2H, Ar—H), 5.84 (d, J=1.9 Hz, 1H, H-3), 4.88 (dd, J=14.0, 2.1 Hz, 1H, H-9), 4.54 (dd, J=14.0, 7.6 Hz, 1H, H-9′), 4.43 (dd, J=8.7, 1.9 Hz, 1H, H-4), 4.31 (t, J=7.6 Hz, 1H, H-8), 4.15 (d, J=10.9 Hz, 1H, H-6), 4.03 (dd, J=10.9, 8.7 Hz, 1H, H-5), 3.44 (d, J=7.6 Hz, 1H, H-7), 1.99 (s, 3H, COCH₃). ¹³C NMR (125 MHz, CD₃OD) δ 175.08, 169.69, 167.90 (C═O), 147.50, 124.53 (Triazole-C), 136.19, 131.49, 126.44 (Ar—C), 111.19 (C-3), 77.37 (C-6), 71.31 (C-7), 69.84 (C-4), 68.24 (C-8), 55.29 (C-9), 51.90 (C-5), 22.82 (COCH₃). HR-MS (ESI) calcd. for C₂₀H₂₁N₄O₉ [M−H]⁻, 461.1309; found 461.1316.

### Example 13: 5-Acetamido-9-(4-biphenyl)-2,6-anhydro-3,5-dideoxy-D-glycero-D-galacto-non-2-enonic acid (7i)

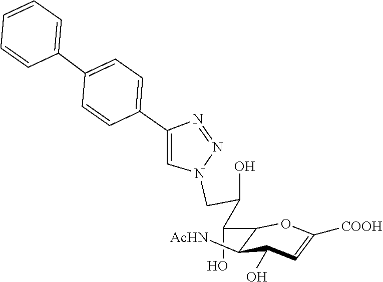

CuAAC reaction gave 77 mg crude protected product, which was deprotected to give the desired final product 52 mg (75%). ¹H NMR (500 MHz, CD₃OD) δ 8.34 (s, 1H, Triazole-H), 7.88 (d, J=8.1 Hz, 2H, Ar—H), 7.65 (d, J=8.1 Hz, 3H, Ar—H), 7.42 (t, J=7.5 Hz, 2H, Ar—H), 7.32 (t, J=7.5 Hz, 1H, Ar—H), 5.74 (d, J=1.9 Hz, 1H, H-3), 4.88 (dd, J=13.9, 2.2 Hz, 1H, H-9), 4.52 (dd, J=13.9, 7.7 Hz, H-9′), 4.39 (dd, J=8.7, 1.9 Hz, H-4), 4.37-4.21 (m, 1H, H-8), 4.13 (d, J=10.8 Hz, 1H, H-6), 4.02 (dd, J=10.8, 8.7 Hz, 1H, H-5), 3.42 (d, J=9.3 Hz, 1H, H-7), 2.00 (s, 3H, COCH₃). ¹³C NMR (125 MHz, CD₃OD) δ 174.93, 169.51 (C═O), 149.28, 123.47 (Triazole-C), 148.20 (C-2), 142.24, 141.76, 130.84, 129.94, 128.54, 128.48, 127.86, 127.10 (Ar—C), 77.14 (C-6), 71.34 (C-7), 69.83 (C-4), 68.49 (C-8), 55.25 (C-9), 52.00 (C-5), 22.77 (COCH₃). HR-MS (ESI) calcd. for C₂₅H₂₅N₄O₇ [M−H]⁻, 493.1723; found 493.1729.

### Example 14: 5-Acetamido-9-(4-phenoxyphenyl)-2,6-anhydro-3,5-dideoxy-D-glycero-D-galacto-non-2-enonic acid (7j)

Click reaction gave 77 mg crude protected product, which was deprotected to give the desired final product 40 mg (43%). ¹H NMR (500 MHz, CD₃OD) δ 8.24 (s, 1H, Triazole-H5), 8.24 (s, 1H), 7.78 (d, J=8.7 Hz, 2H, Ar—H), 7.35 (dd, J=8.7, 7.4 Hz, 2H, Ar—H), 7.12 (t, J=7.4 Hz, 1H, Ar—H), 7.05-6.98 (m, 4H, Ar—H), 5.93 (d, J=2.4 Hz, 1H, H-3), 4.85 (dd, J=14.0, 2.5 Hz, 1H, H-9), 4.50 (dd, J=14.0, 7.7 Hz, 1H, H-9′), 4.42 (dd, J=8.7, 2.4 Hz, 1H, H-4), 4.30 (ddd, J=10.0, 7.7, 2.5 Hz, 1H, H-8), 4.16 (m, 1H, H-6), 4.01 (dd, J=10.8, 8.8 Hz, 1H, H-5), 3.47-3.41 (m, 1H, H-7), 2.00 (s, 3H, COCH₃). ¹³C NMR (125 MHz, CD₃OD) δ 175.18, 166.12 (C═O), 158.95, 158.31, 131.00, 128.28, 126.98, 124.77, 120.19, 119.98 (Ar—C), 148.06 (Triazole-C4), 123.10 (Triazole-C5), 145.90 (C-2), 113.04 (C-3), 77.73 (C-6), 71.32 (C-7), 69.83 (C-4), 67.95 (C-8), 55.25 (C-9), 51.95 (C-5), 22.70 (COCH₃). HR-MS (ESI) calcd. for C₂₅H₂₅N₄O₈ [M−H]⁻, 509.1672; found 509.1674.

### Example 15: 5-Acetamido-9-(3-(4-(benzamidomethyl))-2,6-anhydro-3,5-dideoxy-D-glycero-D-galacto-non-2-enonic acid (26)

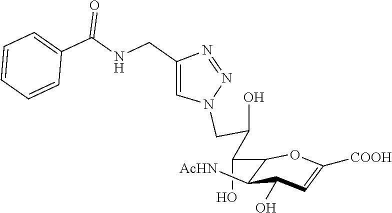

¹H NMR (500 MHz, CD₃OD) δ 7.93 (s, 1H, Triazole-H), 7.86-7.80 (m, 2H, Ar—H), 7.53 (t, J=7.4 Hz, 1H, Ar—H), 7.45 (t, J=7.6 Hz, 2H, Ar—H), 5.94 (d, J=2.5 Hz, 1H, H-3), 4.86-4.81 (m, 1H, H-9), 4.64 (s, 2H, N—CH₂-Triazole), 4.46-4.38 (m, 2H, H-9′, H-4), 4.22 (td, J=8.4, 2.4 Hz, 1H, H-8), 4.14 (d, J=11.1 Hz, 1H, H-6), 3.97 (dd, J=11.1, 8.8 Hz, 1H, H-5, H-5), 3.43 (d, J=8.4 Hz, 1H, H-7), 2.01 (s, 3H, COCH₃). ¹³C NMR (126 MHz, CD₃OD) δ 175.30 (N—C═O), 132.88, 129.62, 128.40 (Ar—C), 113.51 (C-3), 77.73 (C-6), 71.36 (C-7), 69.87 (C-4), 67.85 (C-8), 55.16 (C-9), 51.90 (C-5), 36.21 (N—CH₂-Triazole), 22.69 (COCH₃). HR-MS (ESI) calcd. for C₂₁H₂₄N₅O₈ [M−H]⁻, 474.1630; found 474.1624.

### Example 16: 5-Acetamido-9-(3-(4-(4-benzamidophenyl))-2,6-anhydro-3,5-dideoxy-D-glycero-D-galacto-non-2-enonic acid (27)

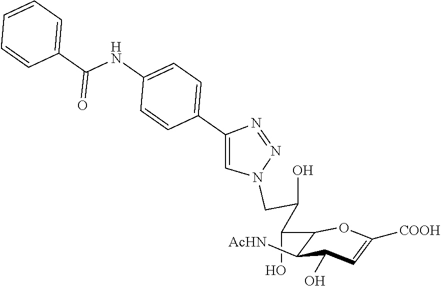

¹H NMR (500 MHz, CD₃OD) δ 8.31 (d, J=7.4 Hz, 1H, Triazole-H), 7.93 (d, J=7.3 Hz, 2H, Ar—H), 7.86-7.80 (m, 2H, Ar—H), 7.79 (d, J=8.7 Hz, 2H, Ar—H), 7.61-7.57 (m, 1H, Ar—H), 7.53 (d, J=6.5 Hz, 2H, Ar—H), 5.97 (s, 1H, H-3), 4.87 (d, J=11.9 Hz, 1H, H-9), 4.54 (dd, J=14.1, 7.4 Hz, 1H, H-9′), 4.44 (d, J=9.2 Hz, 1H, H-4), 4.35-4.27 (m, 1H, H-8), 4.17 (d, J=10.2 Hz, 1H, H-6), 4.03-3.95 (m, 1H, H-5), 3.44 (d, J=8.7 Hz, 1H, H-7), 2.01 (d, J=3.4 Hz, 3H, COCH₃). ¹³C NMR (126 MHz, CD₃OD) δ 133.10, 129.76, 128.66, 127.17, 122.63, 116.42 (Ar—C), 113.69 (C-3), 77.70 (C-6), 71.10 (C-7), 69.76 (C-4), 67.85 (C-8), 55.15 (C-9), 51.85 (C-5), 22.75 (COCH₃). HR-MS (ESI) calcd. for C₂₆H₂₆N₅O₈ [M−H]⁻, 536.1787; found 536.1782.

### Example 17: 2-Methyl-4,5-dihydro-(methyl(7,8-di-O-acetyl-9-(4-(methoxycarbonyl)phenyl)-2,6-anhydro-3,4,5-trideoxy-D-glycero-D-talo-non-2-en)onate)[5,4-d]-1,3-oxazole (11)

A solution of compound 10h (250 mg, 1 eq) in anhydrous pyridine was cooled down to 0° C., followed by dropwise addition of acetic anhydride (230 μl, 4.5 eq). The reaction mixture was allowed to warm to room temperature and kept stirring overnight. After completion, the reaction was quenched with methanol and the solvents were removed under reduced pressure. The residue was dissolved in ethyl acetate and carefully washed with 0.05 M HCl, water, and brine sequentially and dried over Na₂SO₄. The solution was then concentrated and purified by flash chromatography, providing a crude fully protected product, which was used in the next step without further purification. The obtained crude protected product (800 mg, 1 eq, several batches' product of last step) was dissolved in 10 ml ethyl acetate. The solution was warmed to 40° C. and TMSOTf (408 μl, 3 eq) was added drop wisely. The resulting solution was kept stirring at 50° C. for 4 hours. After completion, the solution was added to a vigorously stirred cold saturated sodium bicarbonate solution. The aqueous phase was separated and extracted with ethyl acetate. The organic phase was combined, dried over Na₂SO₄, concentrated and purified by flash chromatography to give the desired product (430 mg, 60%). ¹H NMR (500 MHz, CD₃OD) δ 8.49 (s, 1H, Triazole-H5), 8.03-7.97 (d, J=8.5 Hz, 2H, Ar—H), 7.88 (d, J=8.5 Hz, 2H, Ar—H), 6.39 (d, J=4.0 Hz, 1H, H-3), 5.65-5.57 (m, 2H, H-7, H-8), 5.22 (dd, J=14.8, 2.6 Hz, 1H, H-9), 4.94 (dd, J=9.5, 4.0 Hz, 1H, H-4), 4.79 (m, 1H, H-9′), 4.02 (t, J=9.5 Hz, 1H, H-5), 3.87, 3.79 (2×s, 2×3H, 2×COOCH₃), 3.60 (dd, J=9.5, 2.3 Hz, 1H, H-6), 2.17 (s, 3H, oxazole-CH₃), 1.97, 1.95 (2×s, 2×3H, 2×COOCH₃). ¹³C NMR (125 MHz, CD₃OD) δ 171.55, 171.32, 168.03, 163.34 (C═O), 170.12 (oxazole-O—C═N), 148.09 (Triazole-C4), 124.39 (Triazole-C5), 147.66 (C-2), 136.30, 131.27, 130.78, 126.55 (Ar—C), 109.05 (C-3), 78.35 (C-6), 74.22 (C-4), 73.52 (C-8), 70.86 (C-7), 62.68 (C-5), 53.27, 52.81 (COOCH₃), 51.09 (C-9), 20.73, 20.67 (COCH₃), 14.06 (oxazole-CH₃). HR-MS (ESI) calcd. for C₂₆H₂₈N₄NaO₁₀ [M+Na]⁺, 579.1703; found 579.1697.

### Example 18: Methyl 5-acetamido-7,8-di-O-acetyl-9-(4-(methoxycarbonyl)phenyl)-2,6-anhydro-4-azido-3,4,5-trideoxy-D-glycero-D-galacto-non-2-enonate (12)

To a solution of compound 11 (430 mg, 1 eq) in dry ^{t}BuOH, TMSN₃ (507 μl, 5 eq) was added and the resulting solution was stirred at 80° C. under a nitrogen atmosphere for 12 hours. After completion, the solution was cooled down to room temperature, concentrated and purified by flash chromatography to give the desired product. 470 mg (quant.). ¹H NMR (500 MHz, CD₃OD) δ 8.51 (s, 1H, Triazole-H5), 8.07-8.02 (m, 2H, Ar—H), 7.92-7.87 (m, 2H, Ar—H), 5.97 (d, J=2.2 Hz, 1H, H-3), 5.57-5.56 (dd, J=3.3, 2.2 Hz, 1H, H-4), 5.52 (dt, J=9.1, 2.9 Hz, 1H, H-8), 5.29 (dd, J=14.8, 2.7 Hz, 1H, H-9), 4.71 (dd, J=14.8, 9.1 Hz, 1H, H-9′), 4.49 (dd, J=10.3, 1.9 Hz, 1H, H-6), 4.27-4.18 (m, 2H, H-5, H-7), 3.89, 3.80 (2×s, 2×3H, 2×COOCH₃), 2.13, 1.93, 1.92 (3×s, 3×3H, 3×COCH₃). ¹³C NMR (125 MHz, CD₃OD) δ 173.49, 171.83, 171.42, 168.09, 163.07 (C═O), 147.64 (Triazole-C4), 124.51 (Triazole-C5), 146.32 (C-2), 136.25, 131.26, 130.85, 126.54 (Ar—C), 109.74 (C-3), 78.44 (C-6), 73.96 (C-8), 69.60 (C-7), 60.46 (C-4), 53.21, 52.74 (COOCH₃), 51.17 (C-9), 48.39 (C-5), 22.89, 20.88, 20.63 (COCH₃). HR-MS (ESI) calcd. for C₂₆H₂₉N₇NaO₁₀ [M+Na]⁺, 622.1874; found 622.1866.

### Example 19: 5-Acetamido-9-(4-(methoxycarbonyl)phenyl)-2,6-anhydro-4-azido-3,4,5-trideoxy-D-glycero-D-galacto-non-2-enonic acid (13)

60 mg compound 12 was dissolved in 2 ml 0.5 N NaOH, the solution was stirred under room temperature for 1 hour. After completion, Amberlite™ IR 120 (H⁺) was added to neutralize the solution. The suspension was then filtered, and the filtrate was concentrated and purified by flash chromatography to give the desired product. 32 mg (66%). ¹H NMR (500 MHz, CD₃OD) δ 8.43 (s, 1H, Triazole-H5), 8.06 (d, J=8.2 Hz, 2H, Ar—H), 7.91 (d, J=8.2 Hz, 2H, Ar—H), 5.73 (s, 1H), 4.53 (dd, J=14.0, 7.6 Hz, 1H, H-9′), 4.32-4.25 (m, 2H, H-4, H-8), 4.23 (d, J=10.8 Hz, 1H, H-6), 4.18-4.10 (m, 1H, H-5), 3.45 (d, J=9.3 Hz, 1H, H-7), 1.98 (s, 3H, COCH₃). ¹³C NMR (125 MHz, CD₃OD) δ 174.32 (C═O), 147.53 (Triazole-C4), 124.47 (Triazole-C5), 136.17, 131.48, 126.42 (Ar—C), 104.50 (C-3), 77.02 (C-6), 71.03 (C-8), 69.86 (C-7), 60.23 (C-4), 55.25 (C-9), 48.53 (C-5), 22.78 (COCH₃). HR-MS (ESI) calcd. for C₂₀H₂₀N₇O₈ [M−H]⁻, 486.1373; found 486.1378.

### Example 20: Methyl 5-acetamido-7,8-di-O-acetyl-9-(4-(methoxycarbonyl)phenyl)-2,6-anhydro-4-amino-3,4,5-trideoxy-D-glycero-D-galacto-non-2-enonate (14)

To a solution of compound 12 (50 mg, 1 eq) in THE (2 ml), 0.5 N HCl (200 μl, 2 eq) was added, followed by triphenylphosphine (29 mg, 1.1 eq). The resulting mixture was stirred at room temperature overnight. After completion, solvents were removed under reduced pressure and the residue was purified by flash chromatography, providing the desired product 39 mg (84%). ¹H NMR (500 MHz, CD₃OD) δ 8.57 (s, 1H, Triazole-H5), 8.05 (d, J=8.4 Hz, 2H, Ar—H), 7.92 (d, J=8.4 Hz, 2H, Ar—H), 6.06 (d, J=2.3 Hz, 1H, H-3), 5.63-5.55 (m, 2H, H-7, H-8), 5.29-5.21 (m, 1H, H-9), 4.74 (dd, J=14.7, 8.4 Hz, 1H, H-9′), 4.64 (dd, J=10.0, 1.1 Hz, 1H, H-6), 4.35 (t, J=10.0 Hz, 1H, H-5), 4.15 (dd, J=10.0, 2.3 Hz, 1H, H-4), 3.90, 3.81 (2×s, 2×3H, 2×COOCH₃), 2.12, 1.97, 1.95 (3×s, 3×3H, 3×COCH₃). ¹³C NMR (125 MHz, CD₃OD) δ 174.28, 171.71, 171.31, 168.15, 162.80 (C═O), 147.66 (Triazole-C4), 124.59 (Triazole-C5), 147.29, 136.28, 130.84, 126.56 (Ar—C), 107.15 (C-3), 77.96 (C-6), 73.34 (C-8), 69.49 (C-7), 53.34, 52.78 (COOCH₃), 51.67 (C-4), 51.31 (C-9), 46.73 (C-5), 23.14, 20.89, 20.66 (COCH₃). HR-MS (ESI) calcd. for C₂₆H₃₁N₅NaO₁₀ [M+Na]⁺, 596.1969; found 596.1967.

### Example 21: 5-acetamido-7,8-di-O-acetyl-9-(4-(methoxycarbonyl)phenyl)-2,6-anhydro-4-amino-3,4,5-trideoxy-D-glycero-D-galacto-non-2-enonic acid (15)

35 mg compound 14 was dissolved in 400 μL 1N NaOH and the solution was kept stirring at r.t. for 1 h. After completion, the reaction mixture was neutralized with Amberlite™ IR 120 (H⁺). The suspension was then filtered, and the filtrate was concentrated and purified by flash chromatography to give the desired product. 20 mg (71%). ¹H NMR (500 MHz, CD₃OD) δ 8.47 (s, 1H, Triazole-H5), 8.08 (d, J=8.2 Hz, 2H, Ar—H), 7.94 (d, J=8.2 Hz, 2H, Ar—H), 5.84 (s, 1H, H-3), 4.89 (d, J=14.4 Hz, 1H, H-9), 4.56 (dd, J=14.0, 7.5 Hz, 1H, H-9′), 4.41-4.26 (m, 3H, H-8, H-6, H-5), 4.18 (d, J=7.1 Hz, 1H, H-4), 3.56 (d, J=9.1 Hz, 1H, H-7), 2.03 (s, 3H, COCH₃). ¹³C NMR (125 MHz, CD₃OD) δ 174.86, 169.48 (C═O), 147.50 (Triazole-C4), 124.66 (Triazole-C5), 136.29, 131.48, 131.25, 126.45 (Ar—C), 103.03 (C-3), 77.01 (C-6), 70.71 (C-8), 70.03 (C-7), 55.19 (C-9), 51.29 (C-4), 47.54 (C-5), 23.03 (COCH₃). HR-MS (ESI) calcd. for C₂₀H₂₂N₅O₈ [M−H]⁻, 460.1468; found 460.1482.

### Example 22: Methyl 5-acetamido-9-(4-(methoxycarbonyl)phenyl-1H-1,2,3-triazol-1-yl))-4-[2,3-bis(tert-butoxycarbonyl)guanidino]-7,8-di-O-acetyl-2,6-anhydro-3,4,5-trideoxy-D-glycero-D-galacto-non-2-enonate (16)

To a solution of compound 14 (40 mg, 1 eq) in 2 ml anhydrous DCM, TEA (40 μl, 4 eq) was added. The solution was cooled down to 0° C. and N, N′-Di-Boc-1H-pyrazole-1-carboxamidine (42 mg, 2 eq) added. The reaction mixture was allowed to warm up to room temperature and kept stirring overnight. After completion, the reaction was quenched with water, extracted with ethyl acetate. The organic phase was washed with brine, dried over Na₂SO₄, concentrated and purified by flash chromatography to give the desired product. Crude product, 40 mg (72%). ¹H NMR (500 MHz, CD₃OD) δ 8.55 (s, 1H, Triazole-H5), 8.06 (d, J=8.3 Hz, 2H, Ar—H), 7.92 (d, J=8.3 Hz, 2H), 6.00 (d, J=2.3 Hz, 1H, H-3), 5.58 (d, J=1.5 Hz, 1H, H-7), 5.56-5.52 (m, 1H, H-8), 5.31 (dd, J=14.8, 2.4 Hz, 1H, H-9), 5.02 (dd, J=10.2, 2.3 Hz, 1H, H-4), 4.74 (dd, J=14.8, 9.0 Hz, 1H, H-9′), 4.53 (dd, J=10.2, 1.5 Hz, 1H, H-6), 4.27 (t, J=10.2 Hz, 1H, H-5), 3.91, 3.80 (2×s, 3H, COOCH₃), 2.12, 1.94, 1.85 (3×s, 3×3H, 3×COCH₃), 1.51, 1.46 (2×s, 2×9H, 2×Boc). ¹³C NMR (125 MHz, CD₃OD) δ 173.57, 171.77, 171.41, 168.07, 164.32, 163.39, 158.01 (C═O), 153.82 (C═N), 147.64 (Triazole-C4), 124.50 (Triazole-C5), 145.61 (C-2), 136.34, 131.27, 131.27, 126.55 (Ar—C), 111.83 (C-3), 84.84, 80.57 (^{t}Boc-C(CH₃)₃), 78.89 (C-6), 74.00 (C-8), 69.87 (C-7), 53.07, 52.74 (COOCH₃), 51.27 (C-9), 50.84 (C-4), 47.90 (C-5), 28.59, 28.26 (^{t}Boc-C(CH₃)₃), 22.77, 20.86, 20.65 (COCH₃). HR-MS (ESI) calcd. for C₃₇H₄₉N₇NaO₁₄ [M+Na]⁺, 838.3235; found 838.3226.

### Example 23: 5-Acetamido-9-(4-carboxyphenyl)-2,6-anhydro-4-guanidino-3,4,5-trideoxy-D-glycero-D-galacto-non-2-enonic acid (8a)

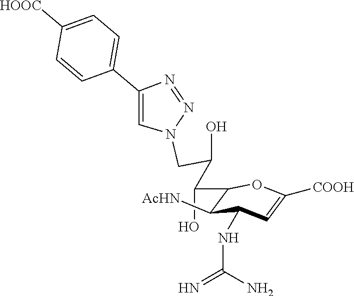

To a solution of compound 16 (40 mg) in 1 ml DCM, 100 μl TFA was added. The solution was then stirred at room temperature for 2 hours. After completion, DCM and TFA were removed under reduced pressure. The residue was dissolved in 2 ml 0.1 N NaOH and stirred at room temperature for 1 hour. After completion, the reaction mixture was added with Amberlite™ IR 120 (H⁺) to adjust the pH of the solution as 7. The suspension was then filtered, and the filtrate was concentrated and purified by flash chromatography to give the desired product. 10 mg (41%). ¹H NMR (500 MHz, D₂O) δ 8.49 (s, 1H, Triazole-H), 8.02 (d, J=8.2 Hz, 2H, Ar—H), 7.93 (d, J=8.2 Hz, 2H, Ar—H), 5.68 (d, J=2.1 Hz, 1H, H-3), 4.91 (dd, J=14.4, 2.8 Hz, 1H, H-9), 4.72 (dd, J=14.4, 6.7 Hz, 1H, H-9′), 4.48 (dd, J=9.5, 2.1 Hz, 1H, H-4), 4.46-4.41 (m, 1H, H-8), 4.40 (d, J=11.0 Hz, 1H, H-7), 4.26 (t, J=9.5 Hz, 1H, H-5), 3.53 (d, J=9.5 Hz, 1H, H-6), 1.99 (s, 3H, COCH₃). ¹³C NMR (125 MHz, D₂O) δ 175.37, 169.94, 163.66 (C═O), 157.96 (C═N), 150.14 (C-2), 147.81, 124.65 (Triazole-C), 133.00, 130.62, 126.33 (Ar—C), 104.76 (C-3), 76.02 (C-6), 69.79 (C-8), 69.00 (C-7), 54.41 (C-9), 51.83 (C-4), 48.65 (C-5), 22.75 (COCH₃). HR-MS (ESI) calcd. for C₂₁H₂₄N₇O₈ [M−H]⁻, 502.1686; found 502.1683.

### Example 24: Methyl 5-acetamido-9-(4-(methoxycarbonyl)phenyl-1H-1,2,3-triazol-1-yl))-4-(3-(3-methoxy-3-oxopropyl)ureido)-7,8-di-O-acetyl-2,6-anhydro-3,4,5-trideoxy-D-glycero-D-galacto-non-2-enonate (17)

A solution of compound 14 (100 mg, 1 eq) and TEA (56 mg, 2 eq) in anhydrous DCM was cooled down to 0° C. and added with 1,1′-Carbonyldiimidazole (39 mg, 1.2 eq). The reaction mixture was then warmed to room temperature and kept stirring for 2 hours until TLC results showed no amine remained. The solution was then cooled down to 0° C., and -alanine methyl ester (56 mg, 2 eq) was added. The solution was warmed to room temperature and kept stirring overnight. After completion, the reaction was quenched with water, extracted by ethyl acetate. The organic layer was washed with water, brine, dried over Na₂SO₄. After concentrated, the residue was purified by flash chromatography to give the desired product. 140 mg (quant.). ¹H NMR (500 MHz, CD₃OD) δ 8.54 (s, 1H, Triazole-H5), 8.07 (d, J=8.7 Hz, 2H, Ar—H), 7.92 (d, J=8.7 Hz, 2H, Ar—H), 5.92 (d, J=2.5 Hz, 1H, H-3), 5.55 (m, 2H, H-8, H-4), 5.31 (dd, J=14.8, 2.6 Hz, 1H, H-9), 4.73 (dd, J=14.8, 8.9 Hz, 1H, H-9′), 4.55 (dd, J=9.9, 2.5 Hz, 1H, H-7), 4.46 (dd, J=10.2, 2.0 Hz, 1H, H-6), 4.10 (t, J=10.2 Hz, 1H, H-5), 3.91, 3.78, 3.66 (3×s, 3×3H, 3×COOCH₃), 3.36 (td, J=6.6, 1.9 Hz, 2H, CH₂), 2.48 (t, J=6.5 Hz, 2H, CH₂), 2.10, 1.93, 1.87 (3×s, 3×3H, 3×COCH₃). ¹³C NMR (125 MHz, CD₃OD) δ 174.12, 173.59, 171.79, 171.41, 168.13, 163.56, 160.42 (C═O), 147.66 (C-2), 145.10 (Triazole-C4), 124.46 (Triazole-C5), 136.30, 131.26, 130.88, 126.54 (Ar—C), 114.06 (C-1), 79.10 (C-6), 73.99 (C-8), 69.97 (C-7), 52.96, 52.70, 52.15 (COOCH₃), 51.26 (C-9), 50.38 (C-5), 36.92, 35.64 (CH₂CH₂), 22.85, 20.84, 20.59 (COCH₃). HR-MS (ESI) calcd. for C₃₁H₃₉N₇N₆O₁₃ [M+Na]⁺, 703.2575; found 703.2571.

### Example 25: 5-Acetamido-9-(4-carboxyphenyl)-2,6-anhydro-4-(3-(2-carboxyethyl)ureido)-3,4,5-trideoxy-D-glycero-D-galacto-non-2-enonic acid (18)

140 mg compound 17 was dissolved in 5 ml 0.1 N NaOH and stirred at room temperature for 1 hour. After completion, the reaction mixture was added with Amberlite™ IR 120 (H⁺) to neutralize the solution. The suspension was then filtered, and the filtrate was concentrated and purified by flash chromatography to give the desired product. 88 mg (77%). ¹H NMR (500 MHz, CD₃OD) δ 8.44 (s, 1H, Triazole-H5), 8.07 (d, J=8.4 Hz, 2H, Ar—H), 7.93 (d, J=8.4 Hz, 2H, Ar—H), 5.66 (d, J=1.9 Hz, 1H, H-3), 4.57 (dd, J=9.8, 1.9 Hz, 1H, H-4), 4.52 (dd, J=14.0, 7.7 Hz, 1H, H-9′), 4.30 (dd, J=12.1, 4.8 Hz, 1H, H-8), 4.19 (d, J=10.8 Hz, 1H, H-6), 4.02 (t, J=10.3 Hz, 1H, H-5), 3.44 (d, J=9.3 Hz, 1H, H-7), 3.37 (t, J=6.4 Hz, 2H, CH₂), 2.46 (t, J=6.4 Hz, 2H, CH₂), 1.94 (s, 3H, COCH₃). ¹³C NMR (125 MHz, CD₃OD) δ 175.74, 174.73, 160.76, 169.64 (C═O), 147.51 (Triazole-C4), 124.40 (Triazole-C5), 136.24, 131.68, 131.45, 126.42 (Ar—C), 109.35 (C-3), 77.81 (C-6), 71.36 (C-8), 69.80 (C-7), 55.31 (C-9), 37.00, 35.78 (CH₂CH₂), 22.74 (COCH₃). HR-MS (ESI) calcd. for C₂₄H₂₇N₆O₁₁ [M−H]⁻, 575.1738; found 575.1738.

### Example 26: Methyl 5-(acetylamino)-4-(tert-butoxycarbonyl)amino-2,6-anhydro-3,5-dideoxy-D-glycero-D-galacto-non-2-enonate (20)

To a solution of compound 19 (600 mg, 1 eq) and TEA (389 μl, 2 eq) in 20 ml anhydrous DCM at 0° C., di-tert-butyl dicarbonate (456 mg, 1.5 eq) was added in dropwise. The mixture was them warmed up to room temperature and kept stirring overnight. After completion, solvent was removed, and the residue was purified by flash chromatography to give the desired compound 350 mg (crude product, 47%). The crude product (350 mg, 1 eq) was dissolved in 10 ml methanol, and cooled down to 0° C., followed by addition of NaOMe (92 mg, 3 eq). The solution was kept stirring at 0° C. for about 1 hour until no starting material remained. Amberlite™ IR 120 (H⁺) was added to neutralize the solution. The suspension was then filtered, and the filtrate was concentrated and purified by flash chromatography to give the desired product. 190 mg (71%). ¹H NMR (500 MHz, CD₃OD) δ 5.82 (d, J=2.2 Hz, 1H, H-3), 4.46 (d, J=10.1 Hz, 1H, H-4), 4.23 (d, J=10.1 Hz, 1H, H-6), 4.05 (t, J=10.1 Hz, 1H, H-5), 3.88 (ddd, J=9.2, 5.4, 2.9 Hz, 1H, H-8), 3.81 (dd, J=11.4, 2.9 Hz, 1H, H-9), 3.65 (dd, J=11.4, 5.4 Hz, 1H, H-9′), 3.58 (dd, J=9.3, 1.1 Hz, 1H, H-7), 1.98 (s, 3H, COOCH₃), 1.44 (s, 9H, ^{t}Boc-C(CH₃)₃). ¹³C NMR (125 MHz, CD₃OD) δ 174.65, 164.26, 158.35 (C═O), 145.71 (C-2), 112.19 (C-3), 80.56 (^{t}Boc-C(CH₃)₃), 78.62 (C-6), 71.13 (C-8), 70.00 (C-7), 64.90 (C-9), 52.79 (C-4), 50.26 (C-5), 28.70 (^{t}Boc- C(CH₃)₃), 22.70 (COCH₃). HR-MS (ESI) calcd. for C₁₇H₂₉N₂O₉ [M+H]⁺, 405.1873; found 405.1875.

### Example 27: Methyl 4-(tert-butoxycarbonyl)amino-5-acetylamino-9-(4-methylbenzenesulfonate)-2,6-anhydro-3,5-dideoxy-D-glycero-D-galacto-non-2-enonate (21)

A solution of compound 20 (190 mg, 1 eq) in anhydrous pyridine was cooled down to 0° C., TsCl (98 mg, 1.1 eq) was then added slowly under stirring. The solution was warmed room temperature and kept stirring overnight. After completion, the reaction was quenched by methanol. The solution was concentrated and purified by flash chromatography to give the desired product. 200 mg (76%). ¹H NMR (500 MHz, CDCl₃) δ 7.78 (d, J=8.1 Hz, 2H, Ar—H), 7.33 (d, J=8.1 Hz, 2H, Ar—H), 6.93, 5.23, 5.11, 3.59 (4×d, 4H, 2×NH, 2×OH), 5.82 (d, J=2.3 Hz, 1H, H-3), 4.55 (td, J=9.6, 2.2 Hz, 1H, H-8), 4.40-4.31 (m, 1H, H-6), 4.21-4.17 (m, 1H, H-5), 4.15-4.11 (m, 2H, H-9′, H-4), 4.01-3.95 (m, 1H, H-9′), 3.71 (s, 3H, COOCH₃), 3.56-3.48 (m, 1H, H-7), 2.43 (s, 3H, PhCH₃), 2.00 (s, 3H, COCH₃), 1.42 (s, 9H, ^{t}Boc-C(CH₃)₃). ¹³C NMR (125 MHz, CDCl₃) δ 174.01, 162.21, 156.80 (C═O), 145.07 (C-2), 144.86, 132.59, 129.91, 128.02 (Ar—C), 109.92 (C-3), 80.60 (^{t}Boc-C(CH₃)₃), 72.56 (C-9), 68.53 (C-7), 67.90 (C-8), 52.38 (C-6), 50.19 (C-4), 48.68 (C-5), 28.26 (^{t}Boc-C(CH₃)₃), 22.86, 21.63 (COCH₃, PhCH₃). HR-MS (ESI) calcd. for C₂₄H₃₅N₂O₁₁S [M+H]⁺, 559.1962; found 559.1966.

### Example 28: Methyl 5-acetamido-7,8-di-O-acetyl-9-azido-2,6-anhydro-4-[2,3-bis(tert-butoxycarbonyl)guanidino]-3,4,5-trideoxy-D-glycero-D-galacto-non-2-enonate (23)

Compound 21 (200 mg, 1 eq) was dissolved in 3 ml acetone-water (2:1) and NaN₃ (117 mg, 5 eq) was added. The solution was heated at 67° C. under N₂ for two days. After completion, the solution was concentrated and purified by flash chromatography to give 100 mg compound 22 (crude product, 75%) which was used in the next step without further purification. The crude product was dissolved in 2 ml anhydrous DCM, and 200 μl TFA was added. The solution was kept stirring at room temperature until no starting material remained. Solvents was then removed under vacuum and the residue was dissolved in 2 ml anhydrous DCM, and TEA (140 μl, 4 eq) was added. After the solution was cooled down to 0° C., N,N′-Di-Boc-1H-pyrazole-1-carboxamidine (150 mg, 2 eq) was added. The reaction mixture was allowed to warm up to room temperature and kept stirring overnight. After completion, the reaction was quenched with water, extracted with ethyl acetate. The organic phase was washed with brine, dried over Na₂SO₄, concentrated and purified by flash chromatography to give the desired product. 108 mg (82%). ¹H NMR (500 MHz, CDCl₃) δ 8.63, 8.15, 7.70, 6.43 (2×d, 2×brs, 4H, 2×NH, 2×OH), 5.82 (d, J=2.4 Hz, 1H, H-3), 5.20 (ddd, J=10.2, 8.1, 2.4 Hz, 1H, H-4), 4.22-4.15 (m, 2H, H-6, H-5), 4.02 (td, J=10.2, 6.1 Hz, 1H, H-8), 3.72 (dd, J=12.6, 2.8 Hz, 1H, H-9), 3.60-3.53 (m, 2H, H-9′, H-7), 2.04 (s, 3H, COCH₃), 1.52 (2×s, 2×9H, 2×^{t}Boc-C(CH₃)₃). ¹³C NMR (125 MHz, CDCl₃) δ 174.04, 162.29, 162.08, 157.56 (C═O), 152.70 (C═N), 146.31 (C-2), 107.51 (C-3), 84.39, 80.11 (^{t}Boc-C(CH₃)₃), 69.14 (C-6), 54.89 (C-9), 52.53 (C-7), 51.53 (C-8), 48.33 (C-5), 28.23, 28.04 (^{t}Boc-C(CH₃)₃), 22.96 (COCH₃). HR-MS (ESI) calcd. for C₂₃H₃₈N₇O₁₀ [M+H]⁺, 572.2680; found 572.2681.

### Example 29: Methyl 5-acetamido-9-(4-biphenyl-1H-1,2,3-triazol-1-yl))-4-[2,3-bis(tert-butoxycarbonyl)guanidino]-2,6-anhydro-4-[2,3-bis(tert-butoxycarbonyl)guanidino]-3,4,5-trideoxy-D-glycero-D-galacto-non-2-enonate (24)

Compound 23 (200 mg, 1 eq) and 4-ethynylbiphenyl (32 mg, 1.5 eq) were taken in to click-reaction as mentioned earlier to give the desired product. 180 mg (69%). ¹H NMR (700 MHz, CDCl₃) δ 8.55, 8.06 (2×d, 2H, 2×NH), 7.94 (s, 1H, Triazole-H5), 7.81 (d, J=8.3 Hz, 2H, Ar—H), 7.57 (dd, J=13.1, 7.8 Hz, 4H, Ar—H), 7.39 (t, J=7.7 Hz, 2H, Ar—H), 7.31 (t, J=7.4 Hz, 1H, Ar—H), 5.75 (d, J=2.3 Hz, 1H, H-3), 5.51 (brs, 1H, OH), 5.16-5.10 (m, 1H, H-4), 4.90 (dd, J=14.0, 1.7 Hz, 1H, H-9), 4.54 (dd, J=14.0, 6.7 Hz, 1H, H-9′), 4.48-4.43 (m, 1H, H-8), 4.20 (d, J=10.4 Hz, 1H, H-6), 3.96 (td, J=10.4, 6.2 Hz, 1H, H-5), 3.69 (s, 3H, COOCH₃), 3.34 (d, J=9.0 Hz, 1H, H-7), 1.90 (s, 3H), 1.47, 1.43 (2×s, 2×9H, 2×^{t}Boc-C(CH₃)₃). ¹³C NMR (176 MHz, CDCl₃) δ 174.10 (COCH₃), 162.26, 162.03 (^{t}Boc-OCO), 157.37 (C-1), 152.66 (C═N), 146.98 (C-2), 146.26 (Triazole-C4), 121.69 (Triazole-C5), 140.63, 140.43, 129.41, 128.77, 127.37, 126.86, 125.99 (Ar—C), 107.54 (C-3), 84.22, 79.96 (^{t}Boc-C(CH₃)₃), 69.33 (C-6), 68.54 (C-4), 53.90 (C-9), 52.42 (C-8), 51.58 (C-7), 48.37 (C-5), 28.17, 27.99 (^{t}Boc-C(CH₃)₃), 22.86 (COCH₃). HR-MS (ESI) calcd. for C₃₇H₄₈N₇O₁₀ [M+H]⁺, 750.3463; found 750.3454.

### Example 30: 5-Acetamido-9-(4-biphenyl-1H-1,2,3-triazol-1-yl))-4-guanidino-2,6-anhydro-4-[2,3-bis(tert-butoxycarbonyl)guanidino]-3,4,5-trideoxy-D-glycero-D-galacto-non-2-enonic acid (8b)

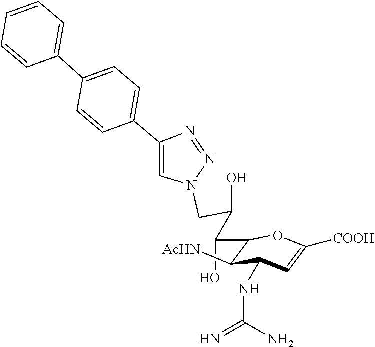

300 μl TFA was added to a solution of compound 24 (180 mg) in 3 ml DCM and the solution was then stirred at room temperature for about 2 hours. After completion, DCM and TFA were removed under reduced pressure. The residue was dissolved in 3 ml 0.1 N NaOH and stirred at room temperature for 1 hour. After completion, Amberlite™ IR 120 (H⁺) was added to neutralize the solution. The suspension was then filtered, and the filtrate was concentrated and purified by flash chromatography to give the desired product. 10 mg (31%). ¹H NMR (500 MHz, CD₃OD) δ 8.34 (s, 1H, Trizaole-H), 7.87 (d, J=8.1 Hz, 2H, Ar—H), 7.66 (d, J=8.1 Hz, 2H, Ar—H), 7.61 (d, J=7.5 Hz, 2H, Ar—H), 7.41 (t, J=7.6 Hz, 2H, Ar—H), 7.32 (t, J=7.3 Hz, 1H, Ar—H), 5.89 (s, 1H, H-3), 4.88 (d, J=14.3 Hz, 1H, H-9), 4.60-4.47 (m, 2H, H-9′, H-6), 4.42 (d, J=10.0 Hz, 1H, H-4), 4.28 (m, 2H, H-5, H-8), 3.57 (d, J=9.0 Hz, 1H, H-7), 1.98 (s, 3H, COCH₃). ¹³C NMR (125 MHz, CD₃OD) δ 174.56, 165.08 (C═O), 158.96 (C═N), 148.25 (C-2), 146.68, 123.70 (Triazole-C), 142.30, 141.70, 130.71, 129.95, 128.58, 128.50, 127.85, 127.12 (Ar—C), 109.14 (C-3), 77.79 (C-6), 71.21 (C-8), 70.06 (C-7), 55.19 (C-9), 51.53 (C-4), 22.72 (COCH₃). HR-MS (ESI) calcd. for C₂₆H₂₈N₇O₆ [M−H]⁻, 534.2101; found 534.2105.

### Example 31: General Procedure for Synthesis of Compounds 25a-d

A solution of compound 19 (1 eq) and TEA (3 eq) in anhydrous DCM was cooled down to 0° C. and corresponding anhydrides or acyl chlorides (3 eq) was added in dropwise. The resulting mixture was warmed to room temperature and kept stirring overnight. After completion, the reaction was quenched with water and extracted with ethyl acetate. The organic layer was collected and washed with saturated NaHCO₃, brine sequentially and dried with NaSO₄. Solvents were removed under reduced pressure and the residue was separated by flash chromatography to give desired crude products. For hydrolysis of the C1-methyl ester, the product obtained above was dissolved in MeOH, and 0.5 M NaOH was added. The mixture was kept stirring at room temperature. After completion, the mixture was neutralized with Amberlite™ IR-120 (H⁺), filtrated and purified by flash chromatography to provide the desired products with yields of 42%-68% (two steps) (FIG. 1E).

### Example 32: 5-Acetamido-2,6-anhydro-4-propionamido-3,4,5-trideoxy-D-glycero-D-galacto-non-2-enonic acid (25a)

(28 mg, 68%)¹H NMR (500 MHz, CD₃OD) δ 5.49 (d, J=2.0 Hz, 1H, H-3), 4.75 (dd, J=9.7, 2.0 Hz, 1H, H-4), 4.20 (d, J=10.8 Hz, 1H, H-6), 4.10-4.06 (m, 1H, H-5), 3.86-3.85 (m, 1H, H-8), 3.79 (dd, J=11.4, 3.0 Hz, 1H, H-9), 3.63 (dd, J=11.4, 5.4 Hz, 1H, H-9′), 3.55 (d, J=9.0 Hz, 1H, H-7), 2.17 (q, J=7.6 Hz, 2H, α-CH₂), 1.93 (s, 3H, COCH₃), 1.09 (t, J=7.6 Hz, 3H, β-CH₃). ¹³C NMR (125 MHz, CD₃OD) δ 177.40, 174.17 (C═O), 106.15 (C-3), 77.49 (C-6), 71.46 (C-8), 70.05 (C-7), 64.88 (C-9), 49.64 (C-5), 30.40 (α-CH₂), 22.78 (COCH₃), 10.59 (β-CH₃). HR-MS (ESI) calcd. for C₁₄H₂₁N₂O₈ [M−H]⁻, 345.1298; found 345.1302.

### Example 33: 5-Acetamido-2,6-anhydro-4-pentanamido-3,4,5-trideoxy-D-glycero-D-galacto-non-2-enonic acid (25b)

(25 mg, 56%). ¹H NMR (500 MHz, CD₃OD) δ 5.47 (d, J=2.2 Hz, 1H, H-3), 4.76 (dd, J=9.8, 2.2 Hz, 1H, H-4), 4.19 (d, J=10.8 Hz, 1H, H-6), 4.09-4.05 (m, 1H, H-5), 3.87-3.85 (m, 1H, H-8), 3.78 (dd, J=11.5, 3.1 Hz, 1H, H-9), 3.66 (dd, J=11.5, 5.1 Hz, 1H, H-9′), 3.57 (t, J=7.8 Hz, 1H, H-7), 2.17 (t, J=7.5 Hz, 2H, α-CH₂), 1.94 (s, 3H, COCH₃), 1.60-1.51 (m, 2H, β-CH₂), 1.34-1.29 (m, 2H, γ-CH₂), 0.90 (t, J=7.4 Hz, 3H, 6-CH₃). ¹³C NMR (125 MHz, CD₃OD) δ 176.55, 174.18, 170.01 (3×C═O), 151.02 (C-2), 105.96 (C-3), 77.51 (C-6), 71.55 (C-8), 69.91 (C-7), 64.71 (C-9), 49.60 (C-4), 49.00 (C-5), 37.02 (α-CH₂), 29.24 (β-CH₂), 23.31 (γ-CH₂), 23.31 (COCH₃), 14.19 (δ-CH₃). HR-MS (ESI) calcd. for C₁₆H₂₅N₂O₈ [M−H]⁻, 373.1611; found 373.1612.

### Example 34: 5-Acetamido-2,6-anhydro-4-cyclopropanecarboxamido-3,4,5-trideoxy-D-glycero-D-galacto-non-2-enonic acid (25c)

(22 mg, 44%). ¹H NMR (500 MHz, CD₃OD) δ 5.54 (d, J=2.1 Hz, 1H, H-3), 4.77 (dd, J=9.8, 2.1 Hz, 1H, H-4), 4.20 (d, J=10.7 Hz, 1H, H-6), 4.11-4.07 (m, 1H, H-5), 3.90-3.82 (m, 1H, H-8), 3.79 (dd, J=11.4, 3.0 Hz, 1H, H-9), 3.66 (dd, J=11.4, 5.2 Hz, 1H, H-9′), 3.57 (d, J=9.0 Hz, 1H, H-7), 1.94 (s, 3H, COCH₃), 1.58-1.53 (m, 1H, α-CH), 0.88-0.78 (m, 2H, β-CH₂), 0.74-0.72 (m, 2H, β-CH₂). ¹³C NMR (125 MHz, CD₃OD) δ 176.90, 174.36, 169.54 (3×C═O), 150.41 (C-2), 106.92 (C-3), 77.64 (C-6), 71.52 (C-8), 69.95 (C-7), 64.77 (C-9), 49.92 (C-4), 49.28 (C-5), 22.87 (COCH₃), 15.05 (α-CH), 7.57, 7.49 (2×β-CH₂). HR-MS (ESI) calcd. for C₁₅H₂₁N₂O₈ [M−H]⁻, 357.1298; found 357.1305.

### Example 35: 5-Acetamido-2,6-anhydro-4-cyclobutanecarboxamido-3,4,5-trideoxy-D-glycero-D-galacto-non-2-enonic acid (25d)

(19 mg, 42%). ¹H NMR (500 MHz, CD₃OD) δ 5.49 (d, J=2.2 Hz, 1H, H-3), 4.75 (dd, J=9.8, 2.2 Hz, 1H, H-4), 4.21 (d, J=10.8 Hz, 1H, H-6), 4.10-4.06 (m, 1H, H-5), 3.88-3.84 (m, m, 1H, H-8), 3.78 (dd, J=11.5, 3.1 Hz, 1H, H-9), 3.66 (dd, J=11.5, 5.2 Hz, 1H, H-9′), 3.56 (d, J=9.3 Hz, 1H, H-7), 3.09-3.02 (m, 1H, α-CH), 2.27-1.77 (m, 6H, 3×CH₂), 1.93 (s, 3H, COCH₃). ¹³C NMR (125 MHz, CD₃OD) δ 178.06, 174.20, 169.63 (3×C═O), 150.52 (C-2), 106.54 (C-3), 77.53 (C-6), 71.55 (C-8), 69.94 (C-7), 64.77 (C-9), 49.57 (C-4), 49.14 (C-5), 40.90 (α-CH), 26.44, 26.02, 19.09 (3×CH₂), 22.88 (COCH₃). HR-MS (ESI) calcd. for C₁₆H₂₃N₂₀ [M−H]⁻, 371.1454; found 371.1458.

### Example 36: General Procedure for Synthesis of C9-Amido Compounds

C9-azido DANA methyl ester was dissolved in THF-H₂O and cooled down to 0° C. with ice water bath. Triphenyl phosphate (TPP) was then added followed with activated carboxylic acids. The solution was allowed to warm to room temperature and kept stirring overnight. After completion, the reaction was quenched with water, concentrated and purified by flash chromatography to give the desired product. The product was then dissolved in MeOH, and 0.5 M NaOH was added (FIG. 1F). The mixture was kept stirring at room temperature. After completion, the mixture was neutralized with Amberlite™ IR-120 (H⁺ form), filtered and purified by flash chromatography to provide the desired products.

### Example 37: 5-Acetamido-9-butyramido-2,6-anhydro-3,5-dideoxy-D-glycero-D-galacto-non-2-enonic acid (49)

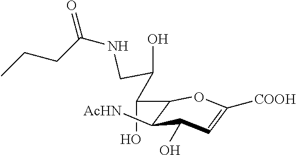

¹H NMR (500 MHz, CD₃OD) 5.93 (d, J=2.5 Hz, 1H, H-3), 4.41 (dd, J=8.7, 2.5 Hz, 1H, H-4), 4.20-4.15 (m, 1H, H-6), 3.96 (dd, J=10.7, 8.7 Hz, 1H, H-5), 3.94-3.88 (m, 1H, H-8), 3.57 (dd, J=14.0, 3.3 Hz, 1H, H-9), 3.41 (dd, J=9.1, 1.1 Hz, 1H, H-7), 2.22-2.15 (m, 2H, α-CH₂), 2.00 (d, J=9.2 Hz, 3H, COCH₃), 1.62 (dd, J=14.8, 7.4 Hz, 2H, β-CH₂), 0.93 (t, J=7.4 Hz, 3H, γ-CH₃). ¹³C NMR (126 MHz, CD₃OD) δ 177.13, 174.87 ((N—C═O)), 165.51 (C-1), 113.36 (C-3), 77.83 (C-6), 71.49 (C-7), 70.17 (C-4), 67.96 (C-8), 52.01 (C-5), 44.40 (C-9), 38.97 (C-α), 22.73 (COCH₃), 20.45 (C-β), 14.05 (C-γ).

### Example 38: 5-Acetamido-9-pentanamido-2,6-anhydro-3,5-dideoxy-D-glycero-D-galacto-non-2-enonic acid (50)

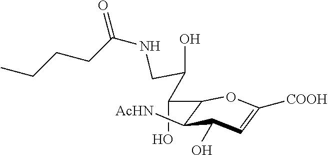

¹H NMR (500 MHz, CD₃OD) δ 5.93 (d, J=1.7 Hz, 1H, H-3), 4.43 (dd, J=8.6, 1.7 Hz, 1H, H-4), 4.18 (d, J=10.8 Hz, 1H, H-6), 4.02-3.95 (m, 1H, H-5), 3.95-3.87 (m, 1H, H-8), 3.59 (dd, J=13.9, 2.9 Hz, 1H, H-9), 3.43 (d, J=9.0 Hz, 1H, H-7), 3.32-3.27 (m, 1H, H-9′), 2.22 (t, J=7.6 Hz, 2H, α-CH₂), 2.03 (s, 3H, COCH₃), 1.64-1.54 (m, 2H, β-CH₂), 1.35 (dd, J=15.0, 7.5 Hz, 2H, γ-CH₂), 0.92 (t, J=7.4 Hz, 3H, δ-CH₂). ¹³C NMR (126 MHz, CD₃OD) δ 177.26, 174.88 (N—C═O), 165.81 (C-1), 145.81 (C-2), 113.17 (C-3), 77.79 (C-6), 71.50 (C-7), 70.24 (C-4), 68.03 (C-8), 51.97 (C-5), 44.40 (C-9), 36.87 (C-α), 29.28 (C-β), 23.44 (γ), 22.86 (COCH₃), 14.22 (C-δ). HRMS (ESI) calcd. for C₁₆H₂₅N₂O₈ [M−H]⁻, 373.1616; found 373.1614.

### Example 39: 5-Acetamido-9-hexanamido-2,6-anhydro-3,5-dideoxy-D-glycero-D-galacto-non-2-enonic acid (51)

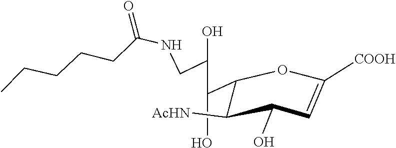

¹H NMR (500 MHz, CD₃OD) δ 5.92 (d, J=2.5 Hz, 1H, H-3), 4.42 (dd, J=8.7, 2.5 Hz, 1H, H-4), 4.17 (dd, J=10.7, 1.0 Hz, 1H, H-6), 3.97 (dd, J=10.7, 8.7 Hz, 1H, H-5), 3.94-3.88 (m, 1H, H-8), 3.59 (dd, J=13.9, 3.3 Hz, 1H, H-9), 3.42 (dd, J=9.0, 1.0 Hz, 1H, H-7), 3.31-3.27 (m, 1H, H-9′), 2.23-2.16 (m, 2H, α-CH₂), 2.01 (s, 3H, COCH₃), 1.59 (dt,J=15.0, 7.6 Hz, 2H, β-CH₂), 1.39-1.25 (m, 4H, γ-CH₂, δ-CH₂), 0.89 (t, J=7.1 Hz, 3H, ε-CH₃). ¹³C NMR (126 MHz, CD₃OD) δ 177.25, 174.87 (N—C═O), 165.63 (C-1), 113.30 (C-3), 77.82 (C-6), 71.55 (C-7), 70.24 (C-4), 67.98 (C-8), 51.97 (C-5), 44.41 (C-9), 37.09 (C-α), 32.58 (C-β), 26.81 (C-γ), 23.45 (C-δ), 22.81 (COCH₃), 14.32 (C-ε). HRMS (ESI) calcd. for C₁₇H₂₇N₂O₈ [M−H]⁻, 387.1773; found 387.1766.

### Example 40: 5-Acetamido-9-heptanamido-2,6-anhydro-3,5-dideoxy-D-glycero-D-galacto-non-2-enonic acid (52)

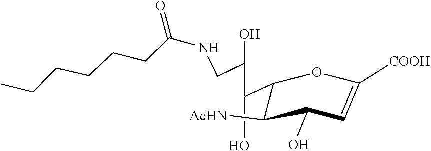

¹H NMR (500 MHz, CD₃OD) δ 5.77 (d, J=2.3 Hz, 1H, H-3), 4.37 (dd, J=8.6, 2.3 Hz, 1H, H-4), 4.16-4.08 (m, 1H, H-6), 3.97 (dd, J=10.7, 8.7 Hz, 1H, H-5), 3.89 (ddd, J=8.7, 7.1, 3.4 Hz, 1H, H-8), 3.58 (dd, J=13.8, 3.4 Hz, 1H, H-9), 3.40 (d, J=8.7 Hz, 1H, H-7), 3.28-3.23 (m, 1H, H-9′), 2.26-2.13 (m, 2H, α-CH₂), 2.01 (s, 3H, COCH₃), 1.64-1.52 (m, 2H, β-CH₂), 1.37-1.23 (m, 6H, γ-CH₂, δ-CH₂, ε-CH₃), 0.88 (dd, J=8.8, 5.1 Hz, 3H, ζ-CH₃). ¹³C NMR (126 MHz, CD₃OD) δ 177.04, 174.70 (N—C═O), 110.32 (C-3), 77.35 (C-6), 71.50 (C-7), 70.44 (C-4), 68.36 (C-8), 51.99 (C-5), 44.23 (C-9), 37.15 (C-α), 32.73 (C-β), 30.07 (C-γ), 27.10 (C-δ), 23.58 (C-ε), 22.80 (C-ζ), 14.39 (COCH₃). HRMS (ESI) calcd. for C₁₈H₂₉N₂O₈ [M−H]⁻, 401.1929; found 401.1931.

### Example 41: 5-Acetamido-9-isobutyramido-2,6-anhydro-3,5-dideoxy-D-glycero-D-galacto-non-2-enonic acid (53)

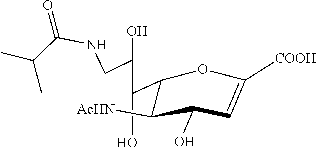

¹H NMR (700 MHz, CD₃OD) δ 5.74 (d, J=2.3 Hz, 1H, H-3), 4.37 (dd, J=8.7, 2.3 Hz, 1H, H-4), 4.14 (dd, J=10.8, 1.0 Hz, 1H, H-6), 3.99 (dd, J=10.8, 8.7 Hz, 1H, H-5), 3.91 (ddd, J=9.1, 6.9, 3.4 Hz, 1H, H-8), 3.58 (dd, J=13.8, 3.4 Hz, 1H, H-9), 3.40 (dd, J=9.1, 1.0 Hz, 1H, H-7), 3.33 (dd, J=13.8, 6.9 Hz, 1H, H-9′), 2.49 (dt, J=13.8, 6.9 Hz, 1H, α-CH₂), 2.02 (s, 3H, COCH₃), 1.12 (dd, J=6.9, 0.5 Hz, 6H, 2×β-CH₃). ¹³C NMR (176 MHz, CD3OD) δ 180.76, 174.56 (N—C═O), 109.51 (C-3), 77.30 (C-6), 71.67 (C-7), 70.20 (C-4), 68.50 (C-8), 52.07 (C-5), 44.23 (C-9), 36.28 (C-α), 22.73, 19.96, 19.90 (2×C-β, COCH₃). HRMS (ESI) calcd. for C₁₅H₂₃N₂O₈ [M−H]⁻, 359.1460; found 359.1458.

### Example 42: 5-Acetamido-9-(3-methylbutanamido)-2,6-anhydro-3,5-dideoxy-D-glycero-D-galacto-non-2-enonic acid (54)

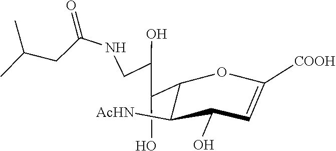

1H NMR (700 MHz, CD₃OD) δ 5.74 (d, J=2.3 Hz, 1H, H-3), 4.37 (dd, J=8.7, 2.3 Hz, 1H, H-4), 4.13 (dd, J=10.8, 1.0 Hz, 1H, H-6), 3.99 (dd, J=10.8, 8.7 Hz, 1H, -5), 3.91 (ddd, J=9.0, 6.9, 3.4 Hz, 1H, H-8), 3.60 (dd, J=13.8, 3.4 Hz, 1H, H-9), 3.43-3.39 (dd, J=13.8, 6.9 Hz, 1H, H-9′), 3.33 (m, 1H, H-7), 2.10-2.09 (m, 2H, α-CH₂), 2.08-2.03 (m, 1H, β-CH), 2.02 (s, 3H, COCH₃), 0.96 (dd, J=6.4, 1.6 Hz, 6H, 2×γ-CH₃). ¹³C NMR (176 MHz, CD₃OD) δ 176.27, 174.60 (N—C═O), 109.43 (C-3), 77.30 (C-6), 71.71 (C-7), 70.26 (C-4), 68.51 (C-8), 52.08 (C-5), 46.32 (C-9), 44.18 (C-α), 27.42 (C-β), 22.77, 22.75, 22.74 (2×C-γ, COCH₃). HRMS (ESI) calcd. for C₁₆H₂₅N₂O₈ [M−H]⁻, 373.1616; found 373.1617.

### Example 43: 5-Acetamido-9-(4-methylpentanamido)-2,6-anhydro-3,5-dideoxy-D-glycero-D-galacto-non-2-enonic acid (55)

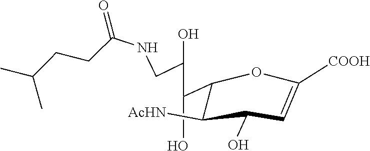

¹H NMR (500 MHz, CD₃OD) δ 5.69 (d, J=2.3 Hz, 1H, H-3), 4.36 (dd, J=8.7, 2.3 Hz, 1H, H-4), 4.10 (dd, J=10.8, 0.8 Hz, 1H, H-6), 3.97 (dd, J=10.8, 8.7 Hz, 1H, H-5), 3.92-3.85 (m, 1H, H-8), 3.58 (dd, J=13.8, 3.3 Hz, 1H, H-9), 3.38 (dd, J=8.9, 0.8 Hz, 1H, H-7), 3.28-3.21 (m, 1H, H-9′), 2.25-2.18 (m, 2H, α-CH₂), 2.01 (s, 3H, COCH₃), 1.60-1.44 (m, 3H, β-CH₂, γ-CH), 0.90 (d, J=6.5 Hz, 6H, 2×δ-CH₃). 13C NMR (126 MHz, CD₃OD) δ 177.19, 174.68 (N—C═O), 108.93 (C-3), 77.12 (C-6), 71.55 (C-7), 70.35 (C-4), 68.59 (C-8), 52.00 (C-5), 44.22 (C-9), 36.12 (C-α), 35.23 (C-β), 29.04 (C-γ), 22.86, 22.74 (2×C-β, COCH₃). HRMS (ESI) calcd. for C₁₇H₂₇N₂O₈ [M−H]⁻, 387.1773; found 387.1765.

### Example 44: 5-Acetamido-9-benzamido-2,6-anhydro-3,5-dideoxy-D-glycero-D-galacto-non-2-enonic acid (56)

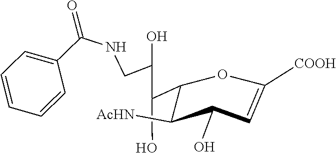

¹H NMR (500 MHz, CD₃OD) δ 7.85-7.79 (m, 2H, Ar—H), 7.54-7.48 (m, 1H, Ar—H), 7.43 (dd, J=10.3, 4.7 Hz, 2H, Ar—H), 5.84 (d, J=2.3 Hz, 1H, H-3), 4.40 (dd, J=8.7, 2.3 Hz, 1H, H-4), 4.18 (d, J=10.8 Hz, 1H, H-6), 4.07-3.96 (m, 2H, H-8, H-5), 3.77 (dd, J=13.8, 3.4 Hz, 1H, H-9), 3.55 (dd, J=13.8, 6.8 Hz, 1H, H-9′), 3.48 (d, J=8.9 Hz, 1H, H-7), 1.96 (s, 3H, COCH₃). ¹³C NMR (126 MHz, CD₃OD) δ 174.79, 170.91 (N—C═O), 135.63, 132.71, 129.57, 128.36 (Ar—C), 111.64 (C-3), 77.53 (C-6), 71.60 (C-7), 70.35 (C-4), 68.20 (C-8), 51.92 (C-5), 45.02 (C-9), 22.74 (COCH₃). HRMS (ESI) calcd. for C₁₈H₂₁N₂O₈ [M−H]⁻, 393.1303; found 393.13.

### Example 45: General Procedure for Synthesis of C5-Amido Compounds

Compound I-25 was dissolved in anhydrous DCM and TEA was added. The mixture was then cooled down to 0° C. and activated carboxylic acids was added in dropwise. The solution was allowed to warm to room temperature and kept stirring overnight. After completion, the reaction was quenched with water, concentrated and purified by flash chromatography to give the desired product. The product was then dissolved in MeOH, and 0.5 M NaOH was added (FIG. 1G). The mixture was kept stirring at room temperature. After completion, the mixture was neutralized with Amberlite™ IR-120 (H⁺ form), filtered and purified by flash chromatography to provide the desired products.

To synthesize compounds 40-48, fully protected 40 (II-82fulpro) was taken to CuAAC as described before with alkynes to give desired product, which was then hydrolyzed using NaOH to give desired final products for enzymatic assay (FIG. 1H).

### Example 46: 5-Propionamido-2,6-anhydro-3,5-dideoxy-D-glycero-D-galacto-non-2-enonic acid (29)

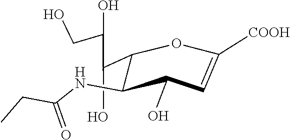

¹H NMR (500 MHz, CD₃OD) δ 5.90 (d, J=2.4 Hz, 1H, H-3), 4.43 (dd, J=8.7, 2.4 Hz, 1H, H-4), 4.15 (dd, J=10.8, 0.9 Hz, 1H, H-6), 4.01-3.95 (m, 1H, H-5), 3.91-3.86 (m, 1H, H-8), 3.81 (dd, J=11.4, 3.0 Hz, 1H, H-9), 3.65 (dd, J=11.4, 5.4 Hz, 1H, H-9′), 3.55 (dd, J=9.2, 0.9 Hz, 1H, H-7), 2.31 (q, J=7.6 Hz, 2H, CH₂), 1.15 (t, J=7.6 Hz, 3H, CH₃). ¹³C NMR (126 MHz, CD₃OD) δ 178.80 (N—C═O), 166.46 (C-1), 112.52 (C-3), 77.98 (C-6), 71.17 (C-8), 70.17 (C-7), 68.10 (C-4), 64.93 (C-9), 51.80 (C-5), 30.20 (C-α), 10.33 (C-β). HRMS (ESI) calcd. for C₁₂H₁₈NO₈ [M−H]⁻, 304.1032; found 304.1039.

### Example 47: 5-Butyramido-2,6-anhydro-3,5-dideoxy-D-glycero-D-galacto-non-2-enonic acid (30)

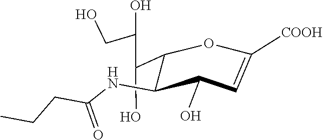

¹H NMR (500 MHz, CD₃OD) δ 5.89 (d, J=2.4 Hz, 1H, H-3), 4.42 (dd, J=8.7, 2.4 Hz, 1H, H-4), 4.15 (d, J=10.8 Hz, 1H, H-6), 3.99 (dd, J=10.8, 8.7 Hz, 1H, H-5), 3.89 (ddd, J=9.0, 5.3, 3.0 Hz, 1H, H-8), 3.81 (dd, J=11.4, 3.0 Hz, 1H, H-9), 3.63 (dd, J=11.4, 5.3 Hz, 1H, H-9′), 3.56 (d, J=9.0 Hz, 1H, H-7), 2.29-2.23 (m, 2H, CH₂), 1.66 (dt, J=13.4, 7.0 Hz, 2H, CH₂), 0.97 (t, J=7.0 Hz, 3H, CH₃). ¹³C NMR (126 MHz, CD₃OD) δ 177.98 (N—C═O), 166.50 (C-1), 112.47 (C-3), 78.00 (C-6), 71.17 (C-7), 70.26 (C-4), 68.09 (C-8), 64.99 (C-9), 51.85 (C-5), 39.04 (C-α), 20.31 (C-β), 14.11 (C-γ). HRMS (ESI) calcd. for C₁₃H₂₀NO₈ [M−H]⁻, 318.1189; found 318.1196.

### Example 48: 5-Pentanamido-9-(4-biphenyl)-2,6-anhydro-3,5-dideoxy-D-glycero-D-galacto-non-2-enonic acid (31)

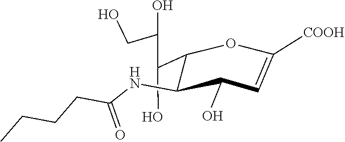

¹H NMR (500 MHz, CD₃OD) δ 5.88 (d, J=2.1 Hz, 1H, H-3), 4.44 (dd, J=8.8, 2.1 Hz, 1H, H-4), 4.15 (d, J=10.8 Hz, 1H, H-6), 3.99 (dd, J=10.8, 8.8 Hz, 1H, H-5), 3.89 (m, 1H, H-8), 3.81 (dd, J=11.4, 2.9 Hz, 1H, H-9), 3.64 (dd, J=11.4, 5.3 Hz, 1H, H-9′), 3.57 (d, J=9.0 Hz, 1H, H-7), 2.29 (t, J=7.6 Hz, 2H, α-CH₂), 1.62 (m, 2H, β-CH₂), 1.37 (dq, J=14.8, 7.4 Hz, 2H, γ-CH₂), 0.93 (t, J=7.4 Hz, 3H, CH₃). ¹³C NMR (126 MHz, CD₃OD) δ 178.13 (N—C═O), 166.89 (C-1), 146.72 (C-2), 112.33 (C-3), 77.91 (C-6), 71.30 (C-7), 70.18 (C-4), 68.09 (C-8), 64.90 (C-9), 51.76 (C-5), 36.92 (C-α), 29.08 (C-β), 23.46 (C-γ), 14.21 (C-δ). HRMS (ESI) calcd. for C₁₄H₂₂NO₈ [M−H]⁻, 332.1345; found 332.1348.

### Example 49: 5-Hexanamido-2,6-anhydro-3,5-dideoxy-D-glycero-D-galacto-non-2-enonic acid (32)

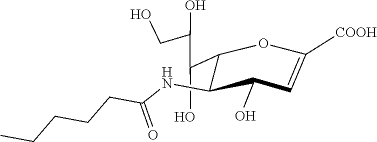

¹H NMR (700 MHz, CD₃OD) δ 5.95 (d, J=2.3 Hz, 1H, H-3), 4.43 (dd, J=8.7, 2.3 Hz, 1H, H-4), 4.16 (dd, J=10.8, 0.8 Hz, 1H, H-6), 3.99 (dd, J=10.8, 8.7 Hz, 1H, H-5), 3.91 (brs, 1H, H-8), 3.83 (dd, J=11.4, 2.9 Hz, 1H, H-9), 3.63 (dd, J=11.4, 5.5 Hz, 1H, H-9′), 3.56 (dd, J=9.3, 0.7 Hz, 1H, H-7), 2.29 (t, J=7.5 Hz, 2H, α-CH₂), 1.69-1.62 (m, 2H, β-CH₂), 1.35 (m, 2×2H, γ-CH₂, δ-CH₂), 0.93 (t, J=7.0 Hz, 3H, CH₃). ¹³C NMR (176 MHz, CD₃OD) δ 178.20 (N—C═O), 113.34 (C-3), 78.14 (C-6), 71.06 (C-7), 70.27 (C-4), 67.95 (C-8), 65.03 (C-9), 51.81 (C-5), 37.06 (C-α), 32.56 (C-β), 26.59 (C-γ), 23.44 (C-δ), 14.27 (C-ε). HRMS (ESI) calcd. for C₁₅H₂₅NO₈ [M−H]⁻, 346.1507; found 346.1506.

### Example 50: 5-Heptanamido-2,6-anhydro-3,5-dideoxy-D-glycero-D-galacto-non-2-enonic acid (33)

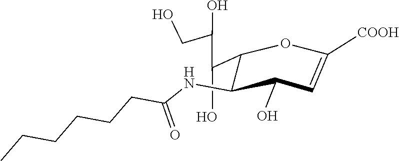

¹H NMR (500 MHz, CD₃OD) δ 5.93 (d, J=2.1 Hz, 1H, H-3), 4.40 (dd, J=8.7, 2.1 Hz, 1H, H-4), 4.13 (d, J=10.8 Hz, 1H, H-6), 3.98 (dd, J=10.8, 8.7 Hz, 1H, H-5), 3.89 (brs, 1H, H-8), 3.81 (dd, J=11.4, 2.7 Hz, 1H, H-9), 3.61 (dd, J=11.4, 5.5 Hz, 1H, H-9′), 3.53 (d, J=8.8 Hz, 1H, H-7), 2.27 (t, J=7.5 Hz, 2H, α-CH₂), 1.69-1.55 (m, 2H, β-CH₂), 1.42-1.16 (m, 3×2H, γ-CH₂, δ-CH₂, ε-CH₂), 0.89 (t, J=7.0 Hz, 3H). ¹³C NMR (126 MHz, CD₃OD) δ 178.23 (N—C═O), 113.38 (C-3), 78.18 (C-6), 71.07 (C-7), 70.32 (C-4), 67.98 (C-8), 65.06 (C-9), 51.84 (C-5), 37.12 (C-α), 32.72 (C-β), 30.06 (C-γ), 26.89 (C-δ), 23.58 (C-ε), 14.40 (C-ζ). HRMS (ESI) calcd. for C₆H₂₆NO₈ [M−H]⁻, 360.1664; found 360.1665.

### Example 51: 5-Isobutyramido-2,6-anhydro-3,5-dideoxy-D-glycero-D-galacto-non-2-enonic acid (34)

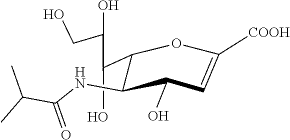

¹H NMR (500 MHz, CD₃OD) δ 5.93 (d, J=2.5 Hz, 1H, H-3), 4.43 (dd, J=8.7, 2.5 Hz, 1H, H-4), 4.15 (dd, J=10.8, 1.1 Hz, 1H, H-6), 3.95 (dd, J=10.8, 8.7 Hz, 1H, H-5), 3.88 (s, 1H, H-8), 3.80 (dd, J=11.4, 2.9 Hz, 1H, H-9), 3.62 (dd, J=11.4, 5.4 Hz, 1H, H-9′), 3.52 (dd, J=9.2, 1.1 Hz, 1H, H-7), 2.57-2.46 (m, 1H, α-CH), 1.14 (dd, J=6.9, 2.4 Hz, 6H, 2×β-CH₃). ¹³C NMR (126 MHz, CD₃OD) δ 182.10 (N—C═O), 165.49 (C-1), 113.48 (C-3), 78.20 (C-6), 71.05 (C-7), 70.21 (C-4), 67.89 (C-8), 64.98 (C-9), 51.68 (C-5), 36.41 (C-α), 20.10, 19.70 (2×C-β). HRMS (ESI) calcd. for C₁₃H₂₁NO₈ [M−H]⁻, 318.1194; found 318.1193.

### Example 52: 5-(3-Methylbutanamido)-2,6-anhydro-3,5-dideoxy-D-glycero-D-galacto-non-2-enonic acid (35)

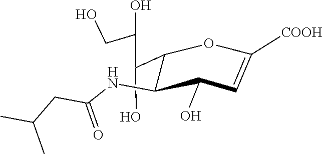

¹H NMR (500 MHz, CD₃OD) δ 5.93 (d, J=2.5 Hz, 1H, H-3), 4.40 (dd, J=8.7, 2.5 Hz, 1H, H-4), 4.14 (dd, J=10.8, 1.1 Hz, 1H, H-6), 3.98 (dd, J=10.8, 8.7 Hz, 1H, H-5), 3.89 (s, 1H, H-8), 3.80 (dd, J=11.4, 2.9 Hz, 1H, H-9), 3.61 (dd, J=11.4, 5.5 Hz, 1H, H-9′), 3.57 (dd, J=9.3, 1.1 Hz, 1H, H-7), 2.17-2.04 (m, 3H, α-CH₂, β-CH₂), 0.96 (dd, J=6.5, 3.8 Hz, 6H, 2×γ-CH₃). ¹³C NMR (126 MHz, CD₃OD) δ 177.53 (N—C═O), 113.50 (C-3), 78.18 (C-6), 71.05 (C-7), 70.34 (C-4), 67.95 (C-8), 65.04 (C-9), 51.87 (C-5), 46.33 (C-α), 27.43 (C-β), 22.89, 22.83 (2×C-γ). HRMS (ESI) calcd. for C₁₄H₂₂NO₈ [M−H]⁻, 332.1351; found 333.1348.

### Example 53: 5-(4-Methylpentanamido)-2,6-anhydro-3,5-dideoxy-D-glycero-D-galacto-non-2-enonic acid (36)

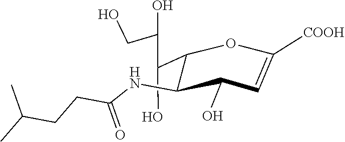

¹H NMR (500 MHz, CD₃OD) δ 5.93 (d, J=2.3 Hz, 1H, H-3), 4.42 (dd, J=8.7, 2.3 Hz, 1H, H-4), 4.14 (d, J=10.8 Hz, 1H, H-6), 3.96 (dd, J=10.8, 8.8 Hz, 1H, H-5), 3.89 (s, 1H, H-8), 3.81 (dd, J=11.4, 2.8 Hz, 1H, H-9), 3.62 (dd, J=11.4, 5.4 Hz, 1H, H-9′), 3.54 (d, J=8.7 Hz, 1H, H-7), 2.32-2.25 (m, 2H, α-CH₂), 1.62-1.47 (m, 3H, β-CH₂, γ-CH), 0.91 (dd, J=6.4, 1.1 Hz, 6H, 2×δ-CH₃). ¹³C NMR (126 MHz, CD₃OD) δ 178.46 (N—C═O), 113.46 (C-3), 78.14 (C-6), 71.07 (C-7), 70.22 (C-4), 67.97 (C-8), 64.99 (C-9), 51.81 (C-5), 35.86 (C-α), 35.19 (C-β), 29.00 (C-γ), 22.75, 22.67 (2×C-β).

HRMS (ESI) calcd. for C₁₅H₂₄NO₈ [M−H]⁻, 346.1507; found 346.1496.

### Example 54: 5-Cyclopropanecarboxamido-2,6-anhydro-3,5-dideoxy-D-glycero-D-galacto-non-2-enonic acid (37)

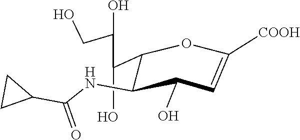

¹H NMR (500 MHz, CD₃OD) δ 8.26 (d, J=8.4 Hz, 1H, NH), 5.88 (d, J=1.5 Hz, 1H, H-3), 4.47 (d, J=8.7 Hz, 1H, H-4), 4.16 (d, J=10.8 Hz, 1H, H-6), 4.01 (m, 1H, H-5), 3.93-3.85 (m, 1H, H-8), 3.80 (dd, J=11.4, 2.6 Hz, 1H, H-9), 3.69-3.62 (m, 1H, H-9), 3.56 (d, J=9.0 Hz, 1H, H-7), 1.69-1.66 (m, 1H, CH), 0.92-0.88 (m, 2H, CH₂), 0.81-0.78 (m, 2H, CH₂). ¹³C NMR (126 MHz, CD₃OD) δ 178.35 (N—C═O), 166.87 (C-1), 112.31 (C-3), 78.06 (C-6), 71.28 (C-7), 70.10 (C-4), 68.19 (C-8), 64.84 (C-9), 51.96 (C-5), 15.10 (C-α), 8.10 (C-β), 7.75 (C-β′). HRMS (ESI) calcd. for C₁₃H₁₈NO₈ [M−H]⁻, 316.1032; found 316.1030.

### Example 55: 5-Cyclobutanecarboxamido-2,6-anhydro-3,5-dideoxy-D-glycero-D-galacto-non-2-enonic acid (38)

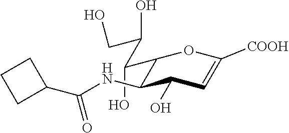

¹H NMR (500 MHz, CD₃OD) δ 5.89 (d, J=1.7 Hz, 1H, H-3), 4.44 (dd, J=8.9, 1.7 Hz, 1H, H-4), 4.15 (d, J=10.6 Hz, 1H, H-6), 3.99 (dd, J=10.6, 8.9 Hz, 1H, H-5), 3.88 (m, 1H, H-8), 3.80 (dd, J=11.4, 2.7 Hz, 1H, H-9), 3.65 (dd, J=11.4, 5.2 Hz, 1H, H-9′), 3.53 (d, J=9.0 Hz, 1H, H-7), 3.19 (p, J=8.5 Hz, 1H, CH), 2.30-2.22 (m, 2H, CH₂), 2.20-2.10 (m, 2H, CH₂), 1.89-1.82 (m, 2H, CH₂). ¹³C NMR (126 MHz, CD₃OD) δ 179.57 (N—C═O), 166.68 (C-1), 112.52 (C-3), 77.96 (C-6), 71.26 (C-7), 70.10 (C-4), 68.04 (C-8), 64.87 (C-9), 51.69 (C-5), 40.83 (C-α), 26.42 (C-β), 26.09 (C-β′), 19.08 (C-γ). HRMS (ESI) calcd. for C₁₄H₂₀NO₈ [M−H]⁻, 330.1189; found 330.1195.

### Example 56: 5-Benzamido-2,6-anhydro-3,5-dideoxy-D-glycero-D-galacto-non-2-enonic acid (39)

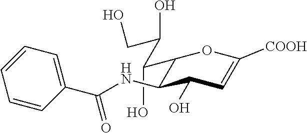

¹H NMR (500 MHz, CD₃OD) δ 7.88 (dd, J=8.3, 1.2 Hz, 2H, Ar—H), 7.57-7.50 (m, 1H, Ar—H), 7.49-7.41 (m, 2H, Ar—H), 5.98 (d, J=2.3 Hz, 1H, H-3), 4.64 (dd, J=8.7, 2.3 Hz, 1H, H-4), 4.32 (d, J=11.0 Hz, 1H, H-6), 4.24 (dd, J=11.0, 8.7 Hz, 1H, H-5), 3.92 (s, 1H, H-8), 3.80 (dd, J=11.5, 2.8 Hz, 1H, H-9), 3.68-3.59 (m, 2H, H-9′, H-7). ¹³C NMR (126 MHz, CD₃OD) δ 171.74 (N—C═O), 135.08, 133.05, 129.55, 128.67 (Ar—C), 113.62 (C-3), 78.17 (C-6), 71.16 (C-7), 70.17 (C-4), 68.06 (C-8), 64.86 (C-9), 52.47 (C-5). HRMS (ESI) calcd. for C₁₆H₁₈NO [M−H]⁻, 352.1038; found 352.1035.

### Example 57: 5-(N-2-azidoacetyl)-2, 3, 5-trideoxy-D-glycero-D-galacto-2-nonulopyranosonic acid (40)

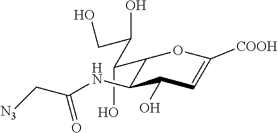

¹H NMR (500 MHz, CD₃OD) δ 5.86 (d, J=1.5 Hz, 1H, H-3), 4.46 (dd, J=8.7, 1.5 Hz, 1H, H-4), 4.24 (d, J=10.8 Hz, 1H, H-6), 4.07 (dd, J=10.8, 8.7 Hz, 1H, H-5), 3.97 (q, J=16.1 Hz, 2H, N—CH₂—CO), 3.88 (br, 1H, H-8), 3.81 (dd, J=11.4, 2.6 Hz, 1H, H-9), 3.66 (dd, J=11.4, 5.2 Hz, 1H, H-9′), 3.57 (d, J=9.0 Hz, 1H, H-7). ¹³C NMR (126 MHz, CD₃OD) δ 171.55 (C═O), 111.68 (C-3), 77.43 (C-6), 71.41 (C-8), 70.04 (C-7), 68.23 (C-4), 64.86 (C-9), 53.01 (CH₂N), 51.93 (C-5). HRMS (ESI) calcd. for C₁₁H₁₅N₄O₈ [M−H]⁻, 331.0890; found 331.0894.

### Example 58: 5-(2-(4-(4-(trifluoromethyl)phenyl)-1H-1,2,3-triazol-1-yl)acetamido))-2, 3, 5-trideoxy-D-glycero-D-galacto-2-nonulopyranosonic acid (41)

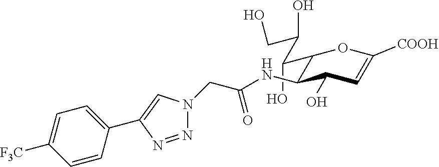

¹H NMR (500 MHz, CD₃OD) δ 8.47 (s, 1H, N—CH═C), 7.97 (d, J=8.1 Hz, 2H, Ar—H), 7.68 (d, J=8.3 Hz, 2H, Ar—H), 5.76 (s, 1H, H-3), 5.32 (s, 2H, N—CH₂—CO), 4.47 (dd, J=8.6, 1.5 Hz, 1H, H-4), 4.27 (d, J=10.8 Hz, 1H, H-6), 4.12 (dd, J=10.8, 8.6 Hz, 1H, H-5), 3.89 (br, 1H, H-8), 3.84-3.76 (m, 1H, H-9), 3.69 (dd, J=11.4, 5.1 Hz, 1H, H-9′), 3.64 (d, J=8.9 Hz, 1H, H-7). ¹³C NMR (126 MHz, CD₃OD) δ 168.79 (C═O), 147.36, 125.07 (Triazole-C), 135.56 (Ar—C), 130.94 (q, J=32.3 Hz, Ar—C), 127.05 (Ar—C), 127.87 (q, J=3.7 Hz, Ar—C), 109.19 (C-3), 76.99 (C-6), 71.60 (C-8), 70.06 (C-7), 68.68 (C-4), 64.80 (9), 53.36 (CH₂N₃), 52.24 (C-5). HRMS (ESI) calcd. for C₂₀H₂₀F₃N₄O₈ [M−H]⁻, 501.1233; found 501.1243.

### Example 59: 5-(2-(4-(4-p-tolyl)-1H-1,2,3-triazol-1-yl) acetamido))-2, 3, 5-trideoxy-D-glycero-D-galacto-2-nonulogyranosonic acid (42)

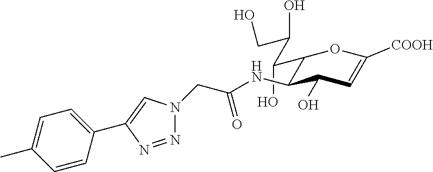

¹H NMR (500 MHz, CD₃OD) δ 8.31 (s, 1H, NC═CH), 7.70 (d, J=8.1 Hz, 2H, Ar—H), 7.24 (d, J=8.0 Hz, 2H), 5.87 (d, J=2.2 Hz, 1H, H-3), 5.29 (s, 2H, N—CH₂—CO), 4.49 (dd, J=8.7, 2.2 Hz, 1H, H-4), 4.29 (d, J=10.8 Hz, 1H, H-6), 4.10 (dd, J=10.7, 8.7 Hz, 1H, H-5), 3.91-3.84 (m, 1H, H-8), 3.81 (dd, J=11.4, 2.9 Hz, 1H, H-9), 3.67 (dd, J=11.4, 5.3 Hz, 1H, H-9′), 3.64 (d, J=9.1 Hz, 1H, H-7), 2.35 (s, 3H, PhCH₃). ¹³C NMR (126 MHz, CD₃OD) δ 168.97 (C═O), 167.13 (C-1), 148.95, 123.68 (Triazole-C), 146.97 (C-2), 139.45, 130.64, 128.89, 126.71 (Ar—C), 111.62 (C-3), 77.42 (C-6), 71.51 (C-8), 70.06 (C-7), 68.30 (C-4), 64.86 (C-9), 53.29 (CH₂N), 52.18 (C-5), 21.37 (PhCH₃). HRMS (ESI) calcd. for C₂₀H₂₃N₄O₈ [M−H]⁻, 447.1516; found 447.1520.

### Example 60: 5-(2-(4-(4-carboxyphenyl)-1H-1,2,3-triazol-1-yl) acetamido))-2, 3, 5-trideoxy-D-glycero-D-galacto-2-nonulopyranosonic acid (43)

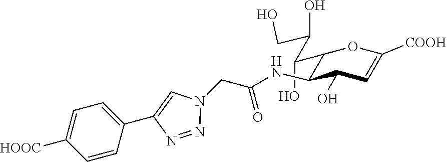

¹H NMR (500 MHz, CD₃OD) δ 8.48 (s, 1H, C═CH—N), 8.10-8.04 (m, 2H, Ar—H), 7.95-7.90 (m, 2H, Ar—H), 5.88 (d, J=2.4 Hz, 1H, H-3), 5.32 (s, 2H, N—CH₂—CO), 4.49 (dd, J=8.7, 2.4 Hz, 1H, H-4), 4.30 (dd, J=10.8, 0.6 Hz, 1H, H-6), 4.11 (dd, J=10.8, 8.7 Hz, 1H, H-5), 3.91-3.86 (m, 1H, H-8), 3.82 (dd, J=11.4, 3.0 Hz, 1H, H-9), 3.68 (dd, J=11.5, 5.4 Hz, 1H, H-9′), 3.64 (dd, J=9.2, 0.6 Hz, 1H, H-7). ¹³C NMR (126 MHz, DMSO-d₆) δ 169.44, 168.85 (C═O), 167.00 (C-1), 147.83, 125.04 (Triazole-C), 146.85 (C-2), 136.10, 131.60, 131.48, 126.53 (Ar—C), 111.73 (C-3), 77.44 (C-6), 71.45 (C-8), 70.09 (C-7), 68.32 (C-4), 64.88 (C-9), 53.32 (CH₂N₃), 52.21 (C-5). HRMS (ESI) calcd. for C₂₀H₂₁N₄O₁₀[M−H]⁻, 477.1258; found 477.1268.

### Example 61: 5-(2-(4-(4-methoxyphenyl)-1H-1,2,3-triazol-1-yl)acetamido))-2, 3, 5-trideoxy-D-glycero-D-galacto-2-nonulopyranosonic acid (44)

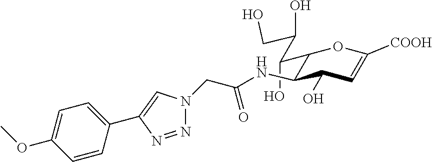

¹H NMR (500 MHz, CD₃OD) δ 8.27 (s, 1H, C═CH—N), 7.74 (d, J=8.7 Hz, 2H, Ar—H), 7.01 (d, J=8.7 Hz, 2H, Ar—H), 5.89 (d, J=1.8 Hz, 1H, H-3), 5.31 (s, 2H, N—CH₂—CO), 4.56-4.45 (m, 1H, H-4), 4.33 (d, J=10.8 Hz, 1H, H-6), 4.11 (dd, J=10.5, 8.9 Hz, 1H, H-5), 3.92-3.86 (m, 1H, H-8), 3.85-3.83 (m, 4H, H-9, OCH₃), 3.70-3.63 (m, 2H, H-7, H-9′). ¹³C NMR (126 MHz, CD₃OD) δ 169.15 (C═O), 167.27 (C-1), 148.92, 123.53 (Triazole-C), 146.62 (C-2), 161.19, 128.28, 124.06 115.61 (Ar—C), 112.08 (C-3), 77.26 (C-6), 71.38 (C-8), 69.81 (C-7), 68.41 (C-4), 64.77 (C-9), 56.20 (PhOCH₃), 53.38 (CH₂N₃), 52.02 (C-5). HRMS (ESI) calcd. for C₂₀H₂₃N₄O₉ [M−H]⁻, 463.1465; found 463.1471.

### Example 62: 5-(2-(4-(4-fluorophenyl)-1H-1,2,3-triazol-1-yl)acetamido))-2,3,5-trideoxy-D-glycero-D-galacto-2-nonulopyranosonic acid (45)

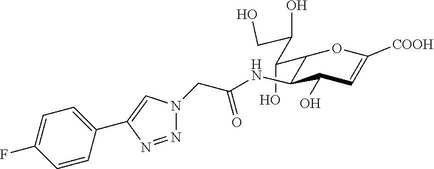

¹H NMR (500 MHz, CD₃OD) δ 8.33 (s, 1H, C═CH—N), 7.81 (dd, J=8.6, 5.4 Hz, 2H, Ar—H), 7.14 (t, J=8.7 Hz, 2H, Ar—H), 5.80 (s, 1H, H-3), 5.29 (s, 2H, N—CH₂—CO), 4.48 (d, J=8.2 Hz, 1H, H-4), 4.27 (d, J=10.7 Hz, 1H, H-6), 4.14-4.07 (m, 1H, H-5), 3.88 (m, 1H, H-8), 3.81 (d, J=11.0 Hz, 1H, H-9), 3.73-3.60 (m, 2H, H-9′, H-7). ¹³C NMR (126 MHz, CD₃OD) δ 168.94 (C-1), 168.64 (C═O), 165.11, 163.15 (d, J=246.3 Hz), 148.28 (C-2), 147.97, 123.91 (Triazole-C), 128.70 (d, J=8.2 Hz, Ar—C), 128.11 (d, J=3.2 Hz, Ar—C), 116.78 (d, J=22.0 Hz, Ar—C), 110.10 (C-3), 77.12 (C-6), 71.64 (C-8), 69.99 (C-7), 68.50 (C-4), 64.75 (C-9), 53.32 (CH₂N), 52.18 (C-5). HRMS (ESI) calcd. for C₁₉H₂₀N₄O₈ [M−H]⁻, 451.1265; found 451.1271.

### Example 63: 5-(2-(4-(4-aminophenyl)-1H-1,2,3-triazol-1-yl) acetamido))-2, 3, 5-trideoxy-D-glycero-D-galacto-2-nonulopyranosonic acid (46)

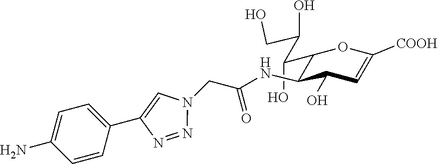

¹H NMR (500 MHz, CD₃OD) δ 8.18 (s, 1H, C═CH—N), 7.57 (d, J=8.6 Hz, 2H, Ar—H), 6.81 (d, J=8.5 Hz, 2H, Ar—H), 5.70 (d, J=2.2 Hz, 1H, H-3), 5.28 (s, 2H, N—CH₂—CO), 4.47 (dd, J=8.7, 2.2 Hz, 1H, H-4), 4.25 (d, J=10.8 Hz, 1H, H-6), 4.09 (dd, J=10.8, 8.7 Hz, 1H, H-5), 3.93-3.86 (m, 1H, H-8), 3.83 (dd, J=11.6, 2.8 Hz, 1H, H-9), 3.66 (dd, J=11.6, 5.7 Hz, 1H, H-9′), 3.60 (d, J=9.3 Hz, 1H, H-7). ¹³C NMR (126 MHz, CD₃OD) δ 169.93 (C-1), 169.04 (C═O), 149.58, 122.78 (Triazole-C), 148.93 (C-2), 127.94, 121.52, 117.07 (Ar—C), 108.57 (C-3), 76.73 (C-6), 71.42 (C-8), 70.02 (C-7), 68.81 (C-4), 64.87 (C-9), 53.35 (CH₂N), 52.19 (C-5). HRMS (ESI) calcd. for C₁₉H₂₂N₅O₈ [M−H]⁻, 448.1468; found 448.1481.

### Example 64: 5-(2-(4-(4-(dimethylamino)phenyl-1H-1,2,3-triazol-1-yl)acetamido))-2,3,5-trideoxy-D-glycero-D-galacto-2-nonulopyranosonic acid (47)

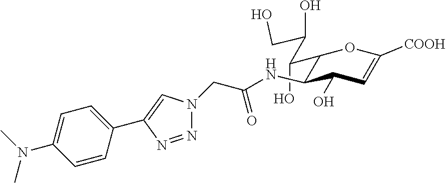

¹H NMR (500 MHz, CD₃OD) δ 8.18 (s, 1H, C═CH—N), 7.57 (d, J=8.6 Hz, 2H, Ar—H), 6.81 (d, J=8.5 Hz, 2H, Ar—H), 5.70 (d, J=2.2 Hz, 1H, H-3), 5.28 (s, 2H, N—CH₂—CO), 4.47 (dd, J=8.7, 2.2 Hz, 1H, H-4), 4.25 (d, J=10.8 Hz, 1H, H-6), 4.09 (dd, J=10.8, 8.7 Hz, 1H, H-5), 3.93-3.86 (m, 1H, H-8), 3.83 (dd, J=11.6, 2.8 Hz, 1H, H-9), 3.66 (dd, J=11.6, 5.7 Hz, 1H, H-9′), 3.60 (d, J=9.3 Hz, 1H, H-7). ¹³C NMR (126 MHz, CD₃OD) δ 169.93 (C-1), 169.04 (C═O), 149.58, 122.78 (Triazole-C), 148.93 (C-2), 127.94, 121.52, 117.07 (Ar—C), 108.57 (C-3), 76.73 (C-6), 71.42 (C-8), 70.02 (C-7), 68.81 (C-4), 64.87 (C-9), 53.35 (CH₂N), 52.19 (C-5). HRMS (ESI) calcd. for C₁₉H₂₂N₅O₈ [M−H]⁻, 448.1468; found 448.1481.

### Example 65: 5-(2-(4-(4-acetamidophenyl)-1H-1,2,3-triazol-1-yl) acetamido))-2, 3, 5-trideoxy-D-glycero-D-galacto-2-nonulopyranosonic acid (48)

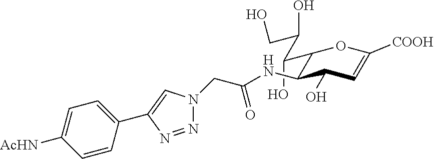

¹H NMR (500 MHz, CD₃OD) δ 8.30 (s, 1H, C═CH—N), 7.75 (d, J=8.6 Hz, 2H, Ar—H), 7.62 (d, J=8.6 Hz, 2H, Ar—H), 5.69 (d, J=2.2 Hz, 1H, H-3), 5.26 (s, 2H, N—CH₂—CO), 4.43 (dd, J=8.6, 2.2 Hz, 1H, H-4), 4.23 (d, J=10.8 Hz, 1H, H-6), 4.10 (dd, J=10.8, 8.6 Hz, 1H, H-5), 3.90-3.84 (m, 1H, H-8), 3.81 (dd, J=11.4, 2.9 Hz, 1H, H-9), 3.67 (dd, J=11.4, 5.3 Hz, 1H, H-9′), 3.57 (d, J=9.2 Hz, 1H, H-7), 2.13 (s, 3H, NAc). ¹³C NMR (126 MHz, CD₃OD) δ 171.70 (C═O), 169.98 (C-1), 168.75 (C═O), 150.14 (C-1), 148.58, 123.59 (Triazole-C), 140.08, 127.37, 127.17, 121.36 (Ar—C), 108.11 (C-3), 76.80 (C-6), 71.44 (C-8), 70.17 (C-7), 68.78 (C-4), 64.92 (C-9), 53.30 (CH₂N₃), 52.23 (C-5), 23.90 (CH₃). HRMS (ESI) calcd. for C₂₁H₂₅N₅O₉ [M−H]⁻, 490.1574; found 490.1584.

### Example 66: General Procedure for Synthesis of C5, 9-Amido Compounds

Fully protected C9-azido DANA (II-34) was dissolved in THF-H₂O and cooled down to 0° C. with ice water bath. Triphenyl phosphate was then added followed with activated carboxylic acids. The solution was allowed to warm to room temperature and kept stirring overnight. After completion, the reaction was quenched with water, concentrated and purified by flash chromatography to give the desired C9-modified product. The product was then dissolved in anhydrous DCM and TEA and then cooled down to 0° C. Corresponding activated carboxylic acids for C5 modifications was added in dropwise. The solution was allowed to warm to room temperature and kept stirring overnight. After completion, the reaction was quenched with water, concentrated and purified by flash chromatography to give the desired C5-modified product. The product was then dissolved in MeOH, and 0.5 M NaOH was added (FIG. 11). The mixture was kept stirring at room temperature. After completion, the mixture was neutralized with Amberlite™ IR-120 (H⁺ form), filtered and purified by flash chromatography to provide the final C5-, C9-modified compounds.

### Example 67: 5-Pentanamido-9-pentanamido-2,6-anhydro-3,5-dideoxy-D-glycero-D-galacto-non-2-enonic acid (57)

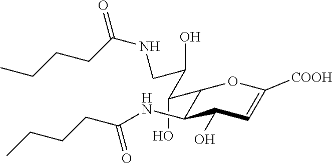

¹H NMR (500 MHz, CD₃OD) δ 5.74 (d, J=1.9 Hz, 1H, H-3), 4.38 (dd, J=8.7, 1.9 Hz, 1H, H-4), 4.11 (d, J=10.8 Hz, 1H, H-6), 3.97 (dd, J=10.7, 8.8 Hz, 1H, H-5), 3.93-3.85 (m, 1H, H-8), 3.57 (dd, J=13.4, 3.1 Hz, 1H, H-9), 3.38 (d, J=8.7 Hz, 1H, H-7), 3.26 (dd, J=13.4, 6.0 Hz, 1H, H-9′), 2.30-2.23 (m, 2H, α-CH₂), 2.23-2.15 (m, 2H, α′-CH₂), 1.59 (tdd, J=15.3, 11.2, 7.5 Hz, 4H, β-CH₂, β′-CH₂), 1.34 (dq, J=22.0, 7.4 Hz, 4H, γ-CH₂, γ′-CH₂), 0.92 (q, J=7.4 Hz, 6H, δ-CH₂, δ′-CH₂). ¹³C NMR (126 MHz, CD₃OD) δ 177.78, 176.97 (N—C═O), 109.98 (C-3), 77.36 (C-6), 71.51 (C-7), 70.36 (C-4), 68.40 (C-8), 51.87 (C-5), 44.27 (C-9), 36.96, 36.89 (C-α, C-α′), 29.24, 29.11 (C-β, C-β′), 23.49, 23.45 (C-γ, C-γ′), 14.19 (COCH₃). HRMS (ESI) calcd. for C₁₉H₃₁N₂O₈ [M−H]⁻, 415.2086; found 415.2081.

### Example 68: General Procedure of Staudinger Reaction for Synthesis of Compounds 58-62

C9-azido DANA methyl ester was dissolved in THF-H₂O and cooled down to 0° C. with ice water bath. Triphenyl phosphate was then added followed with anhydrides or acyl chlorides. The solution was allowed to warm to room temperature and kept stirring overnight. After completion, the reaction was quenched with water, concentrated and purified by flash chromatography to give the desired product. The product was then dissolved in MeOH, and 0.5 M NaOH was added. The mixture was kept stirring at room temperature. After completion, the mixture was neutralized with Amberlite™ IR-120 (H⁺ form), filtered and purified by flash chromatography to provide the desired products.

### Example 69: 5-Acetamido-9-(4-acetamido)benzamido-2,6-anhydro-3,5-dideoxy-d-glycero-d-galacto-non-2-enonic acid (58)

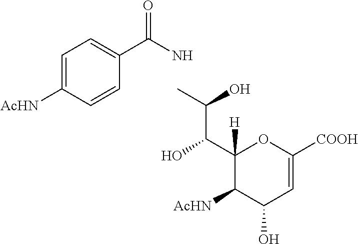

Compound 58 was synthesized from C9-azido DANA methyl ester using 4-acetamidobenzyl chloride. 45 mg (28%(36%×77%, over two steps). ¹H NMR (500 MHz, D₂O) δ 7.80 (d, J=8.2 Hz, 2H, Ar—H), 7.58 (d, J=8.2 Hz, 2H, Ar—H), 5.95 (s, 1H, H-3), 4.54 (d, J=7.8 Hz, 1H, H-4), 4.33 (d, J=10.9 Hz, 1H, H-6), 4.14 (t, J=9.5 Hz, 2H, H-5, H-8), 3.86-3.80 (m, 1H, H-9), 3.68-3.58 (m, 2H, H-7, H-9′), 2.22 (s, 3H, COCH₃), 2.07 (s, 3H, COCH₃). ¹³C NMR (126 MHz, D₂O) δ 175.64, 173.90, 171.46 (3×N—C═O), 168.07 (C-1), 141.49, 130.56, 129.13, 121.72 (Ar—C), 111.40 (C-3), 76.57 (C-6), 70.37 (C-7), 69.56 (C-4), 68.16 (C-8), 50.72 (C-5), 44.12 (C-9), 24.04, 23.02 (2×COCH₃). HRMS (ESI) calcd. for C₁₈H₂₁N₂O₈ [M−H]⁻, 450.1518; found 450.1525.

### Example 70: 5-Acetamido-9-(4-amino)benzamido-2,6-anhydro-3,5-dideoxy-D-glycero-D-galacto-non-2-enonic acid (59)

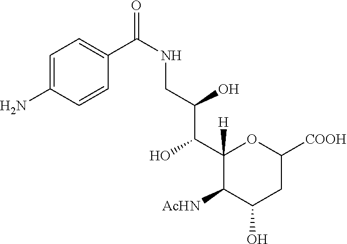 [SMILES: CC(=O)N[C@H]1[C@H]([C@H](O)[C@H](O)CNC(=O)c2ccc(N)cc2)OC(C(=O)O)C[C@@H]1O]

Compound 59 was synthesized from C9-azido DANA methyl ester using N-hydroxysuccinimidyl-4-((tert-butoxycarbonyl) amino) benzoate. 30 mg (20%(45%×45% (yields for two steps of deprotection), over three steps). ¹H NMR (500 MHz, CD₃OD) δ 7.91 (d, J=7.3 Hz, 2H), 7.33 (d, J=7.3 Hz, 2H), 5.94 (s, 1H, H-3), 4.46 (d, J=8.0 Hz, 1H, H-4), 4.21 (d, J=10.6 Hz, 1H, H-6), 4.08-3.96 (m, 2H, H-5, H-8), 3.77 (d, J=12.9 Hz, 1H, H-9), 3.51 (m, 2H, H-9′, H-7), 1.97 (s, 3H, COCH₃). ¹³C NMR (126 MHz, CD₃OD) δ 165.66 (C-1), 145.21, 130.38, 122.41 (Ar—C), 113.68 (C-3), 77.75 (C-6), 71.40 (C-7), 70.28 (C-4), 67.93 (C-8), 51.73 (C-5), 45.05 (C-9), 22.92 (COCH₃). HRMS (ESI) calcd. for C₁₈H₂₁N₂O₈ [M−H]⁻, 408.1412; found 408.1415.

### Example 71: 5-Acetamido-9-(3-acetamido)benzamido-2,6-anhydro-3,5-dideoxy-d-glycero-d-galacto-non-2-enonic acid (60)

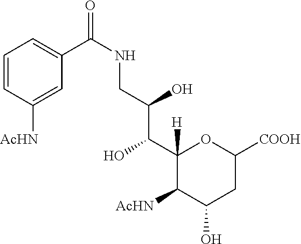 [SMILES: CC(=O)Nc1cccc(C(=O)NC[C@@H](O)[C@@H](O)[C@@H]2OC(C(=O)O)C[C@H](O)[C@H]2NC(C)=O)c1]

Compound 60 was synthesized from C9-azido DANA methyl ester using 3-acetamidobenzyl chloride. 40 mg (30%(36%×82%, over two steps). ¹H NMR (500 MHz, CD₃OD) δ 7.97 (t, J=1.5 Hz, 1H, Ar—H), 7.69 (dd, J=7.9, 1.5 Hz, 1H, Ar—H), 7.52 (d, J=7.9 Hz, 1H, Ar—H), 7.37 (t, J=7.9 Hz, 1H, Ar—H), 5.78 (d, J=2.0 Hz, 1H, H-3), 4.38 (dd, J=8.6, 2.0 Hz, 1H, H-4), 4.16 (d, J=10.8 Hz, 1H, H-6), 4.00 (m, 2H, H-5, H-8), 3.76 (dd, J=13.8, 3.3 Hz, 1H, H-9), 3.53 (dd, J=13.8, 6.8 Hz, 1H, H-9′), 3.47 (d, J=8.8 Hz, 1H, H-7), 2.12 (s, 3H, COCH₃), 1.97 (s, 3H, COCH₃). ¹³C NMR (126 MHz, CD₃OD) δ 174.74, 171.81, 170.56 (3×N—C═O), 140.21, 136.51, 130.03, 124.15, 123.74, 120.14 (Ar—C), 110.38 (C-3), 77.38 (C-6), 71.78 (C-7), 70.21 (C-4), 68.40 (C-8), 51.96 (C-5), 45.07 (C-9), 23.86, 22.77 (2×COCH₃). HRMS (ESI) calcd. for C₁₈H₂₁N₂O₈ [M−H]⁻, 450.1518; found 450.1515.

### Example 72: 5-Acetamido-9-(3-amino)benzamido-2,6-anhydro-3,5-dideoxy-D-glycero-D-galacto-non-2-enonic acid (61)

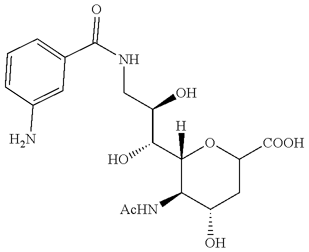 [SMILES: CC(=O)N[C@H]1[C@H]([C@H](O)[C@H](O)CNC(=O)c2cccc(N)c2)OC(C(=O)O)C[C@@H]1O]

Compound 61 was synthesized from C9-azido DANA methyl ester using 3-amidobenzyl chloride. ¹H NMR (500 MHz, CD₃OD) 30 mg (18%(46%×40%, over two steps). δ 7.94 (d, J=7.8 Hz, 1H, Ar—H), 7.89 (s, 1H, Ar—H), 7.63 (t, J=7.8 Hz, 1H, Ar—H), 7.60-7.56 (m, 1H, Ar—H), 5.95 (d, J=2.4 Hz, 1H, H-3), 4.45 (dd, J=8.7, 2.4 Hz, 1H, H-4), 4.21 (d, J=10.9 Hz, 1H, H-6), 4.08-3.97 (m, 2H, H-5, H-8), 3.84-3.79 (m, 1H, H-9), 3.57-3.48 (m, 2H, H-7, H-9′), 1.99 (s, 3H, COCH₃). ¹³C NMR (126 MHz, CD₃OD) δ 175.00, 168.86 (2×N—C═O), 165.60 (C-1), 145.27 (C-2), 137.83, 132.62, 131.55, 128.67, 127.16, 123.51 (Ar—C), 113.61 (C-3), 77.79 (C-6), 71.72 (C-7), 70.03 (C-4), 67.90 (C-8), 51.81 (C-5), 45.29 (C-9), 22.80 (COCH₃). HRMS (ESI) calcd. for C₁₈H₂₁N₂O₈ [M−H]⁻, 408.1412; found 408.1411.

### Example 73: 5-Acetamido-9-(5-(4-acetamidobenzamido))pentanamido-2,6-anhydro-3,5-dideoxy-d-glycero-d-galacto-non-2-enonic acid (62)

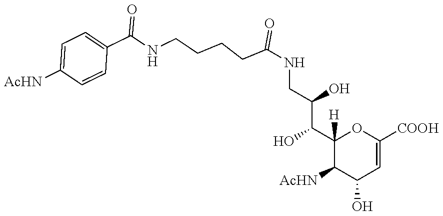 [SMILES: CC(=O)Nc1ccc(C(=O)NCCCCC(=O)NC[C@@H](O)[C@@H](O)[C@@H]2OC(C(=O)O)=C[C@H](O)[C@H]2NC(C)=O)cc1]

Compound 62 was synthesized from C9-azido DANA methyl ester using N-hydroxysuccinimidyl-5-(4-acetamidobenzamido) pentanoate. 30 mg (22%(42%×53%, over two steps). ¹H NMR (500 MHz, CD₃OD) δ 7.77 (d, J=8.7 Hz, 2H, Ar—H), 7.64 (d, J=8.7 Hz, 2H, Ar—H), 5.90 (d, J=2.3 Hz, 1H, H-3), 4.42 (dd, J=8.7, 2.3 Hz, 1H, H-4), 4.19-4.13 (d, J=10.7, 1H, H-6), 3.98 (dd, J=10.7, 8.7 Hz, 1H, H-5), 3.93-3.86 (m, 1H, H-8), 3.59 (dd, J=13.8, 3.1 Hz, 1H, H-9), 3.42 (d, J=8.9 Hz, 1H, H-7), 3.37 (t, J=6.0 Hz, 2H, δ-CH₂), 2.27 (t, J=7.1 Hz, 2H, α-CH₂), 2.13, 2.01 (2×s, 2×3H, 2×COCH₃), 1.73-1.56 (m, 4H, β-CH₂, γ-CH₂). ¹³C NMR (126 MHz, CD₃OD) δ 176.77, 174.82, 171.90, 169.57 (4×N—C═O), 143.07 (C-2), 130.74, 129.15, 120.26 (Ar—C), 112.90 (C-3), 77.69 (C-6), 71.35 (C-8), 70.24 (C-7), 51.81 (C-4), 49.88 (C-5), 44.28 (C-9), 40.53 (δ-CH₂), 36.59 (α-CH₂), 30.04 (γ-CH₂), 24.42 (α-CH₂), 24.03, 22.86 (2×COCH₃). HRMS (ESI) calcd. for C₂₅H₃₃N₄O₁₀ [M−H]⁻, 549.2202; found 549.2207.

### Example 74: 5-Acetamido-9-(4-pentyl)triazolyl-2,6-anhydro-3,5-dideoxy-d-glycero-d-galacto-non-2-enonic acid (63)

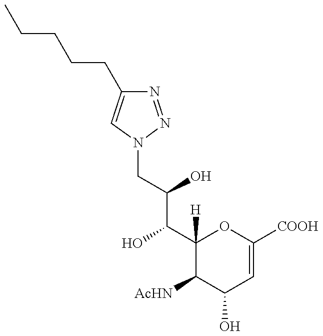 [SMILES: CCCCCc1cn(C[C@@H](O)[C@@H](O)[C@@H]2OC(C(=O)O)=C[C@H](O)[C@H]2NC(C)=O)nn1]

Compound 63: To a solution of methyl 5-acetamido-9-azido-2,6-anhydro-3,5-dideoxy-d-glycero-d-galacto-non-2-enonate (50 mg, 1 eq) and the heptyne (30 mg, 2 eq) in THF-H₂O (2:1), sodium L-ascorbate (5 mg, 0.3 eq) and copper (II) sulfate (3 mg, 0.2 eq) were added sequentially. The reaction mixture was kept stirring at room temperature and monitored by TLC until no azide remained. Silica gel was then added to the reaction mixture and the solvent was removed under reduced pressure. The residue was separated by flash chromatography to provide the desired product 59 mg (92%). To hydrolyze the C1-methyl ester, the product was dissolved in MeOH, and 0.5 M NaOH was added. The mixture was kept stirring at room temperature. After completion, the pH of the solution was adjusted to 2 with Amberlite™ IR-120 (H⁺). The solution was filtered, concentrated and purified by flash chromatography or recrystallization to provide the desired product 40 mg (70%). ¹H NMR (500 MHz, CD₃OD) δ 7.71 (s, 1H, Triazole-H), 5.91 (d, J=2.3 Hz, 1H, H-3), 4.77 (dd, J=14.0, 2.5 Hz, 1H, H-4), 4.44-4.33 (m, 2H, H-6, H-5), 4.24-4.16 (m, 1H, H-8), 4.13 (d, J=10.9 Hz, 1H, H-9), 3.98 (dd, J=10.7, 8.7 Hz, 1H, H-9′), 3.39 (d, J=9.1 Hz, 1H, H-7), 2.66 (t, J=7.7 Hz, 2H, α-CH₂), 2.01 (s, 3H, COCH₃), 1.71-1.59 (m, 2H), 1.39-1.27 (m, 4H, β-CH₂, γ-CH₂), 0.89 (dd, J=9.7, 4.3 Hz, 3H, δ-CH₃). ¹³C NMR (126 MHz, CD₃OD) δ 175.10, 166.11, 145.91, 124.31, 112.97, 77.69, 71.34, 69.86, 67.93, 55.00, 51.92, 32.51, 30.36, 26.29, 23.46, 22.72, 14.35). HRMS (ESI) calcd. for C₁₈H₂₇N₄O₇ [M−H]⁻, 411.1885; found 411.1889.

### Example 75: General Procedure for Synthesis of C5, C9 Double Modified DANA Analogue Compounds 64-70, 73-74

Compound methyl 5-(tert-butoxycarbonyl)amino-9-azido-4,7,8-di-O-acetyl-2,6-anhydro-3,5-dideoxy-D-glycero-D-galacto-non-2-enonate was dissolved in THF-H₂O, and cooled down to 0° C. with ice water bath. Triphenyl phosphate was then added followed with activated carboxylic acids. The solution was allowed to warm to room temperature and kept stirring overnight. After completion, the reaction was quenched with water, concentrated and purified by flash chromatography to give the desired C9-modified product. The product was then dissolved in anhydrous TFA-DCM (10%) and the solution was stirred for 2-4 hours at r.t. Solvents were removed under vacuum and the residue was dissolved in anhydrous DCM and TEA was added. The solution was cooled down to 0° C. and corresponding activated carboxylic acids for C5 modifications was added in dropwise. The solution was allowed to warm to room temperature and kept stirring overnight. After completion, the reaction was quenched with water, concentrated and purified by flash chromatography to give the desired C5-modified product. The product was then dissolved in MeOH, and 0.5 N NaOH was added. The mixture was kept stirring at room temperature. After completion, the mixture was neutralized with Amberlite™ IR-120 (H⁺ form), filtered and purified by flash chromatography to provide the final C5, C9-double modified compounds.

### Example 76: 5-Hexanamido-9-hexanamido-2,6-anhydro-3,5-dideoxy-d-glycero-d-galacto-non-2-enonic acid (64)

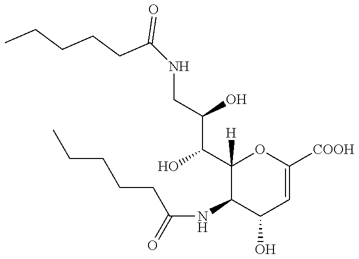 [SMILES: CCCCCC(=O)NC[C@@H](O)[C@@H](O)[C@@H]1OC(C(=O)O)=C[C@H](O)[C@H]1NC(=O)CCCCC]

Compound 64 was synthesized from compound methyl 5-(tert-butoxycarbonyl)amino-9-azido-4,7,8-di-O-acetyl-2,6-anhydro-3,5-dideoxy-D-glycero-D-galacto-non-2-enonate using hexanoic anhydride. 20 mg. (22%(77%×54%×52%), over three steps) ¹H NMR (500 MHz, CD₃OD) δ 5.78 (s, 1H, H-3), 4.38 (d, J=8.5 Hz, 1H, H-4), 4.13 (d, J=10.6 Hz, 1H, H-6), 4.02-3.86 (m, 2H, H-5, H-8), 3.56 (d, J=13.2 Hz, 1H, H-9), 3.42 (d, J=7.9 Hz, 1H, H-7), 2.31-2.12 (m, 4H, α-CH₂, α′-CH₂), 1.68-1.53 (m, 4H, β-CH₂, β′-CH₂), 1.40-1.23 (m, 8H, γ-CH₂, γ′-CH₂, δ-CH₂, δ′-CH₂), 0.90 (q, J=6.9 Hz, 6H, ε-CH₃, ε′-CH₃). ¹³C NMR (126 MHz, CD₃OD) δ 177.85, 177.06 (N—C═O), 110.69 (C-3), 77.49 (C-6), 71.39 (C-7), 70.66 (C-4), 68.30 (C-8), 51.88 (C-5), 44.25 (C-9), 37.18, 37.11 (C-α, C-α′), 32.64, 32.61 (C-β, C-β′), 26.78, 26.66 (C-γ, C-γ′), 23.46 (C-5, C-5′), 14.32, 14.30 (C-ε, C-ε′). HRMS (ESI) calcd. for C₂₁H₃₅N₂O₈ [M−H]⁻, 443.2399; found 443.2396.

### Example 77: 5-Propionamido-9-pentanamido-2,6-anhydro-3,5-dideoxy-D-glycero-D-galacto-non-2-enonic acid (65)

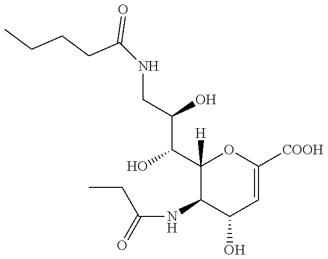 [SMILES: CCCCC(=O)NC[C@@H](O)[C@@H](O)[C@@H]1OC(C(=O)O)=C[C@H](O)[C@H]1NC(=O)CC]

Compound 65 was synthesized from compound methyl 5-(tert-butoxycarbonyl)amino-9-azido-4,7,8-di-O-acetyl-2,6-anhydro-3,5-dideoxy-D-glycero-D-galacto-non-2-enonate using valeric anhydride and propionic anhydride. 15 mg. (18%(60%×53%×58%), over three steps). ¹H NMR (500 MHz, CD₃OD) δ 5.90 (d, J=2.4 Hz, 1H, H-3), 4.41 (dd, J=8.7, 2.3 Hz, 1H, H-4), 4.16 (d, J=10.8 Hz, 1H, H-6), 4.01-3.87 (m, 2H, H-5, H-8), 3.61-3.53 (m, 1H, H-9), 3.41-3.35 (m, 1H, H-7), 2.28 (q, J=7.6 Hz, 2H, α-CH₂), 2.24-2.17 (m, 2H, α-CH₂′), 1.57 (dt, J=13.0, 7.5 Hz, 2H, 3′-CH₂), 1.33 (dq, J=14.7, 7.4 Hz, 2H, γ′-CH₂), 1.14 (t, J=7.6 Hz, 3H, β-CH₃), 0.91 (t, J=7.4 Hz, 3H, 6′-CH₃). ¹³C NMR (126 MHz, CD₃OD) δ 178.61, 177.24 (N—C═O), 112.84 (C-3), 77.83 (C-6), 71.51 (C-7), 70.19 (C-4), 68.00 (C-8), 51.87 (C-5), 44.40 (C-9), 36.82 (C-α′), 30.20 (C-β′), 29.23 (C-γ′), 23.42 (C-α), 14.13 (C-δ′), 10.37 (C-β). HRMS (ESI) calcd. for C₁₇H₂₇N₂O₈ [M−H]⁻, 387.1773; found 387.1770.

### Example 78: 5-Propionamido-9-hexanamido-2,6-anhydro-3,5-dideoxy-D-glycero-D-galacto-non-2-enonic acid (66)

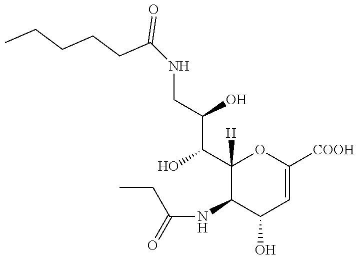 [SMILES: CCCCCC(=O)NC[C@@H](O)[C@@H](O)[C@@H]1OC(C(=O)O)=C[C@H](O)[C@H]1NC(=O)CC]

Compound 66 was synthesized from methyl 5-(tert-butoxycarbonyl)amino-9-azido-4,7,8-di-O-acetyl-2,6-anhydro-3,5-dideoxy-D-glycero-D-galacto-non-2-enonate using hexanoic anhydride and propionic anhydride. 13 mg. (17%(63%×46%×60%), over three steps). ¹H NMR (500 MHz, CD₃OD) δ 5.89 (s, 1H, H-3), 4.40 (d, J=8.7 Hz, 1H, H-4), 4.15 (d, J=10.7 Hz, 1H, H-6), 4.01-3.86 (m, 2H, H-5, H-8), 3.57 (d, J=11.9 Hz, 1H, H-9), 3.39 (d, J=8.6 Hz, 1H, H-7), 2.28 (q, J=7.5 Hz, 2H, α-CH₂), 2.24-2.14 (m, 2H, α′-CH₂), 1.59 (dt, J=14.8, 7.5 Hz, 2H, β′-CH₂), 1.38-1.25 (m, 4H, γ′-CH₂, δ′-CH₃), 1.14 (t, J=7.6 Hz, 3H, β-CH₃), 0.89 (t, J=7.0 Hz, 3H, ε-CH₃′). ¹³C NMR (126 MHz, CD₃OD) δ 178.60, 177.22 (2×N—C═O), 112.68 (C-3), 77.89 (C-6), 71.56 (C-7), 70.25 (C-4), 68.10 (C-8), 51.87 (C-6), 44.40 (C-9), 37.07 (C-α′), 32.58 (C-β′), 30.21 (C-γ′), 26.77 (C-δ′), 23.44 (C-α), 14.27 (C-ε′), 10.37 (C-β). HRMS (ESI) calcd. for C₁₈H₂₉N₂O₈ [M−H]⁻, 401.1929; found 401.1926.

### Example 79: 5-Pentanamido-9-acetamido-2,6-anhydro-3,5-dideoxy-D-glycero-D-galacto-non-2-enonic acid (67)

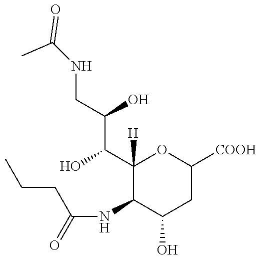 [SMILES: CCCC(=O)N[C@H]1[C@H]([C@H](O)[C@H](O)CNC(C)=O)OC(C(=O)O)C[C@@H]1O]

Compound 67 was synthesized from compound methyl 5-(tert-butoxycarbonyl)amino-9-azido-4,7,8-di-O-acetyl-2,6-anhydro-3,5-dideoxy-D-glycero-D-galacto-non-2-enonate using acetic anhydride and valeric anhydride. 20 mg. (24%(74%×50%×65%), over three steps) ¹H NMR (500 MHz, CD₃OD) δ 5.86 (d, J=2.4 Hz, 1H, H-3), 4.40 (dd, J=8.7, 2.4 Hz, 1H, H-4), 4.14 (d, J=10.7 Hz, 1H, H-6), 3.96 (dd, J=10.7, 8.7 Hz, 1H, H-5), 3.93-3.87 (m, 1H, H-8), 3.58 (dd, J=13.9, 3.2 Hz, 1H, H-9), 3.38 (dd, J=9.0, 0.7 Hz, 1H, H-7), 3.27-3.23 (m, 1H, H-9′), 2.27 (t, 2H, J=7.5 Hz, α-CH₂), 1.95 (s, 3H, COCH₃), 1.61 (dt, J=13.1, 7.5 Hz, 2H, β-CH₂), 1.37 (dt, J=15.0, 7.4 Hz, 2H, γ-CH₂), 0.93 (t, J=7.4 Hz, 3H, δ-CH₃). ¹³C NMR (126 MHz, CD₃OD) δ 177.96, 174.02 (2×N—C═O), 166.67 (C-1), 112.19 (C-3), 77.73 (C-6), 71.69 (C-7), 70.00 (C-4), 68.08 (C-8), 51.87 (C-5), 44.58 (C-9), 36.90 (C-α), 29.13 (C-β), 23.45 (C-γ), 22.58 (COCH₃), 14.16 (C-δ). HRMS (ESI) calcd. for C₁₆H₂₅N₂O₈ [M−H]⁻, 373.1616; found 373.1615.

### Example 80: 5-Hexanamido-9-acetamido-2,6-anhydro-3,5-dideoxy-D-glycero-D-galacto-non-2-enonic acid (68)

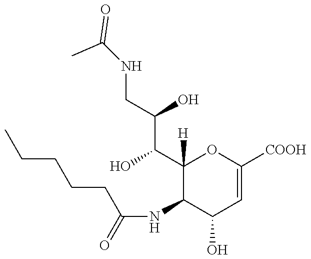 [SMILES: CCCCCC(=O)N[C@H]1[C@H]([C@H](O)[C@H](O)CNC(C)=O)OC(C(=O)O)=C[C@@H]1O]

Compound 68 was synthesized from compound methyl 5-(tert-butoxycarbonyl)amino-9-azido-4,7,8-di-O-acetyl-2,6-anhydro-3,5-dideoxy-D-glycero-D-galacto-non-2-enonate using acetic anhydride and hexanoic anhydride. 24 mg (30%(74%×55%×74%), over three steps). ¹H NMR (500 MHz, CD₃OD) δ 5.74 (d, J=2.3 Hz, 1H, H-3), 4.39 (dd, J=8.7, 2.3 Hz, 1H, H-4), 4.11 (d, J=10.8 Hz, 1H, H-6), 3.96 (dd, J=10.8, 8.7 Hz, 1H, H-5), 3.93-3.87 (m, 1H, H-8), 3.60 (dd, J=13.8, 3.2 Hz, 1H, H-9), 3.40-3.35 (m, 1H, H-7), 3.23 (dd, J=13.8, 7.2 Hz, 1H, H-9′), 2.29-2.23 (m, 2H, α-CH₂), 1.95 (s, 3H, COCH₃), 1.66-1.59 (m, 2H, β-CH₂), 1.37-1.28 (m, 4H, γ-CH₂, δ-CH₃), 0.90 (t, J=7.0 Hz, 3H, ε-CH₃). ¹³C NMR (126 MHz, CD₃OD) δ 178.01, 174.03 (2×N—C═O), 168.67 (C-1), 110.16 (C-3), 77.34 (C-6), 71.71 (C-7), 70.06 (C-4), 68.37 (C-8), 51.82 (C-5), 44.53 (C-9), 37.25 (C-α), 32.61 (C-β), 26.70 (C-γ), 23.45 (C-δ), 22.69 (COCH₃), 14.37 (C-ε). HRMS (ESI) calcd. for C₁₆H₂₅N₂O₈ [M−H]⁻, 387.1773; found 387.1774

### Example 81: 5-Hexanamido-9-propionamido-2,6-anhydro-3,5-dideoxy-d-glycero-d-galacto-non-2-enonic acid (69)

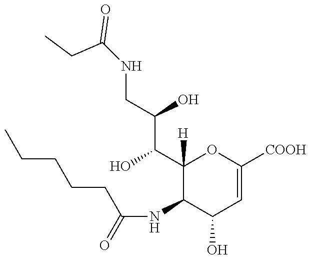 [SMILES: CCCCCC(=O)N[C@H]1[C@H]([C@H](O)[C@H](O)CNC(=O)CC)OC(C(=O)O)=C[C@@H]1O]

Compound 69 was synthesized from compound methyl 5-(tert-butoxycarbonyl)amino-9-azido-4,7,8-di-O-acetyl-2,6-anhydro-3,5-dideoxy-D-glycero-D-galacto-non-2-enonate using propionic anhydride and hexanoic anhydride. 20 mg (30%(96%×41%×75%), over three steps). ¹H NMR (500 MHz, CD₃OD) δ 5.73 (d, J=2.3 Hz, 1H, H-3), 4.36 (dd, J=8.6, 2.3 Hz, 1H, H-4), 4.10 (dd, J=10.8, 0.8 Hz, 1H, H-6), 3.97 (dd, J=10.8, 8.7 Hz, 1H, H-5), 3.89 (ddd, J=9.2, 5.2, 2.7 Hz, 1H, H-8), 3.59 (dd, J=13.8, 3.2 Hz, 1H, H-9), 3.36 (dd, J=9.0, 0.7 Hz, 1H, H-7), 3.25 (dd, J=13.8, 7.1 Hz, 1H, H-9′), 2.29-2.15 (m, 4H, α-CH₂, α′-CH₂), 1.63 (dt, J=14.8, 7.6 Hz, 2H, β-CH₂), 1.38-1.28 (m, 4H, γ-CH₂, δ-CH₂), 1.11 (t, J=7.6 Hz, 3H, 3′-CH₃), 0.91 (t, J=7.0 Hz, 3H, ε-CH₃). ¹³C NMR (126 MHz, CD₃OD) δ 177.78, 177.58 (2×N—C═O), 109.74 (C-3), 77.38 (C-6), 71.81 (C-7), 70.10 (C-4), 68.43 (C-8), 51.87 (C-5), 44.43 (C-9), 37.17 (C-α), 32.63 (C-β), 30.18 (C-α′), 26.67 (C-γ), 23.45 (C-δ), 14.32 (C-ε), 10.55 (C-β′). HRMS (ESI) calcd. for C₁₈H₂₉N₂O₈ [M−H]⁻, 401.1929; found 401.1929.

### Example 82: 5-Hexanamido-9-butanamido-2,6-anhydro-3,5-dideoxy-D-glycero-D-galacto-non-2-enonic acid (70)

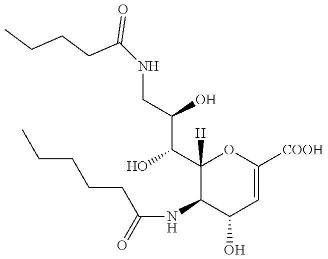 [SMILES: CCCCCC(=O)N[C@H]1[C@H]([C@H](O)[C@H](O)CNC(=O)CCCC)OC(C(=O)O)=C[C@@H]1O]

Compound 70 was synthesized from compound methyl 5-(tert-butoxycarbonyl)amino-9-azido-4,7,8-di-O-acetyl-2,6-anhydro-3,5-dideoxy-D-glycero-D-galacto-non-2-enonate using butyric anhydride and hexanoic anhydride. 22 mg (34%(96%×46%×78%), over three steps). ¹H NMR (500 MHz, CD₃OD) δ 5.70 (d, J=2.2 Hz, 1H, H-3), 4.36 (dd, J=8.6, 2.3 Hz, 1H, H-4), 4.09 (dd, J=10.8, 0.8 Hz, 1H, H-6), 3.97 (dd, J=10.8, 8.7 Hz, 1H, H-5), 3.92-3.85 (m, 1H, H-8), 3.59 (dd, J=13.8, 3.2 Hz, 1H, H-9), 3.36 (d, J=8.9 Hz, 1H, H-7), 3.25 (dd, J=13.8, 7.0 Hz, 1H, H-9′), 2.29-2.21 (m, 2H, α-CH₂), 2.20-2.11 (m, 2H, α′-CH₂), 1.62 (ddd, J=14.9, 7.5, 2.6 Hz, 4H, β-CH₂, β′-CH₂), 1.34-1.32 (m, 4H, γ-CH₂, δ-CH₂), 0.94-0.89 (m, 6H, ε-CH₃, γ′-CH₃). ¹³C NMR (126 MHz, CD₃OD) δ 177.75, 176.70 (2×N—C═O), 109.28 (C-3), 77.31 (C-4), 71.74 (C-7), 70.22 (C-6), 68.50 (C-8), 51.89 (C-5), 44.32 (C-9), 39.04 (C-α), 37.18 (C-α′), 32.63 (C-β), 26.66 (C-γ), 23.46 (C-β′), 20.44 (C-δ), 14.33 (C-ε), 14.08 (C-γ). HRMS (ESI) calcd. for C₁₉H₃₂N₂O₈ [M−H]⁻, 415.2086; found 415.2088.

### Example 83: 5-(2-Ethyl)hexanamido(S/R)-9-acetanamido-2,6-anhydro-3,5-dideoxy-d-glycero-d-galacto-non-2-enonic acid (73)

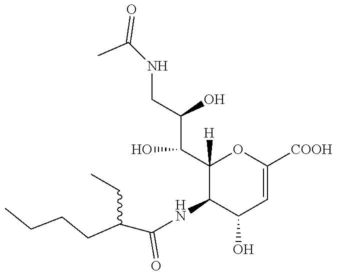 [SMILES: CCCCC(CC)C(=O)N[C@H]1[C@H]([C@H](O)[C@H](O)CNC(C)=O)OC(C(=O)O)=C[C@@H]1O]

Compound 73 was synthesized from compound methyl 5-(tert-butoxycarbonyl)amino-9-azido-4,7,8-di-O-acetyl-2,6-anhydro-3,5-dideoxy-d-glycero-d-galacto-non-2-enonate using acetic anhydride and 2-ethylhexanoyl chloride. The product was obtained as a mixture of diastereoisomers at a position of the hexanamido group. 6.0 mg (24%(96%×67%×38%), over three steps). ¹H NMR (500 MHz, CD₃OD) δ 5.67 (2H), 4.36-4.33 (2H), 4.14-4.07 (2H), 4.01-3.97 (2H), 3.90-3.87 (2H), 3.64-3.56 (2H), 3.41-3.39 (2H), 3.26-3.14 (2H), 2.22-2.13 (4H), 1.93 (3H), 1.94 (3H) 1.63-1.26 (8H), 1.50-1.24 (m, 7H), 0.97-0.84 (6H). ¹³C NMR (126 MHz, CD₃OD) δ 180.53, 173.75, 173.65, 108.90, 79.80, 77.38, 72.33, 72.01, 70.03, 69.94, 68.58, 68.54, 51.97, 51.95, 50.21, 50.14, 44.78, 44.54, 33.69, 33.51, 31.07, 30.78, 27.27, 27.12, 23.77, 22.60, 14.31, 12.68, 12.45. HRMS (ESI) calcd. for C₁₉H₃₁N₂O₈ [M−H]⁻, 415.2086; found 415.2088.

### Example 84: 5-(2-Methyl)hexanamido(S/R)-9-acetanamido-2,6-anhydro-3,5-dideoxy-d-glycero-d-galacto-non-2-enonic acid (74)

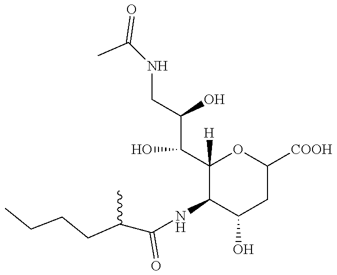 [SMILES: CCCCC(C)C(=O)N[C@H]1[C@H]([C@H](O)[C@H](O)CNC(C)=O)OC(C(=O)O)C[C@@H]1O]

Compound 74 was synthesized from compound methyl 5-(tert-butoxycarbonyl)amino-9-azido-4,7,8-di-O-acetyl-2,6-anhydro-3,5-dideoxy-d-glycero-d-galacto-non-2-enonate using acetic anhydride and 2-methylhexanoyl chloride. The product was obtained as a mixture of diastereoisomers at a position of the hexanamido group. 10.2 mg (54%(96%×70%×60%), over three steps). ¹H NMR (500 MHz, CD₃OD) δ 5.76 (d, J=2.2 Hz, 1H), 5.72 (d, J=2.3 Hz, 1H), 4.43 (dd, J=8.8, 2.3 Hz, 1H), 4.40-4.35 (m, 1H), 4.32 (dd, J=11.0, 1.0 Hz, 1H), 4.17-4.14 (m, 1H), 4.11 (dd, J=10.9, 3.0 Hz, 1H), 3.98-3.93 (m, 1H), 3.92-3.85 (m, 2H), 3.64-3.53 (m, 3H), 3.39-3.30 (m, 2H), 3.20 (dd, J=13.7, 7.5 Hz, 1H), 2.41-2.32 (m, 2H), 1.68-1.58 (m, 2H), 1.42-1.24 (m, 10H), 1.14-1.09 (m, 6H), 0.93-0.86 (m, 6H). ¹³C NMR (126 MHz, CD₃OD) δ 181.38, 174.09, 173.80, 168.70, 110.49, 110.37, 109.65, 77.58, 77.54, 76.27, 72.09, 71.74, 71.39, 70.24, 70.00, 69.96, 68.31, 68.28, 68.21, 52.30, 51.88, 51.69, 44.75, 44.52, 44.35, 42.25, 42.21, 35.05, 34.90, 30.93, 30.74, 23.74, 22.60, 22.57, 22.45, 18.57, 18.30, 14.40, 14.33. HRMS (ESI) calcd. for C₁₃H₂₉N₂O₈ [M−H]⁻, 401.1929; found 401.1926.

### Example 85: General Procedure for Synthesis of Compounds 75 and 72

A solution of compound methyl 5-amino-4,7,8,9-tetra-O-acetyl-2,6-anhydro-3,5-dideoxy-d-glycero-d-galacto-non-2-enonate in anhydrous dichloromethane and triethylamine (4 eq) was cooled down to 0° C. and anhydrides or acyl chlorides (1.5 eq) was added in dropwise. The solution was allowed to warm to room temperature and kept stirring overnight. After completion, the reaction was quenched with water, concentrated and purified by flash chromatography to give the protected product. The protected product was then dissolved in methanol and 0.5 N NaOH was added. The solution was stirred under room temperature until completion. After completion, the solution was neutralized by Amberlite™ IR 120 (H). The suspension was then filtered, and the filtrate was concentrated and purified by flash chromatography or precipitated in a mixture of methanol and ethyl acetate to give the desired product.

### Example 86: 5-(2-Ethyl)hexanamido(S/R)-2,6-anhydro-3,5-dideoxy-d-glycero-d-galacto-non-2-enonic acid (75)

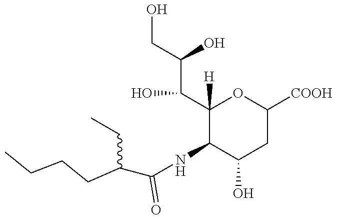 [SMILES: CCCCC(CC)C(=O)N[C@H]1[C@H]([C@H](O)[C@H](O)CO)OC(C(=O)O)C[C@@H]1O]

Compound 75 was synthesized from compound methyl 5-amino-4,7,8,9-tetra-O-acetyl-2,6-anhydro-3,5-dideoxy-d-glycero-d-galacto-non-2-enonate using 2-ethylhexanoyl chloride, and the product was obtained as a mixture of diastereoisomers at a position of the hexanamido group. 18.6 mg (25% (45%×55%), over two steps). ¹H NMR (500 MHz, CD₃OD) δ 5.64 (2H), 4.42 (dd, J=8.8, 2.2 Hz, 1H), 4.34 (dt, J=8.6, 2.6 Hz, 1H), 4.29 (dd, J=11.0, 0.8 Hz, 1H), 4.15 (dd, J=11.0, 8.8 Hz, 1H), 4.09 (ddd, J=10.9, 2.4, 1.1 Hz, 1H), 3.99 (ddd, J=10.8, 8.6, 7.0 Hz, 1H), 3.90-3.77 (m, 4H), 3.63 (dd, J=11.5, 5.5 Hz, 1H), 3.58-3.50 (m, 2H), 3.44 (dd, J=9.2, 0.9 Hz, 1H), 2.17 (dq, J=9.4, 5.0 Hz, 1H), 1.65-1.22 (m, 17H), 0.95-0.84 (m, 12H). ¹³C NMR (126 MHz, CD₃OD) δ 180.56, 180.54, 170.35, 170.19, 159.62, 159.32, 150.18, 150.08, 118.72, 108.41, 107.90, 77.41, 76.07, 71.44, 71.36, 71.30, 70.80, 70.63, 70.16, 68.63, 68.59, 68.50, 65.45, 65.24, 64.84, 52.30, 51.89, 51.83, 50.23, 50.20, 33.70, 33.56, 31.11, 30.79, 27.33, 27.11, 23.83, 23.80, 14.39, 14.32, 12.61, 12.44. HRMS (ESI) calcd. for C₁₇H₂₈NO₈ [M−H]⁻, 374.1820; found 374.1811.

### Example 87: 5-(2-Methyl)hexanamido(S/R)-2,6-anhydro-3,5-dideoxy-d-glycero-d-galacto-non-2-enonic acid (72)

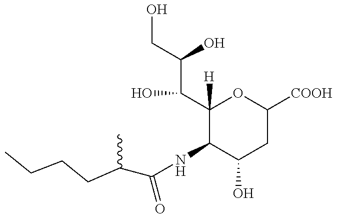 [SMILES: CCCCC(C)C(=O)N[C@H]1[C@H]([C@H](O)[C@H](O)CO)OC(C(=O)O)C[C@@H]1O]

Compound 72 was synthesized from compound methyl 5-amino-4,7,8,9-tetra-O-acetyl-2,6-anhydro-3,5-dideoxy-d-glycero-d-galacto-non-2-enonate using 2-methylhexanoyl chloride, and the product was obtained as a mixture of diastereoisomers at a position of the hexanamido group. 8.7 mg (10% (18%×55%), over two steps). ¹H NMR (500 MHz, CD₃OD) δ 5.70 (d, J=1.5 Hz, 1H), 5.68 (d, J=2.2 Hz, 1H), 4.43 (dd, J=8.8, 2.2 Hz, 1H), 4.36 (ddd, J=8.6, 4.1, 2.4 Hz, 1H), 4.31 (dd, J=11.0, 0.7 Hz, 1H), 4.18-4.15 (m, 1H), 4.12-4.08 (m, 1H), 3.96 (dd, J=10.8, 8.7 Hz, 1H), 3.90-3.78 (m, 4H), 3.65-3.53 (m, 3H), 3.52-3.47 (m, 1H), 3.45 (dd, J=9.3, 0.8 Hz, 1H), 2.40-2.32 (m, 1H), 1.69-1.57 (m, 1H), 1.40-1.21 (m, 12H), 1.15-1.05 (m, 6H), 0.89 (dt, J=7.0, 4.4 Hz, 6H). ¹³C NMR (126 MHz, CD₃OD) δ 181.39, 181.37, 169.55, 169.33, 116.42, 109.43, 109.33, 108.68, 77.57, 76.22, 71.43, 71.26, 71.20, 70.67, 70.41, 70.17, 68.51, 68.46, 68.41, 65.38, 65.11, 64.87, 52.25, 51.81, 51.69, 42.32, 42.23, 35.13, 34.98, 30.96, 30.74, 23.79, 23.76, 18.47, 18.36, 14.38, 14.33. HRMS (ESI) calcd. for C₆H₂₆NO[M−H]⁻, 360.1664; found 360.1663.

### Example 88: Methyl 5-(tert-butoxycarbonyl)amino-4-azido-2,6-anhydro-3,4,5-trideoxy-dideoxy-D-glycero-D-galacto-non-2-enonate (76)

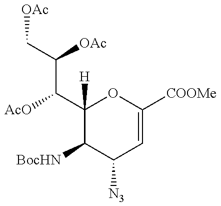

Compound methyl 5-acetamido-4-azido-7,8,9-tri-O-acetyl-2,6-anhydro-3,4,5-trideoxy-D-glycero-D-galacto-non-2-enonate (200 mg, 1 eq), di-tert-butyl dicarbonate (241 mg, 2.7 eq) and 4-dimethylaminopyridine (76 mg, 1.6 eq) were dissolved in 60 ml anhydrous THF. The solution was then refluxed for 2 hours. After completion, solvents were removed under reduced pressure and the residue was separated by flash chromatography, providing crude product 240 mg. The crude product was dissolved in 10 ml MeOH. After the solution was cooled down to 0° C., NaOMe (20 mg, 1 eq) was added slowly. The mixture was stirred under 0° C. for about 1 hour. After completion, Amberlite™ IR 120 (H+) was added to adjust the pH of the solution to 7. After filtration, solvent was removed under reduced pressure and the residue was purified by flash chromatography, providing the desired product 140 mg (82%, over two steps) ¹H NMR (500 MHz, CD₃OD) δ 5.86 (d, J=2.4 Hz, 1H, H-3), 4.31 (dd, J=9.4, 2.4 Hz, 1H, H-4), 4.21 (d, J=10.9 Hz, 1H, H-6), 3.88-3.79 (m, 3H, H-5, H-8, H-9), 3.77 (s, 3H, COOCH₃), 3.71-3.61 (m, 2H, H-7, H-9′), 1.45 (s, 9H, ^{t}Boc). ¹³C NMR (126 MHz, CD₃OD) δ 169.53, 163.95 (C-1), 158.44 (^{t}Boc-OCO), 146.55 (C-2), 108.74 (C-3), 81.25 (^{t}Boc-CCH₃), 78.40 (C-6), 71.28 (C-8), 69.75 (C-7), 64.87 (C-9), 60.08 (C-4), 53.00 (C-5), 50.53 (COOCH₃), 28.57 (^{t}Boc-CH₃). HRMS (ESI) calcd. for C₁₅H₂₄N₄NaO₈ [M−H]⁻, 411.1486; found 411.1487.

### Example 89: Methyl 5-(4-methylpentanamido)-4-azido-7,8,9-tri-O-acetyl-2,6-anhydro-3,4,5-trideoxy-D-glycero-D-galacto-non-2-enonate (77)

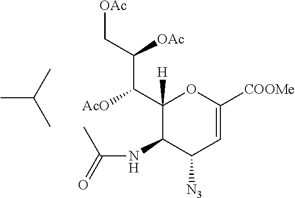

A solution of compound 76 (140 mg, 1 eq) in anhydrous pyridine was cooled down to 0° C. and acetic anhydride (400 μl, 10 eq) was added in dropwise. The mixture was then allowed to warm to temperature and kept stirring overnight. After completion, the reaction was quenched with methanol and solvents were removed under reduced pressure. The residue was dissolved in 200 ml ethyl acetate and carefully washed with 0.1 M HCl, water, brine and dried over Na₂SO₄.

The solution was concentrated to give 160 mg yellow oil, which was dissolved in 20 ml anhydrous DCM and 2 ml TFA was added slowly. The solution was then stirred at room temperature for 2 hours. After completion, DCM and TFA were removed under reduced pressure. The residue was dissolved in 10 ml anhydrous DCM and TEA (124 μl, 3 eq) was added. The mixture was then cooled down to 0° C. and 4-methylpentanoyl chloride (75 mg, 1.2 eq) was added. The solution was allowed to warm to room temperature and kept stirring overnight. After completion, the reaction was quenched with water, concentrated and purified by flash chromatography to give the desired product. 105 mg (52%, over three steps). ¹H NMR (500 MHz, CDCl₃) δ 6.05 (d, J=8.3 Hz, 1H, NH), 5.94 (d, J=2.1 Hz, 1H, H-3), 5.41 (d, J=5.2 Hz, 1H, H-7), 5.29 (td, J=6.5, 2.7 Hz, 1H, H-8), 4.59 (dd, J=12.4, 2.6 Hz, 1H, H-9), 4.54-4.50 (m, H5, H-4), 4.16 (dd, J=12.4, 6.6 Hz, 1H, H-9′), 3.78 (s, 3H, COOCH₃), 2.17 (t, J=7.8 Hz, 2H, α-CH₂), 2.11, 2.03, 2.02 (3×s, 9H, 3×COCH₃), 1.62-1.43 (m, 3H, β-CH₂, γ-CH), 0.88 (d, J=6.3 Hz, 6H, 2×δ-CH₃). ¹³C NMR (126 MHz, CDCl₃) δ 174.00, 170.69, 170.27, 170.08 (4×C═O), 161.55 (C-1), 145.05 (C-2), 107.66 (C-3), 75.58 (C-6), 70.66 (C-8), 67.74 (C-7), 62.00 (C-9), 57.51 (C-4), 52.56 (COOCH₃), 48.74 (C-5), 34.71 (C-β), 34.00 (C-γ), 27.72, 22.24, 22.20 (3×COCH₃), 20.82, 20.72 (2×C-δ). HRMS (ESI) calcd. for C₂₂H₃₂N₄NaO₈ [M−H]⁻, 535.2011; found 535.2003.

### Example 90: Methyl 5-(4-methylpentanamido)-7,8,9-tri-O-acetyl-2,6-anhydro-4-[2,3-bis(tert-butoxycarbonyl)guanidino]-3,4,5-trideoxy-D-glycero-D-galacto-non-2-enonate (78)

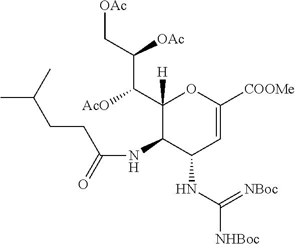

To a solution of compound 77 (50 mg, 1 eq) in THE (2 ml), 1 N HCl (200 μl, 2.2 eq) was added, followed by triphenylphosphine (29 mg, 1.2 eq). The resulting mixture was stirred at room temperature overnight. After completion, solvents were removed under reduced pressure and the residue was purified by flash chromatography, providing crude product 50 mg. The residue was dissolved in 5 ml anhydrous DCM, and TEA (50 μl, 4 eq) was added. The solution was cooled down to 0° C. and N, N′-Di-Boc-1H-pyrazole-1-carboxamidine (600 mg, 2 eq) added. The reaction mixture was allowed to warm up to room temperature and kept stirring overnight. After completion, the reaction was quenched with water, extracted with ethyl acetate. The organic phase was washed with brine, dried over Na₂SO₄, concentrated and purified by flash chromatography to give the desired product. 60 mg (87%, over two steps). ¹H NMR (500 MHz, CDCl₃) δ 8.46 (d, J=8.9 Hz, 1H, NH), 7.76 (brs, 1H, NH), 6.16 (d, J=9.2 Hz, 1H, NH), 5.88 (d, J=2.4 Hz, 1H, H-3), 5.42 (dd, J=4.9, 1.7 Hz, 1H, H-7), 5.28 (ddd, J=7.4, 4.9, 2.7 Hz, 1H, H-8), 5.19 (td, J=9.7, 2.4 Hz, 1H, H-4), 4.67 (dd, J=12.4, 2.7 Hz, 1H, H-9), 4.31 (dd, J=10.5, 9.7 Hz, 1H, H-5), 4.26 (dd, J=10.5, 1.7 Hz, 1H, H-6), 4.15 (dd, J=12.4, 7.4 Hz, 1H, H-9′), 3.79 (s, 3H, COOCH₃), 2.17-1.96 (m, 11H, 3×COCH₃, α-CH₂), 1.56-1.33 (m, 21H, 2×^{t}Boc, β-CH₂, γ-CH), 0.85 (dd, J=6.5, 2.7 Hz, 6H, 2×δ-CH₃). ¹³C NMR (126 MHz, CDCl₃) δ 174.01, 170.56, 170.24, 170.07 (4×C═O), 161.70 (^{t}Boc-OCO), 157.23 (C-1), 152.62 (C═N), 145.07 (C-2), 109.71 (C-3), 83.87, 79.77 (^{t}Boc-C(CH₃)₃), 78.11 (C-6), 71.57 (C-8), 67.76 (C-7), 62.29 (C-9), 52.45 (COOCH₃), 48.88 (C-4), 47.60 (C-5), (COCH₃), 34.67 (C-α), 34.12 (C-β), 28.27, 28.03 (^{t}Boc-C(CH₃)₃), 27.70 (C-γ), 22.32, 22.13 (C-δ), 20.91, 20.87, 20.79 (COCH₃). HRMS (ESI) calcd. for C₃₃H₅₂N₄NaO₁₄ [M+Na]⁺, 751.3372; found 751.3378.

### Example 91: 5-(4-methylpentanamido)-2,6-anhydro-4-guanidino-3, 4, 5-trideoxy-D-glycero-D-galacto-non-2-enonic acid (71)

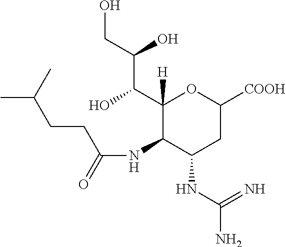 [SMILES: CC(C)CCC(=O)N[C@H]1[C@H]([C@H](O)[C@H](O)CO)OC(C(=O)O)C[C@@H]1NC(=N)N]

To a solution of compound 78 (60 mg) in 5 ml DCM, 500 μl TFA was added. The solution was then stirred at room temperature for 2 hours. After completion, DCM and TFA were removed under reduced pressure. The residue was dissolved in methanol and 2 ml 1 N NaOH was added. The solution was stirred at room temperature for 1 hour. After completion, the reaction mixture was added with Amberlite™ IR 120 (H) to make the pH of the solution as 7. The suspension was then filtered, and the filtrate was concentrated to give a light-yellow oil. The residue was dissolved in minimum methanol and the product was precipitated by ethyl acetate. The product was obtained by filtering as a white solid. 15 mg (48%, over two steps). ¹H NMR (700 MHz, CD₃OD) δ 5.50 (s, 1H, H-3), 4.37 (d, J=8.5 Hz, 1H, H-4), 4.33 (d, J=10.0 Hz, 1H, H-6), 4.19 (t, J=9.4 Hz, 1H, H-5), 3.89-3.79 (m, 2H, H-8, H-9), 3.65 (dd, J=11.3, 5.4 Hz, 1H, H-9′), 3.58 (d, J=9.2 Hz, 1H, H-7), 2.26 (t, J=7.5 Hz, 2H, α-CH₂), 1.61-1.49 (m, 3H, β-CH₂, γ-CH), 0.92 (d, J=6.4 Hz, 6H, 2×δ-CH₃). ¹³C NMR (176 MHz, CD₃OD) δ 177.19 (COCH₃), 169.63 (C-1), 158.78 (C═N), 151.58 (C-2), 103.33 (C-3), 77.04 (C-6), 71.33 (C-8), 70.31 (C-7), 64.88 (C-9), 52.26 (C-4), 35.85 (C-α), 35.21 (C-β), 28.86 (C-γ), 22.72, 22.63 (2×C-δ). HRMS (ESI) calcd. for C₃₃H₅₂N₄NaO₁₄ [M+Na]⁺, 387.1885; found 387.1879.

### Example 92: Material and Methods for Example 93

### Inhibition Assay

Inhibition assays against 4MU-NANA (2′-(4-methylumbellifery)-α-D-N-acetylneuraminic acid) cleavage and GM3 cleavage was performed using protocols reported previously (Zhang, 2013). NEU3 and NEU2 were expressed as N-terminal MBP fusion protein in E. coli and purified as previously reported (Albohy, 2010). NEU4 was expressed as a GST fusion protein in E. coli and purified as previously reported (Albohy, 2011). NEU1 was expressed as His fusion protein in HEK293 cells and cell lysate was used without further purification (Pshezhetsky, 1996). All assays were conducted in 0.1 M sodium acetate buffer at optimum pH for each enzyme (4.5 for NEU1, NEU3 and NEU4; 5.5 for NEU2) (Zhang, 2013). To get comparable IC₅₀ among the four isoenzymes, similar activity units of each enzyme were used in the assay.

For assays using 4MU-NANA as the substrate, inhibitors with of a 3-fold serial dilution of concentrations were incubated with enzyme at 0° C. for 15 min. 4MU-NANA was then added to the mixture, making the final concentration of 4MU-NANA as 50 μM and the total volume of the reaction mixture as 20 μL. After incubation at 37° C. for 30 min, the reaction was quenched with 100 μL of 0.2 M sodium glycine buffer (pH 10.2). The reaction mixture was transferred to 386-well plate and the enzyme activity was determined by measuring fluorescence (λₑₓ=365 nm; λₑₘ=445 nm) using a plate reader (Molecular Devices, Sunnyvale Calif.). Assays were performed with duplicates for each point and IC₅₀ was obtained by plotting the data with Graphpad™ Prism 5.0. For curves that showed less than a 50% decrease in signal, fits were conducted using maximum inhibition values found for DANA.

For inhibition assays against GM3 cleavage, a method developed by Markely and coworkers was adopted (Markely, 2010). The assay was conducted in 0.1 M sodium acetate buffer (pH 4.5). After serial concentrations of inhibitors were incubated with enzyme at 0° C. for 15 min, GM3 was added, making the final concentration of GM3 as 500 μM and the total volume of the reaction mixture as 20 μL. The reaction mixture was incubated at 37° C. for 30 min and quenched with 100 μL of freshly made 0.2 M sodium borate buffer (pH 10.2). 0.8% malononitrile solution (40 μL) was added to form a fluorescent adduct with the free sialic acids. Fluorescence was obtained (λₑₓ=357 nm; λₑₘ=434 nm) and the data was processed using Graphpad™ Prism 5.0. For curves that showed less than a 50% decrease in signal, fits were conducted using maximum inhibition values found for DANA.

### Kᵢ Determinations

Enzymes were incubated with serial concentrations of inhibitors at 0° C. for 15 min and serial concentrations of 4MU-NANA were added. The reaction mixture was transferred to 386-well plate immediately and the rate of product formation was obtained by measuring fluorescence (λₑₓ=315 nm; λₑₘ=450 nm) every 30 s for 30 min. The obtained data was processed with Graphpad™ Prism 5.0 for Kᵢ determination.

### Example 93: Inhibition Assays

To evaluate the inhibitory effects of the compounds against individual isoenzymes, each enzyme was produced recombinantly or purification (Albohy, 2013; Zhang, 2013; Albohy, 2010) and the IC₅₀ tested using an artificial substrate, 2′-(4-methylumbelliferyl)-α-D-N-acetylneuraminic acid (4MU-NANA) (Potier, 1979; Warner, 1979). The inhibitory effects are reported in Table I below.

TABLE I
IC₅₀ data using 4MU-NANA as the substrate
Formula II.

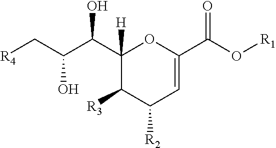

| | | | |
|---|---|---|---|
| | | | Selec- |
| | | | tiv- |
| Structure | IC₅₀ [μM] | Target | ity |

R₃ (at position C5) is CH₃C(O)NH— —

| Compound | | | | | | | | |
|---|---|---|---|---|---|---|---|---|
| of formula II | R₂ (at position C4) | R₄ (at position C9) | NEU1 | NEU2 | NEU3 | NEU4 | | |
| 1 (DANA) | 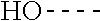 | 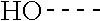 | 49 ± 8 | 37 ± 6 | 7.7 ± 0.8 | 8.3 ± 1.0 | NA | NA |
| 6 (Zanamivir) | 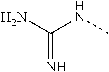 | 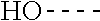 | >500 | 7.8 ± 2.0 | 4.0 ± 0.6 | 47 ± 6 | NEU2/NEU3 | 6 |
| 7a | 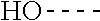 | 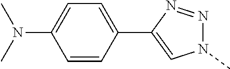 | 190 ± 40 | 250 ± 40 | 9.3 ± 0.8 | 28 ± 3 | NEU3/NEU4 | 7 |
| 7b | 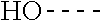 | 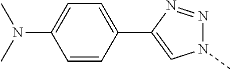 | 59 ± 5 | 78 ± 20 | 5.5 ± 0.5 | 11 ± 1 | NEU3/NEU4 | 5 |
| 7c | 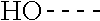 | 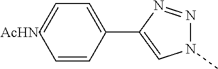 | 76 ± 16 | 350 ± 90 | 8.7 ± 0.9 | 12 ± 1 | NEU3/NEU4 | 6 |
| 7d | 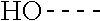 | 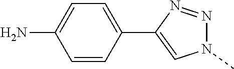 | 51 ± 7 | 290 ± 50 | 8.2 ± 1.3 | 3.9 ± 0.4 | NEU3/NEU4 | 6 |
| 7e | 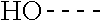 | 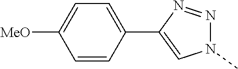 | 47 ± 6 | 430 ± 200 | 5.9 ± 0.4 | 2.6 ± 0.3 | NEU3/NEU4 | 8 |
| 7f | 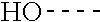 | 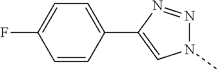 | 100 ± 40 | >500 | 17 ± 2 | 1.3 ± 0.2 | NEU4 | 10 |
| 7g | 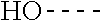 | 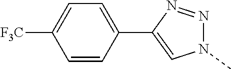 | 140 ± 30 | 360 ± 50 | 3.3 ± 0.5 | 5.0 ± 0.9 | NEU3/NEU4 | 28 |
| 7h | 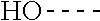 | 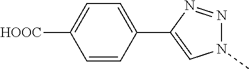 | 240 ± 90 | 110 ± 10 | 1.1 ± 0.1 | 3.0 ± 0.3 | NEU3/NEU4 | 37 |
| 7i | 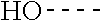 | 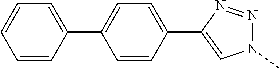 | >500 | 32 ± 5 | 0.70 ± 0.10 | 0.52 ± 0.10 | NEU3/NEU4 | 45 |
| 7j | 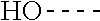 | 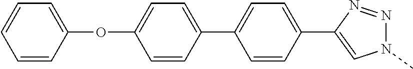 | >500 | 84 ± 24 | 1.0 ± 0.1 | 0.97 ± 0.24 | NEU3/NEU4 | 84 |
| 8a | 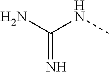 | 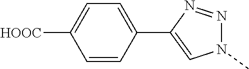 | >500 | 45 ± 5 | 0.61 ± 0.10 | 24 ± 2 | NEU3 | 40 |
| 8b | 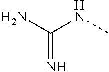 | 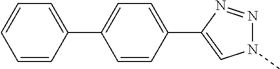 | >500 | 5.9 ± 1.1 | 0.58 ± 0.14 | 5.9 ± 1.4 | NEU3 | 10 |
| 13 | 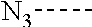 | 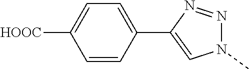 | >500 | >500 | 20 ± 6 | 400 ± 70 | NEU3 | 20 |
| 15 | 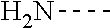 | 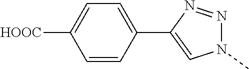 | >500 | >500 | >500 | >500 | NA | NA |
| 18 | 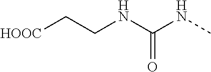 | 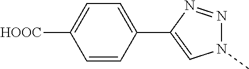 | >500 | 430 ± 300 | >500 | 66 ± 16 | NEU4 | 6 |
| 25a | 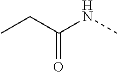 | 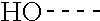 | >500 | >500 | >500 | >500 | NA | NA |
| 25b | 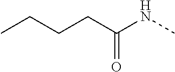 | 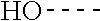 | >500 | >500 | >500 | >500 | NA | NA |
| 25c | 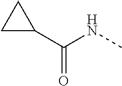 | 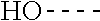 | >500 | >500 | >500 | >500 | NA | NA |
| 25d | 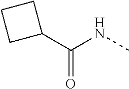 | 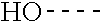 | >500 | >500 | >500 | >500 | NA | NA |
| 26 | 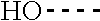 | 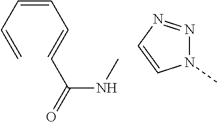 | >500 | 240 ± 50 | 15 ± 1 | 1.1 ± 0.2 | NEU4 | 15 |
| 27 | 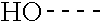 | 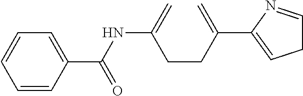 [SMILES: C=C(CCC(=C)C1=CCC=N1)NC(=O)c1ccccc1] | 190 ± 70 | 110 ± 40 | 2.9 ± 0.2 | 3.7 ± 0.4 | NEU3/NEU4 | 30 |
| (C9-4HMT-DANA) 28 | 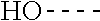 | 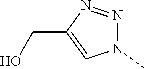 | >500 | >500 | 80 ± 10 | 0.16 ± 0.01 | NEU4 | 500 |
| 58 | 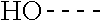 | 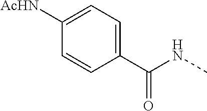 | 1.9 ± 0.4 | 90 ± 20 | 7.2 ± 1.2 | 24 ± 6 | NEU1 | 4 |
| 59 | 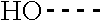 | 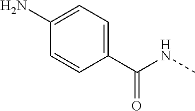 | 29 ± 5 | 190 ± 70 | 31 ± 6 | 210 ± 80 | NEU1/NEU3 | 6 |
| 60 | 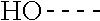 | 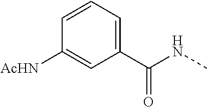 | 6.5 ± 1.1 | 130 ± 10 | 7.1 ± 0.7 | 52 ± 9 | NEU1/NEU3 | 7 |
| 61 | 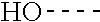 | 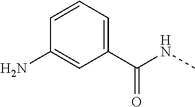 | 6.5 ± 0.7 | 180 ± 40 | 35 ± 6 | 150 ± 40 | NEU1 | 5 |
| 62 | 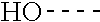 | 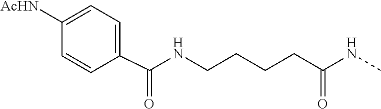 | 240 ± 70 | 390 ± 80 | 31 ± 5 | 46 ± 13 | NEU3/NEU4 | 5 |
| 63 | 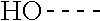 | 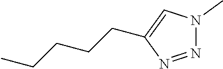 | 77 ± 30 | 450 ± 170 | 6.7 ± 1.2 | 2.6 ± 0.6 | NEU3/NEU4 | 11 |

R₂ (at position C4) is

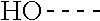

| Compound # | R₃ (at position C5) | R₄ (at position C9) | NEU1 | NEU2 | NEU3 | NEU4 | | |
|---|---|---|---|---|---|---|---|---|
| 29 | 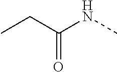 | 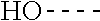 | 18 ± 1 | 86 ± 4 | 60 ± 7 | 87 ± 18 | NEU1 | 3 |
| 30 | 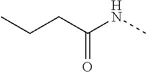 | 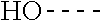 | 8.4 ± 0.5 | 40 ± 5 | 15 ± 2 | 8.4 ± 0.4 | NEU1/NEU4 | 2 |
| 31 | 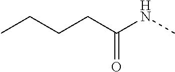 | 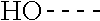 | 0.99 ± 0.07 | 33 ± 2 | 140 ± 10 | 110 ± 20 | NEU1 | 33 |
| 32 | 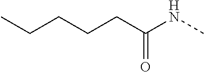 | 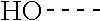 | 0.42 ± 0.06 | 15 ± 2 | 210 ± 60 | 440 ± 150 | NEU1 | 36 |
| 33 | 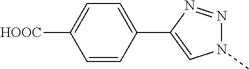 | 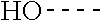 | 2.1 ± 0.2 | 37 ± 6 | 210 ± 70 | 470 ± 200 | NEU1 | 18 |
| 34 | 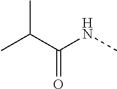 | 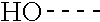 | 5.3 ± 0.8 | 170 ± 50 | >500 | 71 ± 17 | NEU1 | 13 |
| 35 | 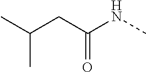 | 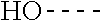 | 32 ± 5 | 39 ± 10 | >500 | >500 | NEU1/NEU2 | 13 |
| 36 | 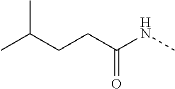 | 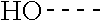 | 1.7 ± 0.1 | 7 ± 1 | 150 ± 20 | 370 ± 160 | NEU1 | 4 |
| 37 | 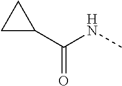 | 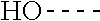 | 6.9 ± 0.4 | 220 ± 10 | 170 ± 40 | 150± 40 | NEU1 | 32 |
| 38 | 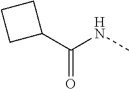 | 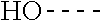 | 12 ± 2 | 60 ± 3 | 100 ± 20 | 81 ± 15 | NEU1 | 5 |
| 39 | 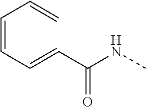 | 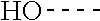 | 11 ± 1 | 41 ± 12 | 480 ± 100 | >500 | NEU1 | 3 |
| 40 | 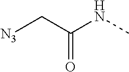 | 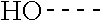 | 24 ± 2 | 21 ± 3 | 4.4 ± 0.9 | 5.6 ± 0.8 | NEU3/NEU4 | 5 |
| 41 | 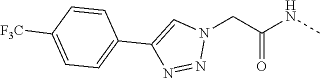 | 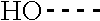 | >500 | 50 ± 7 | >500 | 300 ± 60 | NEU2 | 6 |
| 42 | 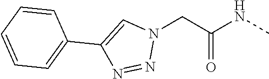 | 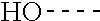 | >500 | 3.3 ± 0.3 | >500 | 110 ± 20 | NEU2 | 30 |
| 43 | 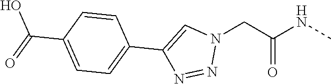 | 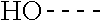 | >500 | 180 ± 30 | >500 | 400 ± 160 | NEU2 | NA |
| 44 | 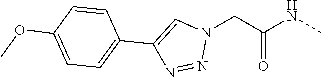 | 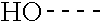 | >500 | 13± 1 | >500 | 87 ± 14 | NEU2 | 6 |
| 45 | 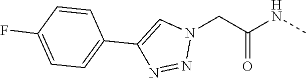 | 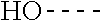 | >500 | 4.6 ± 0.3 | 240 ± 40 | 100 ± 20 | NEU2 | 21 |
| 46 | 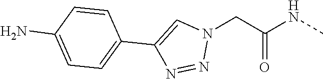 | 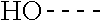 | >500 | 4.4 ± 0.3 | 430 ± 100 | 130 ± 20 | NEU2 | 29 |
| 47 | 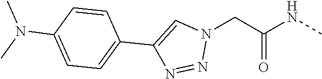 | 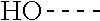 | 110 ± 10 | 76 ± 6 | 46 ± 7 | 36 ± 5 | NEU1/NEU2/ NEU3/NEU4 | NA |
| 48 | 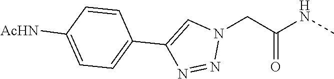 | 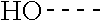 | >500 | 20 ± 3 | >500 | >500 | NEU2 | 25 |
| 49 | 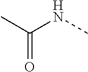 | 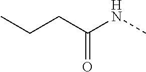 | 4.0 ± 0.5 | >500 | 250 ± 90 | 73 ± 14 | NEU1 | 18 |
| 50 | 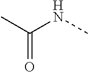 | 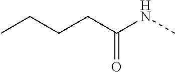 | 3.4 ± 0.2 | >500 | 110 ± 40 | 220 ± 50 | NEU1 | 32 |
| 51 | 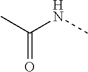 | 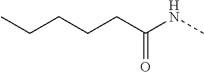 | 2.9 ± 0.2 | >500 | 83 ± 9 | 290 ± 30 | NEU1 | 28 |
| 52 | 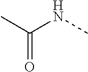 | 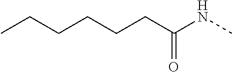 | 9.9 ± 1.3 | 410 ± 110 | 39 ± 8 | 310 ± 30 | NEU1 | 4 |
| 53 | 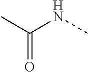 | 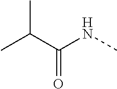 | 250 ± 60 | 220 ± 30 | 96 ± 28 | 230 ± 60 | NA | NA |
| 54 | 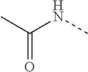 | 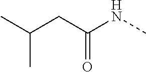 | 2.5 ± 0.3 | 120 ± 20 | 72 ± 20 | 130 ± 40 | NEU1 | 28 |
| 55 | 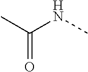 | 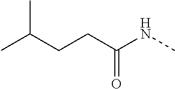 | 3.2 ± 0.3 | 160 ± 40 | 54 ± 6 | 150 ± 50 | NEU1 | 16 |
| 56 | 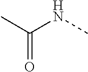 | 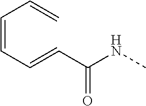 | 2.5 ± 0.3 | >500 | 34 ± 5 | 150 ± 10 | NEU1 | 13 |
| 57 | 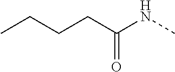 | 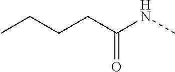 | 1.5 ± 0.2 | 59 ± 26 | >500 | >500 | NEU1 | 39 |
| 64 | 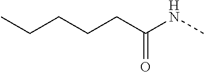 | 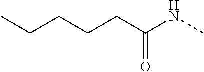 | 4.3 ± 0.8 | 26 ± 6 | >500 | >500 | NEU1 | 6 |
| 65 | 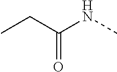 | 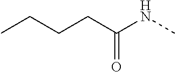 | 1.6 ± 0.2 | 140 ± 30 | >500 | 190 ± 70 | NEU1 | 88 |
| 66 | 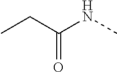 | 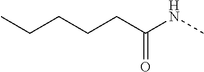 | 1.4 ± 0.2 | 31 ± 8 | 260 ± 70 | 210 ± 90 | NEU1 | 22 |
| 67 | 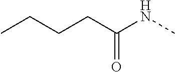 | 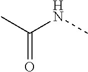 | 0.35 ± 0.03 | 170 ± 70 | >500 | >500 | NEU1 | 486 |
| 68 | 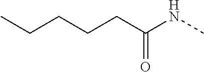 | 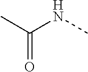 | 0.14 ± 0.01 | 47 ± 14 | >500 | 170 ± 100 | NEU1 | 336 |
| 69 | 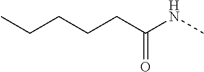 | 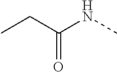 | 0.40 ± 0.10 | 90 ± 10 | >500 | 270 ± 70 | NEU1 | 225 |
| 70 | 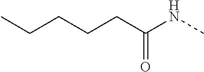 | 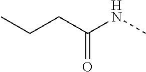 | 1.2 ± 0.1 | 32 ± 4 | >500 | >500 | NEU1 | 27 |
| 72 | 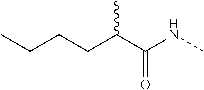 | 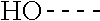 | 0.55 ± 0.07 | | | | | |
| 73 | 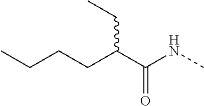 | 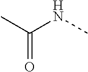 | 1.8 ± 0.2 | | | | | |
| 74 | 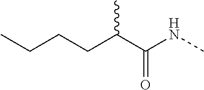 | 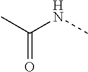 | 0.42 ± 0.07 | | | | | |
| 75 | 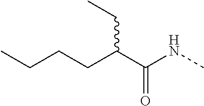 | 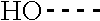 | 0.58 ± 0.03 | | | | | |
| 79 | 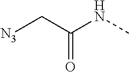 | 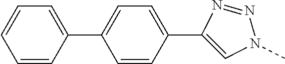 | 88 ± 28 | 9.2 ± 0.9 | 0.49 ± 0.04 | 0.83 ± 0.08 | | |
| 80 | 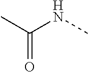 | 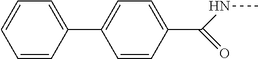 | 240 ± 100 | 90 ± 23 | 1.5 ± 0.1 | 11 ± 1 | | |

R₂ (at position C4) is NH₂C(═NH)NH— —

| | R₃ (at position C5) | R₄ (at position C9) | NEU1 | NEU2 | NEU3 | NEU4 | | |
|---|---|---|---|---|---|---|---|---|
| 71 | 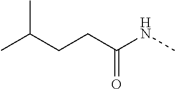 | 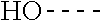 | 14 ± 2 | 2.1±0.1 | 150 ± 20 | 47 ± 7 | NEU2 | 7 |

In Table I, the specificity of a compound it designated as dual (bispecific) e.g., Neu3/Neu4, when the ratio of IC50 of this compound against an enzyme over the IC50 of this compound against another enzyme is of about 3 or less. Compounds 72-75 were tested as a mixture of diasteroisomers. All the others are pure compounds of a single stereochemistry.

### Example 94: Static Adhesion Assays Show T Cells Response to Neu Treatment with T Cells (Jurkat)

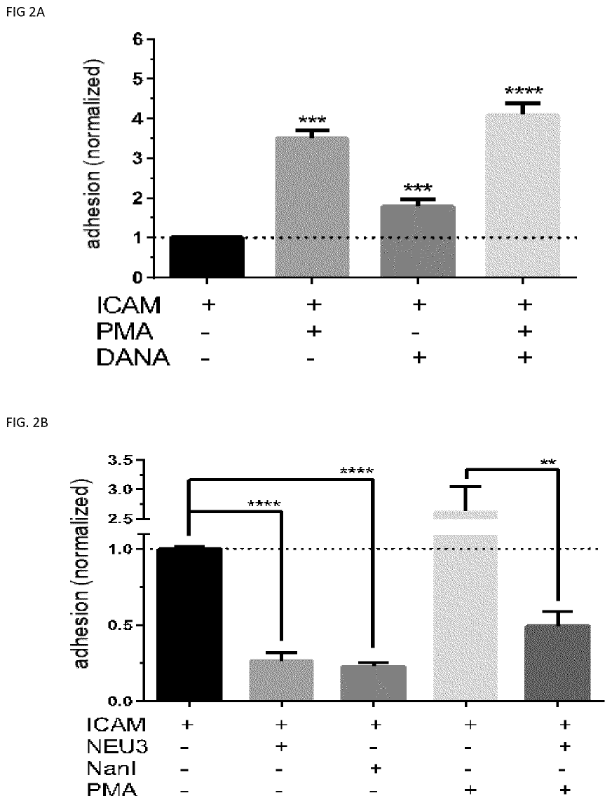

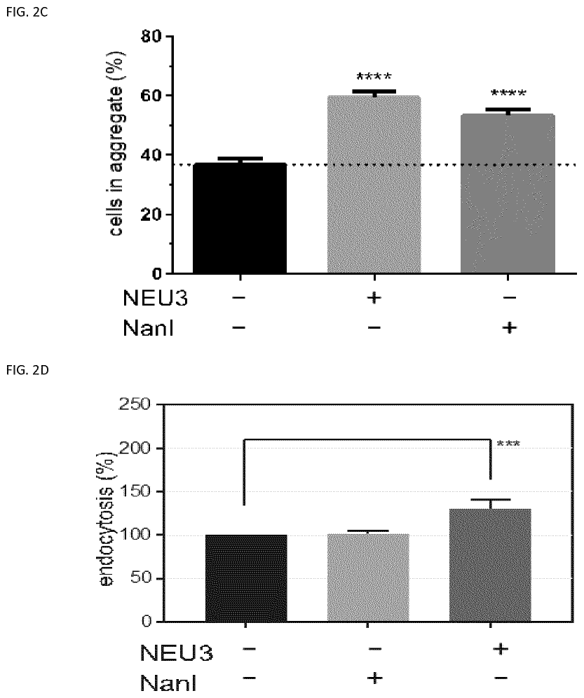

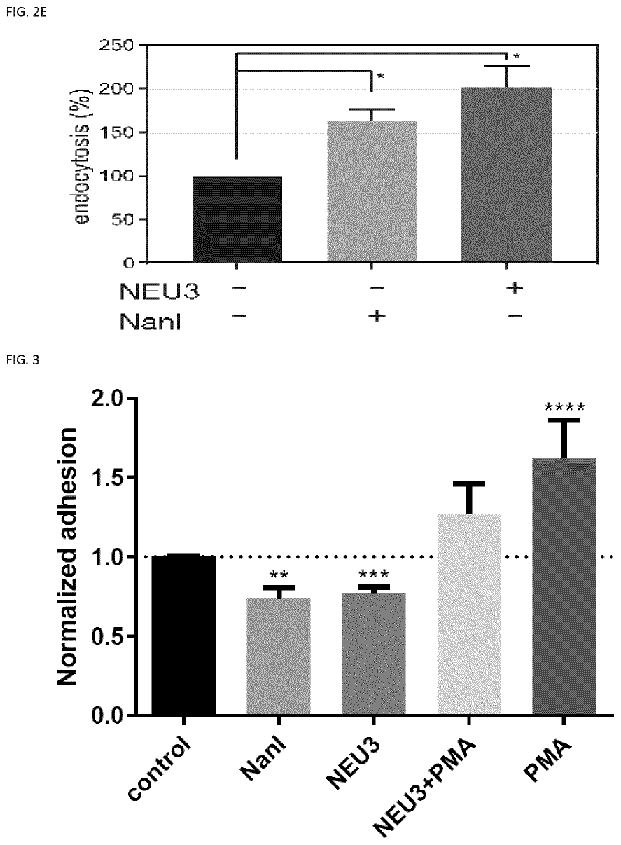

Adhesion of Jurkat cells (T cells) to ICAM-1 was determined using flow cytometry and ICAM-1-coated fluorescent beads (1 μm) under the indicated conditions. Control samples were treated with DMSO (0.05%), used as negative control, phorbol myristate acetate (PMA), known to induce leukocyte adherence and used as a positive control, or the nonspecific neu inhibitor DANA for 30 min (see Dana structure in Table I above). Results are presented in FIG. 2A.

Then, adhesion of Jurkat cells to ICAM-1 was determined using flow cytometry and ICAM-1-coated fluorescent beads; samples were treated with buffer (DMSO) or enzyme (Neu3; and NanI sialidase, another sialidase used as a control) for 3 hrs, followed by incubation with PMA for 30 min. All cytometry data were normalized to the appropriate control after background subtraction (BSA coated beads). Results are shown in FIG. 2B.

Then, homotypic aggregation of Jurkat cells was determined using microscopy. Cells were incubated under the indicated conditions for 3 hrs. Aggregation was determined by imaging and analysis with CellProfiler™ (version 2.1.1) to determine the total number of cells and the number of cells found within aggregates. Aggregation is expressed in FIG. 2C as the percentage of cells in all samples found within an aggregate. FIGS. 2D and E show changes in the endocytosis of β2-integrin (FIG. 2D) and β1-integrin (FIG. 2E) in Jurkat cells after treatment with NanI or NEU3 (30 min at 37° C.).

### Example 95: Static Adhesion Assays of PBMC Confirm Involvement of NEU3 in Leukocyte Adhesion

To confirm the effect of NEU3 on primary cells, PBMC were tested using the static adhesion assay as described in Example 94. Results are shown in FIG. 3. NEU3 treatment of PBMC shows a significant decrease in adhesion. Without being so limited, this data is consistent with a mechanism involving changes to membrane glycolipid composition, which alters the rate of endocytosis of integrins.

### Example 96: Alteration of LFA-1 Epitopes by Neuraminidase Treatment

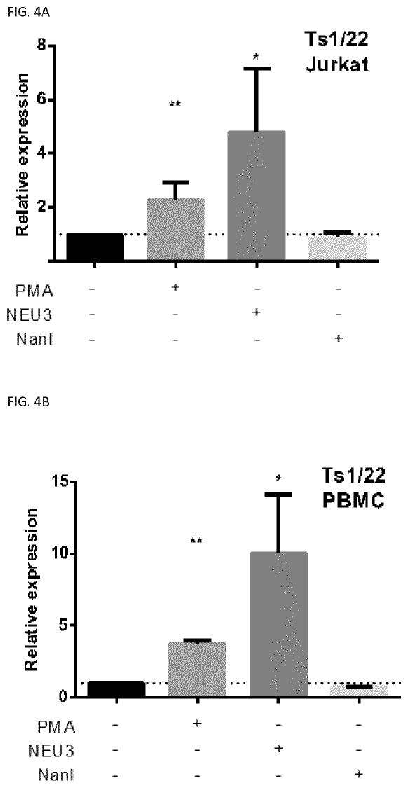

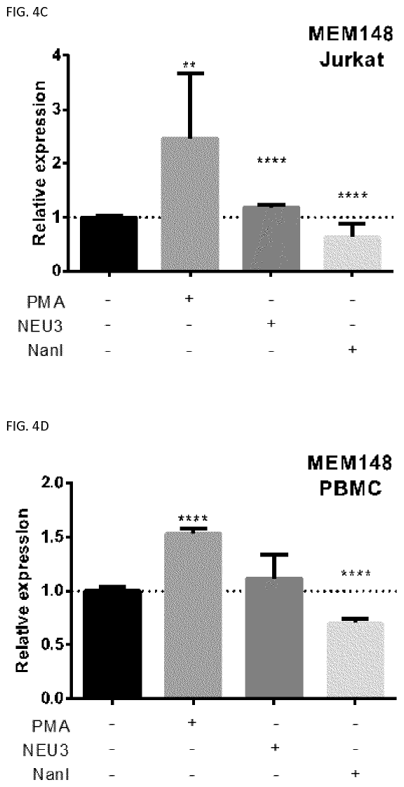

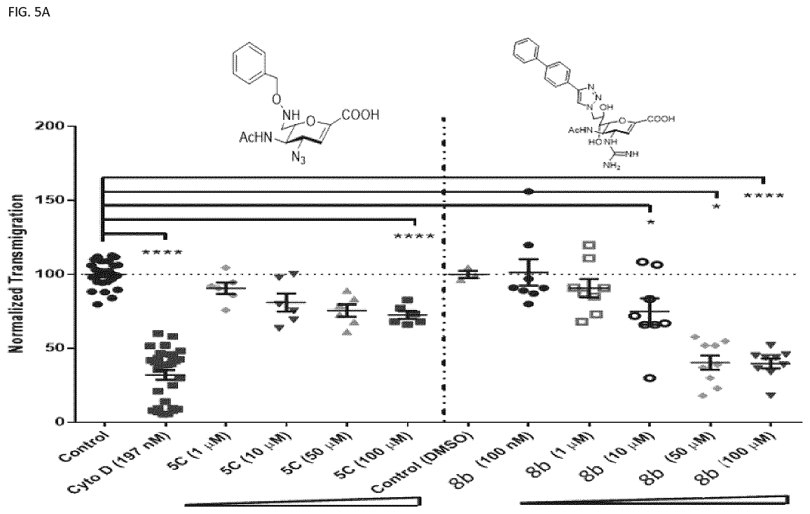

The inventors have then tested the effect of NEU enzymes on LFA-1-ICAM-1 adhesion. Lymphocyte function-associated antigen 1 (LFA-1) is an integrin found on lymphocytes and other leukocytes and is known to bind to ICAM-1. Jurkat T cells (FIGS. 4A and 4C) or PBMC (FIGS. 4B and 4D) were treated with the indicated conditions. Treated cells were labelled with primary antibodies (TS1/22 or MEM148), followed by an AF647-conjugated secondary antibody. Cells were then fixed with 1% PFA, analyzed by flow cytometry, and normalized to control (buffer) treatment.

### Example 97: In Vitro Inflammation—Inhibitors of NEU3 and NEU4 Blocked the Migration of T Cells in an In Vitro Transwell Assay

A transwell assay was implemented with a porous (3 μm pores) membrane coated with fibronectin (FN). The bottom well of the transmigration plate was coated with FBS, TNFα and IL-4 as chemo attractants. Neu3 inhibitors (compound 5c—see structure below; and compound 8b, see structure in Table I above); neu1 inhibitors (compounds 32 and 50, see structures in Table I above) and a neu4 inhibitor (compound 28, see structure in Table I above) were added to the upper well along with T cells (Jurkat) at the indicated concentrations. DMSO buffer was used as the negative control (control on left panel and control (DMSO) on right panel), and Cytochalasin D (Cyto D) was used as a positive control for inhibition of migration. Cells were then incubated for 21 h.

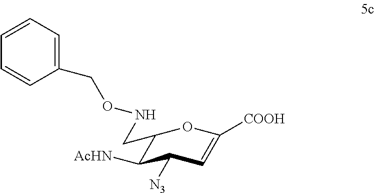

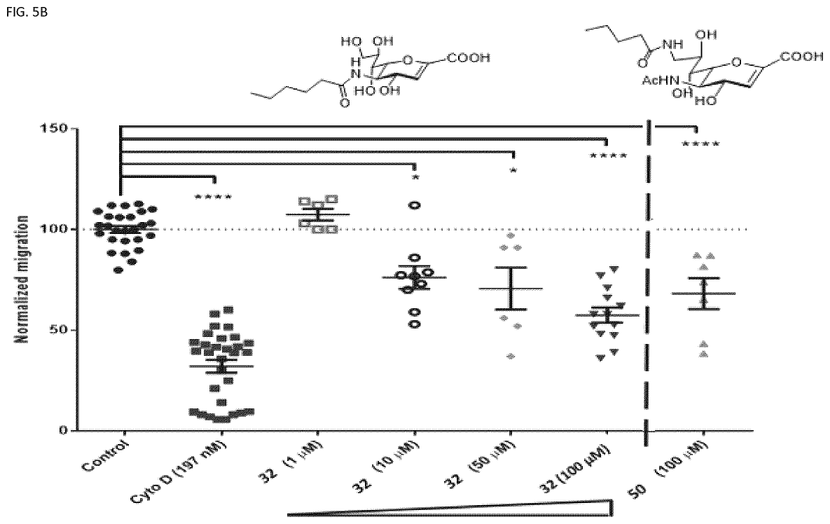

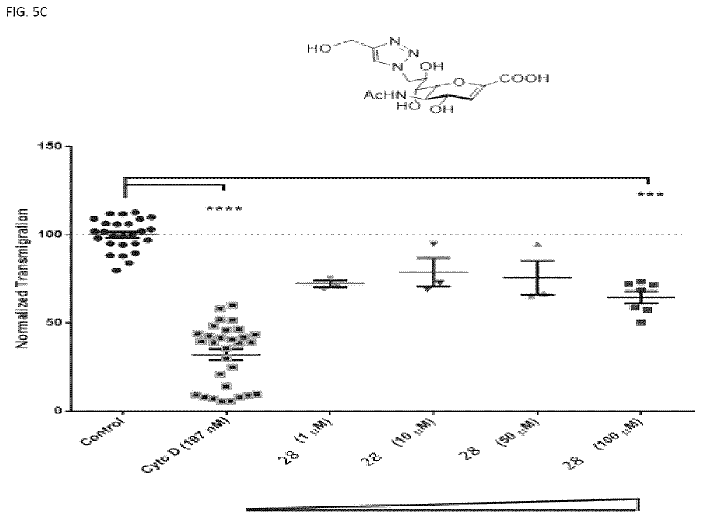

The number of cells migrating into the lower chamber were quantified using FACs and normalized to the control. Results are shown in FIGS. 5A-C.

### Example 98: Material and Methods for Examples 99-100

### Induction of Air Pouches and LPS-Induced Inflammation

Mice were maintained at the Canadian Council on Animal Care (CCAC)-accredited animal facilities of the CHU Sainte-Justine Research Centre, according to the CCAC guidelines.

To induce an air pouch, mice (6-wk-old, male Neu1^{-/-} (KO), Neu3^{-/-} (KO), Neu4^{-/} (KO)⁻, Neu3^{-/-} (KO), Neu4^{-/-} (KO) and C57Bl/6 mice) were injected subcutaneously with 3 cc of air (passed through 0.2 μm filter) on the back two times per week using a 26-gauge needle. Before the procedure, mice were anesthetized with isoflurane (2-3% with oxygen 0.4 L/min) and checked for proper anesthesia initiation. After injections mice were returned to their cages and checked for restoration of normal functions after recovery from anesthesia.

After one week, the air pouch was injected with 1 ml of an inflammatory substance LPS (Sigma-Aldrich, USA) at 1 ug/ml concentration in saline to induce inflammation. 1 ml of saline were injected on the air pouches to the control group of mice. Mice were returned to their cages, where they were allowed to run free for 6 h. At the end of 6 h, the animals were sacrificed using CO₂ asphyxiation. The contents of the pouch were aspirated. Then the air pouches were washed twice with 2 ml of HBSS containing 10 mM EDTA. The exudates were collected and centrifuged at 100 g for 10 min at room temperature. The supernatant were collected and frozen for later analysis. The cells were resuspended in 1 ml of HBSS-EDTA and taken for cell count. The air pouch were dissected from the subcutaneous tissue and stained (hematoxylin and eosin and Giemsa stains) using standard methods. The spleen, bones and other organs were collected and frozen for future analysis.

### Cells Analysis Protocol:

The cells collected by aspiration (in 4 ml of HBSS containing 10 mM EDTA) were aliquoted according to the following segmentation for hemocytometer, cytokine, FACS and Glycolipid analysis.

| | | | |
|---|---|---|---|
| 1. | hemocytometer | −100 μl | |
| 2. | FACS | −300 μl × 3 | |
| 3. | Cytokines & | Glycolipid | −3 ml |

For hemocytometer analysis, cells were centrifuged and spread onto microscopy slides, and stained with Hema stain for quantification of granulocyte and monocytes.

For cytokine and glycolipid analysis fraction was centrifuged 100×g for 10 min at RT. Plasma was separated from the cells, and frozen in liquid N₂. The cells were reconstituted in freezing buffer (RPMI+50% FBS+5% DMSO) and frozen in liquid nitrogen.

For further identification of other subpopulations, 300 μl of the cells were centrifuged 100×g for 10 min. The supernatant was washed once with HBSS. Cells were suspended in blocking solution containing 25 μl of mouse serum (prepared by 1000×g centrifugation of mouse blood collected by cardiac puncture and clotted undisturbed for 30 min at RT) and the pellet was mixed gently with it. For staining 25 μl cell suspension from the previous step was taken and added 25 μl master mix of antibodies (see Table II below for the antibodies and concentration used) and stained at RT for 30 min. Then tubes were centrifuged once at 100×g for 10 min and the supernatant was discard. The pellet was reconstituted to 100 μl using FACS buffer (2% FBS in HBSS) with 4% PFA. FACS analysis was used to quantify different cell types.

TABLE II
lineage-specific markers

| Cell detected | Antigen | Fluro | Clone | Isotype | Cat. No. | Company | Dilution |
|---|---|---|---|---|---|---|---|
| Pan-NK cells | CD49b | PE | DX5 | Rat IgM, κ | 108909 | Biolegend | 1:100 |
| All hematopoietic cells | CD45 | AF700 | 30-F11 | Rat IgG2b, κ | 103107 | Biolegend | 0.5:100 |
| Monocytes | CD115 | APC/CY7 | AFS98 | Rat IgG2a, κ | 135523 | Biolegend | 0.5:100 |
| Monocytes | Ly 6 C | BV421 | HK1.4 | Rat IgG2a, κ | 128031 | Biolegend | 1.5:100 |
| Neutrophils | Ly-6G | PECy7 | 1A8 | Rat IgG2a, κ | 127623 | Biolegend | 0.5:100 |
| Macrophages | F4/80 | APC | BM8 | Rat IgG2a, κ | 123109 | Biolegend | 0.5:100 |
| Neutrophils, | CD11b | FITC | M1/70 | Rat/IgG2b, | 83-0112-41 | Invitrogen | 0.5:100 |
| Monocytes, | | | | kappa | | | |
| Eosinophiles | | | | | | | |

### Macrophage Isolation Protocol:

Femurs and tibias of analyzed mice were flushed and plated into 30% L929 supernatant, 70% DMEM (10% FBS, 4.5 g/L glucose, L-glutamine, Sodium Pyruvate, 1% Pen/Strep), at a concentration of 100 ml/mouse, 10 ml/10 cm tissue culture-treated dish (day 0). Dishes were incubated at 37 C, 5% CO₂ and 1 ml of L929 supernatant is added to each dish on days 2 & 4. Cells were mature by day 7.

### Example 99: In Vivo Infiltration of Leukocytes in Mice

In vivo infiltration of leukocytes was performed using a murine air pouch (as described in Example 98, Sin et al, 1986), as an acute model of bacterial infection with LPS stimulation.

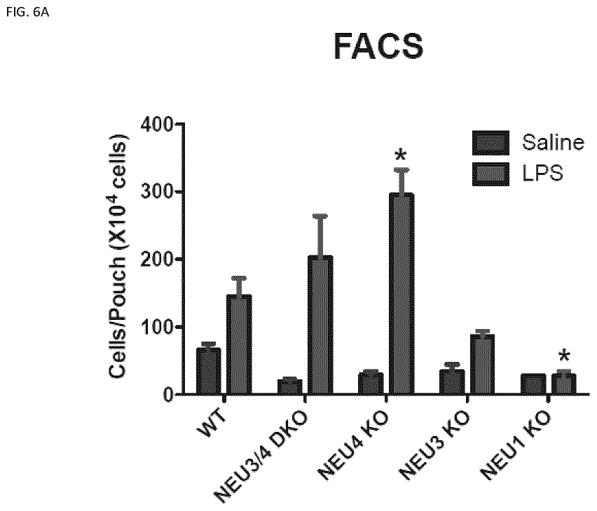

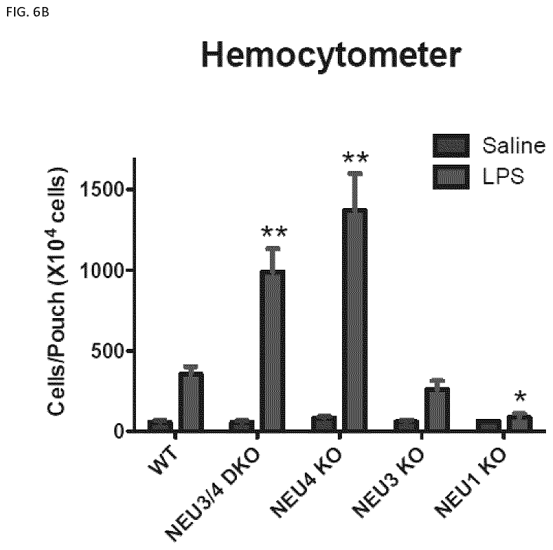

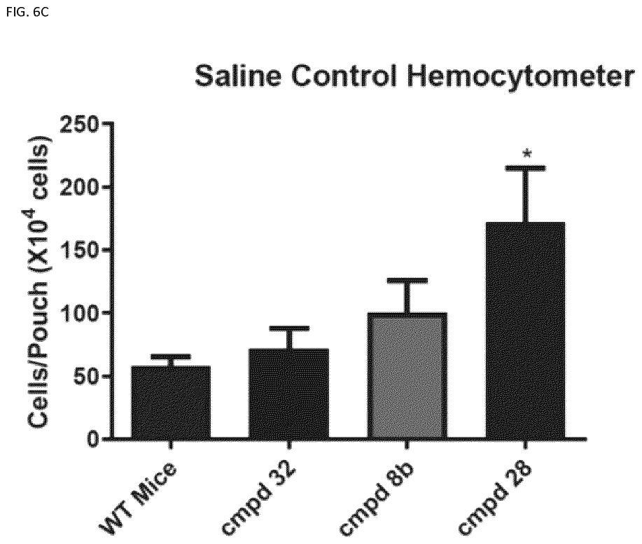

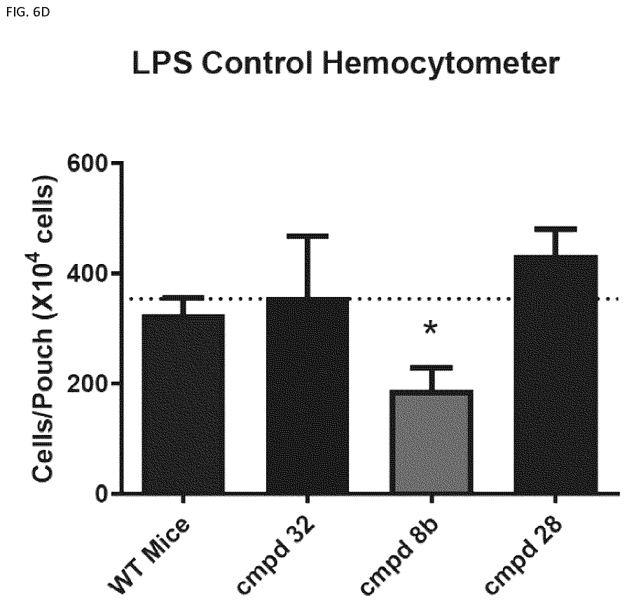

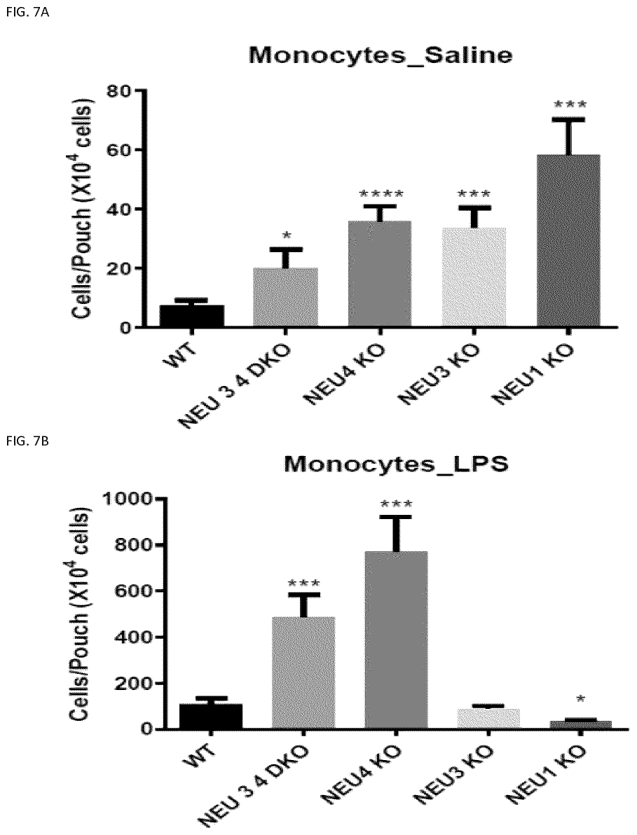

First, murine WT C57BL/6 mice or NEU KO or DKO (NEU1, NEU3, NEU4, NEU3/NEU4) animals were analyzed for changes in leukocyte migration. The air pouch was loaded with saline or LPS to induce leukocyte migration. Cells were washed out of the pouch and counted by hemocytometer and FACs. Significant leukocyte count (migration) increases were observed for NEU4 KO mice after LPS treatment. NEU1 KO mice showed a significant decrease in leukocytes count (migration). Results are shown in FIGS. 6A-B.

Then, using the same air pouch model, murine WT C57BL/6 mice were administered saline or LPS in their pouches to induce leukocyte migration. Cells were washed out of the pouch and counted. The mice were pre-treated with inhibitors of human neuraminidase (i.e. Compounds 32 (neu1-specific), 8b (neu3-specific) and 28 (neu4-specific), see compounds structures in Table I above) 48 h, 24 h prior and in conjunction to injection of LPS or saline. Results are shown in FIGS. 6C-D.

### Example 100: In Vivo Leukocyte Recruitment in NEU KO Animals has Differential Effects by Cell Types

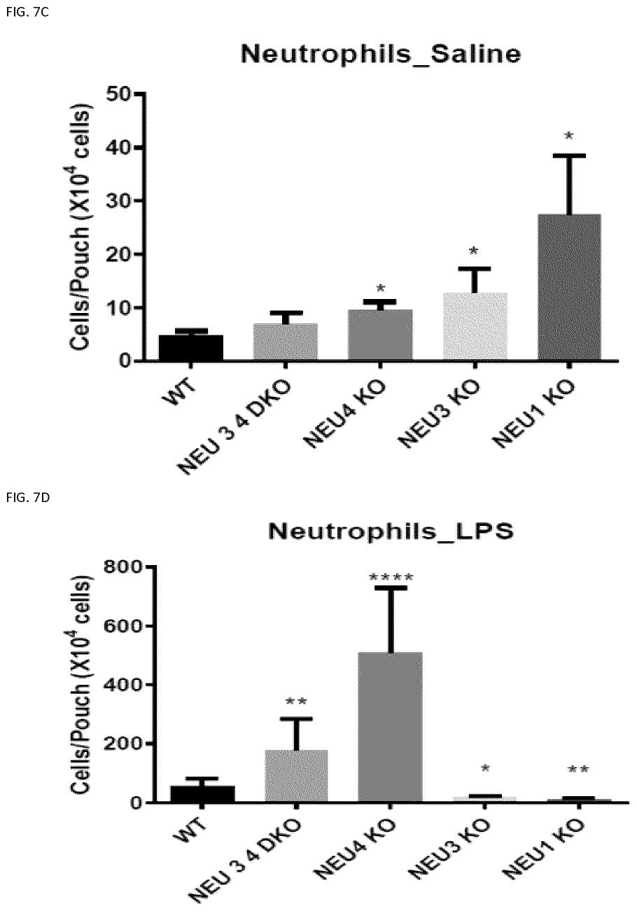

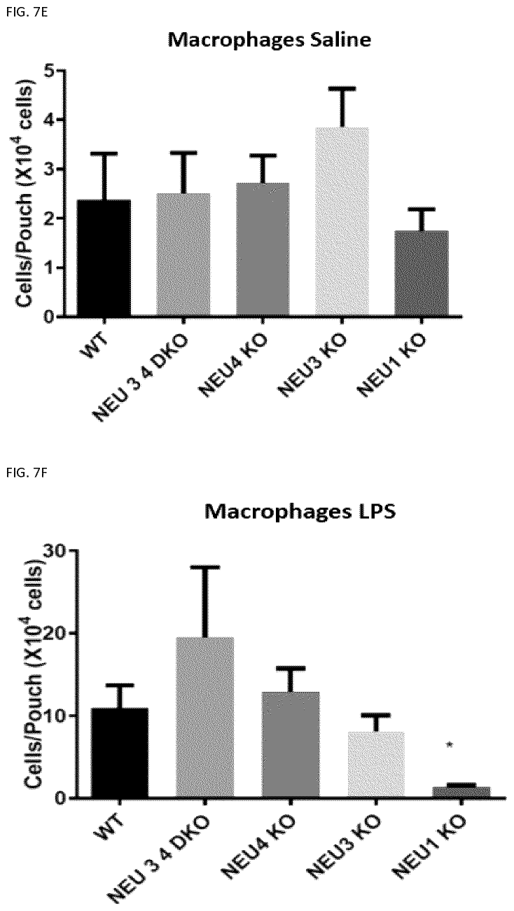

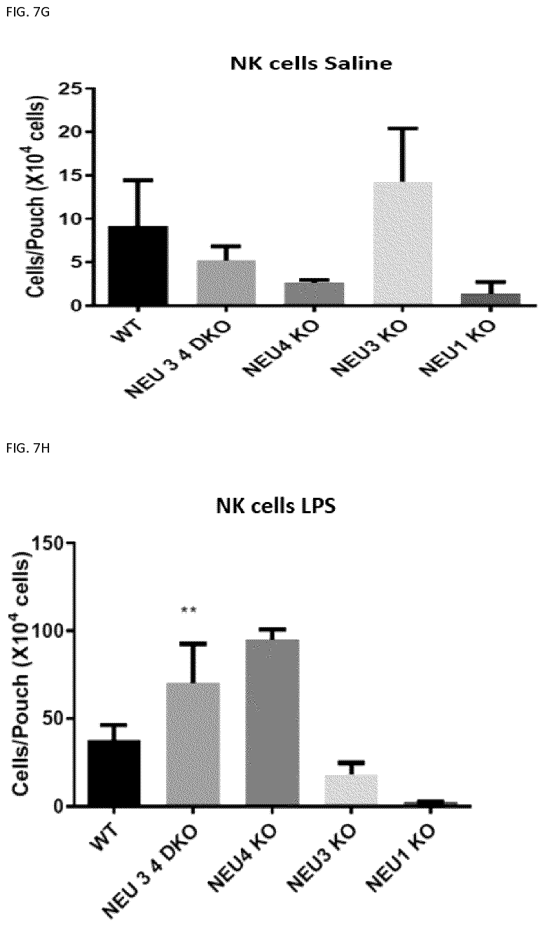

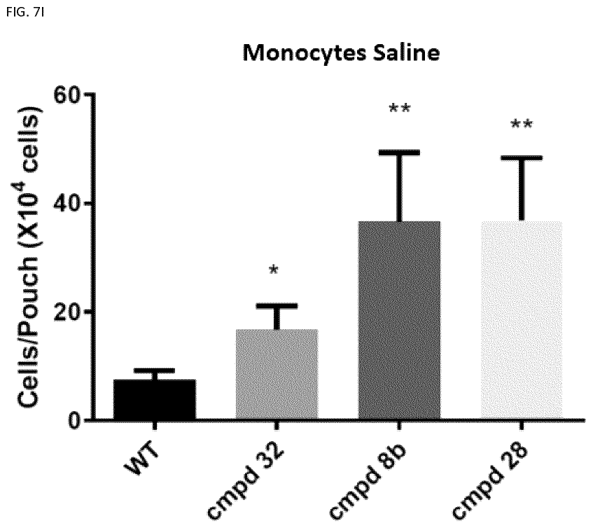

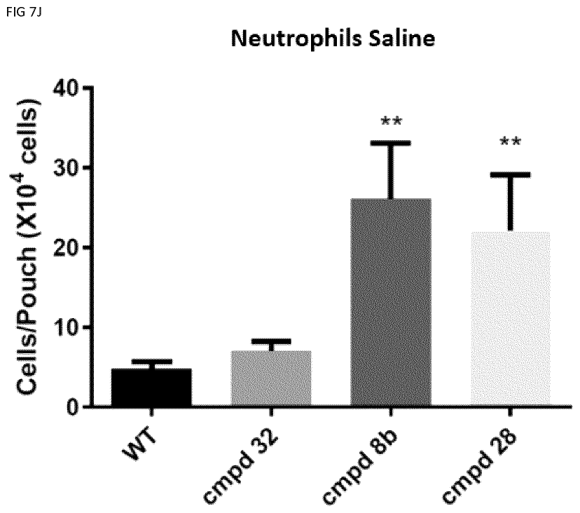

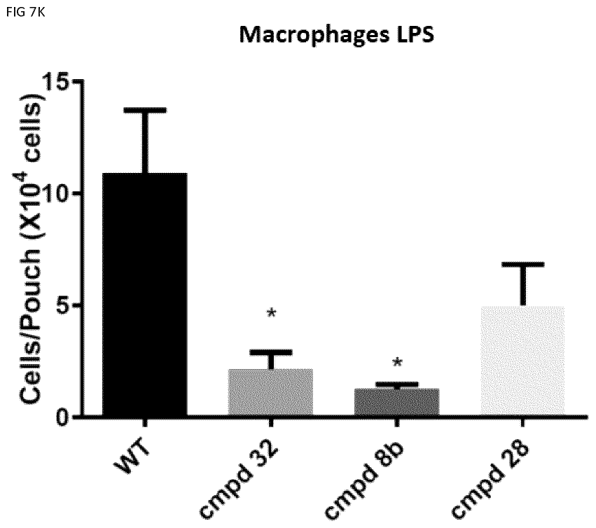

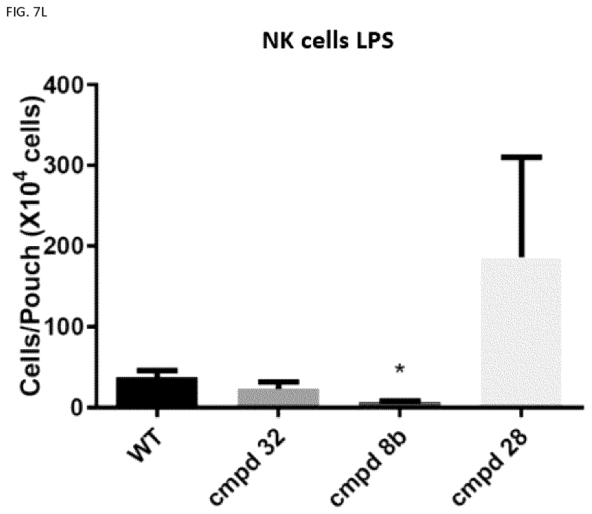

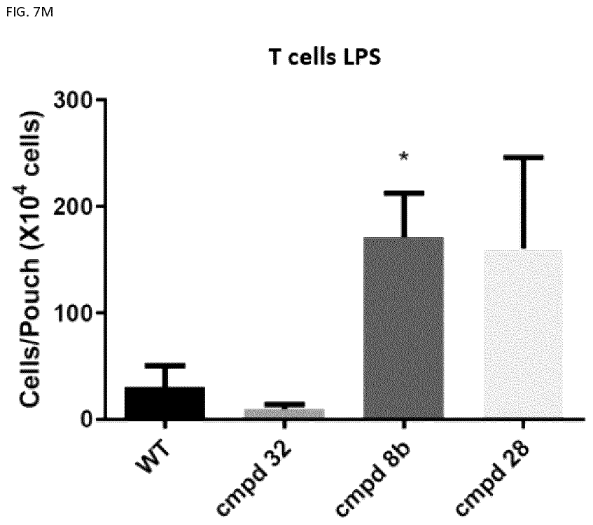

The cells isolated from the air pouch of Example 99 after stimulation with saline or LPS were sorted through flow cytometry with the use of appropriate lineage-specific markers shown in the Table II above to determine the cell subpopulations. The results show that neu3/4, neu4, neu3 and neu1 KO mice along with inhibitors for those neuraminidases modulate (increases or decreases) migrations of members of the leukocytes in saline or in the context of bacterial infection. Results are shown in FIGS. 7A-M.

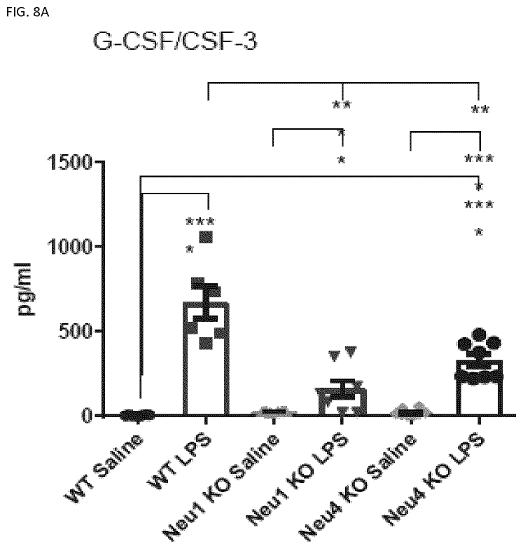

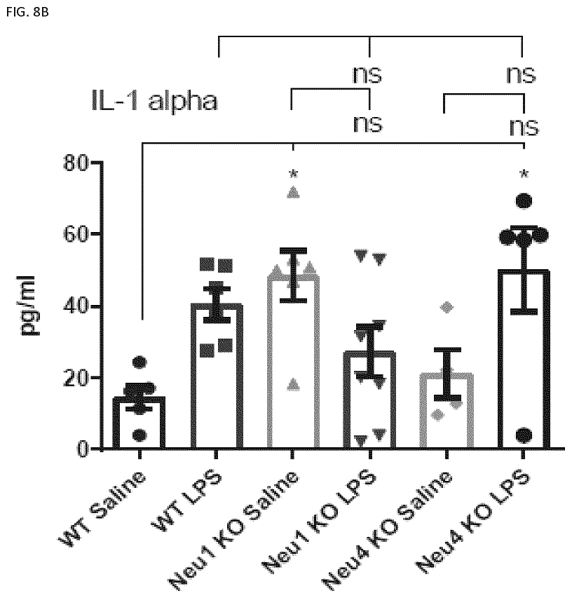

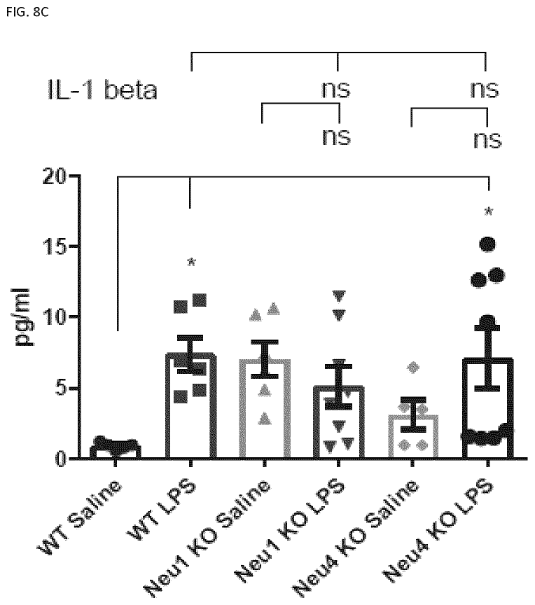

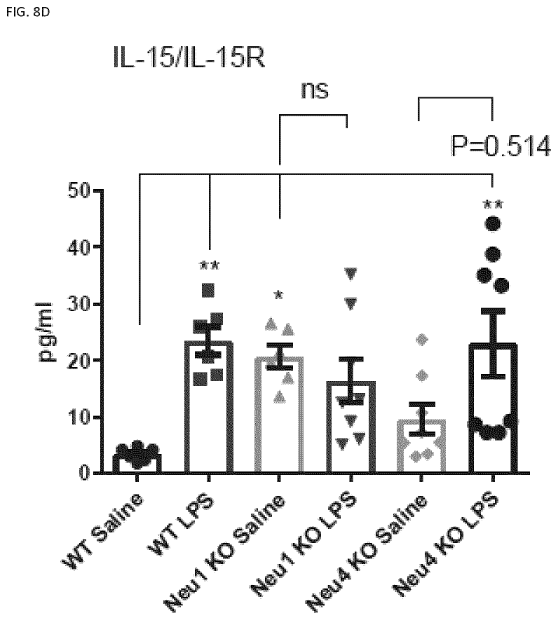

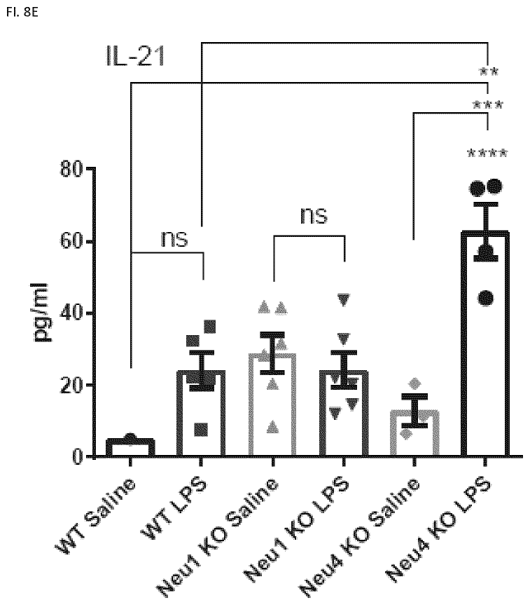

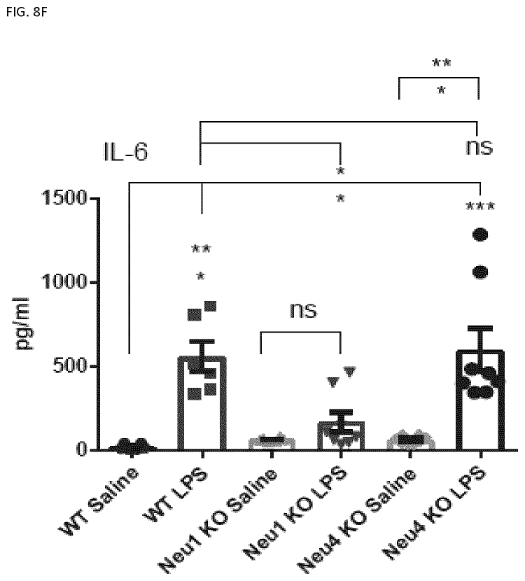

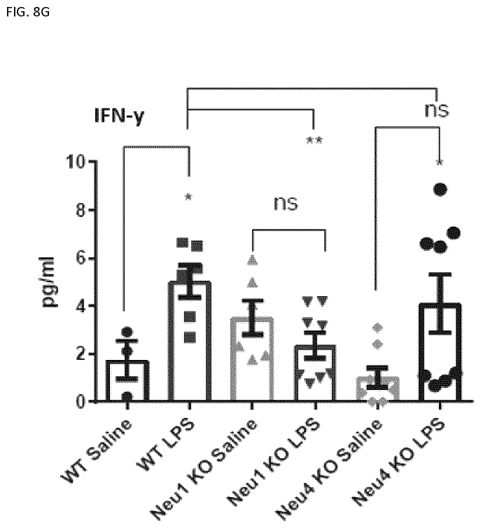

FIGS. 8A-I also show that cytokines are modulated in the neu1 and neu4 KOs mice. For instance, G-CSF/CSF-3 and RANTES plasma levels are reduced in LPS-treated neu4 and neu1 KO mice; that IL-21 is increased in in LPS-treated neu1 KOs mice; and that IL-6 and IFN-γ are reduced in LPS-treated neu1 KO mice. The results show that in saline-treated and LPS-treated mice, cytokine response varies with neu suppression.

### Example 101: NEU1 Deficiency Reduced Platelet Depletion Rate in the Passive Mouse Model of Immunothrombocytopenia

The Passive Antibody Transfer Model of ITP has been extensively used to understand the progression of chronic ITP and to rapidly evaluate the efficacy of various therapeutics (Neschadim et al., 2015). The model is characterized by a rapid onset of the ITP and clear involvement of phagocytic monocytes in platelet destruction. In addition, it provides a possibility to use self-antigens relevant to the human condition. Depending on the type, quantity, and frequency of the administered antibody it is possible to vary the severity and the persistence of the induced ITP by fine-tuning the rate and the level of platelet decline whereas the repeated administration of the monoclonal antibody simulates chronic human IT.

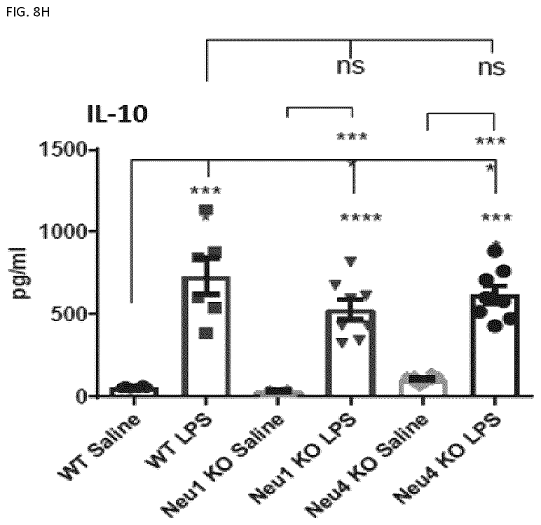

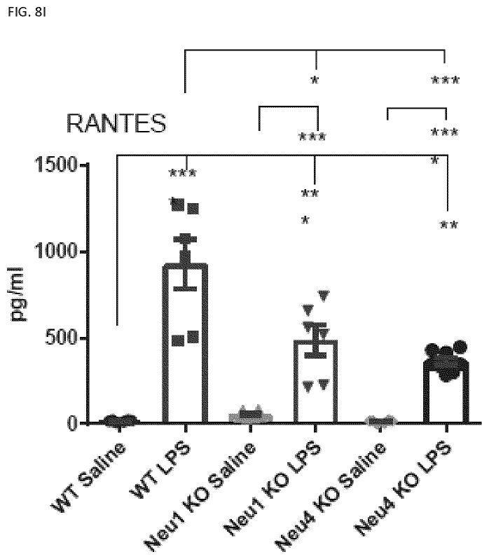

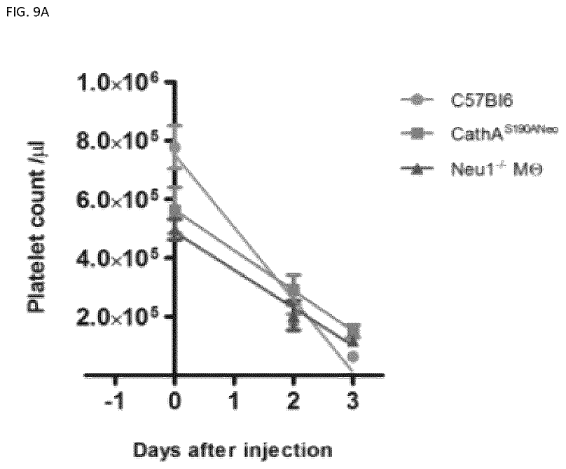

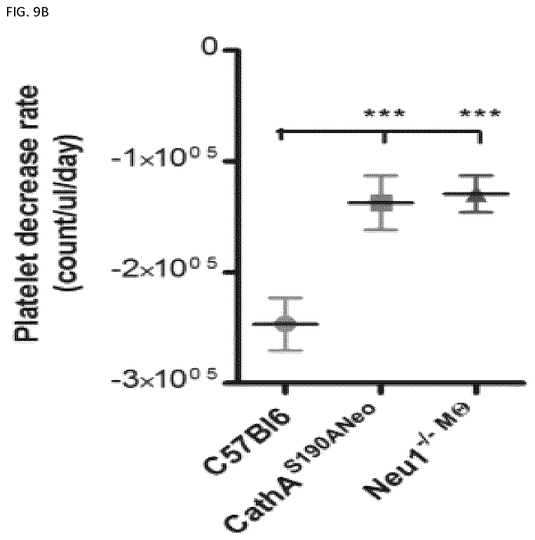

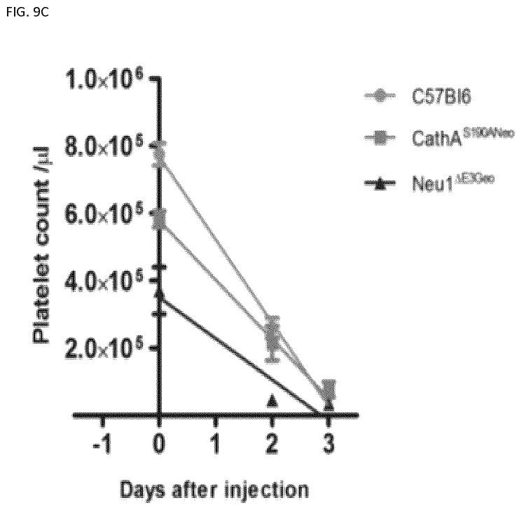

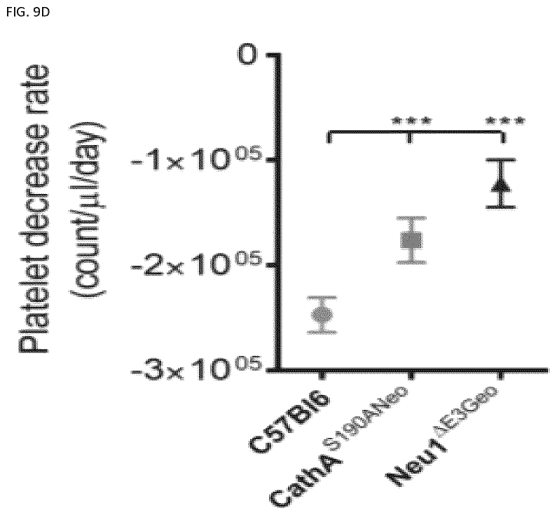

Female 6-week old C57Bl/6NCrI mice and genetically-modified C57Bl/6NCrI mice with neu1 deficiency (Passive Antibody Transfer Model of ITP, Neschadim et al., 2015) received escalating daily doses (68 μg/kg on the 1st and 2d days, 102 μg/kg on the third day) of anti-CD41 anti-platelet monoclonal mouse antibody (clone MWReg30) by intraperitoneal injection. The reticulated platelets were measured daily for three days by flow cytometric analysis. Control mice (C57B16) received injections of saline. In the wt mice antibody injection resulted in rapid (over 2 days) reduction of reticulated platelets from about 1000×10⁹ to less than 50×10⁹ counts/L. No increase in reticulated platelets was observed in this model over time, indicating thrombopoiesis suppression. Lower platelet reduction rates in NEU1-deficient CathA^{S190A-Neo} mice (˜10% of residual neu1 activity) (FIGS. 9A-D), neu1^{ΔE3Geo} mice (constitutive NEU1 KO) (FIGS. 9C-D) and NEU1^{-/-MΘ} (macrophage-specific neu1 knockout) (FIGS. 9A-B) indicated involvement of neu1 in development of immunotrombocytopenia.

Of note, the CathA^{S190A-neo} knock-in mouse is not clinically affected (Seyrantepe et al., 2008), suggesting that a partial pharmacological inhibition of NEU1 may have a preventive or therapeutic effect for ITP without affecting catabolism of sialoglycoconjugates catalyzed by NEU1 in the lysosome.

### Example 102: Regulation of Inflammatory Response to P. aeruginosa Infection

The recognition of LPS by the immune system involves TLR4/MD2, CD14 and LPS-binding protein (Park et al., 2013). Results shown in Examples 94-96 suggest that NEU regulates integrin function.

The mouse model of pulmonary Pseudomonas aeruginosa infection is used. Five mouse strains (wild-type C57Bl6 (WT), Neu3^{-/-}, Neu4^{-/-}; Neu1^{ΔEx3-Geo} (constitutive NEU1 KO); and Neu1^{MΘΔEx3} (macrophage-specific NEU1 KO) are infected intratracheally with a 50 μL suspension of 1.5×10⁷ P. aeruginosa strain PAO1 which produces a severe pneumonia (pulmonary bacterial burden of ˜1×10⁵ colony forming units). Mouse survival remains >90% at 48 hours in this model, permitting adequate sampling of all experimental groups and obviating a healthy survivor bias. Forty-eight hours after infection, mice are sacrificed following bronchoalveolar lavage. Analysis of bronchoalveolar lavage fluid inflammatory markers are performed, including flow cytometry enumeration of leukocyte populations and multiplex ELISA for cytokine levels. Bronchoalveolar lavage fluid LDH levels are quantified as a measure of pulmonary injury. Pulmonary bacterial burden is determined by quantitative culture as the primary virulence outcome. A cut-off of 0.5 log difference in bacterial burden is considered biologically significant and the Wilcoxon-rank sum is used to compare bacterial burden between groups, performed in duplicate with 10 mice the minimum group size.

Pharmacological inhibition of NEU1-4 is tested pulmonary Pseudomonas aeruginosa infection. Selective inhibitors of human and mouse NEU isoenzymes with potency in sub-micromolar range are used. The inhibitors are given intraperitoneally once/day before and during the infection. Inhibitors of NEU1, NEU3 and NEU4 in mice have been tested for the period of up to month which demonstrated that compounds are well tolerated by animals and result in almost complete inhibition in of NEU enzymes in the mouse tissues.

### Example 103: Treatment with Specific NEU1 Inhibitor Compound 50 Reduced Platelet Depletion Rate in the Passive Mouse Model of Immunothrombocytopenia

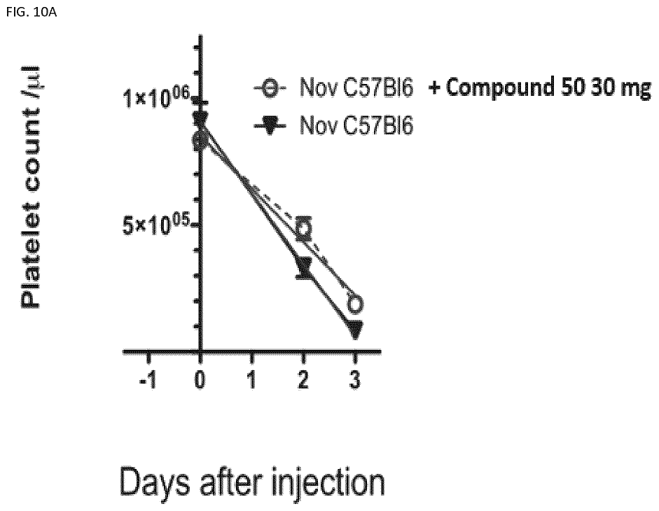

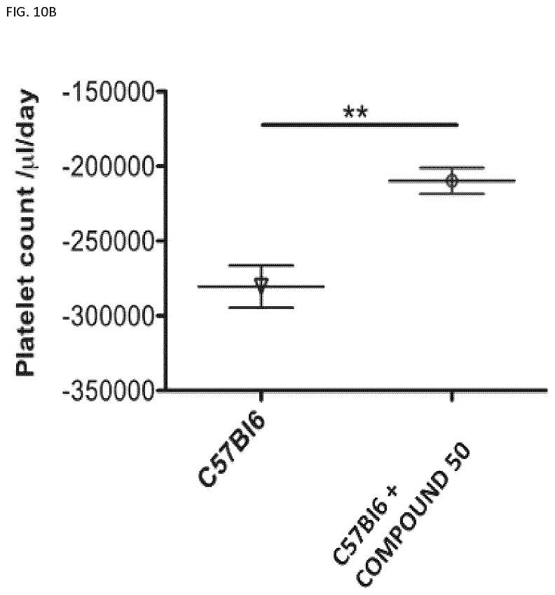

Female 6-week old C57Bl/6NCrI mice (Passive Antibody Transfer Model of ITP) received escalating daily doses (68 μg/kg on the 1st and 2d days, 102 μg/kg on the third day) of anti-CD41 anti-platelet monoclonal mouse antibody (clone MWReg30) by intraperitoneal injection. Experimental group received daily intraperitoneal injections of neu1 inhibitor compound 50 in saline at a dose of 30 μg/kg while control mice (Nov C57B16) received daily intraperitoneal injections of saline. The reticulated platelets were measured daily by flow cytometric analysis. FIGS. 10A-B show a lower platelet reduction rates in mice treated with compound 50.

### Example 104: Generation and Characterization of Macrophage- and Platelet-Specific Models of NEU1 Deficiency

Cell-specific NEU1 KO strains are generated by crossing a conditional Cre NEU1 gene-targeted mouse strain with mice expressing Cre recombinase in the cells of interest.

The inventors have acquired mouse ES line ENSMUSE00000141558 which is targeted with the PG00096_Z_3_G09 vector that allows production of conditional and tissue specific NEU1 KO. They have generated gene-targeted C57Bl6 mice heterozygous for the targeted NEU1 allele. The targeted allele contains LacZ/BactPNeo cassette flanked with FRT sites inserted into the intron 2 of the mouse NEU1 gene. In addition, the exon 3 of the gene is flanked with LoxP sites. The inventors have produced mice homozygous for the ENSMUSE00000141558 allele and showed that they do not express WT NEU1 mRNA. Similarly to previously described NEU1 KO mice (de Geest, 2002) NEU1^{ENSMUSE00000141558} mice showed almost complete deficiency of NEU1 mRNA and sialidase activity in kidney, where the NEU1 is a predominant neuraminidase. These data demonstrate that the NEU1 gene in NEU1^{ENSMUSE00000141558} mice is correctly targeted.

NEU1^{ENSMUSE00000141558} strain was first crossed with “FLP deleter” strain with a global expression of flippase (FLP) recombinase (JaxLab) to remove the FRT-flanked gene trap-cassette from the targeted NEU1 allele allowing normal expression of the gene. Then the mice were crossed with the available mice (JaxLab) expressing Cre recombinase under control of the macrophage specific Cx3cr1 (chemokine C-X3-C motif receptor 1) promoter (Yona et al, 2013) or and will be crossed with the available mice (JaxLab) expressing Cre recombinase under control of the platelet-specific Pf4 (platelet factor 4) promoter (Tiedt et al, 20107). This causes a recombination removing the entire exon 3 from the NEU1 gene only in MO (Neu1^{-/-MΘ}, see FIGS. 9A and B) or in platelets/megakaryocytes, respectively. Since all mouse strains are in C57Bl6 background there is no need to perform backcrosses to these backgrounds.

Heterozygous mice are mated to obtain homozygous and WT siblings (embryonic lethality is not expected since it is not observed in the full NEU1 KO). Expression of NEU1 mRNA in purified platelets and peritoneal and spleen MO as well as in control tissues (liver, kidney, brain, heart) of the homozygous animals are studied by qPCR and the level of neuraminidase activity measured by the fluorescent assay to confirm efficiency of the splicing outcome. The expression of NEU1 protein is studied by Western blot in the same tissues using available antibodies against mouse NEU1. Monocytes and monocyte-derived cells (spleen resident MΘ, liver Kupfer cells, brain microglia (Yona et al., 2013) as well as tissues of visceral organs, lungs, and different areas of brain are studied by light and electron microscopy to assess vacuolization of cells and presence of lysosomal storage bodies. Phenotypic characterization of mice is performed to determine if they show neurological symptoms previously described for NEU1 KO mice (de Geest et al., 2002). General observation, weight measurements, and estimation of life span of animals are performed. Neurological assessment and behaviour study by the open field test are performed to detect signs of neurodegeneration (Martins et al., 2015).

ITP in macrophage-specific NEU1 KO is studied using the Passive Antibody Transfer Model as described in Example 101. ITP in platelet-specific KO is induced by anti-GPIb antibodies that specifically trigger NEU1 induction. If an effect is observed in vivo experiments are performed to decipher the cellular/molecular mechanism by which NEU1 triggers ITP. In particular, the surface sialylation of platelets and MO are studied by SNA/PNA lectin binding and FACS, sialylation of FcγR receptor and platelet surface antigens, by immunoprecipitation and SNA/PNA lectin blot, and platelet activation, by selectin binding and FACS. Platelet uptake by spleen MΘ are directly studied by injecting mice with fluorescently labeled antibody against murine platelets followed by IHC analysis to detect opsonized platelets in spleen tissues stained with anti-F4/80 antibodies specific for MΘ. Profiles of circulating inflammatory cytokines (IL-1 α, -2, -6,-17, -23, GM-CSF, MCP-1, MIP-1 α and β, RANTES, TNFα and IFN γ) are also studied because by inducing their production through activation of TLR-3,4 receptors on MΘ NEU1 can also contribute to severity of ITP.

### REFERENCES

Albohy, A., et al., Insight into substrate recognition and catalysis by the human neuraminidase 3 (NEU3) through molecularmodeling and site-directed mutagenesis. Glycobiology, 2010. 20(9): p. 1127-1138.
Albohy, A.; Mohan, S.; Zheng, R. B.; Pinto, B. M.; Cairo, C. W. Inhibitor selectivity of a new class of oseltamivir analogs against viral neuraminidase over human neuraminidase enzymes. Bioorg. Med. Chem. Lett., 2011. 19(9): p. 2817-2822.
Albohy, A.; Zhang, Y.; Smutova, V.; Pshezhetsky, A. V.; Cairo, C. W. Identification of Selective Nanomolar Inhibitors of the Human Neuraminidase, NEU4. ACS Med Chem Lett, 2013. 4(6): p. 532-7.
Blanchette V S. Prophylaxis in the haemophilia population. Haemophilia: the official journal of the World Federation of Hemophilia 2010; 16 Suppl 5:181-8.
Cox, D., Brennan, M., and Moran, N. (2010) Integrins as therapeutic targets: lessons and opportunities. Nat. Rev. Drug Discovery 9, 804-820.
Crary S E, Buchanan G R. Vascular complications after splenectomy for hematologic disorders. Blood 2009; 114:2861-8.
de Geest N, Bonten E, et al. Systemic and neurologic abnormalities distinguish the lysosomal disorders sialidosis and galactosialidosis in mice. Hum Mol Genet 2002; 11:1455-64.
Hogg, N., Laschinger, M., Giles, K., and McDowall, A. (2003) T-cell integrins: more than just sticking points. J. Cell Sci. 116, 4695-4705.
Hogg, N., Smith, A., McDowall, A., Giles, K., Stanley, P., Laschinger, M., and Henderson, R. (2004) How T cells use LFA-1 to attach and migrate. Immunol. Lett. 92, 51-54
Kampstra, P. (2008) Beanplot: A boxplot alternative for visual comparison of distributions. Journal of Statistical Software 28, 1-9
Katsman Y, Foo A H, et al. Improved mouse models for the study of treatment modalities for immune-mediated platelet destruction. Transfusion 2010; 50:1285-94.
Kuhne T. Idiopathic thrombocytopenic purpura of childhood: a problem-oriented review of the management. Transfusion and apheresis science: official journal of the World Apheresis Association: official journal of the European Society for Haemapheresis 2003; 28:243-8.
Ley, K., Laudanna, C., Cybulsky, M. I., and Nourshargh, S. (2007) Getting to the site of inflammation: the leukocyte adhesion cascade updated. Nat. Rev. Immunol. 7, 678-689
Markely, L. R. A.; Ong, B. T.; Hoi, K. M.; Teo, G.; Lu, M. Y.; Wang, D. I. C. A high-throughput method for quantification of glycoprotein sialylation. Anal. Biochem. 2010, 407, 128-133.
Martins C, Hulkova H, et al. Neuroinflammation, mitochondrial defects and neurodegeneration in mucopolysaccharidosis III type C mouse model. Brain 2015; 138:336-55.
Neschadim A, Branch D R. Mouse models of autoimmune diseases: immune thrombocytopenia. Current pharmaceutical design 2015; 21:2487-97.
Neunert C E. Current management of immune thrombocytopenia. Hematology/the Education Program of the American Society of Hematology American Society of Hematology Education Program 2013; 2013:276-82.
Park, B. S., and Lee, J.-O. (2013) Recognition of lipopolysaccharide pattern by TLR4 complexes. Exp. Mol. Med. 45, e66.
Potier, M.-C.; Mameli, L.; Bélisle, M.; Dallaire, L.; Melancon, S. B. Fluorometric assays of neuraminidase with a sodium (4-methylumbellifery-α-D-N-Acetylneuraminate) substrate. Anal. Biochem. 1979, 94, 287-296.
Pshezhetsky, A. V.; Potier, M. Association of N-acetylgalactosamine-6-sulfate sulfatase with the multienzyme lysosomal complex of β-galactosidase, cathepsin A, and neuraminidase: possible implication for intralysosomal catabolism of keratan sulfate. J. Biol. Chem. 1996, 271 (45), 28359-28365.
Seyrantepe V, Hinek A, et al. Enzymatic activity of lysosomal carboxypeptidase (cathepsin) A is required for proper elastic fiber formation and inactivation of endothelin-1. Circulation 2008; 117:1973-81.
Shidmoossavee, F. S.; Watson, J. N.; Bennet, A. J. Chemical insight into the emergence of influenza virus strains that are resistant to Relenza. J. Am. Chem. Soc. 2013, 135, 13254-13257.
Simmons, D. L. (2005) Anti-adhesion therapies. Curr. Opin. Pharmacol. 5, 398-404.
Sin, Y. M.; Sedgwick, A. D.; Chea, E. P.; Willoughby, D. A Mast cells in newly formed lining tissue during acute inflammation: A six day air pouch model in the mouse. Annals of the Rheumatic Diseases 1986, 45, 873. Smutova, V., et al., Structural basis for substrate specificity of mammalian neuraminidases. PLoS One, 2014. 9(9): p. e106320.
Sin, Y., Sedgwick, A., Chea, E., and Willoughby, D. (1986) Mast cells in newly formed lining tissue during acute inflammation: a six day air pouch model in the mouse. Ann. Rheum. Dis. 45, 873
Tiedt R, Schomber T, et al. Pf4-Cre transgenic mice allow the generation of lineage-restricted gene knockouts for studying megakaryocyte and platelet function in vivo. Blood 2007; 109:1503-6.
Varki, A. (1994) Selectin ligands. Proceedings of the National Academy of Sciences 91, 7390-7397.
von Itzstein, M.; Wu, W.-Y.; Jin, B. The synthesis of 2,3-didehydro-2,4-dideoxy-4-guanidinyl-N-acetylneuraminic acid: a potent influenza virus sialidase inhibitor. Carbohydr. Res. 1994, 259, 301-305.
von Itzstein, M.; Wu, W.-Y.; Kok, G. B.; Pegg, M. S.; Dyason, J. C.; Jin, B.; Phan, T. V.; Smythe, M. L.; White, H. F.; Oliver, S. W.; Colman, P. M.; Varghese, J. N.; Ryan, D. M.; Woods, J. M.; Bethell, R. C.; Hotham, V. J.; Cameron, J. M.; Penn, C. R. Rational design of potent sialidase-based inhibitors of influenza virus replication. Nature 1993 , 363, 418-423.
Warner, T. G.; O'Brien, J. S. Synthesis of 2′-(4-methylumbelliferyl)-.alpha.-D-N-acetylneuraminic acid and detection of skin fibroblast neuraminidase in normal humans and in sialidosis. Biochemistry 1979, 18, 2783-2787.
Yona S, Kim K W, et al. Fate mapping reveals origins and dynamics of monocytes and tissue macrophages under homeostasis. Immunity 2013; 38:79-91.
Yong M, Schoonen W M, et al. Epidemiology of paediatric immune thrombocytopenia in the General Practice Research Database. Br J Haematol 2010; 149:855-64.
Zhang, Y.; Albohy, A.; Zou, Y.; Smutova, V.; Pshezhetsky, A. V.; Cairo, C. W. Identification of selective inhibitors for human neuraminidase isoenzymes using C4,C7-modified 2-deoxy-2,3-didehydro-N-acetylneuraminic acid (DANA) analogues. J. Med. Chem., 2013. 56(7): p. 2948-2958.
Zou, Y.; Albohy, A.; Sandbhor, M.; Cairo, C. W. Inhibition of human neuraminidase 3 (NEU3) by C9-triazole derivatives of 2,3-didehydro-N-acetyl-neuraminic acid. Bioorg. Med. Chem. Lett. 2010, 20, 7529-7533.

1. A method of modulating leukocyte activation, comprising selectively inhibiting the expression or activity of neuraminidase 1 (neu1), neuraminidase 3 (neu3) or neuraminidase 4 (neu4) in a subject in need thereof.

2. The method of claim 1, wherein the inhibiting comprises the administration of a therapeutically effective amount of a specific or a bispecific neu1, neu3, and/or neu4 inhibitor.

3. The method of claim 1 or 2, wherein the leukocyte activation comprises transmigration of monocytes, neutrophils, macrophages, NK cells or T-cells.

4. The method of claim 1 or 2, wherein the inhibiting comprises the administration of a therapeutically effective amount of a specific or a bispecific neu1 and/or neu4 inhibitor

5. The method of claim 4, wherein the leukocyte activation comprises leukocyte adhesion, leukocyte transmigration and/or cytokine response.

6. The method of claim 5, wherein the cytokine is G-CSF/CSF-3, IL-21, IL-6, IFN-γ and/or RANTES.

7. A method of modulating thrombocyte clearance, comprising inhibiting of the expression or activity of neuraminidase 1 (neu1), neuraminidase 3 (neu3) or neuraminidase 4 (neu4) in a subject in need thereof.

8. The method of claim 7, wherein the inhibiting comprises the administration of a therapeutically effective amount of a specific or a bispecific neu1, neu3, and/or neu4 inhibitor.

9. The method of claims 1 to 8, wherein the inhibitor is (i) compound 5c; 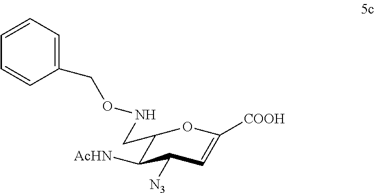
or (ii) a compound of formula I 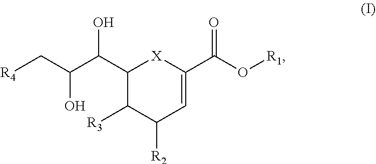
wherein R₁ is H; a C1-C10 alkyl; C1-C10 heteroalkyl; C3-C7 cycloalkyl; C3-C7 heterocycloalkyl; C3-C8 aryl; or C3-C8 heteroaryl; wherein the alkyl, heteroalkyl, cycloalkyl, heterocycloalkyl, aryl and heteroaryl are optionally substituted by at least one substituent, each substituent being independently a C1-C10 alkyl, a C3-C8 cycloalkyl, a C3-C7 aryl, a halogen, an amide or a hydroxyl;
R₂ is H; —OH, —NHC(═NH)NH₂; or azide;
R₃ is —NHC(O)(CH₂)nR₅,
wherein R₅ is H; —OH; C1-C10 alkyl; C1-C10 heteroalkyl; C3-C7 cycloalkyl; C3-C7 heterocycloalkyl; C3-C8 aryl or azide; wherein the alkyl, heteroalkyl, cycloalkyl, heterocycloalkyl, and aryl are optionally substituted by at least one substituent, each substituent being independently a C1-C10 alkyl, a C3-C8 cycloalkyl, a C3-C7 aryl, an halogen, an amide or an hydroxyl; and
n is 0 or 1;
R₄ is H; —OH; —O-alkyl; —C(O)-alkyl-NHC(O)-aryl; —NHC(O)R₆; or 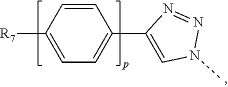
wherein the alkyl and aryl are optionally substituted by at least one substituent, each substituent being independently a C1-C10 alkyl, a C3-C8 cycloalkyl, a C3-C7 aryl, a halogen, an amine, an amide or a hydroxyl,
wherein: R₆ is H, C1-C10 alkyl; or C3-C7 aryl, wherein the C1-C10 alkyl and C3-C7 aryl are optionally substituted by at least one substituent, each substituent being independently a C1-C10 alkyl, a C3-C8 cycloalkyl, a C3-C7 aryl, an halogen, an amide, an amine or an hydroxyl;
R₇ is H; halogen; —O-alkyl; —C(O)OH; amine; acetamide; —C1-C10 alkyl; —O—C3-C7 aryl; or —(CH₂)qNH(CO)aryl, wherein the C1-C10 alkyl and C3-C7 aryl are optionally substituted by at least one substituent, each substituent being independently a C1-C10 alkyl, a C3-C8 cycloalkyl, a C3-C7 aryl, a halogen, an amide, an amine or a hydroxyl, wherein q is 0 or 1; and
p is 0, 1, 2 or 3; and
X is O, CH₂ or S,
with the proviso that when R₂ and R₄ are OH, R₃ is not —NHC(O)CH₃,
or the inhibitor is an ester, solvate, hydrate or pharmaceutical salt of the compound of formula 5c or formula I.

10. The method of claim 9, wherein n is 1.

11. The method of claim 10, wherein R₅ is H, C1-C5 alkyl or azide.

12. The method of any one of claims 9 to 11, wherein R₄ is 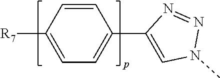
wherein R₇ and p are as defined in claim 9.

13. The method of claim 12, wherein p is 2 and R₇ is H.

14. The method of claim 12, wherein p is 0 or 1.

15. The method of claim 12, wherein p is 0 and R₇ is a hydroxy-substituted C1-C10 alkyl.

16. The method of any one of claims 9 to 11, wherein R₄ is —OH or —NHC(O)R₆.

17. The method of claim 16, wherein R₄ is —NHC(O)R₆ and R₆ is C3-C6 alkyl.

18. The method of any one of claims 9 to 17, wherein R₂ is OH or —NHC(═NH)NH₂.

19. The method of any one of claims 9 to 18, wherein X is O.

20. The method of any one of claims 1 to 6, wherein the inhibiting comprises the administration of a specific or bispecific neu4 inhibitor.

21. The method of claim 20, wherein the specific or bispecific inhibitor is compound 28

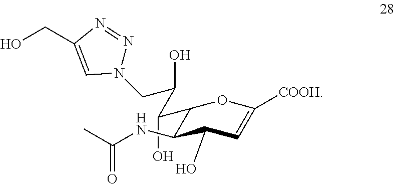

22. The method of claim 20 or 21, wherein the inhibiting increases monocytes and neutrophils transmigration and decreases T cell transmigration in response to bacterial infection.

23. The method of any one of claims 1 to 6, wherein the inhibiting comprises the administration of a specific or bispecific neu3 inhibitor.

24. The method of claim 23, wherein the specific or bispecific neu3 inhibitor is compound 8b or 5c

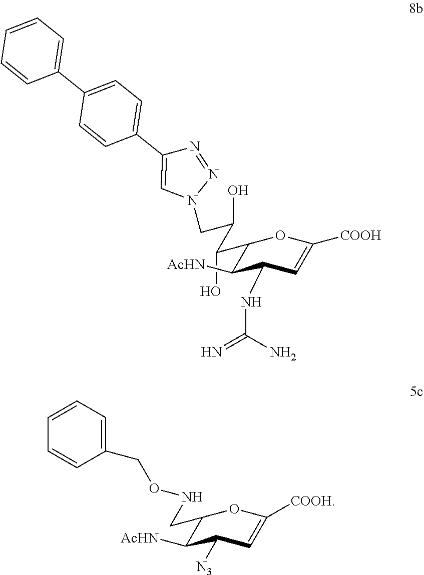

25. The method of claim 23 or 24, wherein the inhibiting increases monocytes and neutrophils transmigration and decreases T cell transmigration in response to bacterial infection.

26. The method of any one of claims 1 to 6, wherein the inhibiting comprises the administration of a specific or bispecific neu1 inhibitor.

27. The method of claim 26, wherein the specific or bispecific neu1 inhibitor is compound 32 or 50

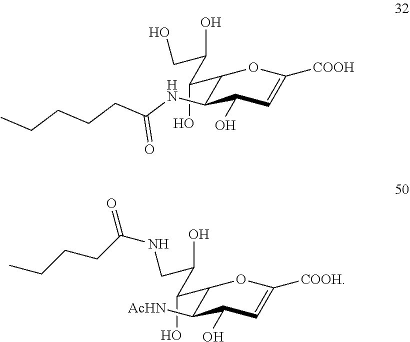

28. The method of claim 26 or 27, wherein the inhibiting decreases monocytes transmigration and increases neutrophils transmigration in response to bacterial infection.

29. The method of any one of claims 1 to 6, wherein the inhibiting comprises the administration of a specific or bispecific neu1 or neu4 inhibitor.

30. The method of claim 29, wherein the inhibiting modulates cytokine response.

31. The method of any one of claims 1 to 6, wherein the inhibiting modulates an inflammatory response to bacterial infection in a subject in need thereof.

32. The method of claim 7 or 8, wherein the inhibiting comprises the administration of a therapeutically effective amount of a specific or a bispecific neu1 inhibitor.

33. The method of claim 32, wherein the inhibitor is compound 50

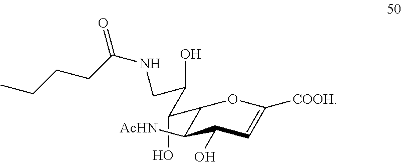

34. An inhibitor compound for use in modulating leukocyte activation or modulating thrombocyte clearance in a subject in need thereof, wherein the inhibitor compound has the structure of formula 5c or I, or is an ester, solvate, hydrate or pharmaceutical salt thereof, 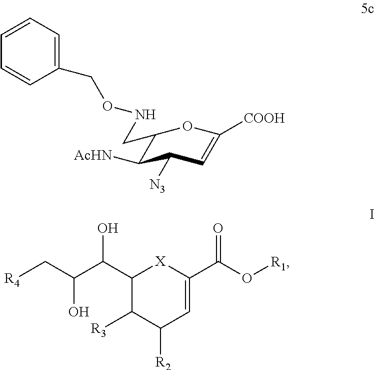
wherein R₁ is H; a C1-C10 alkyl; C1-C10 heteroalkyl; C3-C7 cycloalkyl; C3-C7 heterocycloalkyl; C3-C8 aryl; or C3-C8 heteroaryl; wherein the alkyl, heteroalkyl, cycloalkyl, heterocycloalkyl, aryl and heteroaryl are optionally substituted by at least one substituent, each substituent being independently a C1-C10 alkyl, a C3-C8 cycloalkyl, a C3-C7 aryl, a halogen, an amide or a hydroxyl;
R₂ is H; —OH, —NHC(═NH)NH₂; or azide;
R₃ is —NHC(O)(CH₂)nR₅,
wherein R₅ is H; —OH; C1-C10 alkyl; C1-C10 heteroalkyl; C3-C7 cycloalkyl; C3-C7 heterocycloalkyl; C3-C8 aryl or azide; wherein the alkyl, heteroalkyl, cycloalkyl, heterocycloalkyl, and aryl are optionally substituted by at least one substituent, each substituent being independently a C1-C10 alkyl, a C3-C8 cycloalkyl, a C3-C7 aryl, an halogen, an amide or an hydroxyl; and
n is 0 or 1;
R₄ is H; —OH; —O-alkyl; —C(O)-alkyl-NHC(O)-aryl; —NHC(O)R₆; or 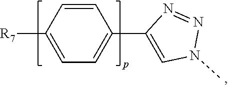
wherein the alkyl and aryl are optionally substituted by at least one substituent, each substituent being independently a C1-C10 alkyl, a C3-C8 cycloalkyl, a C3-C7 aryl, a halogen, an amine, an amide or a hydroxyl,
wherein: R₆ is H, C1-C10 alkyl; or C3-C7 aryl, wherein the C1-C10 alkyl and C3-C7 aryl are optionally substituted by at least one substituent, each substituent being independently a C1-C10 alkyl, a C3-C8 cycloalkyl, a C3-C7 aryl, an halogen, an amide, an amine or an hydroxyl;
R₇ is H; halogen; —O-alkyl; —C(O)OH; amine; acetamide; —C1-C10 alkyl; —O—C3-C7 aryl; or —(CH₂)qNH(CO)aryl, wherein the C1-C10 alkyl and C3-C7 aryl are optionally substituted by at least one substituent, each substituent being independently a C1-C10 alkyl, a C3-C8 cycloalkyl, a C3-C7 aryl, a halogen, an amide, an amine or a hydroxyl, wherein q is 0 or 1; and
p is 0, 1, 2 or 3; and
X is O, CH₂ or S,
with the proviso that when R₂ and R₄ are OH, R₃ is not —NHC(O)CH₃.

35. The compound of claim 34, wherein n is 1.

36. The compound of claim 35, wherein R₅ is H, C1-C5 alkyl or azide.

37. The compound of any one of claims 34 to 36, wherein R₄ is 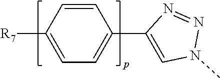
wherein R₇ and p are as defined in claim 34.

38. The compound of claim 37, wherein p is 2 and R₇ is H.

39. The compound of claim 37, wherein p is 0 or 1.

40. The compound of claim 37, wherein p is 0 and R₇ is a hydroxy-substituted C1-C10 alkyl.

41. The compound of any one of claims 34 to 36, wherein R₄ is —OH or —NHC(O)R₆.

42. The compound of claim 41, wherein R₄ is —NHC(O)R₆ and R₆ is C3-C6 alkyl.

43. The compound of any one of claims 34 to 42, wherein R₂ is OH or —NHC(═NH)NH₂.

44. The compound of any one of claims 34 to 43, wherein X is O.
